(19) United States
(12) Patent Application Publication
(10) Pub. No.: US 2025/0114454 A1
(43) Pub. Date: Apr. 10, 2025

(54) MODIFIED IMMUNE CELLS AND METHODS FOR USE THEREOF

(71) Applicants: The Broad Institute, Inc., Cambridge, MA (US); The General Hospital Corporation, Boston, MA (US)

(72) Inventors: Robert Manguso, Boston, MA (US); Kathleen Yates, Cambridge, MA (US)

(21) Appl. No.: 18/931,863

(22) Filed: Oct. 30, 2024

Related U.S. Application Data
(63) Continuation of application No. PCT/US2023/020680, filed on May 2, 2023.
(60) Provisional application No. 63/337,930, filed on May 3, 2022.

(51) Int. Cl. A61K 40/11; A61K 39/00; A61K 40/15; A61K 40/31; A61K 40/42; A61P 35/00; C07K 14/705; C07K 16/28; C12N 5/0783; C12Q 1/6886

(52) U.S. Cl. CPC A61K 40/11; A61K 40/15; A61K 40/31; A61K 40/4211; A61P 35/00; C07K 14/70503; C07K 14/7056; C07K 16/2803; C07K 16/2818; C12N 5/0636; C12N 5/0646; C12Q 1/6886; A61K 2039/505; C12N 2510/00; C12Q 2600/106; C12Q 2600/156; C12Q 2600/158

(57) ABSTRACT

The disclosure features compositions containing chimeric antigen receptor (CAR) immune cells that have been modified to reduce and/or eliminate expression or activity of a natural killer cell lectin A (NKG2A) polypeptide and/or a cluster of differentiation 94 (CD94) polypeptide, and methods for use thereof to treat a neoplasia.

### CROSS-REFERENCE TO RELATED APPLICATION

This application is a continuation of International Application No. PCT/2023/020680, filed on May 2, 2023, which claims priority to and the benefit of U.S. Provisional Application No. 63/337,930, filed May 3, 2022, the entire contents of which are incorporated herein by reference.

### SEQUENCE LISTING

This application contains a Sequence Listing which has been filed electronically in XML format and is hereby incorporated by reference in its entirety. The Sequence Listing XML, created on Dec. 13, 2024, is named 167741-046002US_SL.xml and is 49,457 bytes in size.

### BACKGROUND OF THE INVENTION

Immune checkpoint blockade (ICB) has had remarkable success in several cancer types, suggesting that enhancing anti-tumor immunity is a fundamental strategy to combat cancer. Currently-approved immune checkpoint inhibitors block inhibitory signals to cytotoxic T lymphocytes (CTLs), which can directly recognize and eliminate tumor cells in an adaptive immune response. However, many patients do not respond to immunotherapy with ICB, and there is a lack of understanding of the immune evasion mechanisms active in different cancer contexts. The limitations of existing technologies have thus far precluded the systematic discovery of immune resistance mechanisms active in the multicellular ecosystem of the tumor microenvironment. Further, immune resistance can hinder the effectiveness of treatment of a cancer patient using engineered immune effector cells (e.g., in chimeric antigen receptor (CAR) T-cell therapy). Accordingly, compositions and methods for overcoming tumor immune resistance (e.g., in the context of a CAR T-cell therapy) are urgently required.

Therefore, there remains a need for improved immunotherapies for treating cancers, particularly therapies able to overcome tumor immune evasion.

### SUMMARY OF THE INVENTION

As described below, the present disclosure features compositions and methods for the treatment of cancers that are able to evade the immune system.

In embodiments, the disclosure provides chimeric antigen receptor (CAR) expressing immune cells (e.g., CAR T cells) that have been modified to reduce and/or eliminate expression or activity of a natural killer cell lectin A (NKG2A) polypeptide and/or a cluster of differentiation 94 (CD94) polypeptide, and methods for use of such cells to treat a neoplasia (e.g., skin, colon, pancreas, lung, and kidney cancer).

In one aspect, the disclosure features a modified immune cell containing a chimeric antigen receptor polypeptide. The modified immune cell contains one or more genetic alterations that reduces or eliminates expression and/or activity of a natural killer cell lectin A (NKG2A) polypeptide and/or a cluster of differentiation 94 (CD94) polypeptide.

In another aspect, the disclosure features a method for increasing the anti-tumor activity of an immune cell. The method involves introducing into the genome of the immune cell one or more genetic alterations that reduces or eliminates expression and/or activity of a natural killer cell lectin A (NKG2A) polypeptide and/or a cluster of differentiation 94 (CD94) polypeptide.

In another aspect, the disclosure features a method for treating a neoplasia in a subject. The method involves administering to the subject a modified immune cell containing one or more genetic alterations that reduces or eliminates expression and/or activity of a natural killer cell lectin A (NKG2A) polypeptide and/or a cluster of differentiation 94 (CD94) polypeptide.

In another aspect, the disclosure features a pharmaceutical composition containing the modified immune cell of any of the above embodiments and a pharmaceutically acceptable excipient.

In another aspect, the disclosure features a method for characterizing immune checkpoint blockade sensitivity in a neoplasia. The method involves detecting interferon-stimulated gene (ISG) expression and 6p21.3 copy number in the neoplasia. Loss of 6p21.3 and/or reduced ISG expression levels relative to a reference characterizes the neoplasia as sensitive to immune checkpoint blockade. Presence of intact 6p21.3 and increased ISG expression levels relative to a reference characterizes the neoplasia as resistant to immune checkpoint blockade.

In another aspect, the disclosure features a method for treating a selected patient having a neoplasia. The method involves administering to the selected patient an immune checkpoint blockade. The patient is selected by characterizing loss of 6p21.3 and/or reduced ISG expression levels relative to a reference.

In another aspect, the disclosure features a method for inducing expression of NKG2A and/or CD94 in a T cell. The method involves contacting the cell with an anti-CD3 monoclonal antibody, an anti-CD28 monoclonal antibody, and an IL-12 polypeptide. In embodiments, the method further involves contacting the cell with the anti-CD3 monoclonal antibody, the anti-CD28 monoclonal antibody, and the IL-12 polypeptide a second time. In embodiments, the first contacting and the second contacting each further involve contacting the cell with IL-2, IL-7, and IL-15. In embodiments, the IL-12 polypeptide is an IL-12p70 polypeptide. In embodiments, the second contacting is between about 7 and 14 days after the first contacting.

In any aspect provided herein, or embodiments thereof, the cell is a T cell, a natural killer (NK) cell, a gammadelta T cell, or a natural killer T cell.

In any aspect provided herein, or embodiments thereof, the chimeric antigen receptor specifically binds an antigen present on a neoplastic cell. In embodiments, the antigen is selected from one or more of CD19, BCMA, Mesothelin, MUC1, MUC16, GD2, CD79b19, April, EGFR, EGFRvIII, IL13Ra, HLA-G, and PSMA.

In any aspect provided herein, or embodiments thereof, the cell contains one or more genetic alterations that reduces or eliminates expression of the natural killer cell lectin A (NKG2A) polypeptide.

In any aspect provided herein, or embodiments thereof, the immune cell has reduced susceptibility to interferon-mediated immune inhibition on tumor cells.

In any aspect provided herein, or embodiments thereof, the cell is a T cell, a natural killer (NK) cell, a gammadelta T cell, a natural killer T cell.

In any aspect provided herein, or embodiments thereof, the immune cell further contains one or more genetic alterations that reduces or eliminates expression and/or activity of the natural killer cell lectin A (NKG2A) polypeptide.

In any aspect provided herein, or embodiments thereof, the immune cell is in vivo or in vitro. In any aspect provided herein, or embodiments thereof, the immune cell is a human immune cell. In any aspect provided herein, or embodiments thereof, the subject or patient is a mammal. In embodiments, the mammal is a human.

In any aspect provided herein, or embodiments thereof, the neoplasia is a cancer selected from one or more of skin, colon, pancreas, lung, and kidney cancer. In embodiments, the skin cancer is melanoma. In embodiments, the lung cancer is non-small cell lung cancer. In embodiments, the kidney cancer is renal clear cell carcinoma.

In any aspect provided herein, or embodiments thereof, the method further involves administering to the subject an immune checkpoint blockade therapy. In any aspect provided herein, or embodiments thereof, the immune checkpoint blockade is a PD1, PDL1, or CTLA4 inhibitor. In any aspect provided herein, or embodiments thereof, the immune checkpoint blockade contains an antibody. In embodiments, the antibody is selected from one or more of Nivolumab, Pembrolizumab, Atezolizumab, Avelumab, Durvalumab, Cemiplimab, Dostarlimab, and ipilimumab.

In any aspect provided herein, or embodiments thereof, reference is the level of ISG expression present in a healthy cell or in a neoplasia containing intact 6p21.3.

In any aspect provided herein, or embodiments thereof, ISG expression is increased by at least about 20% relative to a reference.

In any aspect provided herein, or embodiments thereof, detecting ISG expression levels involves determining expression levels for one or more genes selected from one or more of ADAR, APOL6, ARID5B, ARL4A, AUTS2, B2M, BANK1, BATF2, BPGM, BST2, BTG1, C1R, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLR1, CMPK2, CMTR1, CSF2RB, CXCL10, CXCL11, CXCL9, DDX58, DDX60, DHX58, EIF2AK2, EIF4E3, EPSTI1, FAS, FCGR1A, FGL2, FPR1, GBP4, GBP6, GCH1, GPR18, GZMA, HELZ2, HERC6, HIF1A, HLA-A, HLA-B, HLA-DMA, HLA-DQA1, HLA-DRB1, HLA-G, ICAM1, IDO1, IFI27, IFI30, IF135, IF144, IF144L, IFIH1, IFIT1, IFIT2, IFIT3, IFITM2, IFITM3, IFNAR2, IL10RA, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, MARCHF1, METTL7B, MT2A, MTHFD2, MVP, MX1, MX2, MYD88, NAMPT, NCOA3, NFKB1, NFKBIA, NLRC5, NMI, NOD1, NUP93, OAS2, OAS3, OASL, OGFR, P2RY14, PARP12, PARP14, PDE4B, PELI1, PFKP, PIM1, PLA2G4A, PLSCR1, PML, PNP, PNPT1, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SRI, SSPN, ST3GAL5, ST8SIA4, STAT1, STAT2, STAT3, STAT4, TAP1, TAPBP, TDRD7, TNFAIP2, TNFAIP3, TNFAIP6, TNFSF10, TOR1B, TRAFD1, TRIM14, TRIM21, TRIM25, TRIM26, TXNIP, UBE2L6, UPP1, USP18, VAMP5, VAMP8, VCAM1, WARS1, XAF1, XCL1, ZBP1, and ZNFX1.

In any aspect provided herein, or embodiments thereof, detecting 6p21.3 copy number involves detecting a gene selected from one or more of TAP1, TAP2, TAPBP, PSMB8, and PSMB9. In embodiments, failure to detect one or more of the genes identifies a loss of 6p21.3.

In any aspect provided herein, or embodiments thereof, ISG expression level is detected by determining the expression levels for one or more genes selected from one or more of ADAR, APOL6, ARID5B, ARL4A, AUTS2, B2M, BANK1, BATF2, BPGM, BST2, BTG1, C1R, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLR1, CMPK2, CMTR1, CSF2RB, CXCL10, CXCL11, CXCL9, DDX58, DDX60, DHX58, EIF2AK2, EIF4E3, EPSTI1, FAS, FCGR1A, FGL2, FPR1, GBP4, GBP6, GCH1, GPR18, GZMA, HELZ2, HERC6, HIF1A, HLA-A, HLA-B, HLA-DMA, HLA-DQA1, HLA-DRB1, HLA-G, ICAM1, IDO1, IFI27, IFI30, IFI35, IFI44, IFI44L, IFIH1, IFIT1, IFIT2, IFIT3, IFITM2, IFITM3, IFNAR2, IL10RA, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, MARCHF1, METTL7B, MT2A, MTHFD2, MVP, MX1, MX2, MYD88, NAMPT, NCOA3, NFKB1, NFKBIA, NLRC5, NMI, NOD1, NUP93, OAS2, OAS3, OASL, OGFR, P2RYT4, PARP12, PARP14, PDE4B, PELI1, PFKP, PIM1, PLA2G4A, PLSCR1, PML, PNP, PNPT1, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SRI, SSPN, ST3GAL5, ST8SIA4, STAT1, STAT2, STAT3, STAT4, TAP1, TAPBP, TDRD7, TNFAIP2, TNFAIP3, TNFAIP6, TNFSF10, TOR1B, TRAFD1, TRIM14, TRIM21, TRIM25, TRIM26, TXNIP, UBE2L6, UPP1, USP18, VAMP5, VAMP8, VCAM1, WARS1, XAF1, XCL1, ZBP1, and ZNFX1.

In any aspect provided herein, or embodiments thereof, the method further involves administering to the selected patient a modified immune cell containing a chimeric antigen receptor polypeptide. The modified immune cell contains one or more genetic alterations that reduces or eliminates expression and/or activity of a natural killer cell lectin A (NKG2A) polypeptide and/or a cluster of differentiation 94 (CD94) polypeptide. In aspect of the disclosure, or embodiments thereoof, the method further involves administering to the selected patient a modified immune cell containing a chimeric antigen receptor polypeptide if intact 6p21.3 is present in the neoplasia, ISG expression levels are increased in the neoplasia relative to a reference, and/or HLA-E levels are increased in the neoplasia relative to the reference, where the modified immune cell contains one or more genetic alterations that reduces or eliminates expression and/or activity of a natural killer cell lectin A (NKG2A) polypeptide and/or a cluster of differentiation 94 (CD94) polypeptide.

In any aspect provided herein, or embodiments thereof, the modified immune cell is a T cell, a natural killer (NK) cell, a gammadelta T cell, a natural killer T cell.

In any aspect provided herein, or embodiments thereof, ISG expression level is detected by determining the expression levels for one or more genes selected from one or more of those genes listed in FIG. 5E.

The invention provides compositions containing chimeric antigen receptor (CAR) immune cells that have been modified to reduce and/or eliminate expression or activity of a natural killer cell lectin A (NKG2A) polypeptide and/or a cluster of differentiation 94 (CD94) polypeptide, and methods for use thereof to treat a neoplasia (e.g., skin, colon, pancreas, lung, and kidney cancer). Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “6p21.3” is meant locus 21.3 of the short arm (p) of chromosome 6. In embodiments, a cell lacking all or a fragment of a gene encoding TAP1, TAP2, TAPBP, PSMB8, and/or PSMB9 polynucleotide and/or failing to express the same is characterized as having a loss of 6p21.3.

By “cluster of differentiation 19 (CD19) polypeptide” is meant a CD19 protein or fragment thereof, having immunomodulatory activity and having at least about 85% amino acid sequence identity to Genbank Accession No. AAB60697.1 An exemplary CD19 amino acid sequence from Homo sapiens is provided below (GenBank Accession No. AAB60697.1):

>AAB60697.1 CD19 [Homo sapiens]
(SEQ ID NO: 1)
MPPPRLLFFLLFLTPMEVRPEEPLVVKVEGEGDNAVLQCLKGTSDGPTQQ
LTWSRESPLKPFLKLSLGLPGLGIHMRPLASWLFIFNVSQQMGGFYLCQP
GPPSEKAWQPGWTVNVEGSGELFRWNVSDLGGLGCGLKNRSSEGPSSPSG
KLMSPKLYVWAKDRPEIWEGEPPCVPPRDSLNQSLSQDLTMAPGSTLWLS
CGVPPDSVSRGPLSWTHVHPKGPKSLLSLELKDDRPARDMWVMETGLLLP
RATAQDAGKYYCHRGNLTMSFHLEITARPVLWHWLLRTGGWKVSAVTLAY
LIFCLCSLVGILHLQRALVLRRKRKRMTDPTRRFFKVTPPPGSGPQNQYG
NVLSLPTPTSGLGRAQRWAAGLGGTAPSYGNPSSDVQADGALGSRSPPGV
GPEEEEGEGYEEPDSEEDSEFYENDSNLGQDQLSQDGSGYENPEDEPLGP
EDEDSFSNAESYENEDEELTQPVARTMDFLSPHGSAWDPSREATSLGSQS
YEDMRGILYAAPQLRSIRGQPGPNHEEDADSYENMDNPDGPDPAWGGGGR
MGTWSTR.

By “cluster of differentiation 19 (CD19) polynucleotide” is meant a nucleic acid PGP21,DNA molecule encoding a CD19 polypeptide, as well as the introns, exons, 3′ untranslated regions, 5′ untranslated regions, and regulatory sequences associated with its expression, or fragments thereof. In embodiments, a CD19 polynucleotide is the genomic sequence, cDNA, mRNA, or gene associated with and/or required for CD19 expression. An exemplary CD19 nucleotide sequence from Homo sapiens is provided below (GenBank Accession No. AH005421.2):

>AH005421.2: 331-421, 665-931, 1230-1433,
1554-1829, 2057-2167, 2769-2817, 3128-3216,
3591-3704, 3896-4000, 4174-4242, 4343-4399,
4488-4544, 4813-4905, 5231-5322 Homo sapiens CD19
(CD19) gene, complete cds
(SEQ ID NO: 2)
ATGCCACCTCCTCGCCTCCTCTTCTTCCTCCTCTTCCTCACCCCCATGGA
AGTCAGGCCCGAGGAACCTCTAGTGGTGAAGGTGGAAGGTGAGGGAGATA
ACGCTGTGCTGCAGTGCCTCAAGGGGACCTCAGATGGCCCCACTCAGCAG
CTGACCTGGTCTCGGGAGTCCCCGCTTAAACCCTTCTTAAAACTCAGCCT
GGGGCTGCCAGGCCTGGGAATCCACATGAGGCCCCTGGCATCCTGGCTTT
TCATCTTCAACGTCTCTCAACAGATGGGGGGCTTCTACCTGTGCCAGCCG
GGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGG
GCTGTGGCCTGAAGAACAGGTCCTCAGAGGGCCCCAGCTCCCCTTCCGGG
AAGCTCATGAGCCCCAAGCTGTATGTGTGGGCCAAAGACCGCCCTGAGAT
CTGGGAGGGAGAGCCTCCGTGTGTCCCACCGAGGGACAGCCTGAACCAGA
GCCTCAGCCAGGACCTCACCATGGCCCCTGGCTCCACACTCTGGCTGTCC
TGTGGGGTACCCCCTGACTCTGTGTCCAGGGGCCCCCTCTCCTGGACCCA
TGTGCACCCCAAGGGGCCTAAGTCATTGCTGAGCCTAGAGCTGAAGGACG
ATCGCCCGGCCAGAGATATGTGGGTAATGGAGACGGGTCTGTTGTTGCCC
CGGGCCACAGCTCAAGACGCTGGAAAGTATTATTGTCACCGTGGCAACCT
GACCATGTCATTCCACCTGGAGATCACTGCTCGGCCAGTACTATGGCACT
GGCTGCTGAGGACTGGTGGCTGGAAGGTCTCAGCTGTGACTTTGGCTTAT
CTGATCTTCTGCCTGTGTTCCCTTGTGGGCATTCTTCATCTTCAAAGAGC
CCTGGTCCTGAGGAGGAAAAGAAAGCGAATGACTGACCCCACCAGGAGAT
TCTTCAAAGTGACGCCTCCCCCAGGAAGCGGGCCCCAGAACCAGTACGGG
AACGTGCTGTCTCTCCCCACACCCACCTCAGGCCTCGGACGCGCCCAGCG
TTGGGCCGCAGGCCTGGGGGGCACTGCCCCGTCTTATGGAAACCCGAGCA
GCGACGTCCAGGCGGATGGAGCCTTGGGGTCCCGGAGCCCGCCGGGAGTG
GGCCCAGAAGAAGAGGAAGGGGAGGGCTATGAGGAACCTGACAGTGAGGA
GGACTCCGAGTTCTATGAGAACGACTCCAACCTTGGGCAGGACCAGCTCT
CCCAGGATGGCAGCGGCTACGAGAACCCTGAGGATGAGCCCCTGGGTCCT
GAGGATGAAGACTCCTTCTCCAACGCTGAGTCTTATGAGAACGAGGATGA
AGAGCTGACCCAGCCGGTCGCCAGGACAATGGACTTCCTGAGCCCTCATG
GGTCAGCCTGGGACCCCAGCCGGGAAGCAACCTCCCTGGGGTCCCAGTCC
TATGAGGATATGAGAGGAATCCTGTATGCAGCCCCCCAGCTCCGCTCCAT
TCGGGGCCAGCCTGGACCCAATCATGAGGAAGATGCAGACTCTTATGAGA
ACATGGATAATCCCGATGGGCCAGACCCAGCCTGGGGAGGAGGGGGCCGC
ATGGGCACCTGGAGCACCAGGTGA.

By “cluster of differentiation 94 (CD94) polypeptide” is meant a CD94 protein or fragment thereof, capable of dimerizing with an NKG2A polypeptide and having immunomodulatory activity and having at least about 85% amino acid sequence identity to NCBI Accession No. NP_001107868.2. An exemplary CD94 amino acid sequence from Homo sapiens is provided below (NCBI Ref. Seq. Accession No. NP_001107868.2):

>NP_001107868.2 natural killer cells antigen CD94
isoform 1 [Homo sapiens]
(SEQ ID NO: 3)
MAVFKTTLWRLISGTLGIICLSLMSTLGILLKNSFTKLSIEPAFTPGPNI
ELQKDSDCCSCQEKWVGYRCNCYFISSEQKTWNESRHLCASQKSSLLQLQ
NTDELDFMSSSQQFYWIGLSYSEEHTAWLWENGSALSQYLFPSFETFNTK
NCIAYNPNGNALDESCEDKNRYICKQQLI.

By “cluster of differentiation 94 (CD94) polynucleotide” is meant a nucleic acid molecule encoding a CD94 polypeptide, as well as the introns, exons, 3′ untranslated regions, 5′ untranslated regions, and regulatory sequences associated with its expression, or fragments thereof. In embodiments, a CD94 polynucleotide is the genomic sequence, cDNA, mRNA, or gene associated with and/or required for CD94 expression. An exemplary CD94 nucleotide sequence from Homo sapiens is provided below (NCBI Ref. Seq. Accession No.: NM_001114396.3):

>NM_001114396.3:203-742 Homo sapiens killer cell
lectin like receptor D1 (KLRD1), transcript
variant 3, mRNA
(SEQ ID NO: 4)
ATGGCAGTGTTTAAGACCACTCTGTGGAGGTTAATTTCTGGGACCTTAGG
GATAATATGCCTTTCGTTGATGTCTACGTTGGGAATTTTGTTGAAAAATT
CTTTTACTAAACTGAGTATTGAGCCAGCATTTACTCCAGGACCCAACATA
GAACTCCAGAAAGACTCTGACTGCTGTTCTTGCCAAGAAAAATGGGTTGG
GTACCGGTGCAACTGTTACTTCATTTCCAGTGAACAGAAAACTTGGAACG
AAAGTCGGCATCTCTGTGCTTCTCAGAAATCCAGCCTGCTTCAGCTTCAA
AACACAGATGAACTGGATTTTATGAGCTCCAGTCAACAATTTTACTGGAT
TGGACTCTCTTACAGTGAGGAGCACACCGCCTGGTTGTGGGAGAATGGCT
CTGCACTCTCCCAGTATCTATTTCCATCATTTGAAACTTTTAATACAAAG
AACTGCATAGCGTATAATCCAAATGGAAATGCTTTAGATGAATCCTGTGA
AGATAAAAATCGTTATATCTGTAAGCAACAGCTCATTTAA.

By “human leukocyte antigen E (HLA-E) polypeptide” is meant a HLA-E protein or fragment thereof, having immunomodulatory activity and having at least about 85% amino acid sequence identity to GenBank Accession No. ARB08449.1 An exemplary HLA-E amino acid sequence from Homo sapiens is provided below (GenBank Accession No. ARB08449.1):

>ARB08449.1 HLA-E [Homo sapiens]
(SEQ ID NO: 5)
MVDGTLLLLLSEALALTQTWAGSHSLKYFHTSVSRPGRGEPRFISVGYVD
DTQFVRFDNDAASPRMVPRAPWMEQEGSEYWDRETRSARDTAQIFRVNLR
TLRGYYNQSEAGSHTLQWMHGCELGPDRRFLRGYEQFAYDGKDYLTLNED
LRSWTAVDTAAQISEQKSNDASEAEHQRAYLEDTCVEWLHKYLEKGKETL
LHLEPPKTHVTHHPISDHEATLRCWALGFYPAEITLTWQQDGEGHTQDTE
LVETRPAGDGTFQKWAAVVVPSGEEQRYTCHVQHEGLPEPVTLRWKPASQ
PTIPIVGIIAGLVLLGSVVSGAVVAAVIWRKKSSGGKGGSYSKAEWSDSA
QGSESHSL.

By “human leukocyte antigen E (HLA-E) polynucleotide” is meant a nucleic acid molecule encoding a HLA-E polypeptide, as well as the introns, exons, 3′ untranslated regions, 5′ untranslated regions, and regulatory sequences associated with its expression, or fragments thereof. In embodiments, a HLA-E polynucleotide is the genomic sequence, cDNA, mRNA, or gene associated with and/or required for HLA-E expression. An exemplary HLA-E nucleotide sequence from Homo sapiens is provided below (GenBank Accession No.: KY497359.1):

>KY497359.1: 301-364, 495-764, 1009-1284,
1906-2181, 2306-2422, 3173-3205, 3310-3350
Homo sapiens HLA-E (HLA-E) gene, complete cds
(SEQ ID NO: 6)
ATGGTAGATGGAACCCTCCTTTTACTCCTCTCGGAGGCCCTGGCCCTTAC
CCAGACCTGGGCGGGCTCCCACTCCTTGAAGTATTTCCACACTTCCGTGT
CCCGGCCCGGCCGCGGGGAGCCCCGCTTCATCTCTGTGGGCTACGTGGAC
GACACCCAGTTCGTGCGCTTCGACAACGACGCCGCGAGTCCGAGGATGGT
GCCGCGGGCGCCGTGGATGGAGCAGGAGGGGTCAGAGTATTGGGACCGGG
AGACACGGAGCGCCAGGGACACCGCACAGATTTTCCGAGTGAACCTGCGG
ACGCTGCGCGGCTACTACAATCAGAGCGAGGCCGGGTCTCACACCCTGCA
GTGGATGCATGGCTGCGAGCTGGGGCCCGACAGGCGCTTCCTCCGCGGGT
ATGAACAGTTCGCCTACGACGGCAAGGATTATCTCACCCTGAATGAGGAC
CTGCGCTCCTGGACCGCGGTGGACACGGCGGCTCAGATCTCCGAGCAAAA
GTCAAATGATGCCTCTGAGGCGGAGCACCAGAGAGCCTACCTGGAAGACA
CATGCGTGGAGTGGCTCCACAAATACCTGGAGAAGGGGAAGGAGACGCTG
CTTCACCTGGAGCCCCCAAAGACACACGTGACTCACCACCCCATCTCTGA
CCATGAGGCCACCCTGAGGTGCTGGGCCCTGGGCTTCTACCCTGCGGAGA
TCACACTGACCTGGCAGCAGGATGGGGAGGGCCATACCCAGGACACGGAG
CTCGTGGAGACCAGGCCTGCAGGGGATGGAACCTTCCAGAAGTGGGCAGC
TGTGGTGGTGCCTTCTGGAGAGGAGCAGAGATACACGTGCCATGTGCAGC
ATGAGGGGCTACCCGAGCCCGTCACCCTGAGATGGAAGCCGGCTTCCCAG
CCCACCATCCCCATCGTGGGCATCATTGCTGGCCTGGTTCTCCTTGGATC
TGTGGTCTCTGGAGCTGTGGTTGCTGCTGTGATATGGAGGAAGAAGAGCT
CAGGTGGAAAAGGAGGGAGCTACTCTAAGGCTGAGTGGAGCGACAGTGCC
CAGGGGTCTGAGTCTCACAGCTTGTAA.

By “interferon gamma (IFNγ) polypeptide” is meant a IFNγ protein or fragment thereof, having immunomodulatory activity and having at least about 85% amino acid sequence identity to GenBank Accession No. AAB59534.1. An exemplary IFNγ amino acid sequence from Homo sapiens is provided below (GenBank: AAB59534.1):

>AAB59534.1 interferon-gamma [Homo sapiens]
(SEQ ID NO: 7)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ.

By “interferon gamma (IFNγ) polynucleotide” is meant a nucleic acid molecule encoding a IFNγ polypeptide, as well as the introns, exons, 3′ untranslated regions, 5′ untranslated regions, and regulatory sequences associated with its expression, or fragments thereof. In embodiments, a IFNγ polynucleotide is the genomic sequence, cDNA, mRNA, or gene associated with and/or required for IFNγ expression. An exemplary IFNγ nucleotide sequence from Homo sapiens is provided below (GenBank Accession No. J00219.1):

>J00219.1:475-588, 1828-1896, 1992-2174, 4600-4734
Homo sapiens interferon-gamma (IFNG) gene,
complete cds
(SEQ ID NO: 8)
ATGAAATATACAAGTTATATCTTGGCTTTTCAGCTCTGCATCGTTTTGGG
TTCTCTTGGCTGTTACTGCCAGGACCCATATGTAAAAGAAGCAGAAAACC
TTAAGAAATATTTTAATGCAGGTCATTCAGATGTAGCGGATAATGGAACT
CTTTTCTTAGGCATTTTGAAGAATTGGAAAGAGGAGAGTGACAGAAAAAT
AATGCAGAGCCAAATTGTCTCCTTTTACTTCAAACTTTTTAAAAACTTTA
AAGATGACCAGAGCATCCAAAAGAGTGTGGAGACCATCAAGGAAGACATG
AATGTCAAGTTTTTCAATAGCAACAAAAAGAAACGAGATGACTTCGAAAA
GCTGACTAATTATTCGGTAACTGACTTGAATGTCCAACGCAAAGCAATAC
ATGAACTCATCCAAGTGATGGCTGAACTGTCGCCAGCAGCTAAAACAGGG
AAGCGAAAAAGGAGTCAGATGCTGTTTCGAGGTCGAAGAGCATCCCAGTA
A.

By “natural killer cell lectin A (NKG2A) polypeptide” is meant a NKG2A protein or fragment thereof, having immunomodulatory activity and having at least about 85% amino acid sequence identity to NCBI Ref. Seq. Accession No. NP_998823.1. An exemplary NKG2A amino acid sequence from Homo sapiens is provided below (NCBI Ref. Seq. Accession No. NP_998823.1):

>NP_998823.1 KLRC1 [organism-Homo sapiens]
[GeneID = 3821] [isoform = NKG2-A]
(SEQ ID NO: 9)
MDNQGVIYSDLNLPPNPKRQQRKPKGNKSSILATEQEITYAELNLQKASQ
DFQGNDKTYHCKDLPSAPEKLIVGILGIICLILMASVVTIVVIPSTLIQR
HNNSSLNTRTQKARHCGHCPEEWITYSNSCYYIGKERRTWEESLLACTSK
NSSLLSIDNEEEMKFLSIISPSSWIGVFRNSSHHPWVTMNGLAFKHEIKD
SDNAELNCAVLQVNRLKSAQCGSSIIYHCKHKL.

By “natural killer cell lectin A (NKG2A) polynucleotide” is meant a nucleic acid molecule encoding a NKG2A polypeptide, as well as the introns, exons, 3′ untranslated regions, 5′ untranslated regions, and regulatory sequences associated with its expression, or fragments thereof. In embodiments, a NKG2A polynucleotide is the genomic sequence, cDNA, mRNA, or gene associated with and/or required for NKG2A expression. An exemplary NKG2A nucleotide sequence from Homo sapiens is provided below (NCBI Ref. Seq. Accession No. NM_213658.2):

>NM_213658.2: 389-1090 Homo sapiens killer cell
lectin like receptor C1 (KLRC1), transcript
variant 3, mRNA
(SEQ ID NO: 10)
ATGGATAACCAAGGAGTAATCTACTCAGACCTGAATCTGCCCCCAAACCC
AAAGAGGCAGCAACGAAAACCTAAAGGCAATAAAAGCTCCATTTTAGCAA
CTGAACAGGAAATAACCTATGCGGAATTAAACCTTCAAAAAGCTTCTCAG
GATTTTCAAGGGAATGACAAAACCTATCACTGCAAAGATTTACCATCAGC
TCCAGAGAAGCTCATTGTTGGGATCCTGGGAATTATCTGTCTTATCTTAA
TGGCCTCTGTGGTAACGATAGTTGTTATTCCCTCTACATTAATACAGAGG
CACAACAATTCTTCCCTGAATACAAGAACTCAGAAAGCACGTCATTGTGG
CCATTGTCCTGAGGAGTGGATTACATATTCCAACAGTTGTTACTACATTG
GTAAGGAAAGAAGAACTTGGGAAGAGAGTTTGCTGGCCTGTACTTCGAAG
AACTCCAGTCTGCTTTCTATAGATAATGAAGAAGAAATGAAATTTCTGTC
CATCATTTCACCATCCTCATGGATTGGTGTGTTTCGTAACAGCAGTCATC
ATCCATGGGTGACAATGAATGGTTTGGCTTTCAAACATGAGATAAAAGAC
TCAGATAATGCTGAACTTAACTGTGCAGTGCTACAAGTAAATCGACTTAA
ATCAGCCCAGTGTGGATCTTCAATAATATATCATTGTAAGCATAAGCTTT
AG.

By “proteasome 20S subunit beta 8 (PSMB8) polypeptide” is meant a PSMB8 protein or fragment thereof, having immunomodulatory activity and having at least about 85% amino acid sequence identity to NCBI Ref. Seq. Accession No. NP_004150.1. An exemplary PSMB8 amino acid sequence from Homo sapiens is provided below (NCBI Ref. Seq. Accession No. NP_004150.1):

>NP_004150.1 proteasome subunit beta type-8
isoform E1 [Homo sapiens]
(SEQ ID NO: 11)
MLIGTPTPRDTTPSSWLTSSLLVEAAPLDDTTLPTPVSSGCPGLEPTEFF
QSLGGDGERNVQIEMAHGTTTLAFKFQHGVIAAVDSRASAGSYISALRVN
KVIEINPYLLGTMSGCAADCQYWERLLAKECRLYYLRNGERISVSAASKL
LSNMMCQYRGMGLSMGSMICGWDKKGPGLYYVDEHGTRLSGNMFSTGSGN
TYAYGVMDSGYRPNLSPEEAYDLGRRAIAYATHRDSYSGGVVNMYHMKED
GWVKVESTDVSDLLHQYREANQ.

By “proteasome 20S subunit beta 8 (PSMB8) polynucleotide” is meant a nucleic acid molecule encoding a PSMB8 polypeptide, as well as the introns, exons, 3′ untranslated regions, 5′ untranslated regions, and regulatory sequences associated with its expression, or fragments thereof. In embodiments, a PSMB8 polynucleotide is the genomic sequence, cDNA, mRNA, or gene associated with and/or required for PSMB8 expression. An exemplary PSMB8 nucleotide sequence from Homo sapiens is provided below (NCBI Ref. Seq. Accession No. NM_004159.5):

>NM_004159.5: 267-1085 Homo sapiens proteasome 20S
subunit beta 8 (PSMB8), transcript variant 1, mRNA
(SEQ ID NO: 12)
ATGCTCATAGGAACCCCCACCCCGCGTGACACTACTCCCAGCTCCTGGCT
GACTTCTAGTCTTCTGGTTGAAGCTGCGCCTTTAGATGACACGACCCTAC
CCACCCCTGTTTCCAGCGGATGCCCGGGCCTGGAGCCCACAGAATTCTTC
CAGTCCCTGGGTGGGGACGGAGAAAGGAACGTTCAGATTGAGATGGCCCA
TGGCACCACCACGCTCGCCTTCAAGTTCCAGCATGGAGTGATTGCAGCAG
TGGATTCTCGGGCCTCAGCTGGGTCCTACATTAGTGCCTTACGGGTGAAC
AAGGTGATTGAGATTAACCCTTACCTGCTTGGCACCATGTCTGGCTGTGC
AGCAGACTGTCAGTACTGGGAGCGCCTGCTGGCCAAGGAATGCAGGCTGT
ACTATCTGCGAAATGGAGAACGTATTTCAGTGTCGGCAGCCTCCAAGCTG
CTGTCCAACATGATGTGCCAGTACCGGGGCATGGGCCTCTCTATGGGCAG
TATGATCTGTGGCTGGGATAAGAAGGGTCCTGGACTCTACTACGTGGATG
AACATGGGACTCGGCTCTCAGGAAATATGTTCTCCACGGGTAGTGGGAAC
ACTTATGCCTACGGGGTCATGGACAGTGGCTATCGGCCTAATCTTAGCCC
TGAAGAGGCCTATGACCTTGGCCGCAGGGCTATTGCTTATGCCACTCACA
GAGACAGCTATTCTGGAGGCGTTGTCAATATGTACCACATGAAGGAAGAT
GGTTGGGTGAAAGTAGAAAGTACAGATGTCAGTGACCTGCTGCACCAGTA
CCGGGAAGCCAATCAATAA.

By “proteasome 20S subunit beta 9 (PSMB9) polypeptide” is meant a PSMB9 protein or fragment thereof, having immunomodulatory activity and having at least about 95% amino acid sequence identity to NCBI Ref. Seq. Accession No. AQY77063.1. An exemplary PSMB9 amino acid sequence from Homo sapiens is provided below (NCBI Ref. Seq. Accession No. AQY77063.1):

>AQY77063.1 PSMB9 [Homo sapiens]
(SEQ ID NO: 13)
MLRAGAPTGDLPRAGEVHTGTTIMAVEFDGGIVMGSDSRVSAGEAVVNRV
FDKLSPLHERIYCALSGSAADAQAVADMAAYQLELHGIELEEPPLVLAAA
NVVRNISYKYREDLSAHLMVAGWDQREGGQVYGTLGGMLTRQPFAIGGSG
STFIYGYVDAAYKPGMSPEECRRFTTDAIALAMSRDGSSGGVIYLVTITA
AGVDHRVILGNELPKFYDE.

By “proteasome 20S subunit beta 9 (PSMB9) polynucleotide” is meant a nucleic acid molecule encoding a PSMB9 polypeptide, as well as the introns, exons, 3′ untranslated regions, 5′ untranslated regions, and regulatory sequences associated with its expression, or fragments thereof. In embodiments, a PSMB9 polynucleotide is the genomic sequence, cDNA, mRNA, or gene associated with and/or required for PSMB9 expression. An exemplary PSMB9 nucleotide sequence from Homo sapiens is provided below (GenBank Accession No. KY500591.2):

>KY500591.2:1-60, 1909-1976, 3030-3161, 3772-3901,
4131-4272,5173-5300 Homo sapiens isolate PITOUT
PSMB9 gene, complete cds
(SEQ ID NO: 14)
ATGCTGCGGGCGGGAGCACCAACCGGGGACTTACCCCGGGCGGGAGAAGT
CCACACCGGGACCACCATCATGGCAGTGGAGTTTGACGGGGGCATTGTGA
TGGGTTCTGATTCCCGAGTGTCTGCAGGCGAGGCGGTGGTGAACCGAGTG
TTTGACAAGCTGTCCCCGCTGCACGAGCGCATCTACTGTGCACTCTCTGG
TTCAGCTGCTGATGCCCAAGCCGTGGCCGACATGGCCGCCTACCAGCTGG
AGCTCCATGGGATAGAACTGGAGGAACCTCCACTTGTTTTGGCTGCTGCA
AATGTGGTGAGAAATATCAGCTATAAATATCGAGAGGACTTGTCTGCACA
TCTCATGGTAGCTGGCTGGGACCAACGTGAAGGAGGTCAGGTATATGGAA
CCCTGGGAGGAATGCTGACTCGACAGCCTTTTGCCATTGGTGGCTCCGGC
AGCACCTTTATCTATGGTTATGTGGATGCAGCATATAAGCCAGGCATGTC
TCCCGAGGAGTGCAGGCGCTTCACCACAGACGCTATTGCTCTGGCCATGA
GCCGGGATGGCTCAAGCGGGGGTGTCATCTACCTGGTCACTATTACAGCT
GCCGGTGTGGACCATCGAGTCATCTTGGGCAATGAACTGCCAAAATTCTA
TGATGAGTGA.

By “transporter 1, ATP binding cassette subfamily B member (TAP1) polypeptide” is meant a TAP1 protein or fragment thereof, having immunomodulatory activity and having at least about 85% amino acid sequence identity to GenBank Accession No. AAS55412.1. An exemplary TAP1 amino acid sequence from Homo sapiens is provided below (GenBank Accession No. AAS55412.1).

>AAS55412.1 TAP1 [Homo sapiens]
(SEQ ID NO: 15)
MASSRCPAPRGCRCLPGASLAWLGTVLLLLADWVLLRTALPRIFSLLVPT
ALPLLRVWAVGLSRWAVLWLGACGVLRATVGSKSENAGAQGWLAALKPLA
AALGLALPGLALFRELISWGAPGSADSTRLLHWGSHPTAFVVSYAAALPA
AALWHKLGSLWVPGGQGGSGNPVRRLLGCLGSETRRLSLFLVLVVLSSLG
EMAIPFFTGRLTDWILQDGSADTFTRNLTLMSILTIASAVLEFVGDGIYN
NTMGHVHSHLQGEVFGAVLRQETEFFQQNQTGNIMSRVTEDTSTLSDSLS
ENLSLFLWYLVRGLCLLGIMLWGSVSLTMVTLVTLPLLFLLPKKVGKWYQ
LLEVQVRESLAKSSQVAIEALSAMPTVRSFANEEGEAQKFREKLQEIKTL
NQKEAVAYAVNSWTTSISGMLLKVGILYIGGQLVTSGAVSSGNLVTFVLY
QMQFTQAVEVLLSIYPRVQKAVGSSEKIFEYLDRTPRCPPSGLLTPLHLE
GLVQFQDVSFAYPNRPDVLVLQGLTFTLRPGEVTALVGPNGSGKSTVAAL
LQNLYQPTGGQLLLDGKPLPQYEHRYLHRQVAAVGQEPQVFGRSLQENIA
YGLTQKPTMEEITAAAVKSGAHSFISGLPQGYDTEVGEAGSQLSGGQRQA
VALARALIRKPCVLILDDATSALDANSQLQVEQLLYESPERYSRSVLLIT
QHLSLVEQADHILFLEGGAIREGGTHQQLMEKKGCYWAMVQAPADAPE.

By “transporter 1, ATP binding cassette subfamily B member (TAP1) polynucleotide” is meant a nucleic acid molecule encoding a TAP1 polypeptide, as well as the introns, exons, 3′ untranslated regions, 5′ untranslated regions, and regulatory sequences associated with its expression, or fragments thereof. In embodiments, a TAP1 polynucleotide is the genomic sequence, cDNA, mRNA, or gene associated with and/or required for TAP1 expression. An exemplary TAP1 polynucleotide sequence from Homo sapiens is provided below (GenBank Accession No. AY523971.2):

>AY523971.2: 207-2453 Homo sapiens TAP1 (TAP1)
mRNA, TAP1*020101 allele, complete cds
(SEQ ID NO: 16)
ATGGCTAGCTCTAGGTGTCCCGCTCCCCGCGGGTGCCGCTGCCTCCCCGG
AGCTTCTCTCGCATGGCTGGGGACAGTACTGCTACTTCTCGCCGACTGGG
TGCTGCTCCGGACCGCGCTGCCCCGCATATTCTCCCTGCTGGTGCCCACC
GCGCTGCCACTGCTCCGGGTCTGGGCGGTGGGCCTGAGCCGCTGGGCCGT
GCTCTGGCTGGGGGCCTGCGGGGTCCTCAGGGCAACGGTTGGCTCCAAGA
GCGAAAACGCAGGTGCCCAGGGCTGGCTGGCTGCTTTGAAGCCATTAGCT
GCGGCACTGGGCTTGGCCCTGCCGGGACTTGCCTTGTTCCGAGAGCTGAT
CTCATGGGGAGCCCCCGGGTCCGCGGATAGCACCAGGCTACTGCACTGGG
GAAGTCACCCTACCGCCTTCGTTGTCAGTTATGCAGCGGCACTGCCCGCA
GCAGCCCTGTGGCACAAACTCGGGAGCCTCTGGGTGCCCGGCGGTCAGGG
CGGCTCTGGAAACCCTGTGCGTCGGCTTCTAGGCTGCCTGGGCTCGGAGA
CGCGCCGCCTCTCGCTGTTCCTGGTCCTGGTGGTCCTCTCCTCTCTTGGG
GAGATGGCCATTCCATTCTTTACGGGCCGCCTCACTGACTGGATTCTACA
AGATGGCTCAGCCGATACCTTCACTCGAAACTTAACTCTCATGTCCATTC
TCACCATAGCCAGTGCAGTGCTGGAGTTCGTGGGTGACGGGATCTATAAC
AACACCATGGGCCACGTGCACAGCCACTTGCAGGGAGAGGTGTTTGGGGC
TGTCCTGCGCCAGGAGACGGAGTTTTTCCAACAGAACCAGACAGGTAACA
TCATGTCTCGGGTAACAGAGGACACGTCCACCCTGAGTGATTCTCTGAGT
GAGAATCTGAGCTTATTTCTGTGGTACCTGGTGCGAGGCCTATGTCTCTT
GGGGATCATGCTCTGGGGATCAGTGTCCCTCACCATGGTCACCCTGGTCA
CCCTGCCTCTGCTTTTCCTTCTGCCCAAGAAGGTGGGAAAATGGTACCAG
TTGCTGGAAGTGCAGGTGCGGGAATCTCTGGCAAAGTCCAGCCAGGTGGC
CATTGAGGCTCTGTCGGCCATGCCTACAGTTCGAAGCTTTGCCAACGAGG
AGGGCGAAGCCCAGAAGTTTAGGGAAAAGCTGCAAGAAATAAAGACACTC
AACCAGAAGGAGGCTGTGGCCTATGCAGTCAACTCCTGGACCACTAGTAT
TTCAGGTATGCTGCTGAAAGTGGGAATCCTCTACATTGGTGGGCAGCTGG
TGACCAGTGGGGCTGTAAGCAGTGGGAACCTTGTCACATTTGTTCTCTAC
CAGATGCAGTTCACCCAGGCTGTGGAGGTACTGCTCTCCATCTACCCCAG
AGTACAGAAGGCTGTGGGCTCCTCAGAGAAAATATTTGAGTACCTGGACC
GCACCCCTCGCTGCCCACCCAGTGGTCTGTTGACTCCCTTACACTTGGAG
GGCCTTGTCCAGTTCCAAGATGTCTCCTTTGCCTACCCAAACCGCCCAGA
TGTCTTAGTGCTACAGGGGCTGACATTCACCCTACGCCCTGGCGAGGTGA
CGGCGCTGGTGGGACCCAATGGGTCTGGGAAGAGCACAGTGGCTGCCCTG
CTGCAGAATCTGTACCAGCCCACCGGGGGACAGCTGCTGTTGGATGGGAA
GCCCCTTCCCCAATATGAGCACCGCTACCTGCACAGGCAGGTGGCTGCAG
TGGGACAAGAGCCACAGGTATTTGGAAGAAGTCTTCAAGAAAATATTGCC
TATGGCCTGACCCAGAAGCCAACTATGGAGGAAATCACAGCTGCTGCAGT
AAAGTCTGGGGCCCATAGTTTCATCTCTGGACTCCCTCAGGGCTATGACA
CAGAGGTAGGCGAGGCTGGGAGCCAGCTGTCAGGGGGTCAGCGACAGGCA
GTGGCGTTGGCCCGAGCATTGATCCGGAAACCGTGTGTACTTATCCTGGA
TGATGCCACCAGTGCCCTGGATGCAAACAGCCAGTTACAGGTGGAGCAGC
TCCTGTACGAAAGCCCTGAGCGGTACTCCCGCTCAGTGCTTCTCATCACC
CAGCACCTCAGCCTGGTGGAGCAGGCTGACCACATCCTCTTTCTGGAAGG
AGGCGCTATCCGGGAGGGGGGAACCCACCAGCAGCTCATGGAGAAAAAGG
GGTGCTACTGGGCCATGGTGCAGGCTCCTGCAGATGCTCCAGAATGA.

By “transporter 2, ATP binding cassette subfamily B member (TAP2) polypeptide” is meant a TAP2 protein or fragment thereof, having immunomodulatory activity and having at least about 85% amino acid sequence identity to GenBank Accession No. AHW47975.1. An exemplary TAP2 amino acid sequence from Homo sapiens is provided below (GenBank Accession No. AHW47975.1):

>AHW47975.1 TAP2 [Homo sapiens]
(SEQ ID NO: 17)
MRLPDLRPWTSLLLVDAALLWLLQGPLGTLLPQGLPGLWLEGTLRLGGLW
GLLKLRGLLGFVGTLLLPLCLATPLTVSLRALVAGASRAPPARVASAPWS
WLLVGYGAAGLSWSLWAVLSPPGAQEKEQDQVNNKVLMWRLLKLSRPDLP
LLVAAFFFLVLAVLGETLIPHYSGRVIDILGGDFDPHAFASAIFFMCLFS
FGSSLSAGCRGGCFTYTMSRINLRIREQLFSSLLRQDLGFFQETKTGELN
SRLSSDTTLMSNWLPLNANVLLRSLVKVVGLYGFMLSISPRLTLLSLLHM
PFTIAAEKVYNTRHQEVLREIQDAVARAGQVVREAVGGLQTVRSFGAEEH
EVCRYKEALEQCRQLYWRRDLERALYLLVRRVLHLGVQMLMLSCGLQQMQ
DGELTQGSLLSFMIYQESVGSYVQTLVYIYGDMLSNVGAAEKVFSYMDRQ
PNLPSPGTLAPTTLQGVVKFQDVSFAYPNRPDRPVLKGLTFTLRPGEVTA
LVGPNGSGKSTVAALLQNLYQPTGGQVLLDEKPISQYEHCYLHSQVVSVG
QEPVLFSGSVRNNIAYGLQSCEDDKVMAAAQAAHADDFIQEMEHGIYTDV
GEKGSQLAAGQKQRLAIARALVRDPRVLILDEATSALDVQCEQALQDWNS
RGDRTVLVIAHRLQTVQRAHQILVLQEGKLQKLAQL.

By “transporter 2, ATP binding cassette subfamily B member (TAP2) polynucleotide” is meant a nucleic acid molecule encoding a TAP2 polypeptide, as well as the introns, exons, 3′ untranslated regions, 5′ untranslated regions, and regulatory sequences associated with its expression, or fragments thereof. In embodiments, a TAP2 polynucleotide is the genomic sequence, cDNA, mRNA, or gene associated with and/or required for TAP2 expression. An exemplary TAP2 nucleotide sequence from Homo sapiens is provided below (GenBank Accession No. KJ657697.1):

>KJ657697.1: c530822-530330, c530240-530126,
c528362-528232, c527948-527743, c525413-525216,
c525050-524922, c523395-523207, c523029-522856,
c522678-522519, c522125-521989, c521623-521495
Homo sapiens clone HIP1009 major
histocompatibility complex class II gene locus,
partial sequence
(SEQ ID NO: 18)
ATGCGGCTCCCTGACCTGAGACCCTGGACCTCCCTGCTGCTGGTGGACGC
GGCTTTACTGTGGCTGCTTCAGGGCCCTCTGGGGACTTTGCTTCCTCAAG
GGCTGCCAGGACTATGGCTGGAGGGGACCCTGCGGCTGGGAGGGCTGTGG
GGGCTGCTAAAGCTAAGAGGGCTGCTGGGATTTGTGGGGACACTGCTGCT
CCCGCTCTGTCTGGCCACCCCCCTGACTGTCTCCCTGAGAGCCCTGGTCG
CGGGGGCCTCACGTGCTCCCCCAGCCAGAGTCGCTTCAGCCCCTTGGAGC
TGGCTGCTGGTGGGGTACGGGGCTGCGGGGCTCAGCTGGTCACTGTGGGC
TGTTCTGAGCCCTCCTGGAGCCCAGGAGAAGGAGCAGGACCAGGTGAACA
ACAAAGTCTTGATGTGGAGGCTGCTGAAGCTCTCCAGGCCGGACCTGCCT
CTCCTCGTTGCCGCCTTCTTCTTCCTTGTCCTTGCTGTTTTGGGTGAGAC
ATTAATCCCTCACTATTCTGGTCGTGTGATTGACATCCTGGGAGGTGATT
TTGACCCCCATGCCTTTGCCAGTGCCATCTTCTTCATGTGCCTCTTCTCC
TTTGGCAGCTCACTGTCTGCAGGCTGCCGAGGAGGCTGCTTCACCTACAC
CATGTCTCGAATCAACTTGCGGATCCGGGAGCAGCTTTTCTCCTCCCTGC
TGCGCCAGGACCTCGGTTTCTTCCAGGAGACTAAGACAGGGGAGCTGAAC
TCACGGCTGAGCTCGGATACCACCCTGATGAGTAACTGGCTTCCTTTAAA
TGCCAATGTGCTCTTGCGAAGCCTGGTGAAAGTGGTGGGGCTGTATGGCT
TCATGCTCAGCATATCGCCTCGACTCACCCTCCTTTCTCTGCTGCACATG
CCCTTCACAATAGCAGCGGAGAAGGTGTACAACACCCGCCATCAGGAAGT
GCTTCGGGAGATCCAGGATGCAGTGGCCAGGGCGGGGCAGGTGGTGCGGG
AAGCCGTTGGAGGGCTGCAGACCGTTCGCAGTTTTGGGGCCGAGGAGCAT
GAAGTCTGTCGCTATAAAGAGGCCCTTGAACAATGTCGGCAGCTGTATTG
GCGGAGAGACCTGGAACGCGCCTTGTACCTGCTCGTAAGGAGGGTGCTGC
ACTTGGGGGTGCAGATGCTGATGCTGAGCTGTGGGCTGCAGCAGATGCAG
GATGGGGAGCTCACCCAGGGCAGCCTGCTTTCCTTTATGATCTACCAGGA
GAGCGTGGGGAGCTATGTGCAGACCCTGGTATACATATATGGGGATATGC
TCAGCAACGTGGGAGCTGCAGAGAAGGTTTTCTCCTACATGGACCGACAG
CCAAATCTGCCTTCACCTGGCACGCTTGCCCCCACCACTCTGCAGGGGGT
TGTGAAATTCCAAGACGTCTCCTTTGCATATCCCAATCGCCCTGACAGGC
CTGTGCTCAAGGGGCTGACGTTTACCCTACGTCCTGGTGAGGTGACGGCG
CTGGTGGGACCCAATGGGTCTGGGAAGAGCACAGTGGCTGCCCTGCTGCA
GAATCTGTACCAGCCCACAGGGGGACAGGTGCTGCTGGATGAAAAGCCCA
TCTCACAGTATGAACACTGCTACCTGCACAGCCAGGTGGTTTCAGTTGGG
CAGGAGCCTGTGCTGTTCTCCGGTTCTGTGAGGAACAACATTGCTTATGG
GCTGCAGAGCTGCGAAGATGATAAGGTGATGGCGGCTGCCCAGGCTGCCC
ACGCAGATGACTTCATCCAGGAAATGGAGCATGGAATATACACAGATGTA
GGGGAGAAGGGAAGCCAGCTGGCTGCGGGACAGAAACAACGTCTGGCCAT
TGCCCGGGCCCTTGTACGAGACCCGCGGGTCCTCATCCTGGATGAGGCTA
CTAGTGCCCTAGATGTGCAGTGCGAGCAGGCCCTGCAGGACTGGAATTCC
CGTGGGGATCGCACAGTGCTGGTGATTGCTCACAGGCTGCAGACAGTTCA
GCGCGCCCACCAGATCCTGGTGCTCCAGGAGGGCAAGCTGCAGAAGCTTG
CCCAGCTCTAG.

By “TAP binding protein (TAPBP) polypeptide” is meant a TAPBP protein or fragment thereof, having immunomodulatory activity and having at least about 85% amino acid sequence identity to GenBank Accession No. AQY77142.1. An exemplary TAPBP amino acid sequence from Homo sapiens is provided below (GenBank: AQY77142.1):

>AQY77142.1 TAPBP [Homo sapiens]
(SEQ ID NO: 19)
MKSLSLLLAVALGLATAVSAGPAVIECWFVEDASGKGLAKRPGALLLRQG
PGEPPPRPDLDPELYLSVHDPAGALQAAFRRYPRGAPAPHCEMSRFVPLP
ASAKWASGLTPAQNCPRALDGAWLMVSISSPVLSLSSLLRPQPEPQQEPV
LITMATVVLTVLTHTPAPRVRLGQDALLDLSFAYMPPTSEAASSLAPGPP
PFGLEWRRQHLGKGHLLLAATPGLNGQMPAAQEGAVAFAAWDDDEPWGPW
TGNGTFWLPRVQPFQEGTYLATIHLPYLQGQVTLELAVYKPPKVSLMPAT
LARAAPGEAPPELLCLVSHFYPSGGLEVEWELRGGPGGRSQKAEGQRWLS
ALRHHSDGSVSLSGHLQPPPVTTEQHGARYACRIHHPSLPASGRSAEVTL
EVAGLSGPSLEDSVGLFLSAFLLLGLFKALGWAAVYLSTCKDSKKKAE.

By “TAP binding protein (TAPBP) polynucleotide” is meant a nucleic acid molecule encoding a TAPBP polypeptide, as well as the introns, exons, 3′ untranslated regions, 5′ untranslated regions, and regulatory sequences associated with its expression, or fragments thereof. In embodiments, a TAPBP polynucleotide is the genomic sequence, cDNA, mRNA, or gene associated with and/or required for TAPBP expression. An exemplary TAPBP nucleotide sequence from Homo sapiens is provided below (GenBank Accession No. KY500670.2):

>KY500670.2: 1-37, 178-348, 565-825, 8646-9044,
9395-9736, 9816-9905, 10044-10078, 12264-12275
Homo sapiens isolate COX TAPBP gene, complete cds
(SEQ ID NO: 20)
ATGAAGTCCCTGTCTCTGCTCCTCGCTGTGGCTTTGGGCCTGGCGACCGC
CGTCTCAGCAGGACCCGCGGTGATCGAGTGTTGGTTCGTGGAGGATGCGA
GCGGAAAGGGCCTGGCCAAGAGACCCGGTGCACTGCTGTTGCGCCAGGGA
CCGGGGGAACCGCCGCCCCGGCCGGACCTCGACCCTGAGCTCTATCTCAG
TGTACACGACCCCGCGGGCGCCCTCCAGGCTGCCTTCAGGCGGTATCCCC
GGGGCGCCCCCGCACCACACTGCGAGATGAGCCGCTTCGTGCCTCTCCCC
GCCTCTGCGAAATGGGCCAGCGGCCTGACCCCCGCGCAGAACTGCCCGCG
GGCCCTGGATGGGGCTTGGCTGATGGTCAGCATATCCAGCCCAGTCCTCA
GCCTCTCCAGCCTCTTGCGACCACAGCCAGAGCCTCAGCAGGAGCCTGTT
CTCATCACCATGGCAACAGTGGTACTGACTGTCCTCACCCACACCCCTGC
CCCTCGAGTGAGACTGGGACAAGATGCTCTGCTGGACTTGAGCTTTGCCT
ACATGCCCCCCACCTCCGAGGCCGCCTCATCTCTGGCTCCGGGTCCCCCT
CCCTTTGGGCTAGAGTGGCGACGCCAGCACCTGGGTAAGGGACATCTGCT
CCTGGCTGCAACTCCTGGGCTGAATGGCCAGATGCCAGCAGCCCAAGAAG
GGGCCGTGGCATTTGCTGCTTGGGATGATGATGAGCCATGGGGCCCATGG
ACCGGAAATGGGACCTTCTGGCTGCCTAGAGTTCAACCCTTTCAGGAGGG
CACCTATCTGGCCACCATACACCTGCCATACCTGCAAGGACAGGTCACCC
TGGAGCTTGCTGTGTACAAACCCCCCAAAGTGTCCCTGATGCCAGCAACC
CTTGCACGGGCCGCCCCAGGGGAGGCACCCCCGGAATTGCTCTGCCTTGT
GTCCCACTTCTACCCTTCTGGGGGCCTGGAGGTGGAGTGGGAACTCCGGG
GTGGCCCAGGGGGCCGCTCTCAGAAGGCCGAGGGGCAGAGGTGGCTCTCG
GCCCTGCGCCACCATTCCGATGGCTCTGTCAGCCTCTCTGGGCACTTGCA
GCCGCCCCCAGTCACCACTGAGCAGCATGGGGCACGCTATGCCTGTCGAA
TTCACCATCCCAGCCTGCCTGCCTCGGGGCGCAGCGCTGAGGTCACCCTG
GAGGTAGCAGGTCTTTCAGGGCCCTCCCTTGAGGACAGCGTAGGCCTTTT
CCTGTCTGCCTTTCTTCTGCTTGGGCTCTTCAAGGCACTGGGCTGGGCTG
CTGTCTACCTGTCCACCTGCAAGGATTCAAAGAAGAAAGCAGAGTGA.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change in the structure, expression levels or activity of a polynucleotide or polypeptide as detected by standard art known methods such as those described herein. The alteration can be an increase or a decrease. As used herein, an alteration includes a 10% change in expression levels, a 25% change, a 40% change, a 50% or a greater change in expression levels. In embodiments, an alteration in structure is a genetic alteration. In embodiments, the genetic alteration is a missense mutation, deletion, or insertion that results in a loss of function.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” or “antigen-binding domain” refers to an immunoglobulin molecule or a fragment thereof that specifically binds to, or is immunologically reactive with, a particular antigen. Non-limiting examples of antibodies or antigen-binding domains include polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)2, Fab, Fv, rlgG, and scFv fragments, as well as engineered antibodies, which include CrossMabs (e.g., CrossMab^{Fabs}, CrossMab^{CH1-CL} and CrossMab^{VH-VL} formats), or fragments thereof. Moreover, unless otherwise indicated, the term “monoclonal antibody” (mAb) is meant to include both intact molecules, as well as antibody fragments (such as, for example, Fab and F(ab′)2 fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

By “antigen” is meant an agent to which an antibody or other polypeptide capture molecule specifically binds. In an embodiment, the antigen is a tumor antigen. Exemplary antigens include small molecules, carbohydrates, proteins, and polynucleotides.

By “Chimeric Antigen Receptor” or alternatively a “CAR” is meant a polypeptide capable of providing an immune effector cell with specificity for a target cell. In embodiments, the target cell is a cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one embodiment, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

By “chemotherapeutic agent” is meant an agent that inhibits cancer cell proliferation, inhibits cancer cell survival, increases cancer cell death, inhibits and/or stabilizes tumor growth, or that is otherwise useful in the treatment of cancer. In embodiments, chemotherapeutic agents provided herein are used as part of an immunotherapy. In embodiments, chemotherapeutic agents provided herein contain an immune checkpoint blockade (ICB). In embodiments, the ICB contains a PD-1/PD-L1 checkpoint inhibitor (e.g., atezolizumab, avelumab, BMS-936559, MDX-1105, cemiplimab, durvalumab, nivolumab, and/or pembrolizumab). In embodiments, an PD-1/PD-L1 checkpoint inhibitor contains an anti-CTLA-4 and/or anti-PD-1 antibody. In embodiments, the chemotherapeutic agents provided herein contain a CAR-T that has been modified to reduce or eliminate expression or activity of an NKG2A and/or CD94 polypeptide.

One of skill in the art can readily identify a chemotherapeutic agent of use in a method for treating a cancer described herein (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments of any of the aspects, the combination of agents provided herein decrease cancer cell proliferation or survival by at least 10%, 20%, 30%, 40%, 50%, 60%, 70%, 80%, 90%, 95%, or 100%, and includes inducing cell death (apoptosis) in a cell or cells within a cell mass.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In embodiments, the disease is a neoplasia. Exemplary neoplasias include, but are not limited to, cancers of the skin (e.g., melanoma), colon, pancreas, lung (non-small cell lung cancer), and kidney.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunomodulatory activity” is meant increasing, decreasing, or participating in an immune response.

By “immunotherapy” is meant a treatment that involves supplementing or stimulating the immune system. Non-limiting examples of immunotherapies include treatments involving administration of immune checkpoint blockades and/or CAR T cells.

By “immune checkpoint blockade” is meant an agent that blocks a checkpoint protein from binding it's partner. In embodiments, the agent is an antibody. In some cases, the polynucleotide and/or pathway functions in inhibiting an immune response. In some instances, an immune checkpoint inhibitor inhibits PD-1/PD-L1, CTLA-4, NKG2A, and/or CD94. In embodiments, an immune checkpoint blockade inhibits the interaction of a receptor (e.g., PD-1) with its respective ligand (e.g., PD-L1).

By “increase” is meant to alter positively by at least 5% relative to a reference. An increase may be by 5%, 10%, 25%, 30%, 50%, 75%, or even by 100%.

By “interferon-stimulated gene” is meant a gene with expression levels that increase when a cell containing the gene is contacted with an interferon. In embodiments, the interferon is IFNγ. Exemplary ISGs include, but are not limited to, ADAR, APOL6, ARID5B, ARL4A, AUTS2, B2M, BANK1, BATF2, BPGM, BST2, BTG1, C1R, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLR1, CMPK2, CMTR1, CSF2RB, CXCL10, CXCL11, CXCL9, DDX58, DDX60, DHX58, EIF2AK2, EIF4E3, EPSTI1, FAS, FCGR1A, FGL2, FPR1, GBP4, GBP6, GCH1, GPR18, GZMA, HELZ2, HERC6, HIF1A, HLA-A, HLA-B, HLA-DMA, HLA-DQA1, HLA-DRB1, HLA-G, ICAM1, IDO1, IF127, IF130, IF135, IF144, IF144L, IFIH1, IFIT1, IFIT2, IFIT3, IFITM2, IFITM3, IFNAR2, IL10RA, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, MARCHF1, METTL7B, MT2A, MTHFD2, MVP, MX1, MX2, MYD88, NAMPT, NCOA3, NFKB1, NFKBIA, NLRC5, NMI, NOD1, NUP93, OAS2, OAS3, OASL, OGFR, P2RY14, PARP12, PARP14, PDE4B, PELI1, PFKP, PIM1, PLA2G4A, PLSCR1, PML, PNP, PNPT1, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SRI, SSPN, ST3GAL5, ST8SIA4, STAT1, STAT2, STAT3, STAT4, TAP1, TAPBP, TDRD7, TNFAIP2, TNFAIP3, TNFAIP6, TNFSF10, TOR1B, TRAFD1, TRIM14, TRIM21, TRIM25, TRIM26, TXNIP, UBE2L6, UPP1, USP18, VAMP5, VAMP8, VCAM1, WARS1, XAF1, XCL1, ZBP1, and ZNFX1. In various embodiments, expression of an ISG increases by at least about 10, 20, 30, 40, 50, 60, 70, 80, or 90 percent relative to a reference. In other embodiments, the expression of an ISG increases by at least about 1.1×, 1.2×, 1.3×, 1.4×, 1.5×, 2×, 3×, 4×, or 5×. In embodiments, ISGs level are considered as being “high” in a subject if the hallmark IFN gene expression signature for the subject is within the top 25% of that observed in a population (e.g., a patient cohort or a cohort of healthy subjects). In embodiments, expression is measured using RNAseq. In some instances, the increase is statistically significant (e.g., a p value cutoff of p<0.1, p<0.05, or p<0.01)

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a developmental state, condition, disease, or disorder.

As used herein, “obtaining” as in “obtaining an agent” includes synthesizing, purchasing, or otherwise acquiring the agent.

By “neoplasia” is meant a disease or disorder characterized by excess proliferation or reduced apoptosis. In embodiments, a neoplasia is a cancer or tumor. Illustrative neoplasms include breast cancer, esophageal cancer, head-and-neck cancer, pancreatic cancer, skin cancer, colorectal cancer, hepatocellular cancer, bladder cancer, bile duct cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, liver cancer, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In embodiments, the neoplasia may be colon adenocarcinoma (COAD), stomach adenocarcinoma (STAD), stomach cancer, and uterine corpus endometrial carcinoma (UCEC). In embodiments, the neoplasia may be a liquid tumor such as, for example, leukemia or lymphoma. In embodiments, the cancer is a colon, kidney, lung, pancreatic, renal (e.g., renal cell carcinoma or clear renal cell carcinoma), or skin cancer (e.g., a melanoma).

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively by at least 5% relative to a reference. A reduction may be by 5%, 10%, 25%, 30%, 50%, 75%, or even by 100%.

By “reference” is meant a standard or control condition. Non-limiting examples of references include a healthy subject, a subject prior to a change in treatment or administration of an agent, and an unmodified cell. In some instances, a reference is a cell (e.g., an immune cell, such as a CAR T cell) that expresses a functional NKG2A and/or CD94 polypeptide.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature.

As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G provide schematic diagrams, plots, and a visualization of a network showing in vivo genome-scale screens revealed mechanisms of immunotherapy resistance and sensitization. FIG. 1A provides a schematic diagram of screen design. FIG. 1B provides plots showing genes ranked by immune checkpoint blockade (ICB) vs NOD SCID I12rg^{-/-} (NSG)-normalized fold change with circle size corresponding to −log 10 FDR. The top genes with FDR<0.1 at each end are listed and arranged by statistical significance. FIG. 1C provides plots showing tumor volume over time for B16 cells transduced with Cas9 and sgRNAs targeting control or Ccar1 (top panel) or Calr (bottom panel) implanted into mice treated with GVAX and anti-PD-1; n=5-10. FIG. 1D provides plots showing tumor volume over time for KPC (top) and Renca (bottom panel) cells transduced with control or H2-T23 sgRNA implanted into mice treated with anti-PD-1; n=5-10. FIG. 1E provides plots showing tumor volume over time (top) and survival (bottom) for MC38 cells overexpressing hCD19 or Qa-1^{b} from a constitutive (EF1a) or interferon-inducible (Irf1) promoter implanted into mice treated with anti-PD-1; n=5-10. FIG. 1F provides a plot showing core pathway enrichment for depleted genes across all screens with general biological pathway annotation (right). Circle shade corresponds to the odds ratio for overrepresentation calculated by Fisher's exact test while circle size corresponds to −log 10 adjusted p-value. FIG. 1G provides a visualization of a network showing STRING network analysis of genes that were depleted with FDR<0.25 in two or more models, with circle size scaled by aggregate screen score. Clusters were determined by Markov clustering. Pooled data are represented as mean±SEM (c-e); two-sided Student's t-test; *p<0.05, **p<0.01, ***p<0.001.

FIGS. 2A-2E provide plots, a schematic diagram, and a bar graph showing interferon (IFN)-mediated inhibition of anti-tumor immunity was dependent on MHC-I presentation. FIG. 2A provides a plot showing aggregate screen scores for combined data from immune checkpoint blockade (ICB)-treated vs NOD SCID I12rg^{-/-} (NSG) genome-scale in vivo screens plotted against CTL screens performed by Lawson et al 2020. Covariance ellipse shows 4 standard deviations. Circle size is scaled by Euclidean distance from the origin and circle color corresponds to pathways highlighted in FIG. 1F. FIG. 2B provides a plot showing tumor volume over time for KPC cells transduced with control (grey lines) or Jak1-targeting (red lines) sgRNA implanted into untreated mice (left) or mice treated with 200 μg anti-PD-1 on days 6, 9, and 12 following implantation (right); n=5-10. FIG. 2C provides a scatter plot of IFNγ-induced gene expression change (x-axis) by enrichment or depletion in immune checkpoint blockade (ICB)-treated mice vs NOD SCID I12rg^{-/-} mice (NSG) comparison across in vivo screens (y-axis) for all genes included in the genome-scale screening library. Circle size indicates aggregate screen score. FIG. 2D provides a schematic diagram of IFNγ-MHC epistasis competition design. FIG. 2E provides a bar graph showing log fold change in the ratio of tumor cells with sgRNAs targeting Ifngr1 or Jak1 vs control sgRNA within KPC (left panels) or CT26 (right panels) tumors in a control (light grey) or Tap1-deficient (dark grey) genetic background, normalized to the ratio for tumors implanted in NOD SCID I12rg^{-/-} (NSG) mice. Tumors were implanted into WT C57BL/6 (KPC) or Balb/C (CT26) mice that were either untreated or treated with 200 μg anti-PD-1 on days 6, 9, and 12; n=5. Pooled data are represented as mean±SEM (b). Two-sided Student's t-test; ns p>0.05, *p<0.05, **p<0.01, ***p<0.001, ****p<0.0001.

FIGS. 3A-3H provide plots and bar graphs showing immune checkpoint blockade (ICB) activated CD4⁺ T cells and NK cells to eliminate interferon (IFN) sensing-deficient tumors. FIG. 3A provides plots showing tumor volume over time for control (light grey lines) or Jak1-deficient (dark grey lines) KPC tumors in WT mice treated with 200 μg anti-PD-1 on days 6, 9, and 12 with no depletion or with 200 μg anti-CD8, anti-NK1.1, or anti-CD4 depleting antibody every 4 days starting 1 day prior to tumor implantation as indicated by the arrows. FIG. 3B provides a plot showing tumor volume over time for Jak1-deficient KPC cells implanted into anti-PD-1-treated mice (light grey) with NK depletion starting 1 day prior to tumor implantation (dark grey) or concurrently with anti-PD-1 (mid-shade grey). FIG. 3C provides a plot showing UMAP projection of CD4⁺ T cell populations isolated from scRNAseq of KPC-tumor infiltrating lymphocytes (top left); UMAP density projections showing shifts in CD4⁺ T cell populations in response to anti-PD-1 and anti-CTLA-4 treatment (top middle and right panels); overlaid heat map showing relative expression of CD4 lineage-specific, exhaustion, and cytotoxic markers (lower panels). FIG. 3D provides a bar graph showing quantification of CD4⁺ T cell population changes with immune checkpoint blockade (ICB) from FIG. 3C. FIG. 3E provides contour plots showing PD-1 cell surface expression on CD4⁺, CD8⁺, and NK1.1⁺ TILs isolated from KPC tumors measured by flow cytometry (left panels), and quantification of PD-1 expression across tumor replicates (right panel). FIG. 3F provides a plot showing tumor volume over time for KPC tumors in control untreated WT mice (light grey lines) or treated with 200 μg anti-PD-1 on days 6, 9, and 12 alone (mid-shade grey lines) or in combination with NKG2D blockade (200 μg anti-NKG2D every 4 days starting 1 day prior to tumor implantation; dark-grey lines lines). FIG. 3G provides a bar graph showing log fold change in the ratio of control (light grey; left), Tap1-null (mid-shade grey; center), or H2-K1-null (dark grey; right) KPC tumor cells transduced with Jak1 or control sgRNAs cultured for 48 hours with activated NK cells at effector:target ratios of 0:1, 4:1, and 8:1, normalized to the 0:1 ratio for each condition. FIG. 3H provides a scatter plot of in vivo KPC screening data showing average fold change by gene for the immune checkpoint blockade (ICB) vs NOD SCID I12rg^{-/-} (NSG) sub-genome-scale screen (x-axis) against the immune checkpoint blockade (ICB)+anti-NK1.1 vs NSG screen (y-axis). Circle size is scaled to false discovery rate (FDR) in the immune checkpoint blockade (ICB)+anti-NK1.1 vs NSG screen. Pooled data are represented as mean±SEM. Student's t test for n=10 (a); n=5 (b); n=3 (c-d). Data are representative of 2 independent experiments (FIG. 3A); 1 experiment (FIGS. 3B-3E). *p<0.05, **p<0.01, ***p<0.001, ****p<0.0001.

FIGS. 4A-4F provide plots, bar graphs, and a schematic diagram showing Qa-1^{b}/NKG2A is an immune checkpoint blockade (ICB)-induced immune checkpoint for CD8⁺ T cells. FIG. 4A provides plots showing tumor volume over time for WT C57BL/6 (KPC) cells transfected with Cas9 and control (light-grey lines) or H2-T23 (dark-grey lines) sgRNAs and implanted into WT mice treated with 200 μg anti-PD-1 days 6, 9, and 12 post-implantation with or without either 200 μg anti-NK1.1 (middle) or 200 μg anti-CD8b (right) depleting antibodies administered every 4 days starting 1 day prior to tumor injection. FIG. 4B provides plots showing UMAP projection of CD8⁺ T cell populations isolated from scRNAseq of KPC-tumor infiltrating lymphocytes (top left); UMAP density projections showing shifts in CD8⁺ T cell populations in response to anti-PD-1 and anti-CTLA-4 treatment (top middle and right panels); UMAP projection of CD8⁺ T cells with overlaid heatmap showing differential expression of marker genes Sell (CD62L), Pdcd1 (PD-1), Prf1 (perforin), Gzmb (Granzyme B), Klrc1 (NKG2A) and Klrd1 (CD94) used to label cell populations (lower panels). FIG. 4C provides bar graphs showing quantification of immune checkpoint blockade (ICB)-induced changes in CD8+ T cell subset frequencies from top panels with each point representing immune infiltrate from a different tumor (n=4 per group). FIG. 4D provides a bar graph showing log fold change in the ratio of CD19⁺ KPC tumor cells transduced with H2-T23 sgRNA or control sgRNA stimulated with IFNγ for 24 hours and then co-cultured with CD19 CAR-T cells at effector:target ratios of 1:4, 1:2, 1:1, 2:1, and 4:1 for 72 hours. Normalized to no T cells. FIG. 4E provides a schematic diagram for an in vivo competitive CAR-T assay. FIG. 4F provides a bar graph showing log fold change in the ratio of Jak1 versus control sgRNA CD19⁺ KPC tumor cells transduced with Jak1 or control sgRNA on either a control (black outline; left) or Qa-1^{b}-deficient (grey outline; right) genetic background, implanted subcutaneously into NOD SCID I12rg^{-/-} (NSG) mice which received I.V. injection of either 1e6 activated CD19 CAR-T or untransduced T cells on day 7, normalized to no T cell transfer. Pooled data are represented as mean±SEM. Student's t test for n=10 (a); n=4 (b). Data are representative of 2 independent experiments for a, d. *p<0.05, **p<0.01, ***p<0.001, ****p<0.0001.

FIGS. 5A-5F provides plots, a bar graph, and a heat map showing a high interferon signature predicted therapeutic resistance in renal clear cell carcinoma and melanoma. FIGS. 5A and 5B provide plots showing stratification of overall survival by IFNγ signature score in (FIG. 5A) clear cell renal cell carcinoma (ccRCC) and (FIG. 5B) melanoma cohorts, selected by 6p21.3 copy number status. P-values determined by log-rank test. FIG. 5C provides a plot showing Cox proportional hazards regression modeling of expanded melanoma cohort, showing interaction between various features and IFN signature in predicting survival. FIG. 5D provides a bar graph showing log fold change in serum protein expression of 50 interferon-stimulated genes (ISGs) in responding (R) versus non-responding (NR) patients 6 weeks and 6 months post-immune checkpoint blockade (ICB)-treatment. Left bar at each time point=R; right bar at each time point=NR. FIG. 5E provides a heatmap showing normalized fold change in serum protein expression of 50 ISGs in patients with melanoma 6 months post immune checkpoint blockade (ICB) treatment and GSEA enrichment of non-responders in high interferon-stimulated gene (ISG) samples. FIG. 5F provides plots showing changes in serum protein expression of IFNγ and HLA-E. NR=lines with the lowest NPX at 6-months; R=lines with the highest NPX (Olink's relative protein quantification unit on a log 2 scale) at 6-months. Statistical significance was assessed by Mann-Whitney U-test. Data are mean±s.d. **p<0.005. For patients, high interferon-stimulated gene (ISG) samples were those with aggregate ISG expression scores falling in the top 25% of the patient cohort, where the ISG expression scores were determined using mRNA expression levels calculated using RNAseq, and where the ISG expression scores were calculated using the hallmark IFN gene expression signature.

FIG. 6 provides plots showing tumor growth kinetics for the indicated cell lines from genome-scale screens. Tumor volume was measured over time for in vivo screening groups: cells were injected into NSG, WT, or WT IBC-treated mice as indicated. Pooled data are represented as mean±SEM.

FIGS. 7A-7D provide plots relating to quality control metric for genome-scale screens. FIG. 7A provides plots showing in vitro and in vivo library recovery. Dotted lines represent abundance at 5th and 95th percentile for each library. FIG. 7B provides plots showing replicate autocorrelation analysis. Pearson's correlations are calculated for the library distribution in one 10-tumor replicate versus any other replicate, two averaged replicates versus any other two, and so on. The mean of all possible combinations is plotted. FIG. 7C provides plots showing an effect size model for sgRNAs. A natural cubic spline (solid line) was fit between NOD SCID I12rg^{-/-} (NSG) and WT or immune checkpoint blockade (ICB) sgRNA abundances, and the effect size is calculated as the residual from the spline. FIG. 7D provides plots showing normalized effect size distributions for the WT versus NSG and immune checkpoint blockade (ICB) versus NSG comparisons; distributions for control sgRNAs are shown in dotted lines, distributions for gene-targeting sgRNAs are shown in solid lines.

FIGS. 8A-8D provide plots and bar graphs showing additional analyzes of tumor-immune dependencies relating to genome-scale screens. FIG. 8A provides a plot of genes ranked by WT vs NOD SCID I12rg^{-/-} (NSG) normalized fold change with circle size corresponding to −log 10 FDR. The top genes with FDR<0.1 at each end are listed and arranged by statistical significance. FIG. 8B provides a bar graph showing a comparison of the number of depleted gene deletions with FDR<0.25 in the immune checkpoint blockade (ICB) vs NSG comparison that scored across combinations of models. FIG. 8C provides a bar graph showing a comparison of the number of enriched versus depleted genes recovered at FDR<0.25 for immune checkpoint blockade (ICB) vs NSG or WT vs NSG comparisons. FIG. 8D, Paired fold change for all genes that were enriched or depleted with false discovery rate (FDR)<0.25 in the immune checkpoint blockade (ICB) vs NSG or WT vs NSG comparisons. Statistical significance was assessed using a one-sided paired t test *p<0.05, **p<0.005, ***p<0.0005.

FIGS. 9A and 9B provide plots relating to sub-genome screens. FIG. 9A provides plots showing pre-ranked GSEA of enriched and depleted sub-genome screen hits in the corresponding genome screen. Gene sets in sub-genome screens were defined with FDR<0.25 threshold separately for enriched and depleted genes, and GSEA enrichment was performed by ranking genes by signed STARS score in the genome screen. FIG. 9B provides plots showing genes ranked by immune checkpoint blockade (ICB) vs NSG normalized fold change with circle size corresponding to −log 10 FDR. The top genes with FDR<0.1 at each end are listed and arranged by statistical significance.

FIGS. 10A-10E provide plots, a heat map, and a histogram showing Qa-1^{b} was an immune inhibitory ligand downstream of interferon (IFN). FIG. 10A provides a plot showing average normalized fold change by aggregate STARS score for the immune checkpoint blockade (ICB) vs NOD SCID I12rg^{-/-} (NSG) comparison. FIG. 10B provides a plot showing average normalized fold change by aggregate STARS score for the WT vs NSG comparison. FIG. 10C provides a plot showing tumor volume over time for YUMMER tumor cells transfected with Cas9 and sgRNA targeting control (light-grey lines) or H2-T23 (dark-grey lines) and implanted into male B6 mice that were treated with 100 μg anti-PD-1 and 100 μg anti-CTLA-4 on days 6, 9, and 12 post-implantation. FIG. 10D provides a heat map showing relative transcript abundance of H2-T23 mRNA across cell lines treated with and without IFNγ, measured by RNAseq. FIG. 10E provides histograms showing cell surface expression of Qa-1^{b} measured by flow cytometry on MC38 cells transduced with hCD19 or constitutive or IFNγ-inducible Qa-1^{b} overexpression constructs. Pooled data are represented as mean±SEM. Student's t test for n=5 (c) **p<0.01.

FIGS. 11A and 11B provide a plot and histograms showing MHC-I was a potential immune inhibitory ligand downstream of interferon (IFN). FIG. 11A provides a scatter plot of IFNγ-induced gene expression change (x-axis) by enrichment or depletion in WT vs NOD SCID I12rg^{-/-} (NSG) comparison across in vivo screens (y-axis) for all genes included in the genome-scale screening library. Dot size indicates aggregate screen score. FIG. 11B provides histograms showing cell surface expression of H2-D^{b} and H2-K^{b} on KPC Cas9 cells transduced with control, Tap1, or Jak1 sgRNA and cultured with or without IFNγ in vitro.

FIGS. 12A-12F provide plots, heat maps, and a bar graph relating to an scRNAseq analysis. FIG. 12A provides a plot showing all cells labeled by cell type according to marker gene expression. FIG. 12B provides a heatmap of differentially expressed marker genes used to label cell populations. FIG. 12C provides violin plots showing expression of NKG2 and Ly49 receptor family members in all cell populations. Y-axis represents log-transformed expression. FIG. 12D provides violin plots showing expression of Klrc1 in CD8⁺ T cell, NK cell, and innate lymphoid cell populations. Populations are ordered by mean expression, which is indicated by the solid line. FIG. 12E provides plots showing UMAP density projections showing shifts in the innate lymphoid cell compartment in response to 100 μg anti-PD-1 and 100 μg CTLA-4 treatment (ICB) on days 6 and 9. FIG. 12F provides a bar graph showing quantification of cell population changes in FIG. 12E. Each point represents immune infiltrate from a different tumor (n=4 per group). *p<0.05, **p<0.01.

FIGS. 13A and 13B provides a bar graph and a plot showing tumor interferon (IFN) sensing inhibited NK cell cytotoxicity via upregulation of classical MHC-I. FIG. 13A provides a bar graph showing log fold change in the ratio of control (light-grey) or Tap1-null (dark-grey) CT26 tumor cells transduced with Jak1 or control sgRNAs cultured for 48 hours with activated NK cells at effector:target ratios of 0:1, 4:1, and 8:1, normalized to the 0:1 ratio for each condition. FIG. 13B provides a scatter plot of in vivo CT26 screening data showing average fold change by gene for the immune checkpoint blockade (ICB) vs. NOD SCID I12rg^{-/-} (NSG) sub-genome-scale screen (x-axis) against the immune checkpoint blockade (ICB)+anti-asialo GM1 vs NSG screen (y-axis). Circle size is scaled to FDR in the immune checkpoint blockade (ICB)+anti-asialo GM1 vs NSG screen. Student's t test for n=5 (a); n=3 (b, c). *p<0.05, **p<0.01, ***p<0.001, ****p<0.0001.

FIGS. 14A-14D provide plots and histograms showing the Qa-1^{b}/NKG2A axis was an interferon-dependent T cell regulator. FIG. 14A provides a dot plot showing expression of NKG2 family and activation and exhaustion marker gene transcripts on subpopulations of CD8⁺ T cells. FIG. 14B provides a plot showing a gating strategy for flow cytometry analysis of NKG2A expression on CD8⁺ T cells and NK cells in tumor infiltrating lymphocytes from KPC tumors treated with 100 μg anti-PD-1 on days 6 and 9 post-implantation and harvested on day 12 post-implantation. FIG. 14C provides histograms showing cell surface staining for NKG2A measured by flow cytometry on isolated splenic CD8⁺ T cells stimulated with anti-CD8 and anti-CD28 and cultured in IL-2 alone (top) or IL-2 and IL-12 (bottom). FIG. 14D provides histograms showing cell surface expression of PD-L1 or Qa-1^{b} measured by flow cytometry on KPC cells transfected with Cas9 and sgRNA targeting Jak1 or control with and without IFNγ treatment.

FIGS. 15A-15C provide plots showing 6p21.3 loss in human cancers. FIG. 15A provides plots showing frequency of 6p21.3 deletion across The Cancer Genome Atlas (TCGA) histopathologies and their hazard ratios as calculated by Cox proportional hazard models. Error bars show 95% confidence intervals. FIG. 15B provides Kaplan-Meier plots of overall survival in the Liu and Braun patient cohorts, stratified by 6p21.3 copy number status. FIG. 15C provides a plot showing univariate Cox proportional hazard models for the Braun data set, calculated for the nivolumab treated patient cohort.

FIG. 16 provides a heat map showing serum protein expression in melanoma patients pre- and post-immune checkpoint blockade (ICB). The heatmap shows normalized fold changes in serum protein expression of 50 ISGs in patients with melanoma 6 weeks post immune checkpoint blockade (ICB) treatment and GSEA enrichment of non-responders.

FIGS. 17A-17C provide plots relating to in vivo genome-scale screens revealing mechanisms of immunotherapy resistance and sensitization. FIGS. 17A and 17B provide plots showing tumor volume over time for B16 cells transduced with Cas9 and sgRNAs targeting control or Ccar1 (FIG. 17A), Calr (FIG. 17B, left panel) or Med16 (FIG. 17B, right panel) implanted into mice treated with GVAX and anti-PD-1. FIG. 17C provides plots showing tumor volume over time for CT26 (left panel), Panc02 (mid panel) or B16 cells (right panel) transduced with Cas9 and sgRNAs targeting control (upper lines) or Rnf31 (lower lines) implanted into mice treated with ICB as indicated. Data are representative of 2 independent experiments (c-e). *p<0.05, **p<0.01, ***p<0.001, ****p<0.0001.

FIGS. 18A and 18B provide plots showing that loss of IFN signaling sensitized tumors to ICB. FIGS. 18A and 18B provide plots showing volume over time for KPC (FIG. 18A) or CT26 (FIG. 18B) cells transduced with control, Ifnar1-, Ifngr1- or Jak1-targeting sgRNA implanted into untreated mice (left) or mice treated with ICB (right) (200 μg anti-PD-1 on days 6, 9, 12, and 15 following implantation for KPC; 100 μg anti-PD-1 and anti-CTLA-4 on days 9, 12, and 15 following implantation for CT26. *p<0.05, **p<0.01, ***p<0.001, ****p<0.0001.

FIGS. 19A-19C provide histograms, bar graphs, and a schematic diagram showing IFN-mediated inhibition of anti-tumor immunity was dependent on MHC-I presentation. FIG. 19A provides histograms showing cell surface expression of H2-K, H-2D, Qa-1^{b} or PD-L1 measured by flow cytometry on CT26, KPC, Panc02 and MC38 cells with (rightmost shaded curves) and without (leftmost shaded curves) IFNγ stimulation. FIG. 19B provides bar graphs showing log fold change in the ratio of tumor cells with sgRNAs targeting Ifngr1 or Jak1 vs control sgRNA within KPC tumors in a control (left in each set of three bars), Tap1-deficient (middle in each set of three bars) or B2m-deficient (right of each set of three bars) genetic background, normalized to the ratio for tumors implanted in NSG mice. KPC tumor cells were implanted into WT C57BL/6 mice that were either untreated or treated with 200 μg anti-PD-1 on days 6, 9, and 12. FIG. 19C provides a schematic representation of 6p21.32 region.

FIGS. 20A-20F provide plots, schematic diagrams, contour plots, and bar graphs showing Qa-1^{b}/NKG2A was an ICB-induced immune checkpoint for CD8⁺ T cells. FIG. 20A provides a plot showing tumor volume over time for KPC (left) Renca (mid) and YUMMER cells (right panel) transduced with control or H2-T23 sgRNA implanted into mice treated with ICB. FIG. 20B provides a bar graph showing quantification of ICB-induced changes from top panels with each point representing immune infiltrate from a different tumor (n=4 per group).

FIG. 20C provides a schematic diagram showing design of competitive killing assays using CAR-T cells. FIG. 20D provides contour plots and bar graphs showing representative populations of Jak1 sgRNA versus control sgRNA CD19⁺ KPC tumor cells cultured with CD19 CAR-T cells at effector to target ratios of 1:4, 1:2, 1:1, 2:1, and 4:1 for 72 hours with or without either anti-PD-1 or anti-NKG2A antibodies (5 μg/mL) on either a control or Qa-1^{b}-deficient genetic background (right panels) and log fold changes in the ratios of Jak1 sgRNA versus control sgRNA cells in the competitive killing assay. FIG. 20E provides a schematic diagram for in vivo competitive CAR-T assay. FIG. 20F provides bar graph showing log fold change in the ratio of Jak1 versus control sgRNA CD19⁺ KPC tumor cells transduced with Jak1 or control sgRNA on either a control (left in each set of bars) or Qa-1^{b}-deficient (right in each set of bars) genetic background, implanted subcutaneously into NSG mice which received I.V. injection of either 1e6 activated CD19 CAR-T or untransduced T cells on day 7, normalized to no T cell transfer. Pooled data are represented as mean±SEM. Student's t test for n=10 (a); n=4 (b). Data are representative of 2 independent experiments for a, d. *p<0.05, **p<0.01, ***p<0.001, ****p<0.0001.

FIGS. 21A-21C provide a schematic diagram and plots showing interferon inflammation in post-treatment serum protein analysis was associated with immunotherapy resistance in melanoma. FIG. 21A provides a schematic diagram of study design for proteomic analysis of differentially expressed plasma proteins in metastatic melanoma patients post-ICB compared to baseline. FIG. 21B provides a plot showing Cox proportional hazards modeling of overall and progression-free survival based on mean fold change in serum ISG proteins at 6 weeks and 6 months relative to baseline measurements. Patient age and sex were used as covariates. Error bars indicate 95% confidence intervals. FIG. 21C provides a plot showing a Kaplan-Meier survival analysis of progression-free survival, stratified by median fold change in serum ISG protein at 6 weeks. Significance was assessed by log-rank test.

FIGS. 22A-22C provide heatmaps, histograms, and bar graphs showing MHC-I was a potential immune inhibitory ligand downstream of IFN. FIG. 22A provides a heatmap showing gene expression fold change of NK ligands, IFN signaling pathway, antigen processing and presentation machinery, classical and non-classical class I MHC, and immune inhibitory receptor genes in IFNγ or IFNβ stimulation compared to baseline expression. FIG. 22B provides histograms showing cell surface expression of H2-K, H-2D, Qa-1^{b} or PD-L1 measured by flow cytometry on CT26 and KPC cells with (rightmost shaded curves) and without (leftmost shaded curves) IFNβ stimulation. FIG. 22C provides bar graphs showing log fold change in the ratio of tumor cells with sgRNAs targeting Ifngr1 or Jak1vs control sgRNA within CT26 tumors in a control (left in each pair of bars) or Tap1-deficient (right in each pair of bars) genetic background, normalized to the ratio for tumors implanted in NSG mice. CT26 tumor cells were implanted into WT Balb/C mice that were either untreated or treated with 200 μg anti-PD-1 on days 6, 9, and 12.

FIGS. 23A and 23B provide plots showing Qa-1b was an immune inhibitory ligand downstream of IFN. FIG. 23A provides a plot showing tumor volume over time for KPC (left), Renca (middle) or YUMMER (right) cells transduced with control or H2-T23-targeting sgRNA implanted into untreated WT mice. FIG. 23B provides a plot showing tumor volume over time for MC38 cells overexpressing hCD19 or Qa-1b from a constitutive (EF1a) or interferon-inducible (Irf1) promoter implanted into untreated mice. Pooled data are represented as mean±SEM. Student's t test for n=5 (c) **p<0.01.

FIGS. 24A-24D provide bar graphs, contour plots, and a schematic diagram showing the Qa-1^{b}/NKG2A axis was an interferon-dependent T cell regulator. FIG. 24A provides a bar graph showing log fold change in the ratio of EF1a-Qa1 to CD19-overexpressing MC38 Ova tumor cells co-cultured for 48 hours with activated OT-1 T cells at effector:target ratios of 1:20, 1:10, 1:5, and 1:1. FIG. 24B provides a schematic diagram showing an in vivo competitive OT-1 transfer assay. FIG. 24C provides a bar graph showing log fold change in the ratio of MC38-Ova tumor cells overexpressing EF1a-Qa1 versus CD19 implanted in NSG mice that were injected intravenously with 3e6 activated OT-1 CD8⁺ T cells on day 6 post-implantation. FIG. 24D provides contour plots showing representative populations of control KPC tumor cells and mCD19⁺ KPC tumor cells co-cultured for 72 hours with untransduced (UTD) T cells or mCD19 CAR-T cells at an effector to target ratio of 2:1.

FIG. 25 presents a schematic diagram summarizing the design of an experiment to evaluate polypeptide expression in tumor-infiltrating lymphocytes (TILs) in tumors in NSG-SGM3 mice. In FIG. 25, “TME” indicates “tumor microenvironment,” and “PBMC” indicates “peripheral blood mononuclear cells.”

FIGS. 26A and 26B present flow cytometry scatter plots and flow cytometry histograms showing that human CD8+ tumor-infiltrating lymphocytes (TILs) expressed high levels of NKG2A and CD94 in vivo in mice containing an A375 melanoma (FIG. 26A) or an HT29 colorectal adenocarcinoma (FIG. 26B). In FIGS. 26A and 26B, “FMO” indicates a “Fluorescence Minus One (FMO)” control, and “PBMC” indicates “peripheral blood mononuclear control” cells, “TIM3” indicates “T-cell immunoglobulin mucin-3,” and FITC, AF750, PE, and APC represent fluorophores. The numbers within flow cytometry scatter plots of FIGS. 26A and 26B represent the percent of total cells counted falling within the indicated region or quadrant.

FIG. 27 presents bar graphs showing that KLRC1 (gene encoding an NKG2A polypeptide) and KLRD1 (gene encoding a CD94 polypeptide) were upregulated on tumor-infiltrating human chimeric antigen receptor (CAR) T cells in vivo in NSG mice bearing A375 tumors. Gene expression was analyzed using RNAseq. In FIG. 27 “D0” indicates expression measured at time of injection of the CAR T cells into the mice, D7 indicates expression measured 7 days post-injection of the CAR T cells into the mice, and D14 indicates expression measured 14 days post-injection of the CAR T cells into the mice. CD8+ T cells were subsetted based on expression of PD-1+ single-positive (activated T cells; “CD8_PD1+”) or PD-1+ and CD39+ double-positive (terminally exhausted/effector T cells; “CD8_PD1+CD39+”)). In FIG. 27, “CD8” indicates cells expressing CD8, “PDT+” and “PDT-” indicate cells that do or do not express PD-1, respectively, “CD39+” and “CD39-” indicate cells that do or do not express CD39, respectively, and “log 2 TPM” indicates the log 2 change in “Transcript Count Per Million.”

FIGS. 28A and 28B present plots showing that KLRC1 (gene encoding an NKG2A polypeptide) and KLRD1 (gene encoding a CD94 polypeptide) were among the most differentially expressed genes on tumor-infiltrating human CAR T cells in vivo at day 7 and day 14 post-injection of the CAR T cells into NSG mice bearing A375 tumors.

FIG. 29 presents a series of flow cytometry scatter plots showing the induction of human T cells in vitro to express NKG2A and CD94 after two stimulations using a combination of anti-CD3, anti-CD28, IL-2, IL-7, IL-15, and IL-12 stimulation. The numbers within each quadrant of each flow cytometry scatter plot of FIG. 29 represent the percent of all cells counted falling within the indicated quadrant.

FIG. 30 presents a series of bar graphs showing that deletion of HLA-E sensitized human tumor cell lines (HT-29, 786-O, SU.86.86, and PANC-1) to primary natural killer (NK) cell killing in vitro. In FIG. 30, “CTRL” indicates “control” cells that expressed both HLA-E and B2M, “HLA-E KO” indicates HLA-E knockout cells, and “B2M KO” indicates beta-2-microglobulin knockout cells. In FIG. 30, the x-axis of each plot represents effector (human PBMC-NK cells) to target (cancer cell line) ratios evaluated in co-cultures of the effector cells with the indicated target cells. In FIG. 30, the y-axis indicates the log 2 relative change in ratio of target cells to control cells in the co-culture relative to a similar co-culture grown in the absence of effector cells (i.e., NK cells). The cocultures initially contained approximately equal counts of control cells and one of control cells, HLA-E KO cells, or B2M KO cells (i.e., CTRL: CTRL; HLA-E KO: CTRL; and B2M KO: CTRL, respectively). Cells were counted using flow cytometry. In FIG. 30, the term “Target sg” indicates an sgRNA expressed by a target cell, and “Control sg” indicates an sgRNA expressed by a control cell, where sgRNA expression was used as a means to delete target genes with CRISPR/Cas9 technology and identify cells in the co-culture.

FIGS. 31A and 31B present bar graphs showing reversal of preferential natural killer (NK) cell killing of HLA-E knockout human cancer cells (HT-29 or SU.86.86) in vitro through NKG2A blockade with Monalizumab. In FIGS. 31A and 31B the term “ug/ml” indicates “μg/ml,” “CTRL” indicates “control” cells expressing HLA-E, and “HLA-E KO” indicates HLA-E knockout. In FIGS. 31A and 31B, the x-axis of each plot represents effector (NK-92MI or Primary NK cells) to target (HT-29 or SU.86.86 cells) ratios evaluated in co-cultures of the effector cells with the indicated target cells. In FIGS. 31A and 31B, the y-axis indicates the log 2 relative change in ratio of target cells to control cells in the co-culture relative to a similar co-culture grown in the absence of effector cells (i.e., NK cells). The cocultures initially contained approximately equal counts of control cells and one of control cells (bars to the left of each vertical line) or HLA-E KO cells (bars to the right of each vertical line).

FIGS. 32A and 32B present bar graphs and schematic diagrams showing that NKG2A+ primary human CD8+ T cells preferentially killed HLA-E knockout (KO) HT-29 tumor cells in vitro, as evaluated using a CD19/CAR T cells killing assay (FIG. 32A) and a redirected T cell killing assay (FIG. 32B). In FIGS. 32A and 32B, “CTRL” indicates “control” tumor cells that expressed both HLA-E and B2M, “HLA-E KO” indicates HLA-E knockout cells, and “B2M KO” indicates beta-2-microglobulin knockout cells. In FIGS. 32A and 32B, the x-axis of each plot represents effector (CD8+ T cells) to target (HT-29 cells) ratios evaluated in co-cultures of the effector cells with the target cells. In FIGS. 32A and 32B, the y-axis indicates the log 2 relative change in ratio of target cells to control cells in the co-culture relative to a similar co-culture grown in the absence of effector cells (i.e., T cells). (i.e., tumor cells expressing both HLA-E and B2M). The cocultures initially contained approximately equal counts of control cells and one of control cells, HLA-E KO cells, or B2M KO cells (i.e., CTRL: CTRL; HLA-E KO: CTRL; and B2M KO: CTRL, respectively). Cells were counted using flow cytometry. In the CD19/CAR T cell killing assay, the T cells expressed an anti-CD19 chimeric antigen receptor and the HT-29 cells expressed truncated human CD19 (see schematic diagram to the left of FIG. 32A). In the redirected T cell killing assay, the T cells expressed CD3 and CD28 and the HT-29 cells expressed a membrane-tethered anti-CD3 scFv and human CD80 (see schematic diagram to the left of FIG. 32B). The T cells were induced to express NKG2A and CD94 as described in FIG. 29 prior to being co-cultured with the target cells. In each of FIGS. 32A and 32B, the leftmost four bars correspond to “CTRL: CTRL,” the middle set of four bars correspond to “HLA-E KO: CTRL,” and the rightmost set of four bars corresponds to “B2M KO: CTRL.” In FIGS. 32A and 32B, the term “Target sg” indicates an sgRNA expressed by a target cell, and “Control sg” indicates an sgRNA expressed by a control cell, where sgRNA expression was used as a means to delete target genes using CRISPR/Cas9 and to identify cells in the co-culture.

FIGS. 33A and 33B present bar graphs showing that loss of HLA-E sensitized human tumor cells (HT29 or A375 cells) to T cell killing more than or comparable to loss of PD-L1 (CD274). In FIGS. 33A and 33B the bars to the left of the dotted line represent data corresponding to HLA-E knockout tumor cells and the bars to the right of the dotted line represent data corresponding to PD-L1 (CD274) knockout tumor cells. In FIGS. 33A and 33B, the y-axis indicates the log 2 change in ratio of target cells to control cells relative to the ratio observed in mice that did not contain effector cells (i.e., T cells). In FIGS. 33A and 33B, “NSG-SGM3” indicates NSG-SGM3 mice with the indicated tumor cells (i.e., HLA-E knockout or CD274 knockout HT29 or A375 tumor cells) but without any T cells administered, “NSG-SGM3+ T cells” indicates NSG-SGM3 mice containing human T cells and administered the indicated tumor cells, and “NSG-SGM3+ T cells+anti-PD-1” indicates NSG-SGM3 mice containing human T cells, administered the indicated tumor cells, and administered an anti-PD-1 antibody. In FIGS. 33A and 33B, “input” indicates relative abundance of knockout tumor cells to control cells at time of injection and “output” indicates the relative abundance of knockout tumor cells to control cells for a co-culture where the knockout tumor cells and control cells (i.e, the “input”) were grown in vitro for the same amount of time that cells were allowed to grow in the in vivo experiments. All mice were initially administered and all of the co-cultures initially contained approximately equal counts of the target HLA-E or CD274 knockout tumor cells and control tumor cells (i.e., tumor cells expressing both HLA-E and CD274). All data plotted in FIG. 33A was normalized to relative abundance of target:control cells in the NSG-SGM3 mice that did not receive T cells.

### DETAILED DESCRIPTION OF THE INVENTION

The disclosure features compositions containing chimeric antigen receptor (CAR) immune cells that have been modified to reduce and/or eliminate expression or activity of a natural killer cell lectin (NKG2) polypeptide and/or a cluster of differentiation 94 (CD94) polypeptide, and methods for use thereof to treat a neoplasia (e.g., skin, colon, pancreas, lung, and kidney cancer).

The invention is based, at least in part, upon the discovery that loss of IFNγ signaling by tumor cells sensitizes most (i.e., 6 of 8) cancer models to Immune Checkpoint Blockade (ICB). Using in vivo screening data, transcriptional profiling, and genetic interaction studies, it was revealed that the immune-inhibitory effects of tumor IFN sensing are the direct result of tumor upregulation of classical and nonclassical MHC-I genes. The interferon-MHC-I axis can inhibit anti-tumor immunity through two mechanisms: first, upregulation of classical MHC-I inhibits the cytotoxicity of natural killer cells, which are activated by ICB. Second, IFN-mediated upregulation of Qa-1b directly inhibits cytotoxicity by effector CD8+ T cells via the NKG2A/CD94 receptor, which is induced on CD8+ T cells by ICB. Finally, it was shown that high interferon-stimulated gene expression in is associated with decreased survival or poor response in ICB-treated ccRCC and advanced melanoma patients. The studies described in the Examples below reveal the underlying mechanism to explain the inhibitory role of tumor IFN sensing, demonstrating that IFN-mediated upregulation of classical and non-classical MHC-I inhibitory checkpoints can facilitate immune escape.

The findings described herein resulted from studies designed to comprehensively assess immune evasion strategies active in the tumor microenvironment in tumors with distinct genetic drivers and tissues of origin. To identify immune-related dependencies across preclinical tumor models, genome-scale and sub-genome-scale in vivo loss-of-function CRISPR screens were conducted across eight transplantable mouse tumor models representing a diversity of cancer types. These screens identified many new immunotherapy targets and resistance mechanisms, displaying both tumor specific and common patterns of dependency. Strikingly, loss of tumor-intrinsic interferon sensing sensitized most tumors to immune checkpoint blockade (ICB), a surprising finding given the importance of IFNγ for immune surveillance of cancer, and the association of loss-of-function mutations in Jak1 and Jak2 with immune checkpoint blockade (ICB) resistance.

Given the importance of IFNγ to anti-tumor immunity, screening data, transcriptional profiling, and in vivo mouse models were leveraged to determine the mechanism of IFN-mediated resistance to anti-tumor immunity and ICB. It was demonstrates that tumor IFN sensing inhibits NK cells through the upregulation of classical MHC-I and CD8+ T cells via the non-classical MHC-I Qa-1b. Using patient data, it was shown that the prognostic effect of IFN inflammation on survival is dependent on the nature of the immune response to the tumor, with interferon-stimulated gene (ISG) expression predicting response in T cell-dominant tumors and resistance in tumors with transcriptional profiling, and in vivo mouse models to determine the mechanism of IFN-mediated resistance to anti-tumor immunity and ICB. We demonstrate that tumor IFN sensing inhibits NK cytotoxicity. It was shown that tumor IFN sensing inhibits higher NK cell surveillance. Serum proteomics analysis of anti-PD-1-treated advanced melanoma patients revealed that IFN-mediated resistance is pronounced in on-treatment samples and high IFN signaling is predictive of disease progression. This study reveals that resistance to immune checkpoint blockade (ICB) mediated by tumor interferon (IFN) sensing and upregulation of MHC-I and Qa-1^{b} (HLA-E) was a conserved adaptive resistance mechanism at play in most tumor microenvironments and that blocking these inhibitory axes can overcome resistance to immune checkpoint blockade. In particular, it was found that immune cells modified to reduce or eliminate expression or activity of a natural killer cell lectin (NKG2) polypeptide and/or a cluster of differentiation 94 (CD94) polypeptide are less susceptible to inhibition by neoplastic cells.

The invention is also based, at least in part, upon the discovery that loss of 6p21.3 and/or low ISG expression levels relative to a reference predicted that a neoplasia would be responsive to immunotherapy, and presence of 6p21.3 and high ISG expression levels relative to a reference neoplasia predicted that the neoplasia would be resistant to immunotherapy. Also, interferon-stimulated gene (ISG) expression predicted response in T cell-dominant tumors and resistance in tumors with higher NK cell surveillance.

Accordingly, the present disclosure provides CAR T cells that have improved resistance to inhibition by neoplastic cells, where the CAR T cells have been modified to reduce or eliminate expression or activity of a natural killer cell lectin A (NKG2A) polypeptide and/or a cluster of differentiation 94 (CD94) polypeptide.

### NKG2A/CD94 Receptor

In various aspects, the present disclosure provides CAR T cells that have been modified to reduce and/or eliminate expression and/or activity of NKG2A and/or CD94. NKG2A/CD94 is an inhibitory receptor that has HLA-E (Qa-1 in mice) as its ligand. NKG2A is a transmembrane protein type II that dimerizes with CD94 to form a functional heterodimeric receptor. CD94 contains a short cytoplasmic domain and is responsible for signal transduction. When the NKG2A/CD94 receptor on the surface of an immune cell (e.g., an NK cell or a T cell) is bound by HLA-E, the immune cell becomes inhibited. It is demonstrated in the Examples provided herein that one way that a neoplasia can evade immune cells is by expressing HLA-E on the surface thereof to inactivate immune cells by way of the NKG2A/CD94 receptor. Therefore, in various aspects, the invention provides CAR T cells that have been modified to reduce or eliminate expression and/or activity of NKG2A and/or CD94 to improve the ability of the CAR T cells to resist inactivation by neoplastic cells.

### Genome Editing

In various embodiments, immune cells of the present disclosure are modified using genome editing. Immune cells can be modified by knocking out (e.g., by deletion) a target gene(s) (e.g., an NKG2A or CD94 gene). Gene editing tools provide the ability to manipulate the DNA sequence of a cell (e.g., to delete a target gene) at a specific chromosomal locus, without introducing mutations at other sites of the genome. This technology effectively enables the researcher to manipulate the genome of a subject's cells in vitro or in vivo.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule may be introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. In some embodiments, no donor DNA molecule is introduced and the double-stranded break is repaired by the error-prone non-homologous end joining NHEJ pathway leading to knock-out or deletion of the target gene (e.g., through the introduction of indels or nonsense mutations). In some embodiments, an endonuclease(s) can be targeted to at least two distinct chosen sites located within a gene sequence so that chromosomal double strand breaks at the distinct sites leads to excision and deletion of a nucleotide sequence flanked by the two distinct sites.

In some embodiments, the chosen site is associated with or disposed within a nucleotide sequence encoding a gene selected from Klrc1 (NKG2A) and (Klrd1) CD94.

Current genome editing tools use the induction of double strand breaks (DSBs) to enhance gene manipulation of cells, including the deletion or knockout of genes. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for ˜18 bp sequences in the genome.

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121):823-6). Genetic manipulation using engineered nucleases has been demonstrated in tissue culture cells and rodent models of rare diseases.

CRISPR has been used in a wide range of organisms including baker's yeast (S. cerevisiae), zebra fish, nematodes (C. elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (E. coli, Y. pest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

Guide RNA (gRNA)

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

### Chimeric Antigen Receptors and CAR-T Cells

The invention provides immune cells that express chimeric antigen receptors (CARs) and that have been modified to reduce or eliminate expression or activity of an NKG2A polypeptide and/or a CD94 polypeptide. Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a neoplastic cell. Because the CAR-T cells can act independently of major histocompatibility complex (MHC), activated CAR-T cells can kill the neoplastic cell expressing the antigen.

Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating a neoplasia (e.g., skin, colon, pancreas, lung, and kidney cancer). In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

One technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the invention are effector T cells. In some embodiments, the effector T cell is a naïve CD8⁺ T cell, a cytotoxic T cell, a natural killer T (NKT) cell, a natural killer cell, a gammadelta T cell (γδ T cell), or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, the immune effector cell is any other subset of T cells.

Chimeric antigen receptors as contemplated in the present invention comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death of the antigen expressing cell. In some embodiments of the present invention, the chimeric antigen receptor further comprises a linker.

In various embodiments, the CAR specifically binds CD19 or any other antigen that can be targeted by a chimeric antigen receptor (CAR), such as BCMA, Mesothelin, MUC1, MUC16, GD2, CD79b19, April, EGFR, EGFRvIII, IL13Ra, HLA-G, or PSMA. Further non-limiting examples of antigens that can be bound by a CAR of the present disclosure include those described in Xu, et al. “The development of CAR design for tumor CAR-T cell therapy,” Oncotarget, 9(17) doi: 10.18632/oncotarget.24179, the disclosure of which is incorporated herein by reference in its entirety for all purposes.

Chimeric antigen receptors, or any polypeptide of the present disclosure, can be delivered to an immune cell using a polynucleotide encoding the chimeric antigen receptor or polypeptide. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

### Extracellular Binding Domain

The chimeric antigen receptors of the invention include an extracellular binding domain. The extracellular binding domain of a chimeric antigen receptor contemplated herein comprises an amino acid sequence of an antibody, or an antigen binding fragment thereof, that has an affinity for a specific antigen. In some embodiments, the antigen is CD19.

In some embodiments the chimeric antigen receptor comprises an amino acid sequence of an antibody. In some embodiments, the chimeric antigen receptor comprises the amino acid sequence of an antigen binding fragment of an antibody. The antibody (or fragment thereof) portion of the extracellular binding domain recognizes and binds to an epitope of an antigen. In some embodiments, the antibody fragment portion of a chimeric antigen receptor is a single chain variable fragment (scFv). An scFv comprises the light and variable fragments of a monoclonal antibody. In other embodiments, the antibody fragment portion of a chimeric antigen receptor is a multichain variable fragment, which can comprise more than one extracellular binding domains and therefore bind to more than one antigen simultaneously. In a multiple chain variable fragment embodiment, a hinge region may separate the different variable fragments, providing necessary spatial arrangement and flexibility.

In other embodiments, the antibody portion of a chimeric antigen receptor comprises at least one heavy chain and at least one light chain. In some embodiments, the antibody portion of a chimeric antigen receptor comprises two heavy chains, joined by disulfide bridges and two light chains, wherein the light chains are each joined to one of the heavy chains by disulfide bridges. In some embodiments, the light chain comprises a constant region and a variable region. Complementarity determining regions residing in the variable region of an antibody are responsible for the antibody's affinity for a particular antigen. Thus, antibodies that recognize different antigens comprise different complementarity determining regions. Complementarity determining regions reside in the variable domains of the extracellular binding domain, and variable domains (i.e., the variable heavy and variable light) can be linked with a linker or, in some embodiments, with disulfide bridges.

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell. For example, in some embodiments, the antigen is expressed in a neoplastic cell.

### Transmembrane Domain

The chimeric antigen receptors of the invention include a transmembrane domain. The transmembrane domain of the chimeric antigen receptors described herein spans the CAR-T cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell.

### Intracellular Signaling Domain

The chimeric antigen receptors of the invention include an intracellular signaling domain. The intracellular signaling domain is the intracellular portion of a protein expressed in a T cell that transduces a T cell effector function signal (e.g., an activation signal) and directs the T cell to perform a specialized function. T cell activation can be induced by a number of factors, including binding of cognate antigen to the T cell receptor on the surface of T cells and binding of cognate ligand to costimulatory molecules on the surface of the T cell. A T cell co-stimulatory molecule is a cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule. Activation of a T cell leads to immune response, Such as T cell proliferation and differentiation (see, e.g., Smith-Garvin et al., Annu. Rev. Immunol., 27:591-619, 2009). Exemplary T cell signaling domains are known in the art.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

### Characterizing Cells

In various aspects, the methods of the disclosure involve characterizing a neoplasia (e.g., skin, colon, pancreas, lung, and kidney cancer). In some instances, the characterization of a neoplasia involves determining whether or not the neoplasia has a loss of 6p21.3 and/or measuring expression levels of one or more interferon-stimulated genes (ISGs) in the neoplasia. Such characterization and measurements can be carried out using methods familiar to one of skill in the art, which include, but are not limited to, those described herein.

In some cases, the methods provided herein can be used to detect loss of expression of a polypeptide (e.g., NKG2A (Klrc1) and/or CD94 (Klrd1)) in a cell (e.g., a modified immune cells, such as a CAR T cell). The methods can also be used to detect expression of a heterologous polypeptide in a cell (e.g., a chimeric antigen receptor).

In some instances, measuring expression levels of one or more interferon-stimulated genes (ISGs) involves measuring expression levels for about, at least about, or no more than about 1, 2, 3, 4, 5, 6, 7, 8, 9, 10, 11, 12, or more of the following genes: ADAR, APOL6, ARID5B, ARL4A, AUTS2, B2M, BANK1, BATF2, BPGM, BST2, BTG1, C1R, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLR1, CMPK2, CMTR1, CSF2RB, CXCL10, CXCL11, CXCL9, DDX58, DDX60, DHX58, EIF2AK2, EIF4E3, EPSTI1, FAS, FCGR1A, FGL2, FPR1, GBP4, GBP6, GCH1, GPR18, GZMA, HELZ2, HERC6, HIF1A, HLA-A, HLA-B, HLA-DMA, HLA-DQA1, HLA-DRB1, HLA-G, ICAM1, IDO1, IFI27, IFI30, IFI35, IFI44, IFI44L, IFIH1, IFIT1, IFIT2, IFIT3, IFITM2, IFITM3, IFNAR2, IL10RA, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, MARCHF1, METTL7B, MT2A, MTHFD2, MVP, MX1, MX2, MYD88, NAMPT, NCOA3, NFKB1, NFKBIA, NLRC5, NMI, NOD1, NUP93, OAS2, OAS3, OASL, OGFR, P2RY14, PARP12, PARP14, PDE4B, PELI1, PFKP, PIM1, PLA2G4A, PLSCR1, PML, PNP, PNPT1, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SRI, SSPN, ST3GAL5, ST8SIA4, STAT1, STAT2, STAT3, STAT4, TAP1, TAPBP, TDRD7, TNFAIP2, TNFAIP3, TNFAIP6, TNFSF10, TOR1B, TRAFD1, TRIM14, TRIM21, TRIM25, TRIM26, TXNIP, UBE2L6, UPP1, USP18, VAMP5, VAMP8, VCAM1, WARS1, XAF1, XCL1, ZBP1, and ZNFX1. In some cases, measuring expression levels of one or more ISGs involves measuring expression levels for IFNγ and/or HLA-E. In embodiments, the methods of the disclosure involve determining an ISG score based upon measured levels of one or more ISG genes. In some cases, a high ISG score relative to a reference indicates that a neoplasia will be or has an increased probability of being resistant to immunotherapy. In embodiments, the ISG score is an IFNγ score calculated using ssGSEA (Barbie, D. A. et al. Systematic RNA interference reveals that oncogenic KRAS-driven cancers require TBK1. Nature 462, 108-112 (2009)) using the Hallmark Interferon Gamma Response gene set (Liberzon, A. et al. The Molecular Signatures Database (MSigDB) hallmark gene set collection. Cell Syst 1, 417-425 (2015)). In embodiments, an alteration in expression of any of the aforementioned genes is by at least about 5, 10, 15, 20, 25, 30, 35, 40, 45, 50, 60, 70, 80, 90, or 100%. In another embodiment, the alteration is a significant increase or reduction in the level or activity of any of the aforementioned genes or their associated polypeptides.

In various cases, loss of 6p21.3 is detected as a loss of expression of and/or a gene encoding one or more of a TAP1, TAP2, TAPBP, PSMB8, and/or PSMB9 polypeptide. The presence or absence of a gene can be determined using methods familiar to one of skill in the art including, but not limited to, the gene sequencing methods described herein. In embodiments, loss of 6p21.3 indicates that a neoplasia is likely to be responsive to immunotherapy; whereas, presence of 6p21.3 indicates that a neoplasia is likely to be resistant to immunotherapy.

Gene expression levels can be detected using biomarkers (e.g., polynucleotides or polypeptides). In some cases a biomarker is a polynucleotide (e.g., mRNA, a portion of a genome, and/or a gene). The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are describe below.

Detection by Sequencing and/or Probes

In particular embodiments, the biomarkers of the invention are measured by a sequencing- and/or probe-based technique (e.g., RNA-seq).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24; 109(4):1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Detecting a target polynucleotide sequence or fragment thereof associated with a biomarker that hybridizes to a probe sequence may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various detection methods may be used and are described as follows.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a biomarker.

Detection of the expression level of a biomarker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

In embodiments, levels of biomarkers in a sample are quantified using targeted sequencing. Methods for targeted sequencing are well known in the art (see, e.g., Rehm, “Disease-targeted sequencing: a cornerstone in the clinic”, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, β-galactosidase, urease, alkaline phosphatase or peroxidase, avidin/biotin complex.

Methods used to detect or quantify binding of a probe to a target biomarker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the invention.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays (e.g., flow cytometry), and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In embodiments, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In embodiments, a sample is analyzed by means of a protein biochip (also known as a protein microarray). Such biochips are useful in high-throughput low-cost screens to identify alterations in the expression or post-translation modification of a biomarker, or a fragment thereof. In embodiments, a protein biochip of the invention binds a biomarker present in a sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions include, as non-limiting examples, temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the biomarkers of this invention are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the biomarker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Treatments

The methods provided herein can be used for treating a subject for a neoplasia (e.g., skin, colon, pancreas, lung, and kidney cancer) and/or for selecting a subject an agent (e.g., using CAR T cells and/or an immune checkpoint blockade (ICB)) to administer to a subject to treat a neoplasia (e.g., skin, colon, pancreas, lung, and kidney cancer). In embodiments, the methods provided herein can be used for selecting a subject for treatment using an immunotherapy. In embodiments, a subject is administered, for example, checkpoint blockade therapy comprising a PD-1/PD-L1 checkpoint inhibitor (e.g., Nivolumab, Pembrolizumab, Atezolizumab, Avelumab, Durvalumab, Cemiplimab, Dostarlimab). Non-limiting examples of checkpoint inhibitors include anti-PD-1 and anti-CTLA-4 antibodies (e.g., ipilimumab). Thus, the methods provided herein include methods for the treatment of a neoplasia (e.g., cancer, such as skin, colon, pancreas, lung, and kidney cancer). Generally, the methods provided herein include administering a therapeutically effective amount of a treatment as provided herein, to a subject who is in need of, or who has been determined to be in need of, such treatment. The treatments can be selected based upon interferon-stimulated gene expression levels (e.g., an IFNγ score). Treatments can be selected based upon loss of 6p21.3. In some cases, a high IFNγ score selects a patient for administration of an immune checkpoint blockade comprising an NKG2A/CD94 receptor inhibitor.

Non-limiting examples of NKG2A/CD94 receptor inhibitors include anti-NKG2A and/or anti-CD94 antibodies, such as Monalizumab.

In embodiments, the methods provided herein can be used for selecting a subject for inclusion in or exclusion from a clinical trial. In embodiments, the clinical trial is designed to test the efficacy of an immunotherapy. The methods provided herein can assist in selecting patients likely to respond to a particular agent for inclusion in a clinical trial for the study of patient response to the agent. In embodiments, the methods of the invention involve using interferon-stimulated gene expression levels and/or loss of 6p21.3 to separate subjects likely to respond to an agent from those likely not to respond to the agent.

The methods provided herein include selecting a subject for and/or administering to a subject having or having a propensity to develop a neoplasia a treatment that includes a therapeutically effective amount of an immunotherapeutic agent, such as a CAR T cell of the disclosure (e.g., CAR T cells modified to reduce or eliminate expression or activity of NKG2A and/or CD94) and/or an immune checkpoint blockade. In some cases, the immune checkpoint blockade agent comprises a PD-1/PD-L1 pathway inhibitor.

The protein, Programmed Death 1 (PD-1), is an inhibitory member of the CD28 family of receptors, that also includes CD28, CTLA-4, ICOS and BTLA. PD-1 is expressed on activated B cells, T cells, and myeloid cells. The structure and function of PD-1 is further described in e.g., Okazaki et al., Curr. Opin. Immunol., 14:779-782 (2002); and Bennett et al., J. Immunol., 170:711-718 (2003), the teachings of each of which are incorporated herein by reference in their entireties. Non-limiting examples of PC-1/PD-L1 pathway-inhibitors include anti-PD-1 antibodies and/or anti-CTLA-4 antibodies.

Two ligands for PD-1 include PD-L1 (B7-H1, also called CD274 molecule) and PD-L2 (b7-DC). The PD-L1 ligand is abundant in a variety of human cancers. The interaction of PD-L1 with PD-1 generally results in a decrease in tumor infiltrating lymphocytes, a decrease in T-cell receptor mediated proliferation, and immune evasion by the cancerous cells. See, e.g., Dong et al., Nat. Med., 8:787-789 (2002); Blank et al., Cancer Immunol. Immunother., 54:307-314 (2005); and Konishi et al., Clin. Cancer Res., 10:5094-5100 (2004), the teachings of each of which have been incorporated herein by reference in their entireties.

Inhibition of the interaction of PD-1 with PD-L1 can restore immune cell activation, such as T-cell activity, to reduce tumorigenesis and metastasis, making PD-1 and PD-L1 advantageous cancer therapies. See, e.g., Yang J., et al., J Immunol. August 1: 187(3): 1113-9 (2011), the teachings of which has been incorporated herein by reference in its entirety.

Non-limiting examples of PD-1/PD-L1 inhibitors that can be administered to a subject in need of treatment include Atezolizumab (Tecentriq, MPDL3280A, RG7446); Avelumab (Bavencio, MSB0010718C); BMS-936559 (MDX-1105); Cemiplimab (Libtayo REGN-2810, REGN2810, cemiplimab-rwlc); Durvalumab (MED14736, MEDI-4736); Nivolumab (Opdivo ONO-4538, BMS-936558, MDX1106); and Pembrolizumab (Keytruda, MK-3475).

In some embodiments, the agent(s) provided herein (e.g., CAR T cells modified to reduce or eliminate expression or activity of NKG2A and/or CD94) is administered in combination with an additional chemotherapeutic agent. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time.

An effective amount of an agent can be administered in one or more administrations, applications or dosages. A therapeutically effective amount of a therapeutic compound or agent (i.e., an effective dosage) depends on the therapeutic compounds or agents selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents provided herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test agent which achieves a half-maximal inhibition of symptoms) as determined in cell culture. Such information can be used to more accurately determine useful doses in humans. Levels in plasma may be measured, for example, by high performance liquid chromatography.

Dosages and desired drug concentration of pharmaceutical compositions of the present disclosure may vary depending on the particular use envisioned. The determination of the appropriate dosage or route of administration (e.g., oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes) is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. In some embodiments, the dose amount of a CAR T cell is about, at least about, and/or no more than about 1e5 cells, 1e6 cells, 1e7 cells, 1e8 cells, 1e9 cells, 1e10 cells, 1e11 cells, 1e12 cells, 1e13 cells, 1e14 cells, 1e15 cells, or 1e16 cells. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Methods for characterizing the efficacy of a treatment for a neoplasia are well known in the art (e.g., computerized tomography (CT) scan, bone scan, magnetic resonance imaging (MRI), position emission tomography (PET) scan, ultrasound X-ray, biopsy, etc.).

### Pharmaceutical Compositions

Provided also are pharmaceutical compositions for use in treating a neoplasia (e.g., skin, colon, pancreas, lung, and kidney cancer). In an embodiment, the compositions include CAR T cells modified to reduce or eliminate expression or activity of an NKG2A and/or CD94 polypeptide, as described herein and an acceptable carrier, excipient, or diluent.

The agents of the disclosure (e.g., chemotherapeutic agents, CAR T cells, and/or immune checkpoint blockades (ICBs)) may be contained in any appropriate amount in any suitable carrier substance, and is/are generally present in an amount of 0.01-95% by weight of the total weight of the composition. The pharmaceutical composition may be provided in a form that is suitable for a parenteral (e.g., subcutaneous, intravenous, intramuscular, or intraperitoneal) administration route, such that the agent, such as a vector described herein, is systemically delivered.

The pharmaceutical compositions of the present invention can be prepared in accordance with known techniques. See, e.g., Remington, The Science And Practice of Pharmacy (21st ed. 2005). In general, the immune cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

The skilled artisan can readily determine the number of cells and amount of optional additives, vehicles, and/or carriers in compositions and to be administered in methods of the invention. Typically, additives are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein, and the time for sequential administrations can be ascertained without undue experimentation.

Pharmaceutical compositions may be formulated to release an agent substantially immediately upon administration or at any predetermined time or time after administration. The latter types of compositions are generally known as controlled release formulations, which include (i) compositions that create a substantially constant concentration of the agent within the body over an extended period of time; (ii) compositions that after a predetermined lag time create a substantially constant concentration of the drug within the body over an extended period of time; (iii) compositions that sustain action during a predetermined time period by maintaining a relatively constant, effective level in the body with concomitant minimization of undesirable side effects associated with fluctuations in the plasma level of the active substance (sawtooth kinetic pattern); (iv) compositions that localize action by, e.g., spatial placement of a controlled release composition adjacent to or in contact with a target site or location, e.g., in a region of a tissue or organ; (v) compositions that allow for convenient dosing, such that doses are administered, for example, once every one, two, or several weeks; and (vi) compositions that target a specific tissue or cell type.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. The pharmaceutical composition may be administered systemically.

The pharmaceutical composition may be in the form of a solution, a suspension, an emulsion, an infusion device, or a delivery device for implantation, or it may be presented as a dry powder to be reconstituted with water or another suitable vehicle before use. Apart from the agent (e.g., CAR T cells, immune checkpoint blockade (ICB), or other chemotherapeutic agent), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the pharmaceutical composition are formulated for intravenous delivery. As noted above, the compositions according to the described embodiments may be in a form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl, or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, compositions in accordance with the present disclosure can be used for treatment of any of a variety of diseases, disorders, and/or conditions.

### Hardware and Software

The present disclosure also relates to a computer system involved in carrying out the methods of the disclosure relating to both computations and sequencing. In the methods described herein, analyses (e.g., calculating expression levels and/or calculation of an ISG score and/or IFNγ score) can be performed on general-purpose or specially-programmed hardware or software. One can then record the results on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of sequences will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., aneuploidy, translocations, and/or MM driver mutations) observed in the data. In aspects, software used to analyze the data can include code that applies an algorithm to the analysis of the results. The software also can also use input data (e.g., biomarker measurements) to determine an ISG score.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in characterizing and/or treating a neoplasia (e.g., skin, colon, pancreas, lung, and kidney cancer). Kits of the instant disclosure may include one or more containers comprising an agent for characterization of a neoplasia and/or for treatment of a neoplasia. In some embodiments, the kits further include instructions for use in accordance with the methods of this disclosure. In some embodiments, these instructions comprise a description of use of the agent to characterize a neoplasia and/or use of the agent (e.g., CAR T cells) for treatment of a neoplasia (e.g., skin, colon, pancreas, lung, and kidney cancer). The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: In Vivo Loss of Function Screens Reveal Tumor-Intrinsic Sensitizers to Immunotherapy

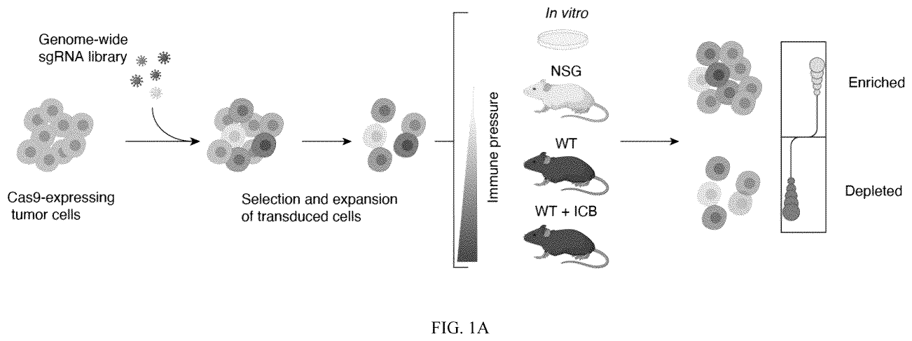

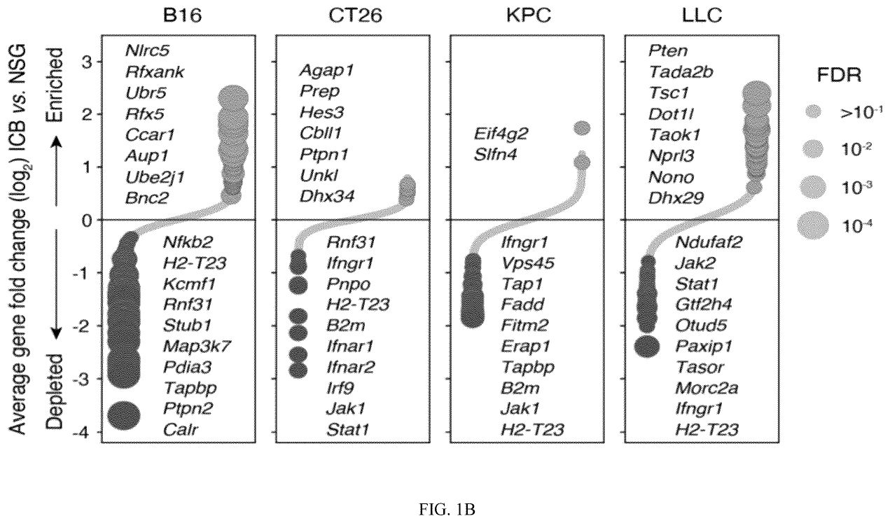

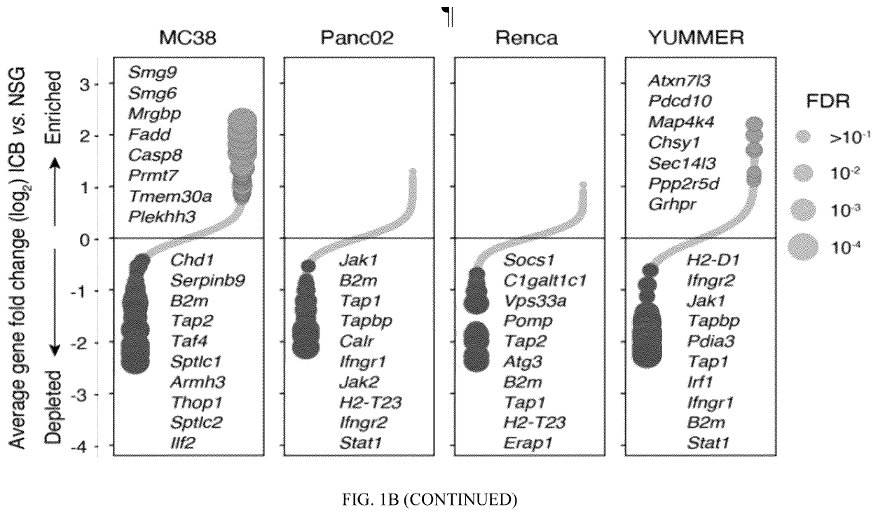

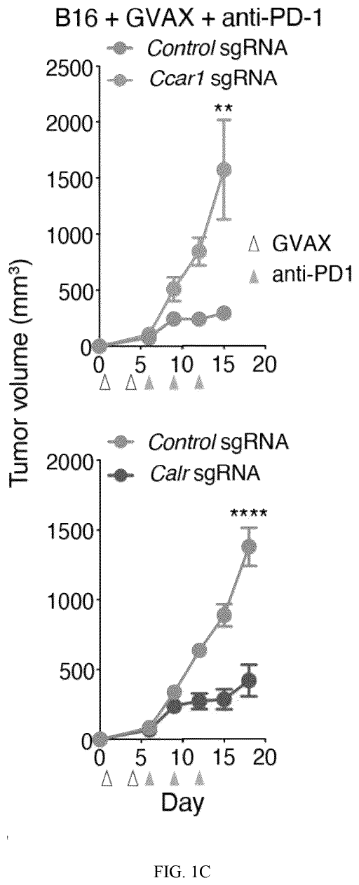

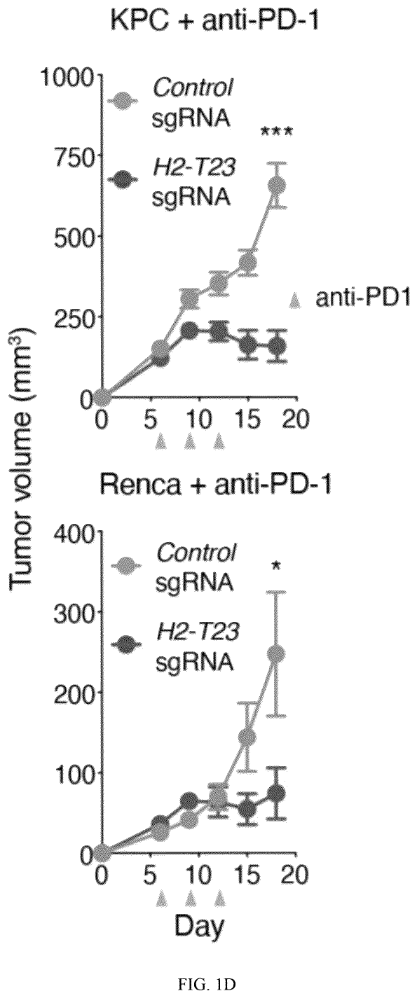

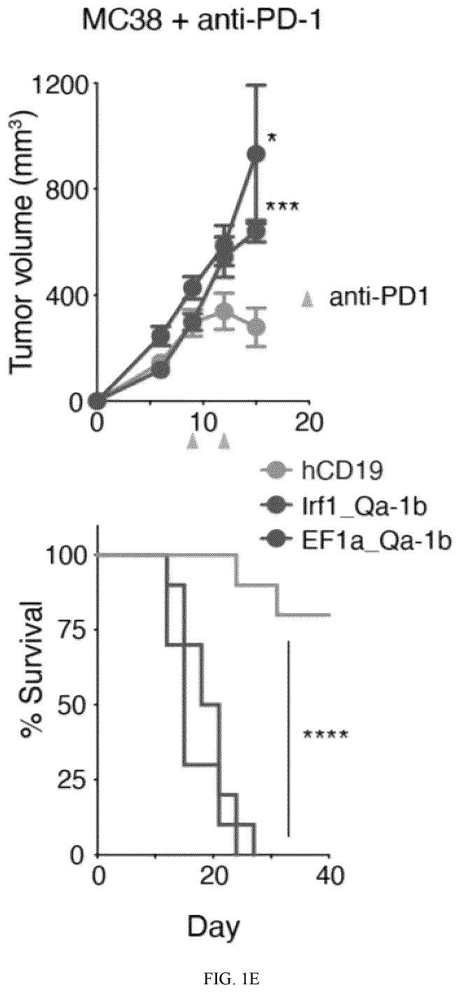

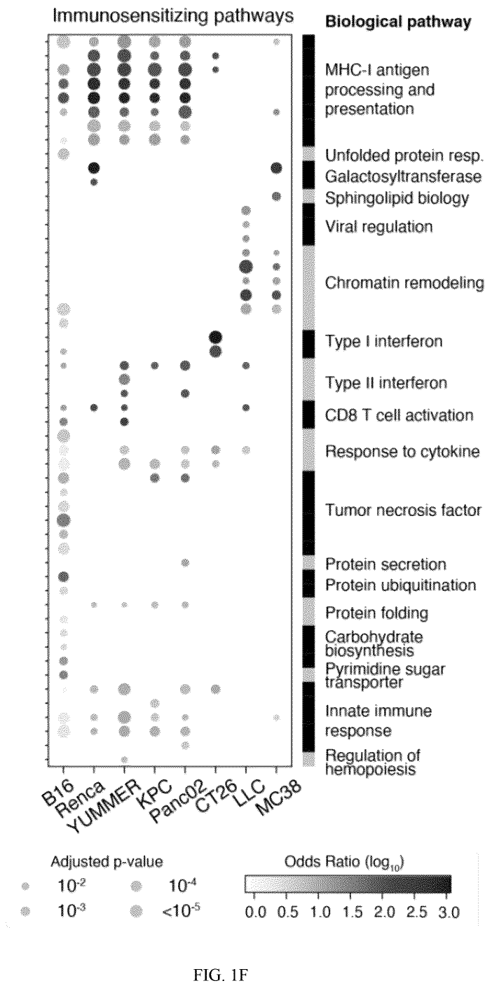

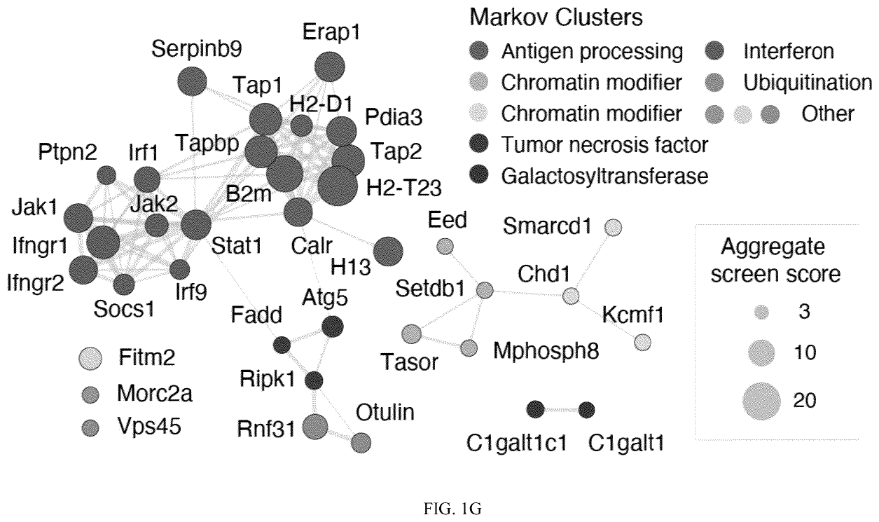

An in vivo pooled CRISPR screening approach (FIG. 1A) was used to systematically identify immune evasion genes in tumor cells under immune selective pressure (Manguso et al. Nature 547, 413-418 (2017)). Eight (8) genome-scale in vivo screens were performed in mouse transplantable tumor models treated with immune checkpoint blockade (ICB). In the process of optimizing screens at genome-scale, sub-genome scale in vivo screens were performed in a subset of the cancer models and the analysis of that data, including 2 sub-genome screens published using the same library (Manguso, R. T. et al. In vivo CRISPR screening identifies Ptpn2 as a cancer immunotherapy target. Nature 547, 413-418 (2017); and Dubrot, J. et al. In vivo screens using a selective CRISPR antigen removal lentiviral vector system reveal immune dependencies in renal cell carcinoma. Immunity (2021) doi:10.1016/j.immuni.2021.01.001), for a total dataset of 14 in vivo screens. The genome-scale library consisted of 19,280 genes, each targeted by 4 sgRNAs delivered in 4 separate cohorts (Doench, J. G. et al. Optimized sgRNA design to maximize activity and minimize off-target effects of CRISPR-Cas9. Nat. Biotechnol. 34, 184-191 (2016)). The sub-genome library targeted 2,368 genes with 4 sgRNAs per gene as described in Manguso, R. T. et al. In vivo CRISPR screening identifies Ptpn2 as a cancer immunotherapy target. Nature 547, 413-418 (2017); and Doench, J. G. et al. Optimized sgRNA design to maximize activity and minimize off-target effects of CRISPR-Cas9. Nat. Biotechnol. 34, 184-191 (2016). The 8 transplantable tumor models represented spontaneous, carcinogen-induced, and genetically engineered (GEM) backgrounds from 5 different cancer types (melanoma, pancreatic, lung, renal, and colon) used for preclinical studies (Table 1).

TABLE 1
Summary of the models used for in vivo screens: cell line origin,
mouse strain, and immune checkpoint blockade (ICB) regimen.

| Cell line | Tissue | Origin | Strain | ICB treatment (GW screens) |
|---|---|---|---|---|
| B16 | Skin | Spontaneous | C57BL6 | anti-PD-1 (100 ug at d 6, 9, 12) + GVAX (d 1, 4) |
| CT26 | Colon | Carcinogen | BALB/c | anti-PD-1 + anti-CTLA-4 (200 μg ea. at d 6, 9, |
| | | | | 12) |
| KPC | Pancreas | GEM | C57BL6 | anti-PD-1 + anti-CTLA-4 (100 μg ea. at d 6, 9, |
| | | | | 12) |
| LLC | Lung | Spontaneous | C57BL6 | anti-PD-1 + anti-CTLA-4 (200 μg ea. at d 6, 9, |
| | | | | 12) |
| MC38 | Colon | Carcinogen | C57BL6 | anti-PD-1 (50 ug at d 12, 15) |
| Panc02 | Pancreas | Carcinogen | C57BL6 | anti-PD-1 + anti-CTLA-4 (100 μg ea. at d 6, 9, |
| | | | | 12) |
| Renca | Kidney | Spontaneous | BALB/c | anti-PD-1 + anti-CTLA-4 (100 μg ea. at d 6, 9, |
| | | | | 12) |
| YUMMER | Skin | GEM | C57BL6 | anti-PD-1 + anti-CTLA-4 (100 μg ea. at d 6, 9, |
| | | | | 12) |

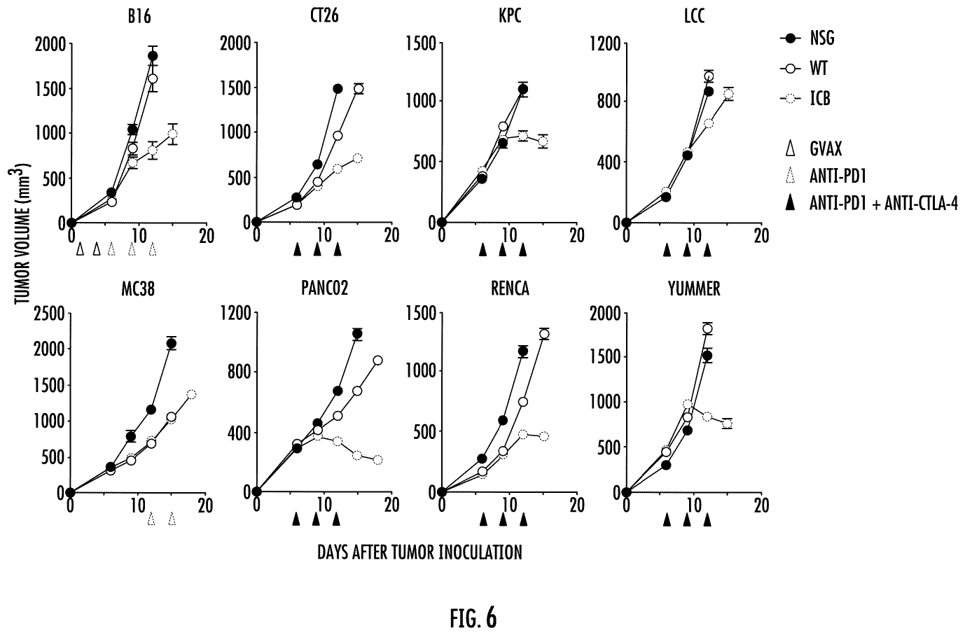

To identify sgRNAs with immune-dependent fitness effects, tumors were implanted subcutaneously into immunocompetent wild-type mice (WT) and immune checkpoint blockade (ICB)-treated WT mice, with immunodeficient NOD SCID I12rg^{-/-} (NSG) mice as controls. For immunotherapy treatment, B16 melanoma was treated with an irradiated GM-CSF-expressing tumor vaccine (GVAX) and anti-PD-1, and MC38 colon adenocarcinoma was treated with anti-PD-1 alone. All other tumor models were treated with a combination of anti-PD-1 and anti-CTLA-4 (Table 1). Endogenous anti-tumor immunity or an immunotherapy-dependent inhibition of tumor growth was observed in each model (FIG. 6).

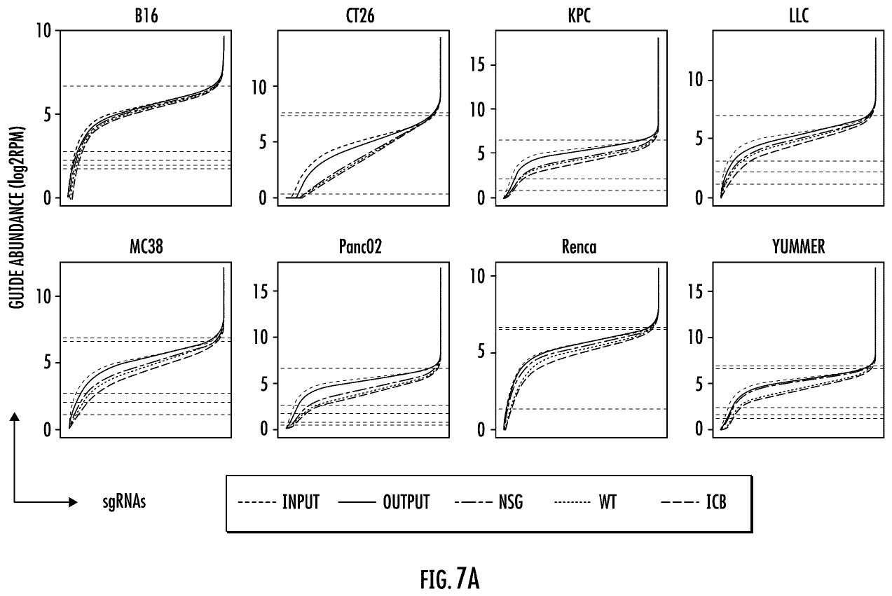

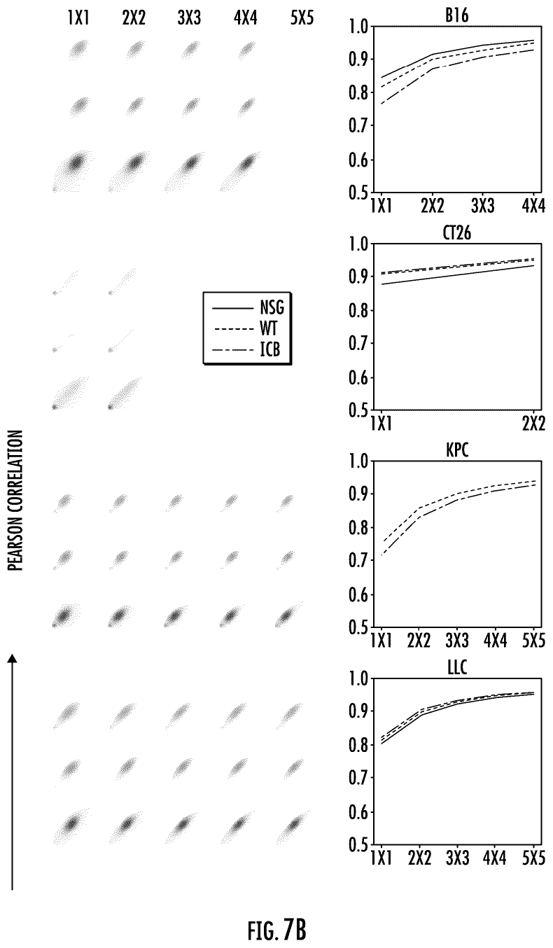

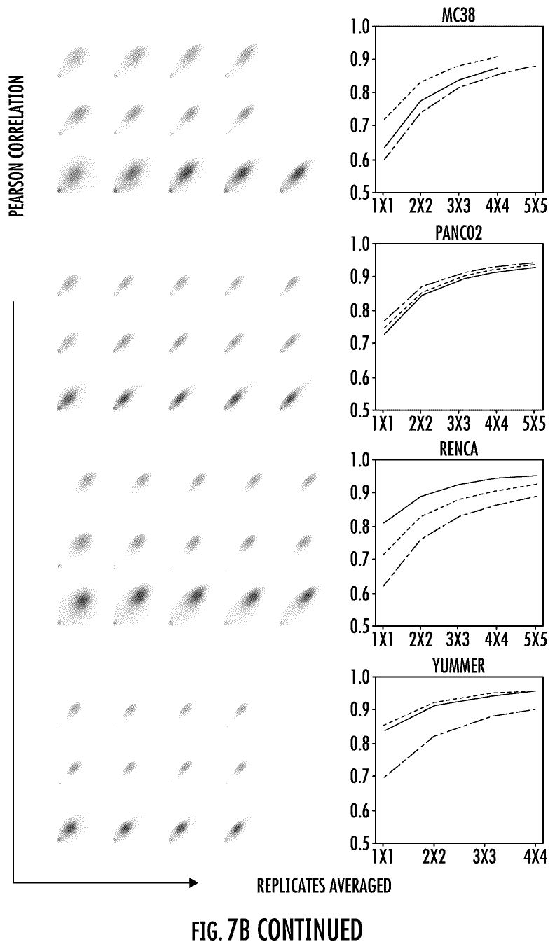

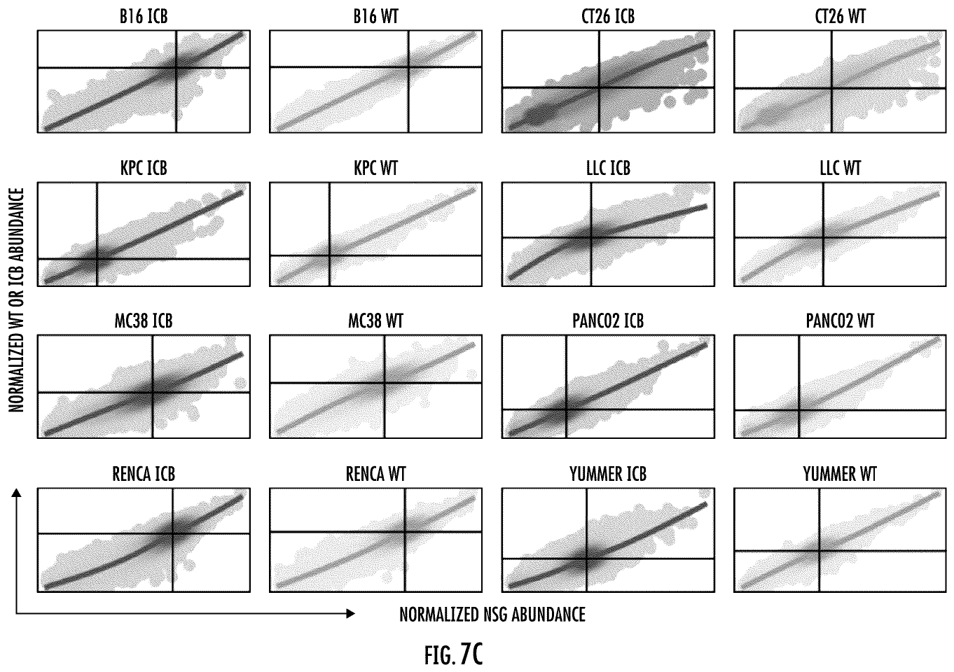

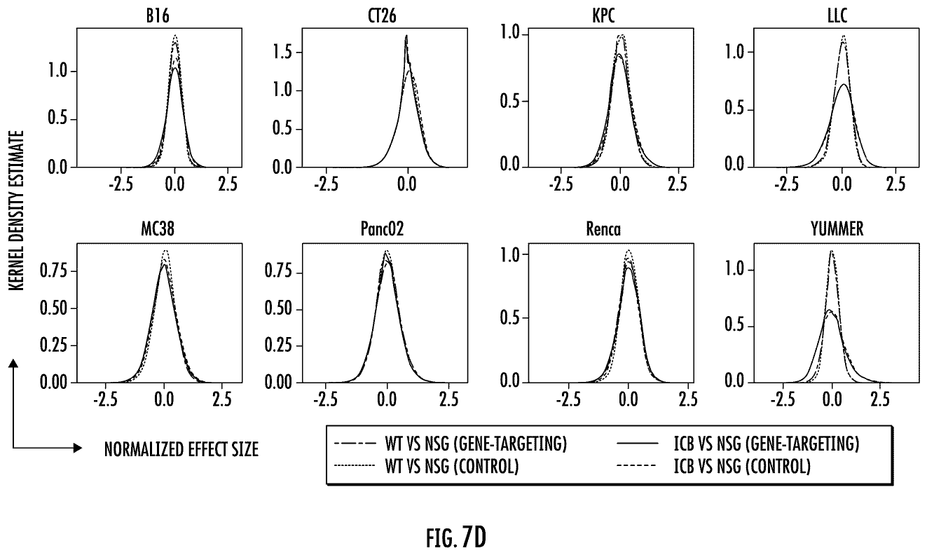

Quality control analyses indicated good screen performance. Across all screens, the majority of sgRNAs were well represented in all experimental conditions (FIG. 7A). Recovery of the in vivo libraries for each screen was confirmed by replicate autocorrelation saturation (Pearson correlation>0.8; FIG. 7B). To identify immune-dependent regulators, z-score normalization was carried out based on the control sgRNA distribution and fold change was calculated as the residual from a natural cubic spline fit to the wild type (WT) or ICB-treated groups and the NSG group (Wei, J. et al. Genome-wide CRISPR Screens Reveal Host Factors Critical for SARS-CoV-2 Infection. Cell 184, 76-91.e13 (2021)), resulting in normalized fold changes distributed around zero, with negative scores signifying that loss of gene expression sensitized to immune pressure and positive scores signifying that loss of gene expression conferred resistance (FIGS. 7C and 7D).

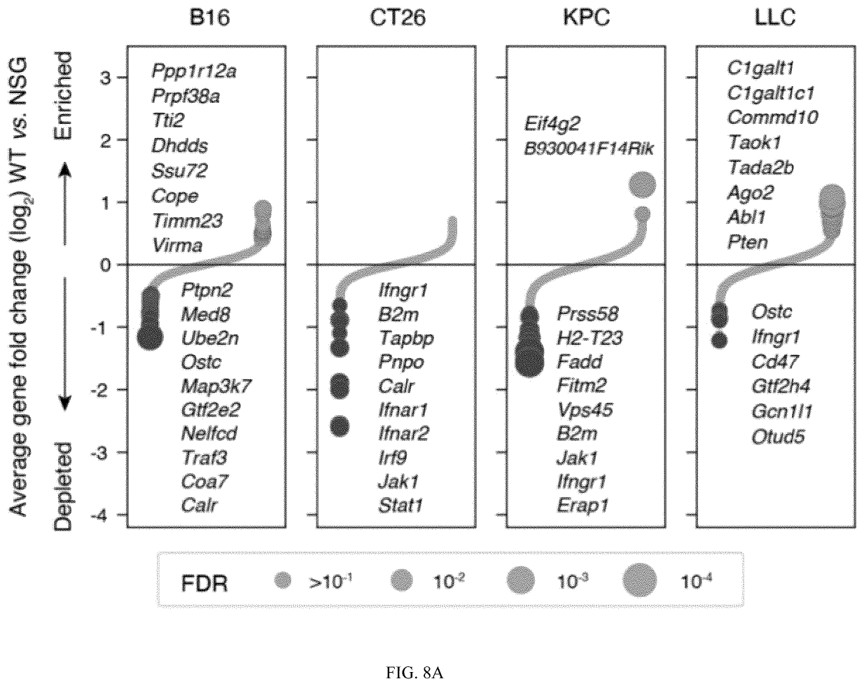

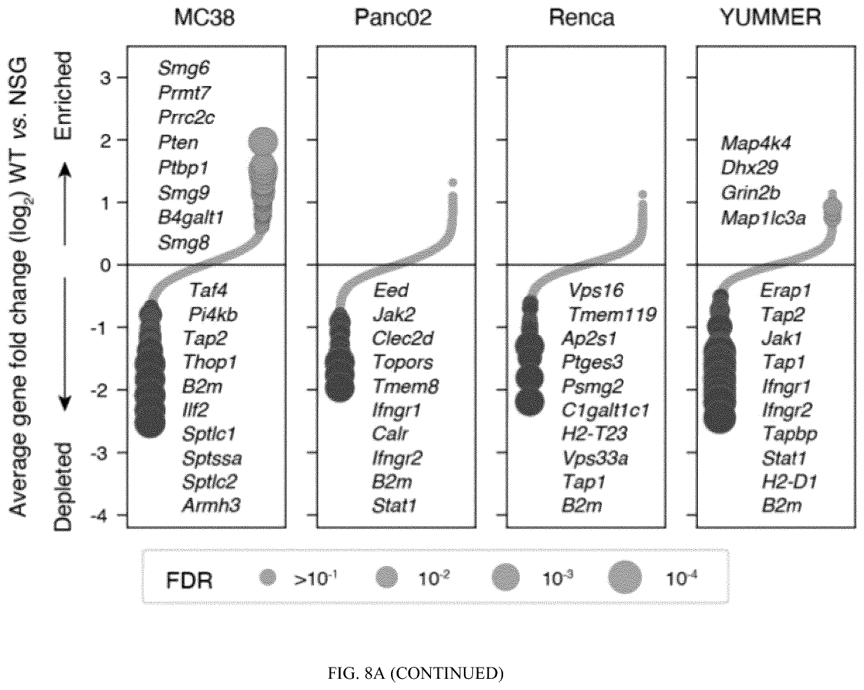

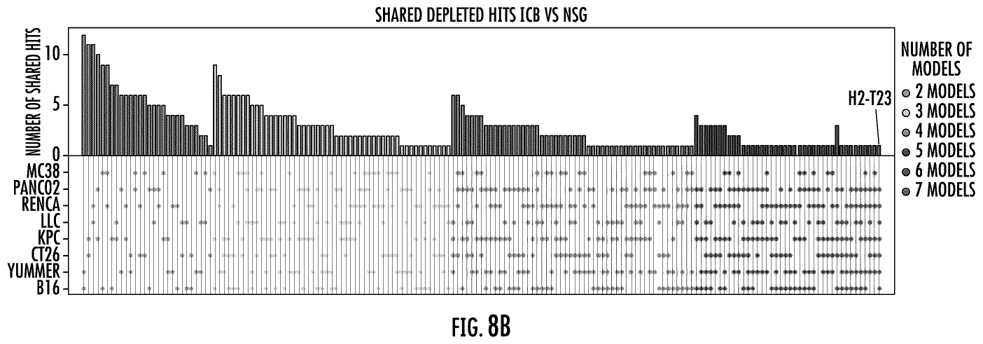

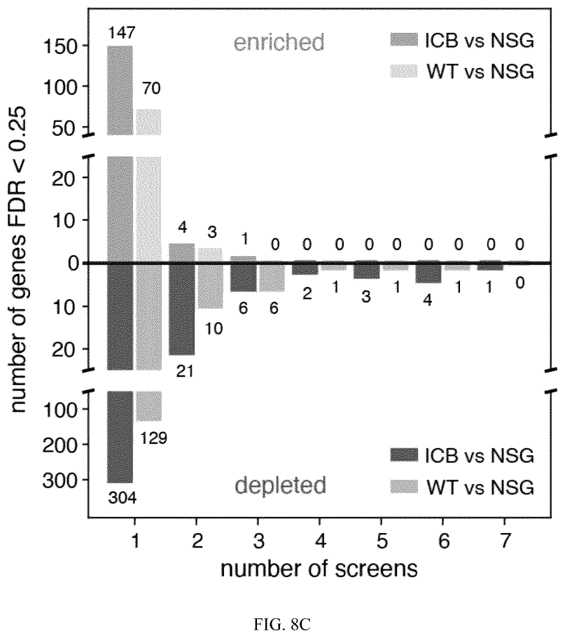

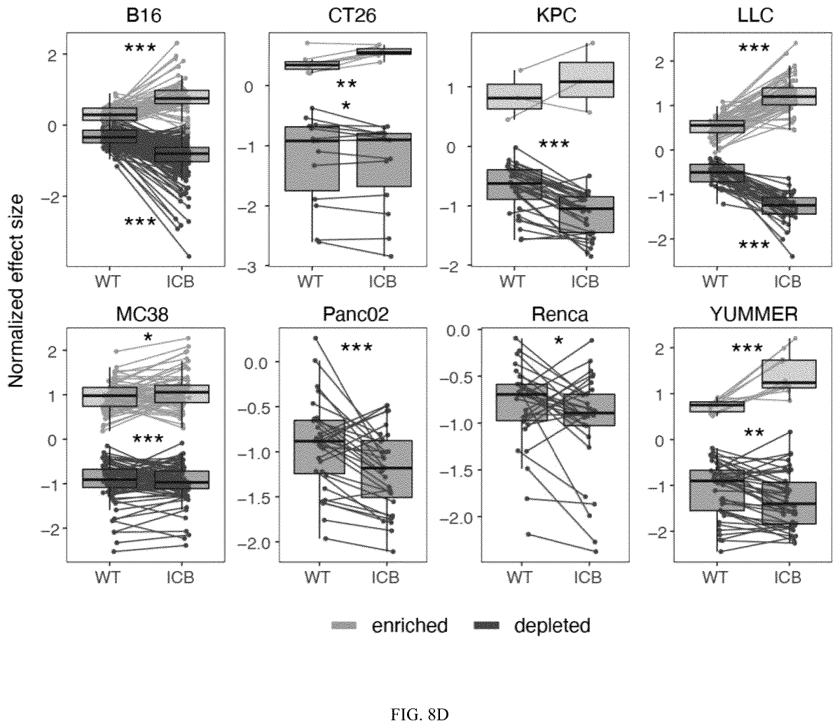

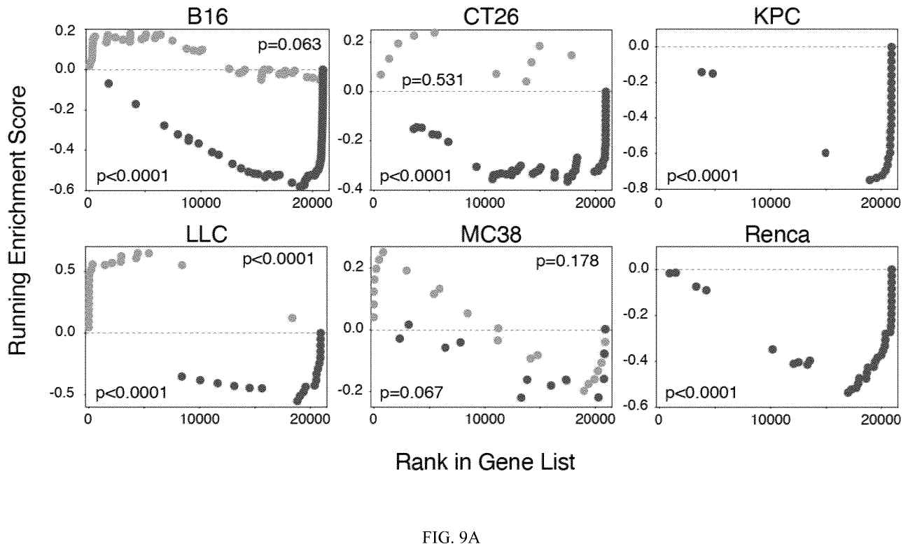

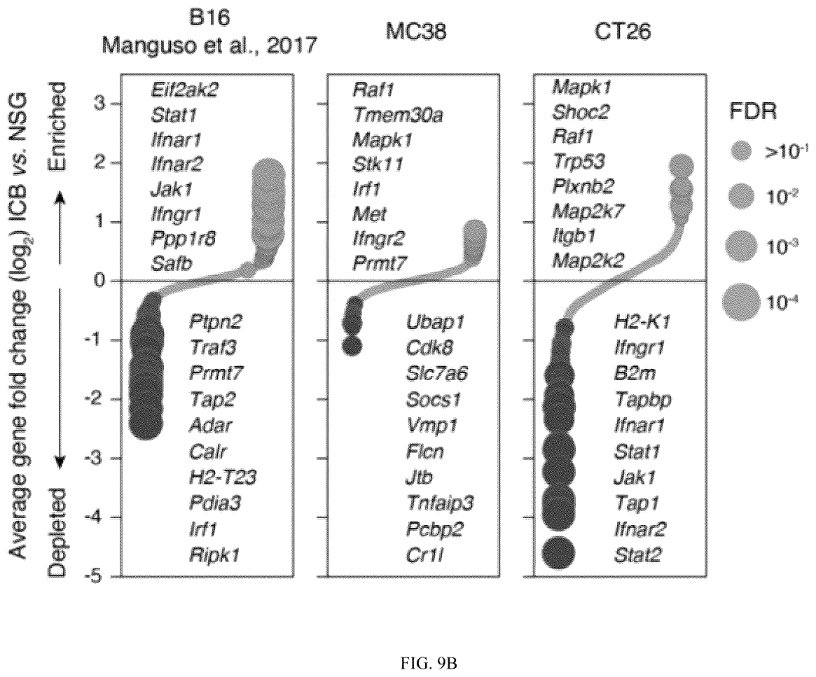

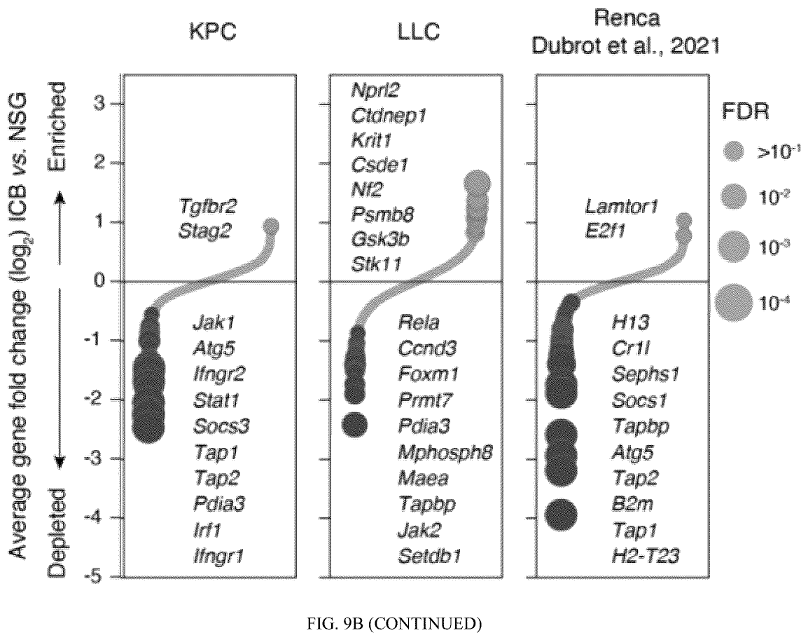

All screens revealed many genes that, when deleted, sensitize tumors to immune-dependent selective pressure, including gene deletions that sensitized two or more models to immunotherapy (FIGS. 1B, 8A, and 8B). For the 6 models that were screened at both genome and sub-genome scale, a highly significant enrichment of the hits from the sub-genome screens was observed in the genome-scale screening data from the same model, despite different sets of guides being used to target each gene in the two libraries, indicating strong technical reproducibility across screens (FIG. 9A). The number of enriched (resistance-enhancing) gene targets varied greatly by cancer model; for instance, Panc02 and Renca produced no statistically significant enriched hits despite a large number of significant depleted (sensitizing) sgRNAs (FIGS. 1B and 8A). For each model with the exception of YUMMER, immune checkpoint blockade (ICB) treatment produced more significant depleted or enriched sgRNAs than the untreated condition, though significant overlap suggests that selective pressures applied by endogenous anti-tumor immunity were similar to immunity enhanced by the use of immune checkpoint blockade (ICB) (p<2e-15, Fisher's exact test; FIG. 8C). The effect size of enrichment or depletion was greater in the immune checkpoint blockade (ICB)-treated condition, consistent with immune checkpoint blockade (ICB) enhancing the selective pressures applied by endogenous anti-tumor immunity (FIG. 8D).

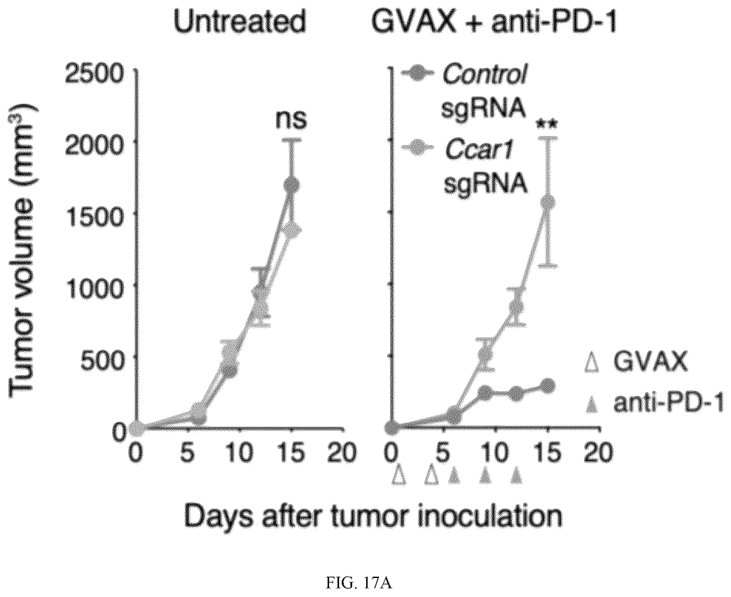

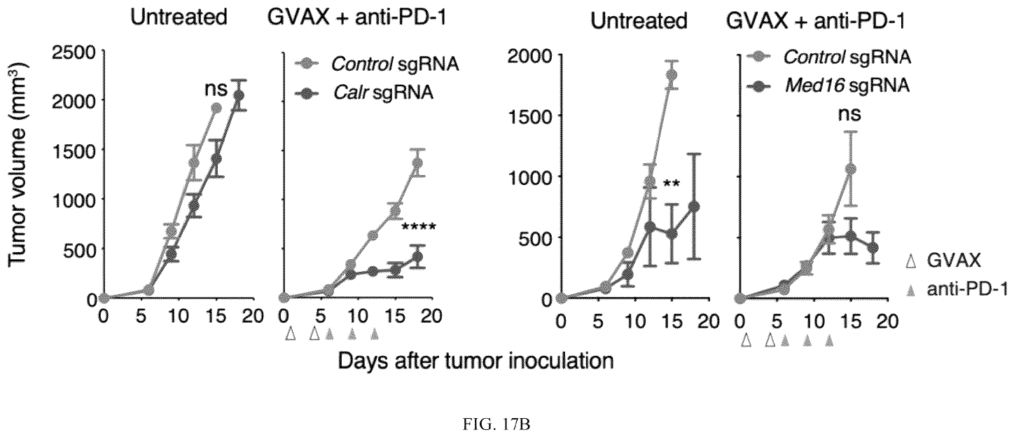

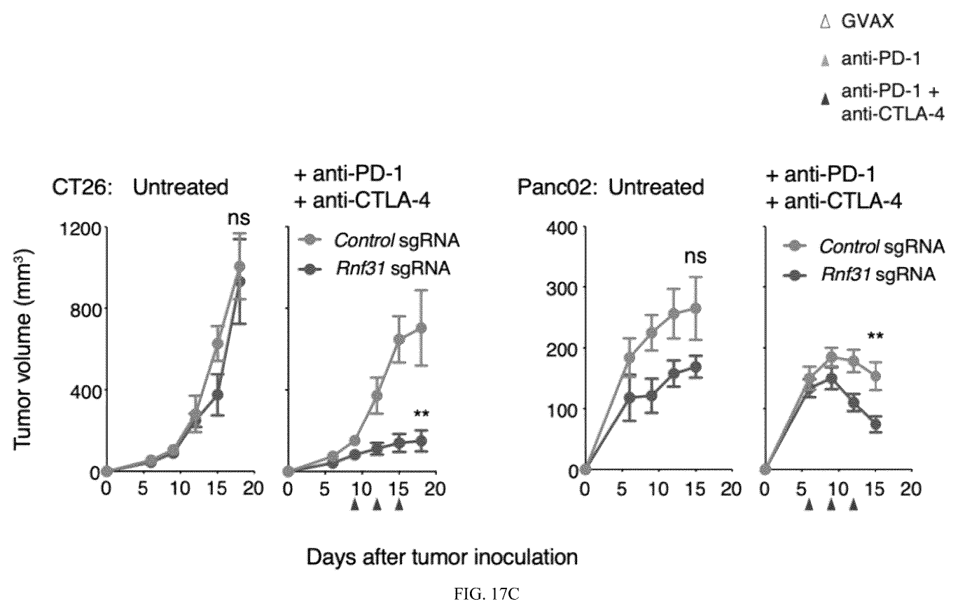

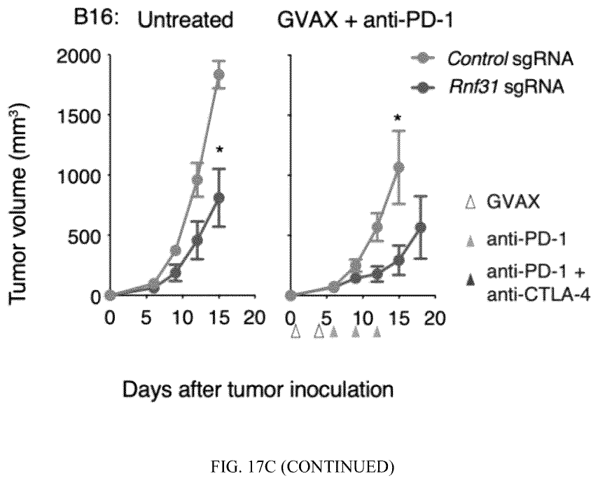

In addition to identifying many genes previously proposed to cause immune checkpoint blockade (ICB) resistance when lost such as Nlrc5, Pten, and Casp8 (FDR<0.05), several novel potential immune checkpoint blockade (ICB) resistance mechanisms were identified such as loss of Ccar1, Ubr5, Dotl1, Smg9, and Pdcd10 in the genome-scale screens (FIG. 1B; FDR<0.05). The sub-genome screens revealed additional resistance mechanisms such as loss of Raf1, Mapk1, Stkl1, and Nf2 (FIG. 9B FDR<0.05). Experiments were undertaken to validate novel resistance mechanisms, and it was shown in B16 melanoma that deletion of Ccar1, an enriched hit with no known role in tumor immunity, caused resistance to immune checkpoint blockade (ICB) (FIGS. 1C and 17A; Student's t test p<0.01).

Gene targets that were depleted in the sub-genome and genome immune checkpoint blockade (ICB)-treated cohorts relative to the NSG cohorts revealed previously reported mechanisms of immunotherapy sensitization, including Ptpn2, TNF signaling genes Traf2 and Ripk1, granzyme inhibitor Serpinb9, phagocytosis inhibitor Cd47, and the autophagy pathway genes Atg5 and Atg7 (FIG. 1B). In addition, many new genes were identified that sensitized tumors to immune checkpoint blockade (ICB) when lost, such as Calr, Rnf31, Vps45, Ilf2, Pnpo, Kcmf1, Fitm2, Morc2a, and Thop1 (FIGS. 1B and 9B; FDR<0.05).

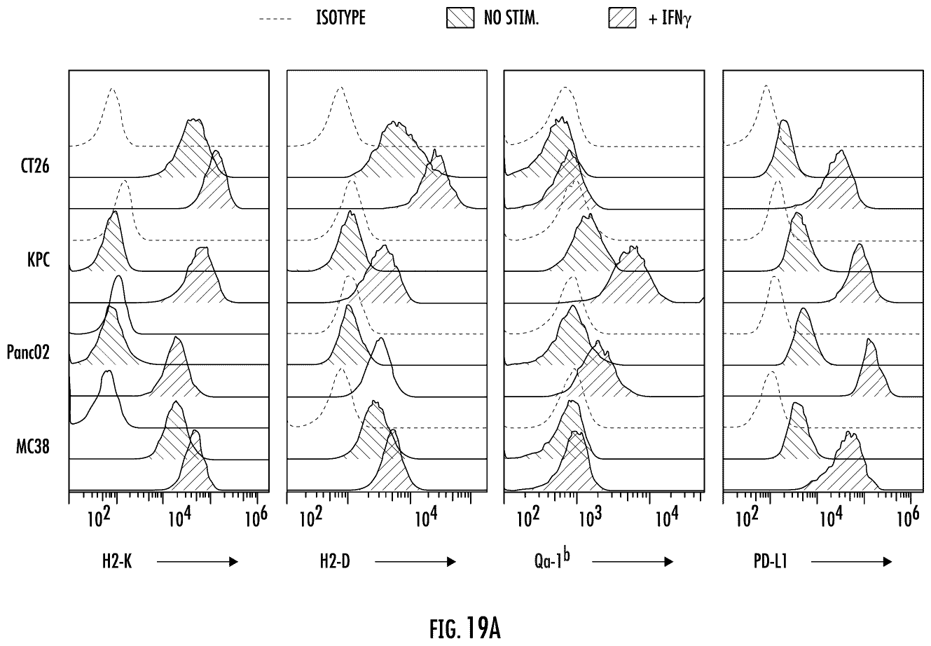

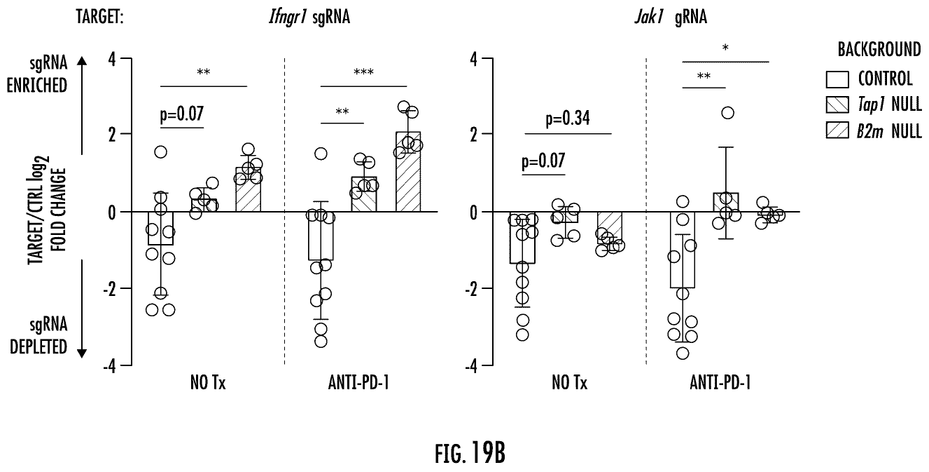

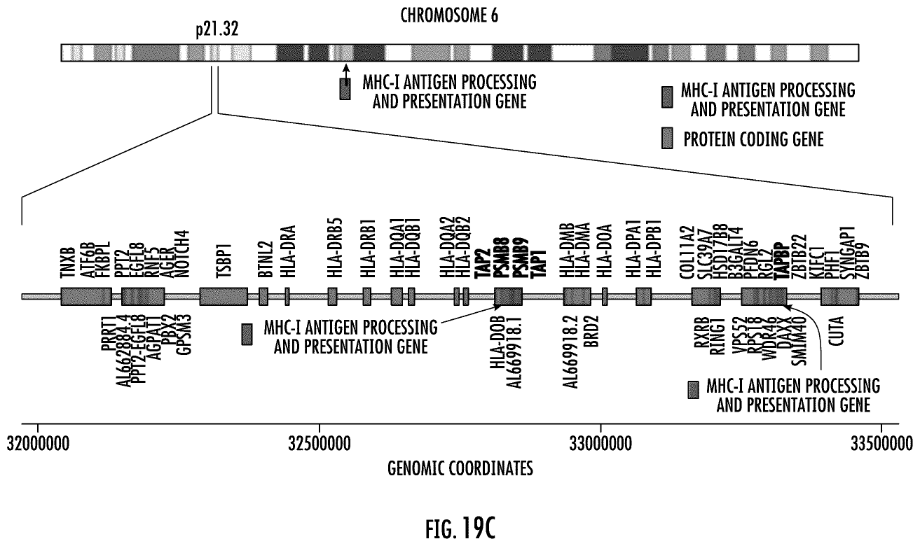

Many of these novel targets were specific to a subset of the cancer models screened rather than universally depleted across screens (FIG. 19A). To validate novel targets that sensitize B16 tumors to immunotherapy when lost, single gene validation was performed for Calr, an ER-resident chaperone and member of the antigen presentation pathway and Med16, a member of the mediator complex. Deletion of Calr or Med16 rendered B16 tumors highly sensitive to immune checkpoint blockade (ICB) (FIGS. 1C and 17B; Student's t test p<0.01). The screens also identified the ring finger protein Rnf31, a member of the linear ubiquitin assembly (LUBAC) complex, in multiple models. Indeed, deletion ofRnf31 in the CT26, Panc02, and B16 cell lines, sensitized to ICB in vivo (FIG. 17C; Student's t test p<0.01). Thus, in vivo genome-scale screens identified genes with novel roles in modulating immune checkpoint blockade (ICB) sensitivity.

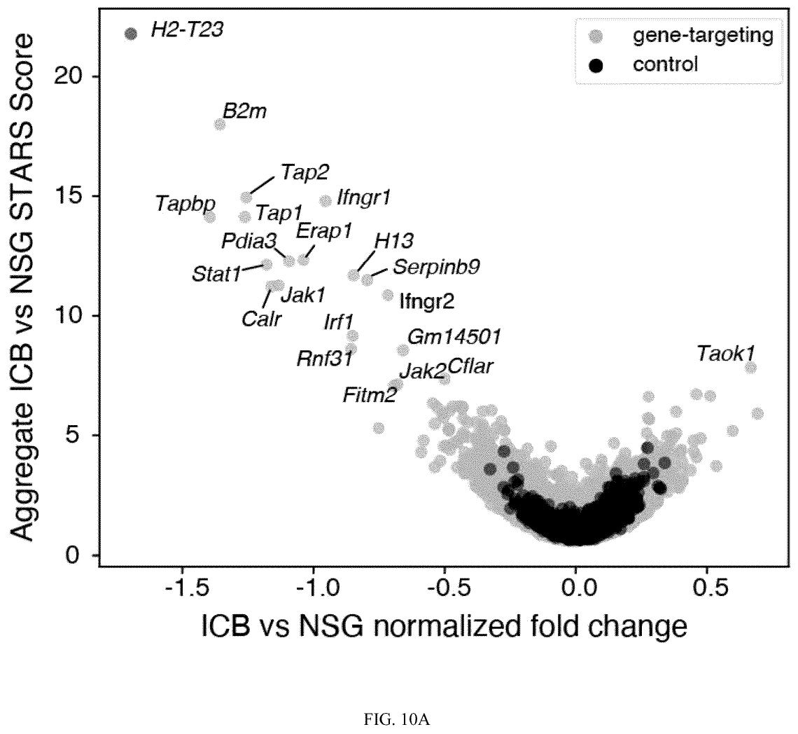

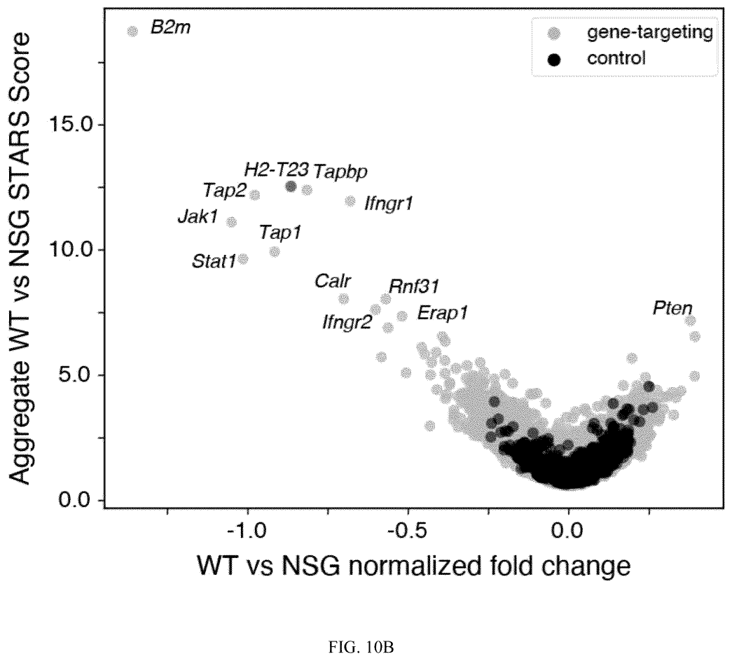

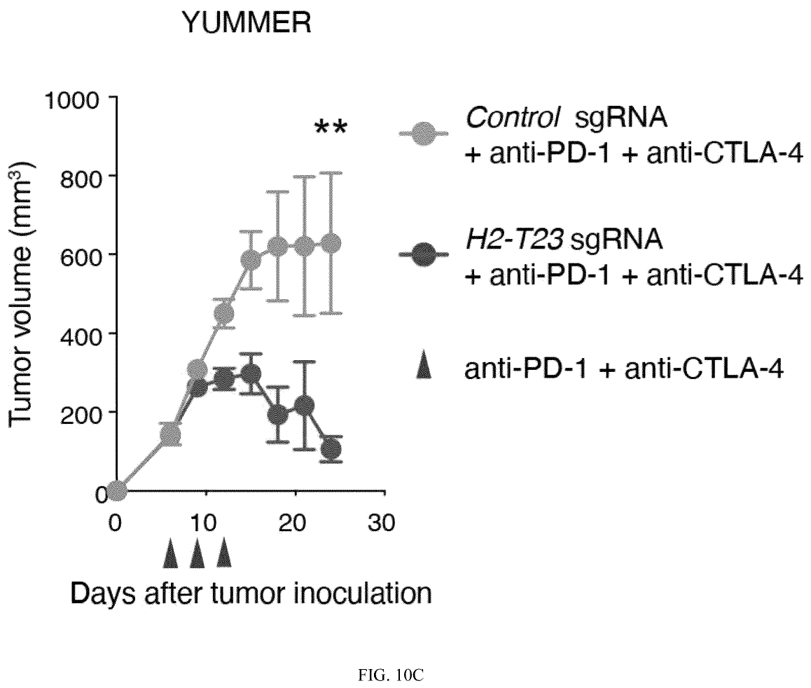

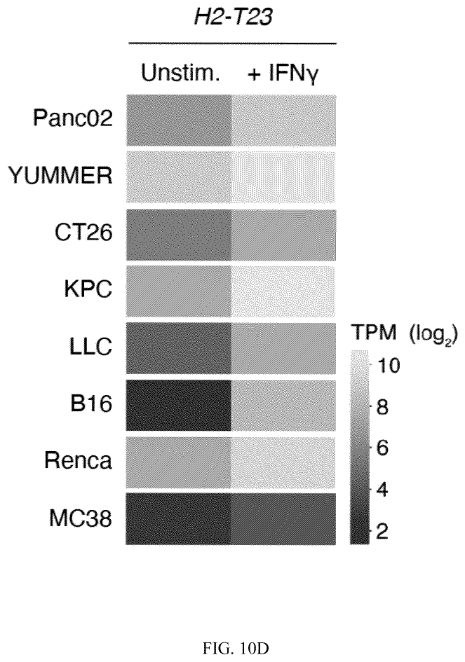

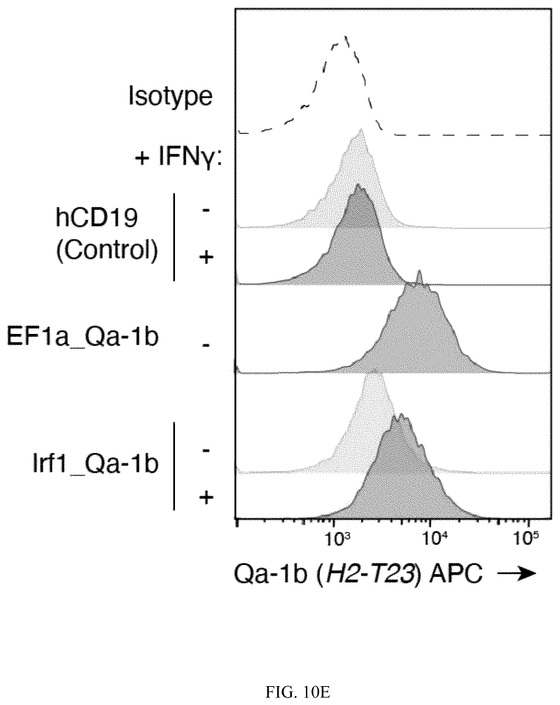

H2-T23, the mouse ortholog of human HLA-E, scored in the top 15 genetic dependencies in 7 out of the 8 models and was the top depleted hit overall, suggesting that this molecule is a potent negative regulator of anti-tumor immunity across cancer types (FIGS. 1B, 10A and 10B). Qa-1^{b}, the protein encoded by H2-T23, loss was validated as an immunotherapy sensitizer across cancer models by generating control or H2-T23-deficient KPC, Renca, and YUMMER cell lines and implanting them in WT mice treated with immune checkpoint blockade (ICB). In each model, H2-T23-deficient tumors had an enhanced response to immune checkpoint blockade (ICB) and were cured at a higher rate than control tumors (FIGS. 1D and 10C; Student's t test, p<0.05). MC38 colon carcinoma was the only cancer model that did not demonstrate an H2-T23 dependency in vivo. Interestingly, RNA sequencing revealed that H2-T23 expression was increased after stimulation with IFNγ in all models except MC38 (FIG. 10D). The lack of MC38 cell surface expression of Qa-1^{b} was evaluated by flow cytometry (FIG. 10E). To determine if forced expression of Qa-1^{b} is sufficient to inhibit anti-tumor immunity in MC38, MC38 cells were engineered to express Qa-1^{b} from either a constitutively active or IFNγ-inducible promoter and implanted into WT mice treated with immune checkpoint blockade (ICB) (FIG. 1E). Compared to control CD19⁺ MC38, MC38 tumors expressing either constitutive or IFN-inducible Qa-1^{b} (FIG. 10E) were resistant to immune checkpoint blockade (ICB) and mice showed poor survival following immune checkpoint blockade (ICB) treatment (FIG. 1E; Student's t test, p<0.05). Thus, Qa-1^{b} expression inhibited anti-tumor immunity in all murine cancer models tested and was the top scoring immune evasion gene in vivo.

To more systematically identify common and context-specific mechanisms of immune evasion, gene set overrepresentation analysis of depleted sgRNAs from each genome-scale screen was performed and network and tensor decomposition methods were used to derive a core set of 52 gene sets representative of 21 pathways (FIG. 1F). Most pathways only sensitized a subset of the models to immunotherapy; for example, pathways involved in chromatin modification were depleted in the B16, LLC and MC38 genome-scale screens (FIG. 1F adj. p<0.025), whereas unfolded protein response and protein ubiquitination gene sets were depleted only in the B16 screen (FIG. 1F, adj. p<0.0002). Surprisingly, sgRNAs targeting MHC-I antigen processing and presentation pathway genes were depleted in all models except LLC (FIG. 1F, adj. p<0.0002), and sgRNA targeting genes involved in response to cytokine, type II interferon, and innate immune response were also depleted across several screens (FIG. 1F, adj. p<0.0002, <0.02, <0.02, respectively).

Markov clustering of STRING network annotations of a set of 37 genes depleted across multiple genome-scale screens yielded 6 modules with 3 singletons (FIG. 1G). This analysis highlighted novel roles for chromatin modifiers, galactosyltransferases, and the vacuolar sorting protein Vps45 in driving immune evasion (FIG. 1G). Two pathways emerged as central regulators of tumor cell sensitivity to immunotherapy: the interferon (IFN) sensing and signaling pathway (Jak1, Jak2, Stat1, Ifngr1, Ifngr2) and antigen processing and presentation genes (Tap1, Tap2, Tapbp, Calr, Pdia3, Erap1, B2m, H2-T23) (FIG. 1G; FDR<0.002).

### Example 2: The Immune-Inhibitory Effects of Tumor Interferon (IFN) Sensing were Dependent on MHC-I

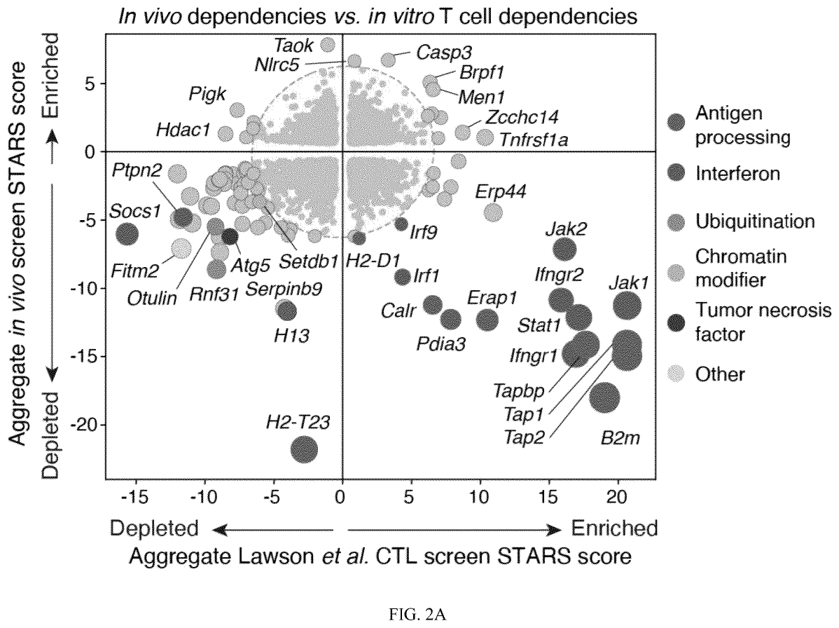

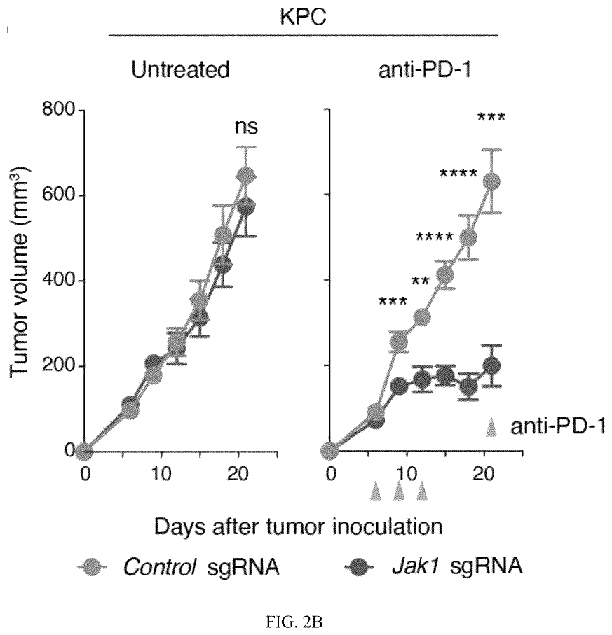

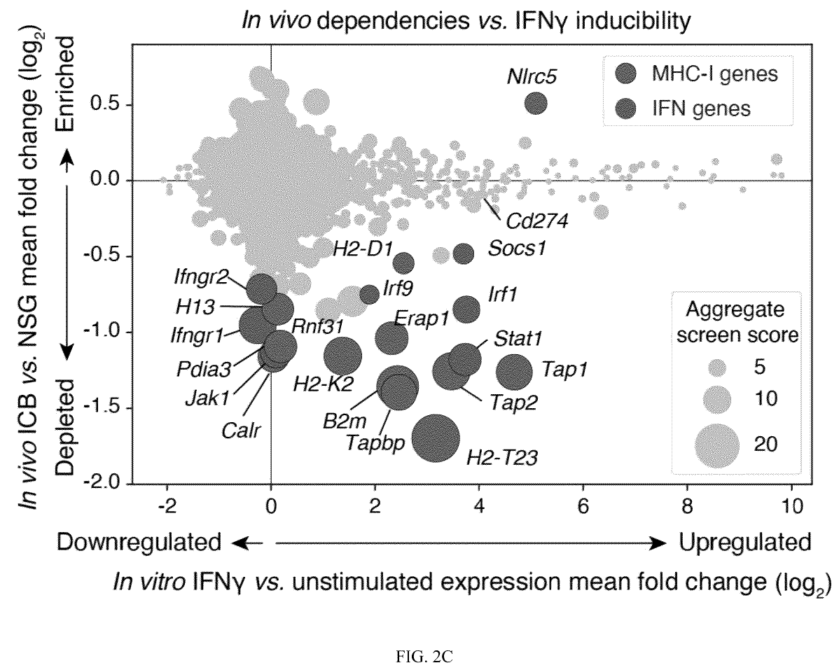

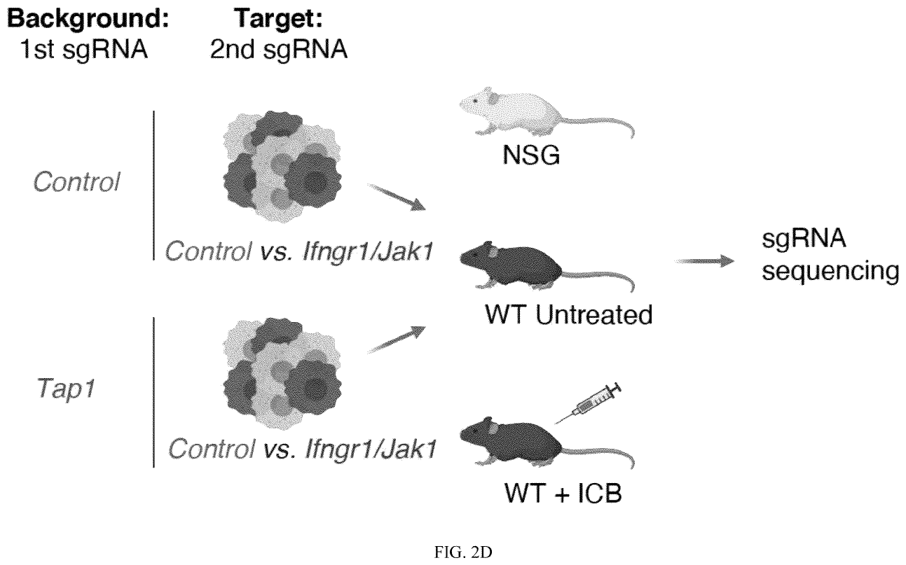

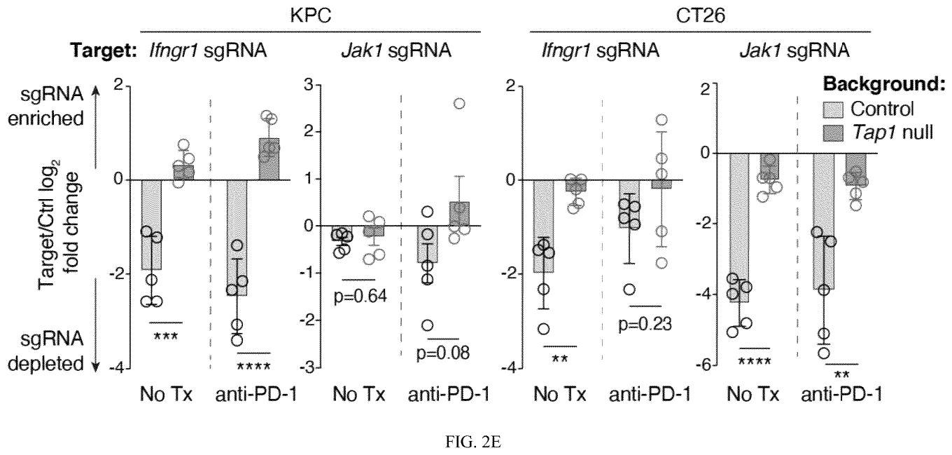

The observation of interferon (IFN)-sensing genes as depleted targets was surprising given the essential role for IFNγ in tumor control and previous reports of immunotherapy resistance mediated by loss of interferon (IFN) sensing. Interferon (IFN) signaling increases tumor expression of MHC-I antigen presentation molecules, which is required for specific recognition by CTLs. Indeed, in vitro CRISPR screens have identified that loss of interferon and antigen presentation in similar murine cancer models caused resistance to CD8⁺ T cell cytotoxicity (FIG. 2A) (Lawson, K. A. et al. Functional genomic landscape of cancer-intrinsic evasion of killing by T cells. Nature 586, 120-126 (2020)). Their in vitro screening data was plotted against the in vivo screening data to identify common and divergent hits across the datasets. While many commonly sensitizing and resistance-causing hits were identified, several members of the IFN sensing and antigen presentation pathways were divergent between the datasets, causing resistance to T cell killing but sensitizing in vivo (FIG. 2A). However, IFNγ sensing has also been associated with immune evasion in cancer patients and in preclinical models. Tumor sensing of type I interferon has also been associated with resistance to anti-tumor immunity via induction of Serpinb934 or Nos235. There is no consensus for the contexts in which interferon (IFN) signaling is inhibitory or the mechanism by which it mediates this effect. The identification that the loss of interferon (IFN) sensing scored as commonly sensitizing across models in vivo was surprising and suggested a conserved role in immune evasion that is specific to the tumor microenvironment.

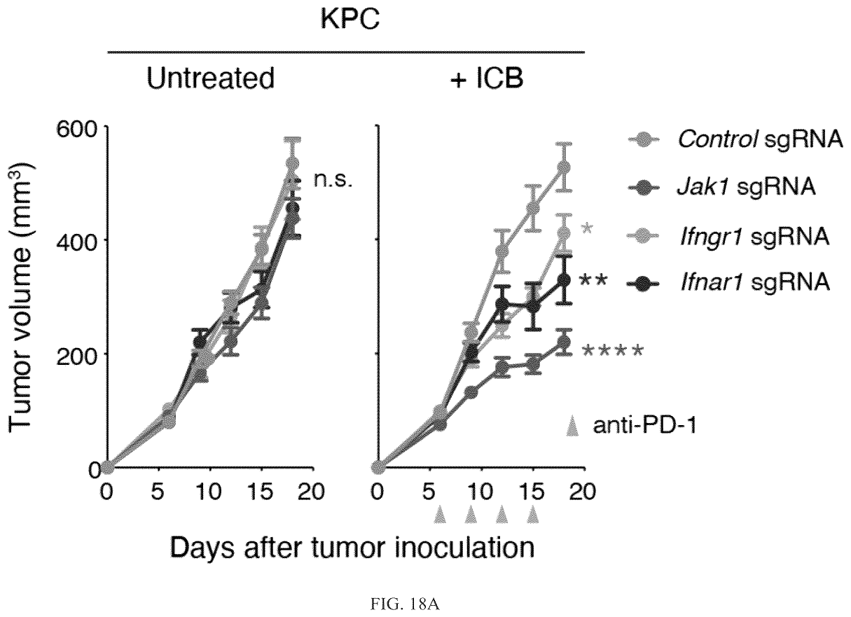

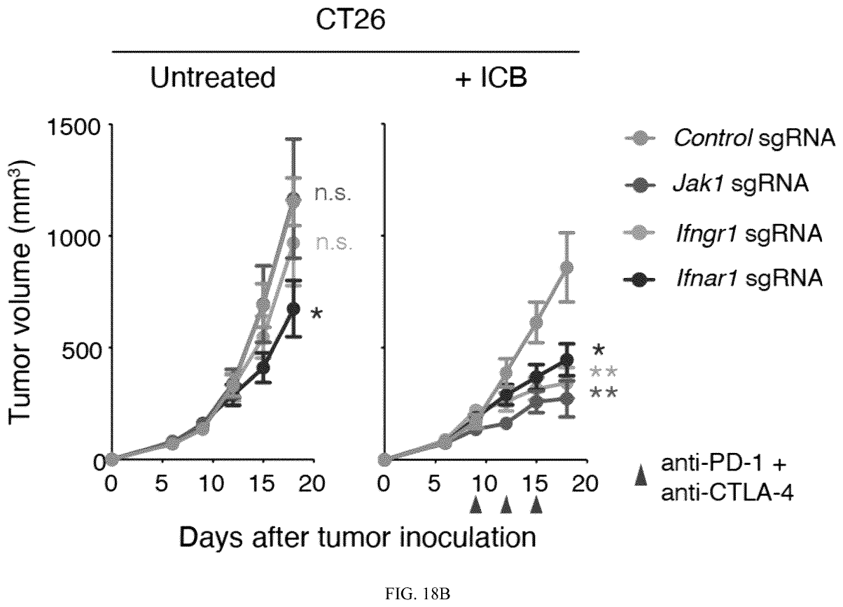

Experiments were undertaken to validate this observation in the WT C57BL/6 mice (KPC) pancreatic cancer and CT26 carcinoma models, which showed a marked dependency on interferon sensing genes in the in vivo screens. Jak1-, Ifngr1-, and Ifnar1-deficient KPC and CT26 cells were created and it was demonstrated that loss of either type I or type II IFN sensing significantly enhanced the response to ICB. Notably, for both KPC and CT26, the effects of deleting Jak1, which ablates both type I and type II IFN sensing, were more pronounced than deleting either Ifnar1 or Ifngr1 alone, confirming that tumor-intrinsic loss of both type I and type II interferon signaling increased sensitivity to anti-tumor immunity elicited by immune checkpoint blockade (ICB) (FIGS. 2B, 18A, and 18B).

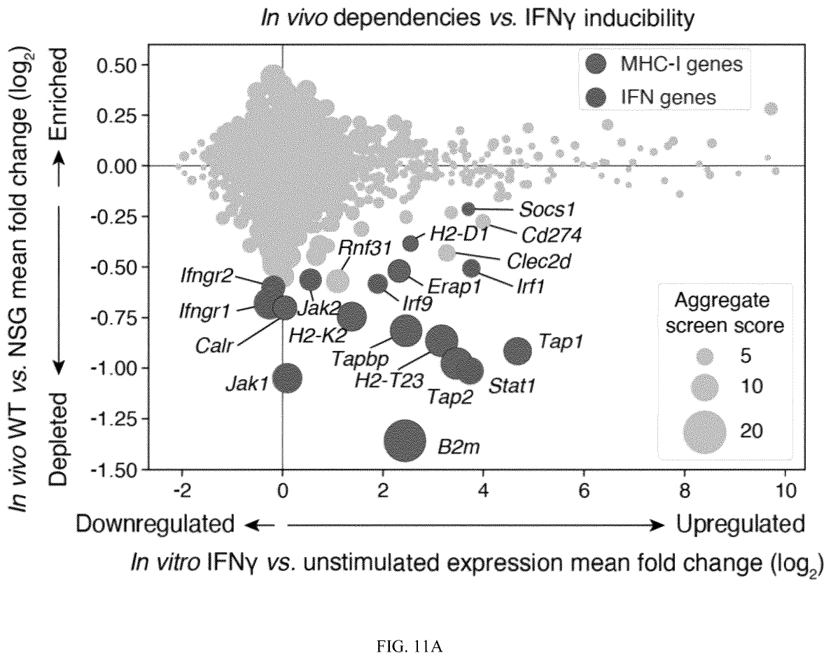

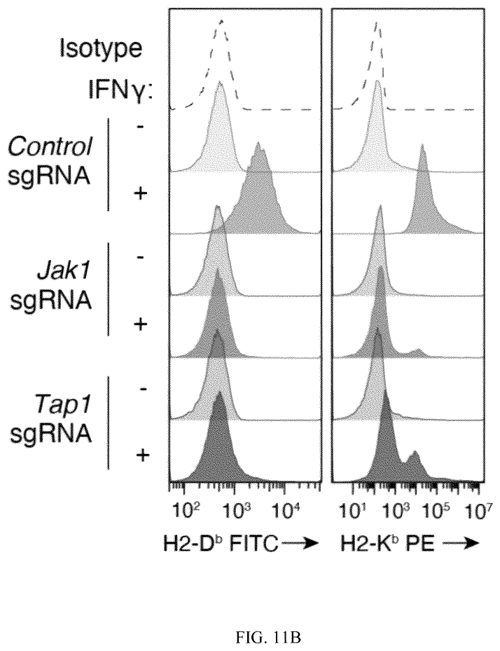

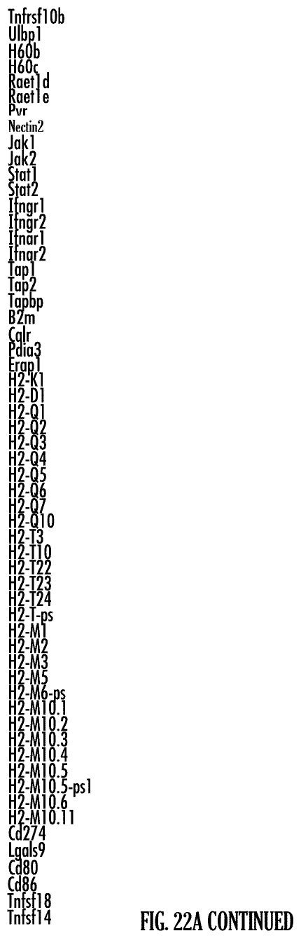

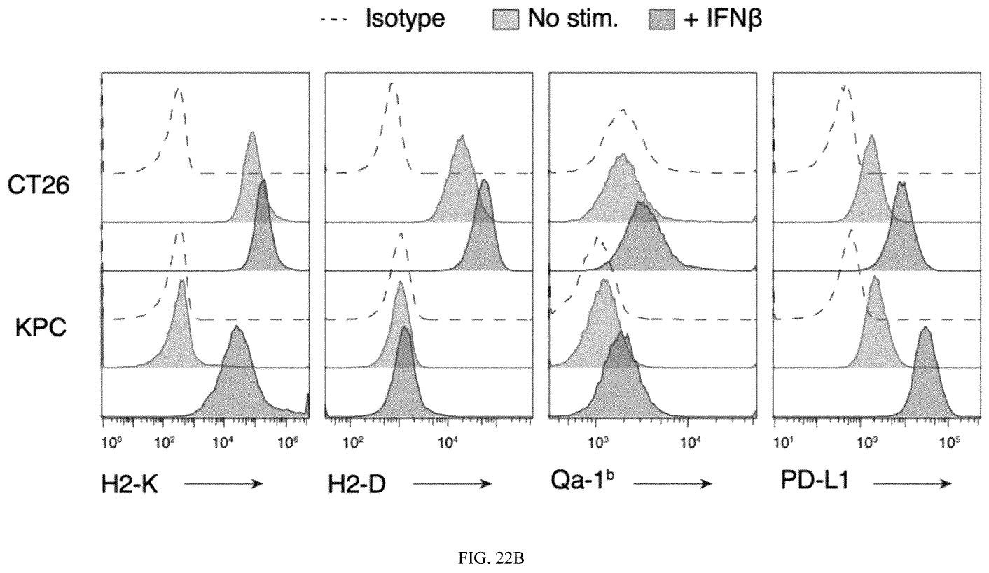

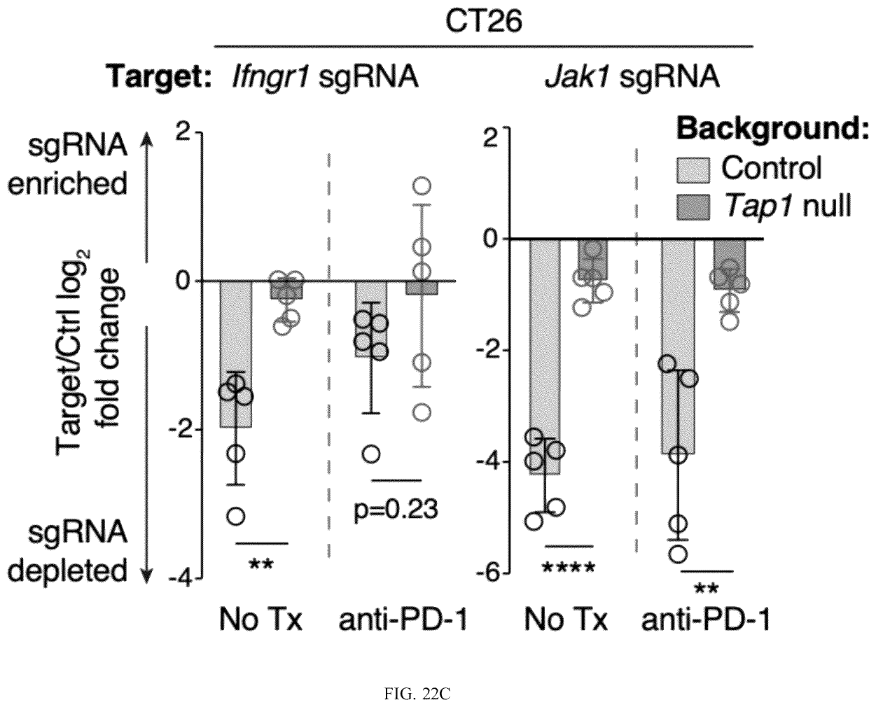

To find immune inhibitory mechanisms downstream of interferon sensing, RNAseq was used to profile the transcriptional response to IFN in vitro in all 8 cancer models and IFN-regulated genes were identified that were enriched or depleted in the genome-scale screens (FIGS. 2C, 11A, and 20A). This analysis revealed that genes from the antigen processing and presentation pathway (H2-T23, B2m, Tap1, Tap2, Tapbp, Erap1) were enriched among interferon (IFN)-induced genes with a strong in vivo immunotherapy dependency (FIGS. 2C and 11A). It was confirmed that surface expression of classical MHC (H2K and H2D), Qa-1b, and PD-L1 was increased following stimulation with IFNγ or IFNβ (FIGS. 19A, 22A, and 22B). Not intending to be bound by theory, this suggested that the inhibitory effect of tumor interferon sensing was mediated by the upregulation of MHC-I genes.

To test this hypothesis, an in vivo competition assay was designed to test for genetic epistasis between IFNγ sensing genes and the MHC-I pathway by deleting Tap1 or B2m, which reduces cell surface expression of MHC-I and scored as sensitizing to immune checkpoint blockade (ICB) in the in vivo screens (FIGS. 1G and 11B). Using the KPC model, 1:1 mixes were prepared of control and Ifngr1 or Jak1 sgRNA-transduced cells on either a control or Tap1-null or B2m-null background (FIG. 2D). Consistent with the screening and validation data, in the control background Ifngr1- or Jak1-null cells were depleted in vivo in untreated and immune checkpoint blockade (ICB)-treated mice (all conditions) (FIGS. 2E and 19B). However, this effect was completely lost in the Tap1- and B2m-null backgrounds, where Ifngr1- or Jak1-deficient cells had either no fitness disadvantage or were selectively enriched upon anti-PD-1 treatment (FIGS. 2E and 19B; Student's t test p<0.01 for CT26 Jak1). Similar results were observed using this competition assay in CT26 colon carcinoma (FIG. 22C, p<0.01 for CT26 Ifngr1 untreated, p<0.01 for CT26 Jak1). Thus, IFNγ-mediated inhibition of anti-tumor immunity was dependent on TAP and MHC-I presentation.

To test this hypothesis, an in vivo competition assay was designed to test for genetic epistasis between IFNγ sensing genes and the MHC-I pathway by deleting Tap1 or B2m, which reduces cell surface expression of MHC-I and scored as sensitizing to ICB in our in vivo screens (1G and 4B). Using the KPC model, 1:1 mixes of control and Ifngr1 or Jak1 sgRNA-transduced cells were made on either a control, Tap1-null, or B2m-null background (FIG. 2D). Consistent with the screening and validation data, in the control background Ifngr1- or Jak1-null cells were depleted in vivo in untreated and ICB-treated mice (all conditions) (FIG. 19B). However, this effect was completely lost in the Tap1- and B2m-null backgrounds, where Ifngr1- or Jak1-deficient cells had either no fitness disadvantage or were selectively enriched upon anti-PD-1 treatment (FIG. 19B; Student's t test p<0.001 for Ifngr1, p<0.01 for Jak1). Similar results were observed using this competition assay in CT26 colon carcinoma (FIG. 22C, p<0.01 for CT26 Ifngr1 untreated, p<0.01 for CT26 Jak1). Thus, while not intending to be bound by theory, IFN-mediated inhibition of anti-tumor immunity was dependent on TAP and MHC-I presentation.

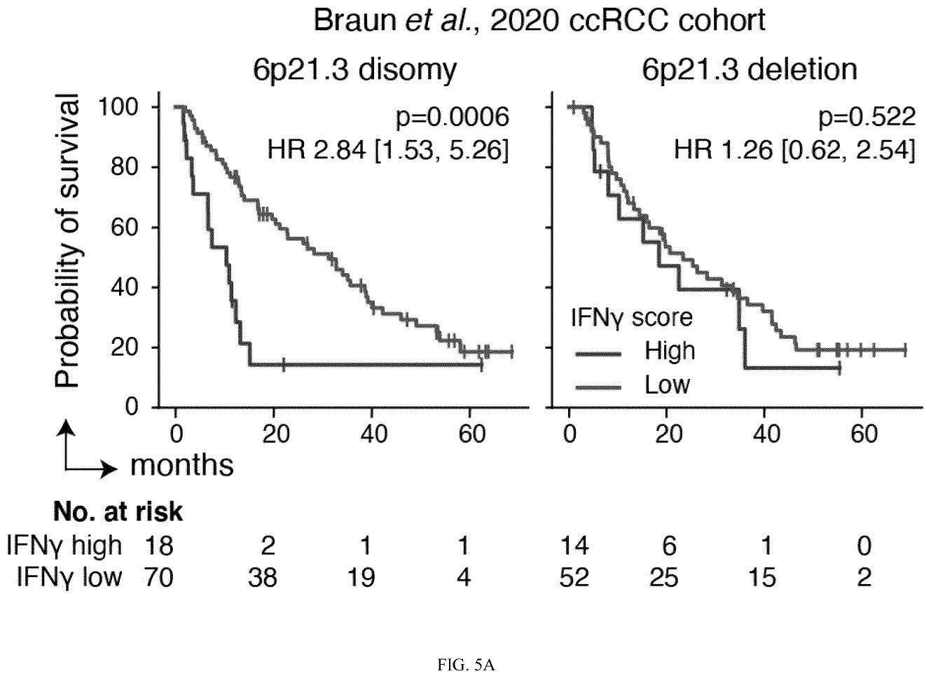

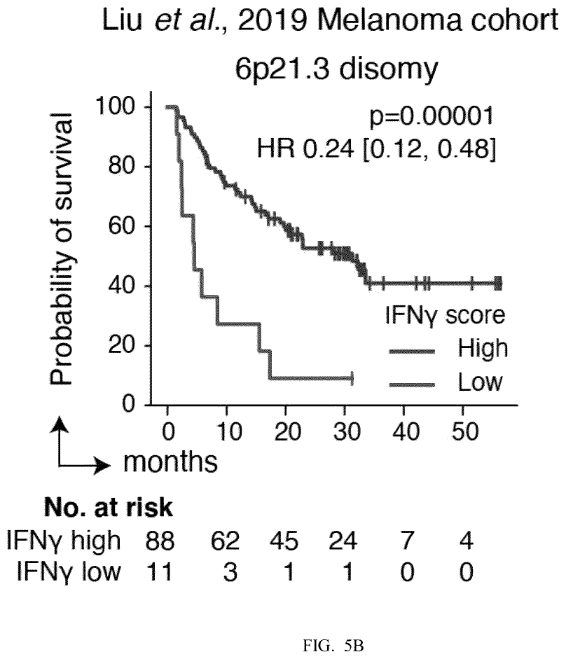

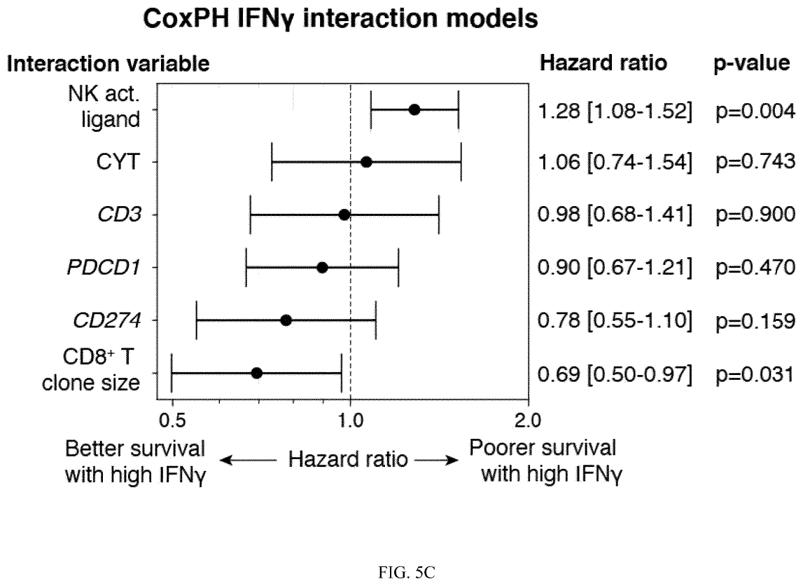

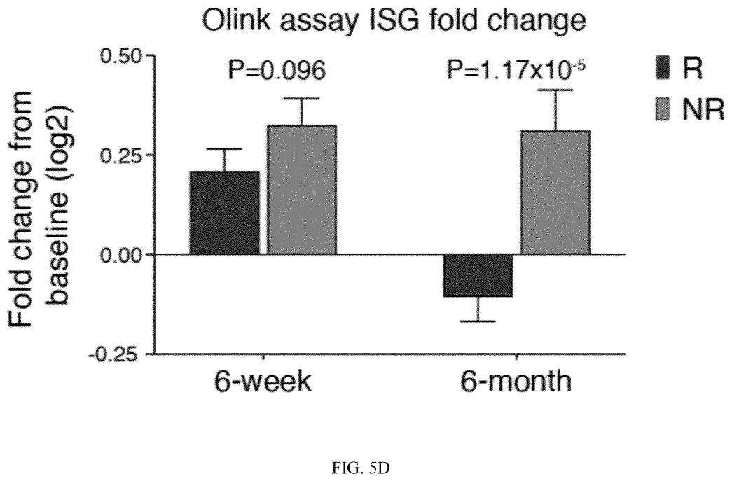

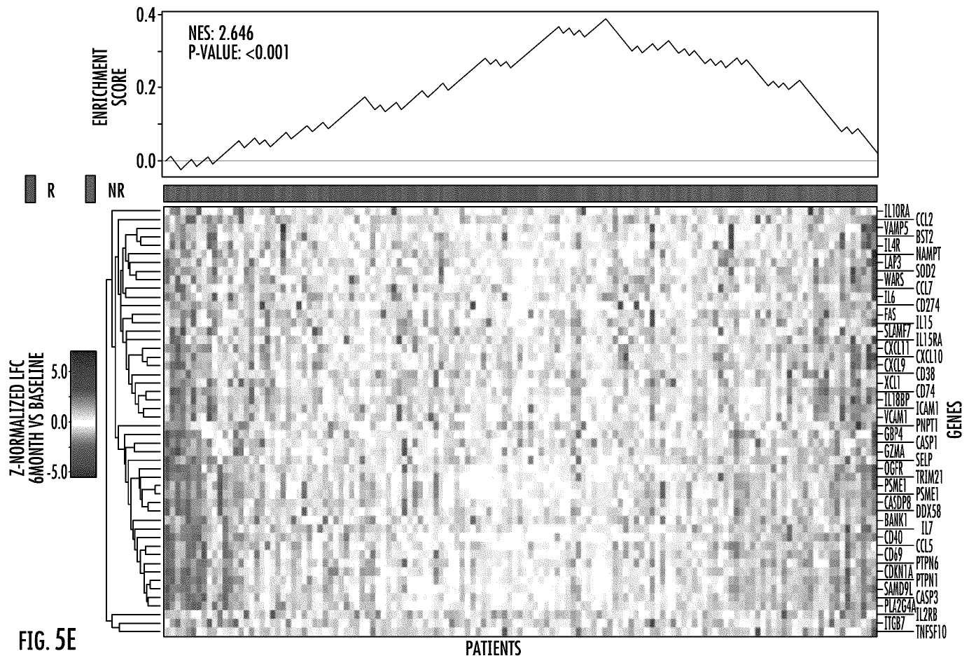

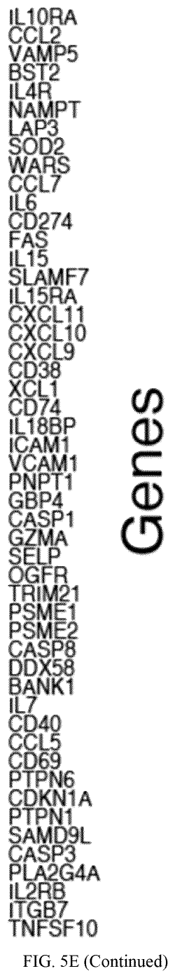

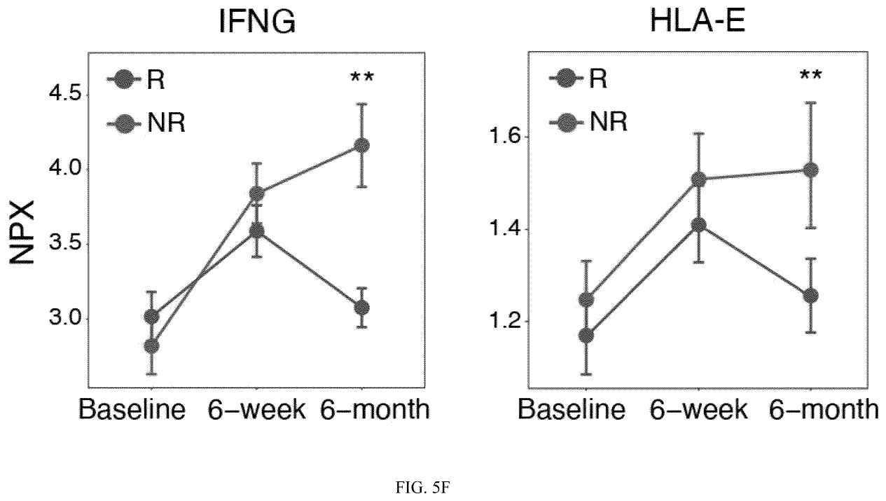

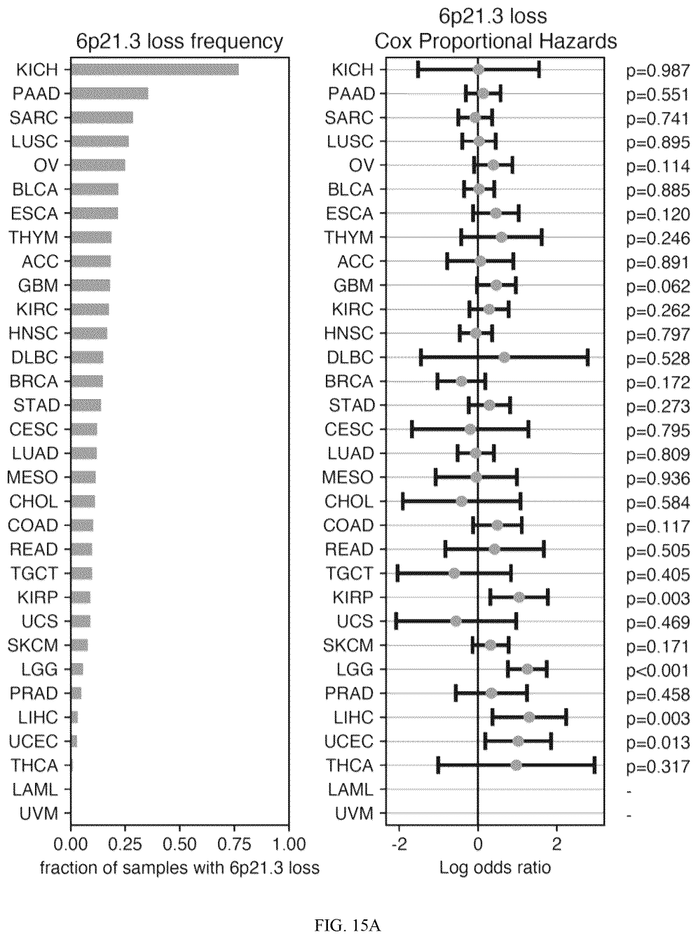

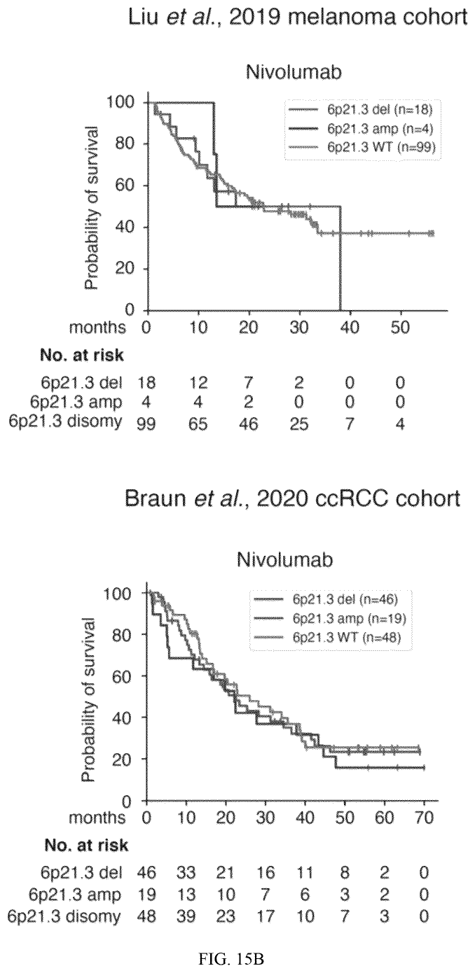

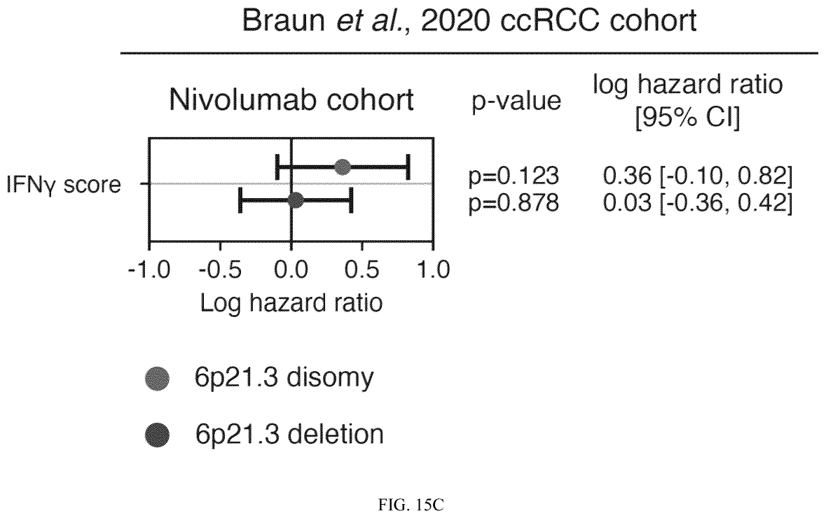

In order to examine the effect of disrupted MHC-I presentation on tumor interferon signaling in human cancers, a recurring deletion in the 6p21.3 genomic locus was identified across human cancers in TCGA (FIG. 15A). The 6p21.3 locus encodes several genes that are essential for the cell surface expression of MHC-I, including antigen processing transporter genes (TAP1, TAP2, TAPBP) and immunoproteasome genes (PSMB8, PSMB9) (FIG. 19C), and genomic deletion of this region impairs MHC-I presentation by tumor cells. The frequency of 6p21.3 deletion across cancers varies, but many cancer types show loss of 6p21.3 in >10% of samples in TCGA (FIG. 15A). 6p21.3 deletion alone did not stratify survival in most tissue types in TCGA (FIG. 15A). Therefore, the effect of ISG expression was examined, a proxy for tumor interferon levels, on survival in tumors with either an intact or deleted 6p21.3 locus. In a cohort of clear cell renal cell carcinoma (ccRCC) patients treated with anti-PD-1 therapy, a high ISG signature was significantly associated with poor overall survival in tumors with intact 6p21.3 loci (FIG. 5A; p=0.0006). Similar to TCGA analysis, 6p21.3 status did not stratify survival in this ccRCC cohort (FIG. 15B). However, it was observed that in tumors carrying 6p21.3 deletions, a high ISG signature was no longer associated with poor patient survival (FIGS. 5A and 15C; p=0.522), suggesting that interferon-mediated inhibition was dependent on the expression of MHC-I in ccRCC.

### Example 3: Immune Checkpoint Blockade (ICB) Activated CD4⁺ T Cells and NK Cells to Eliminate Interferon (IFN) Sensing-Deficient Tumors

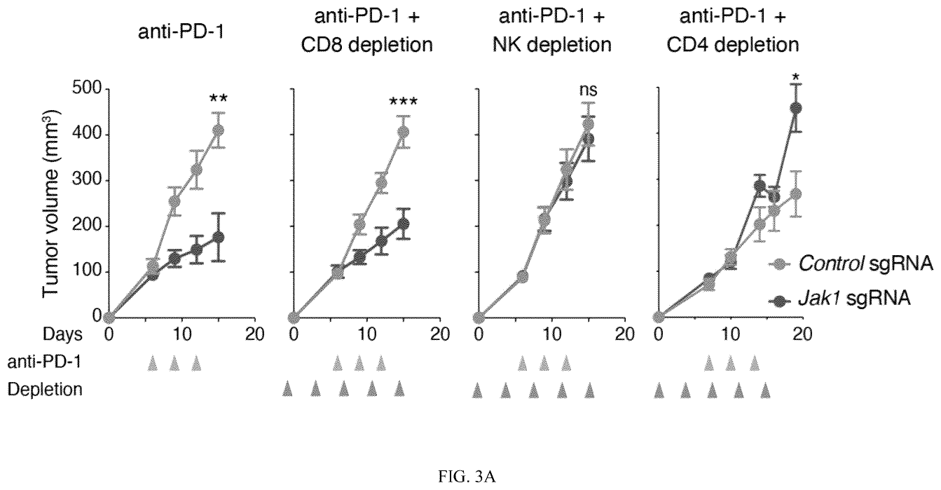

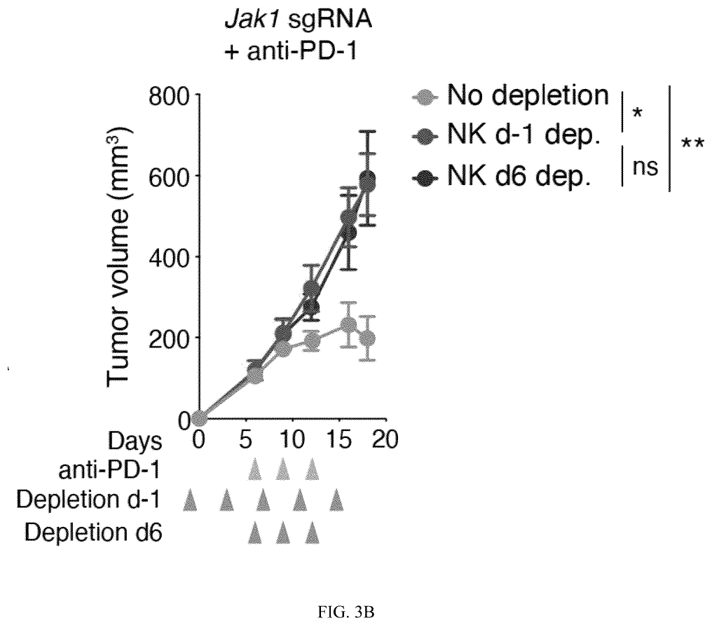

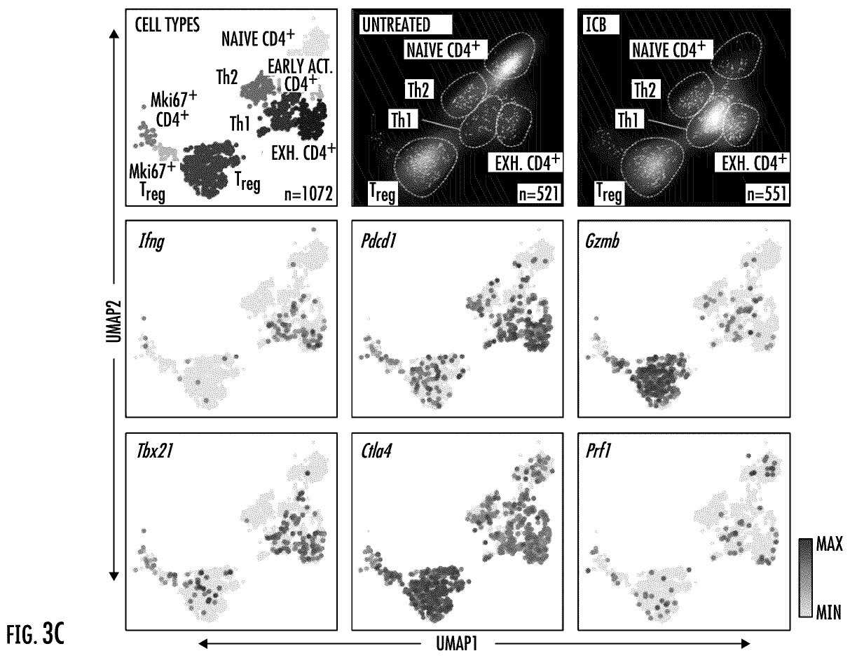

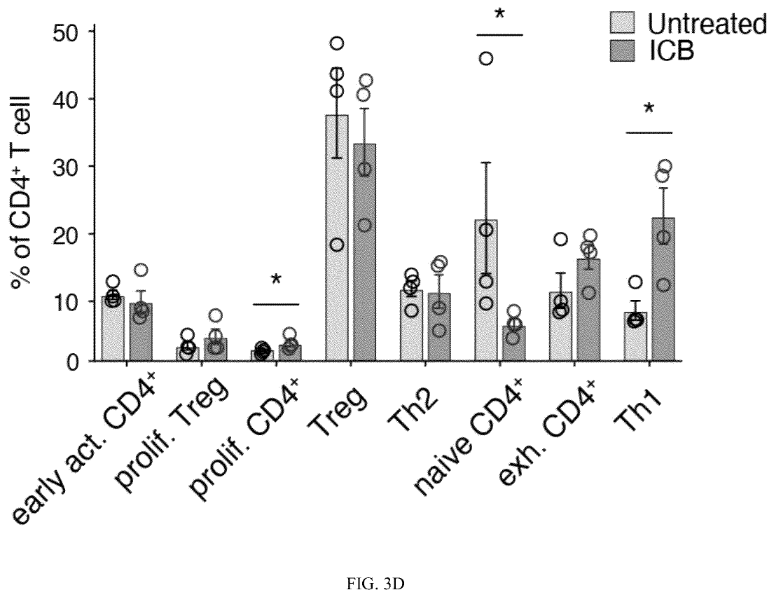

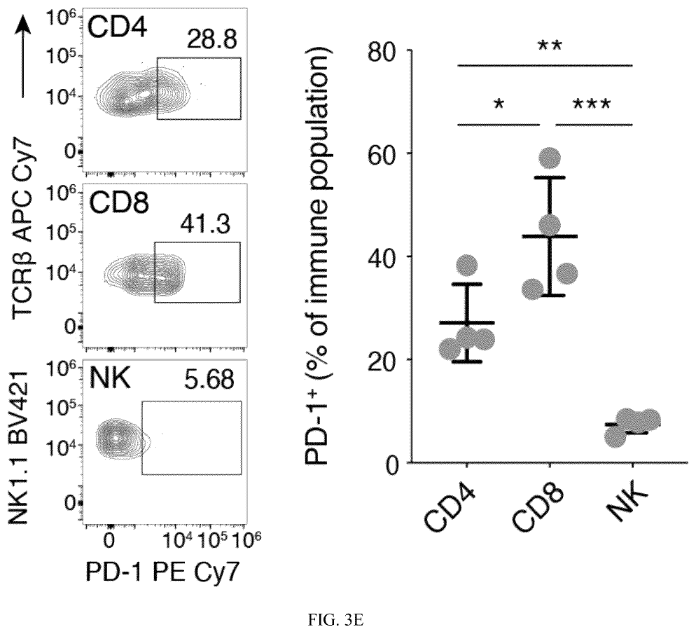

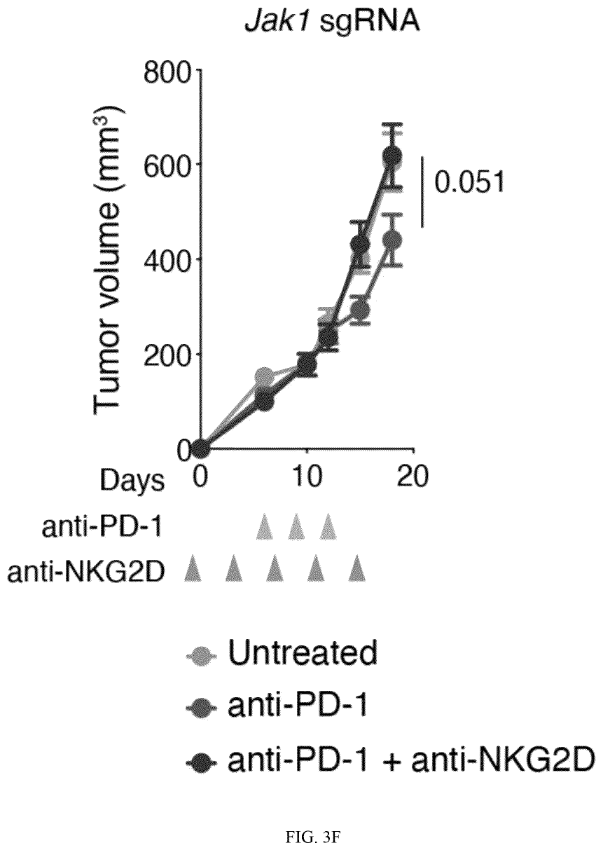

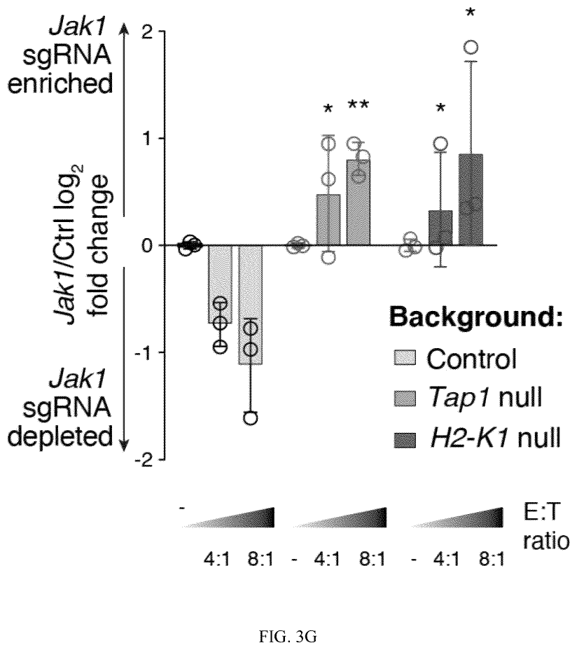

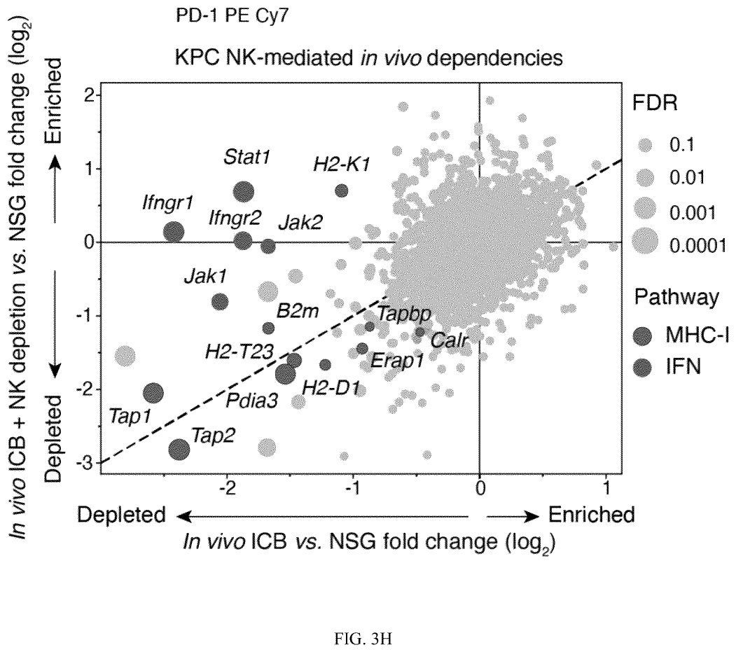

Both innate and adaptive immune cells, including natural killer (NK) cells, CD4⁺ T cells, and CD8⁺ T cells, have roles in anti-tumor immunity. NK cells, in particular, are known to eliminate cells lacking self MHC-I expression, and their cytotoxicity is inhibited by expression of MHC-I on target cells. However, because PD-1 and CTLA-4 are not expressed on most NK cells, the role of NK cells in immune checkpoint blockade (ICB)-mediated tumor destruction is not clear. To identify the immune subsets responsible for preferential killing of interferon sensing-null tumors in vivo, Jak1-deficient or control KPC cells were injected into immune checkpoint blockade (ICB)-treated WT mice with or without depleting antibodies for CD8⁺ T cells, CD4⁺ T cells, or NK cells. Depletion of CD8⁺ T cells had no effect on the enhanced response of Jak1-null tumors to PD-1 blockade (FIG. 3A, left). However, depletion of either CD4⁺ T cells or NK cells abrogated the response (FIG. 3A, right). NK cells are innate immune effector cells capable of rapid responses and do not require antigen-specific priming. Thus, not intending to be bound by theory, it is possible that NK cells primarily eliminated Jak1-deficient cells during tumor implantation and engraftment, prior to an immune checkpoint blockade (ICB)-induced immune response but did not play a significant role in controlling established tumors. Therefore, the depletion of NK cells in mice bearing Jak1-null tumors was delayed until day 6 post implantation, after tumor engraftment and concurrent with administration of PD-1 blockade. Even with delayed depletion of NK cells, Jak1-null tumors grew progressively and did not respond to immune checkpoint blockade (ICB), suggesting that NKs play a role in the immune checkpoint blockade (ICB)-mediated control of interferon (IFN) sensing-deficient tumors beyond initial engraftment (FIG. 3B).

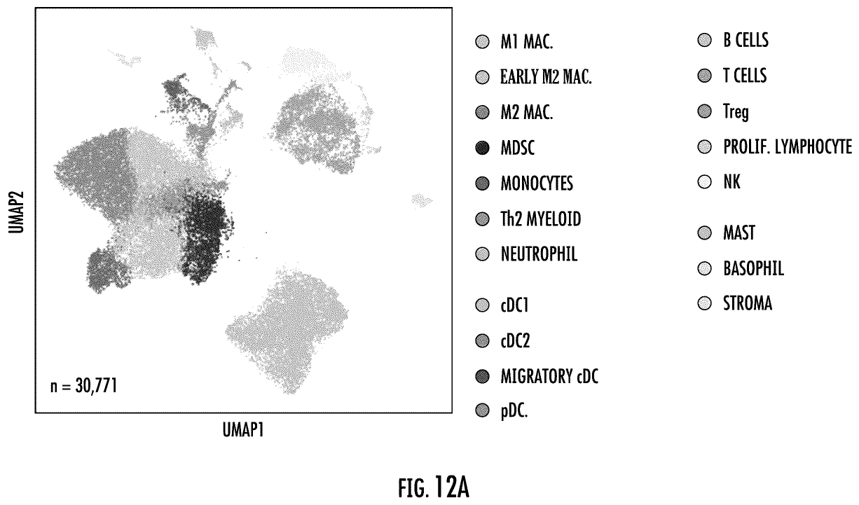

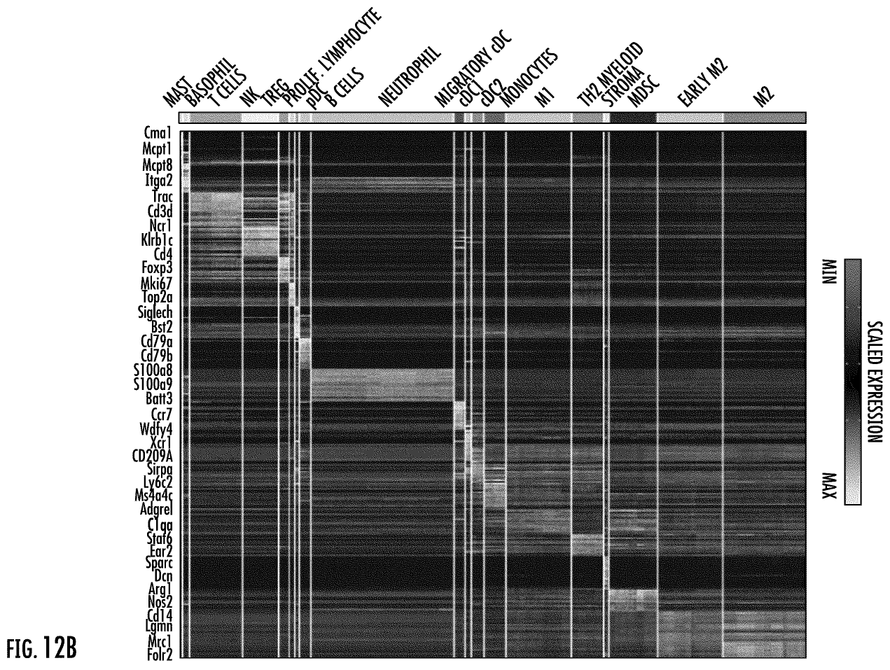

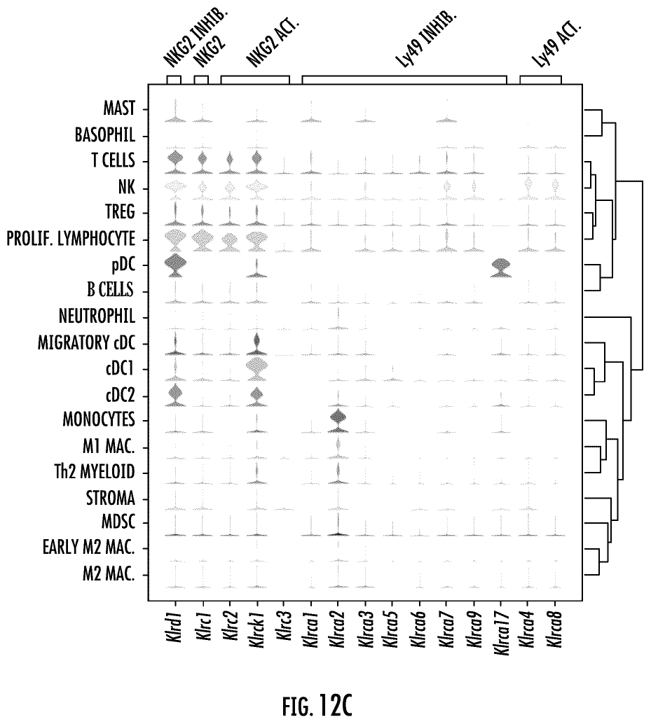

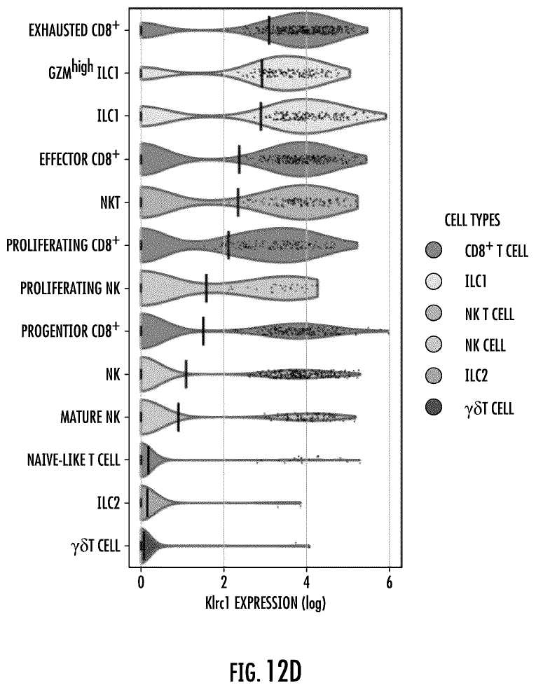

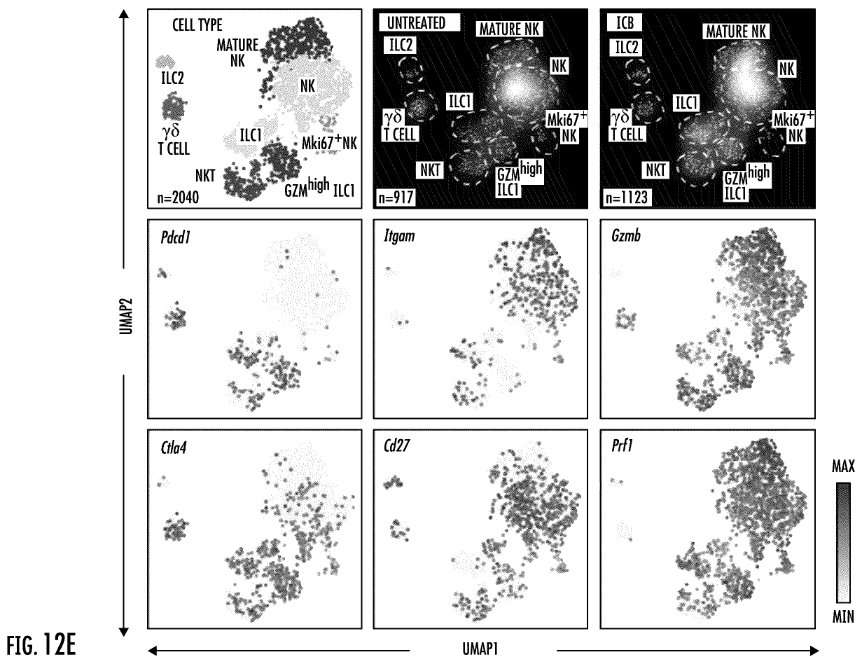

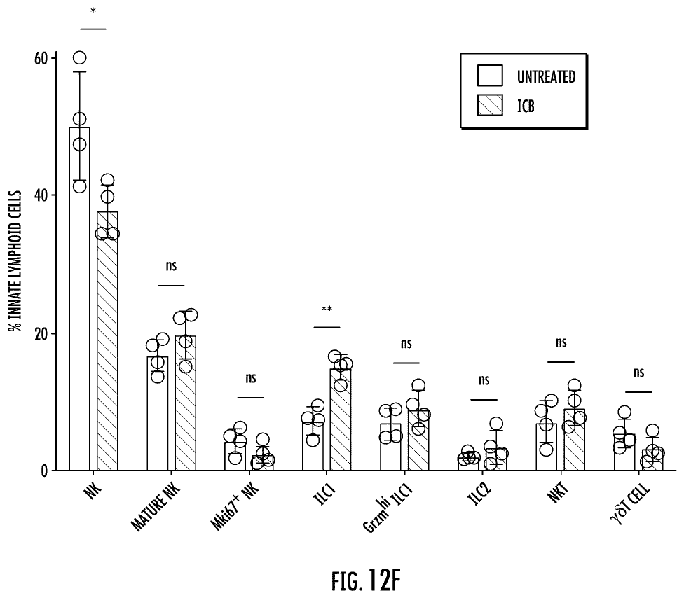

Having established the functional importance of both CD4⁺ T cells and NK cells in the response of interferon sensing-deficient tumors to immune checkpoint blockade (ICB), experiments were undertaken to determine how immune checkpoint blockade (ICB) impacts these immune subsets using single cell transcriptional profiling. CD45Y tumor-infiltrating lymphocytes (TILs) from untreated and immune checkpoint blockade (ICB)-treated KPC tumors (FIGS. 3C and 12A-12F) were examined using single-cell RNA sequencing (scRNAseq). Specific reclustering of NK1.1⁺ (Klrb1c) cells revealed a large population of conventional NK cells expressing high levels of cytotoxic molecules and small subsets of innate lymphoid cells (ILCs) and NKT cells (FIG. 12E). The ILC and NKT cell populations expressed both PD-1 and CTLA-4 and thus could be activated by ICB directly (FIG. 12E). Conversely, the large population of cytotoxic NK cells lacked expression of PD-1 and CTLA-4, and showed no significant change in abundance following treatment, suggesting that immune checkpoint blockade (ICB) was unlikely to act directly on these cells (FIG. 12E). However, reclustering of CD4⁺ T cells revealed an activated Th1-polarized (Ifng⁺, Tbx21⁺) population expressing both PD-1 and CTLA-4 that significantly increased in abundance after immune checkpoint blockade (ICB) therapy (FIGS. 3C and 3D). The expression of PD-1 on CD4⁺ T cells but not NK cells in KPC tumors was confirmed using flow cytometry (FIG. 3E).

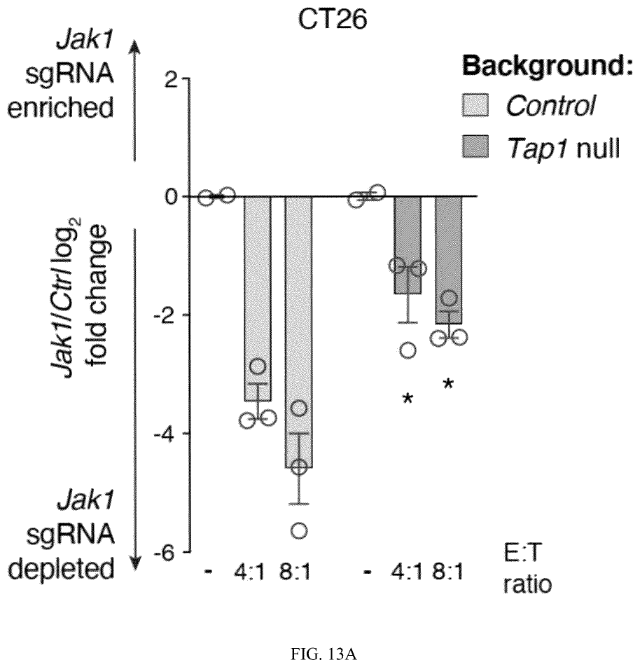

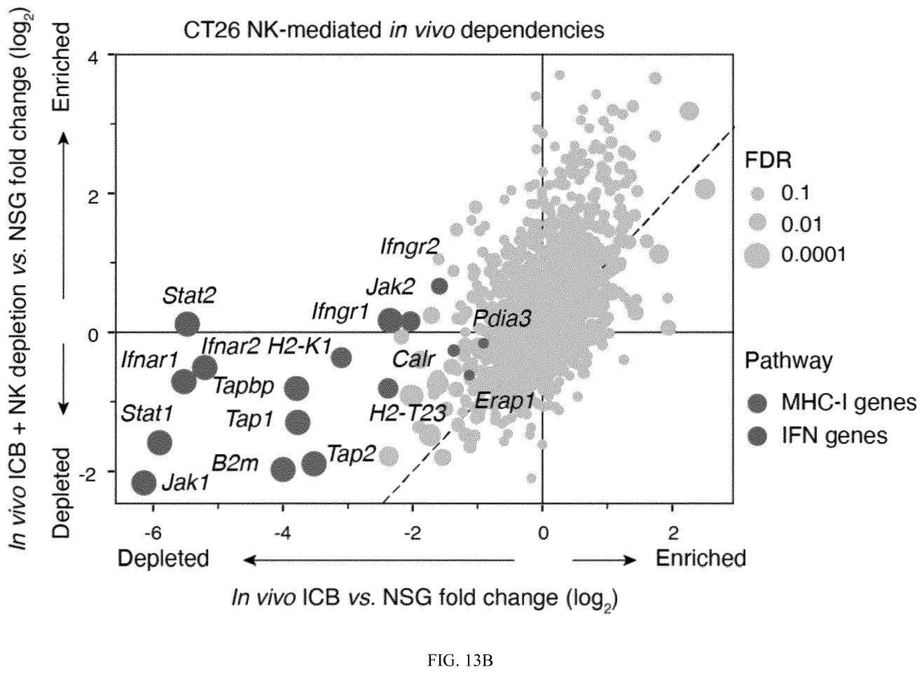

Only low levels of cytotoxic gene expression in the activated CD4⁺ T cells could be detected (FIG. 3C), and MHC-II genes did not appear enriched in the in vivo screens (FIGS. 1A-1G), consistent with the observation that CD4⁺ T cells generally do not aid in tumor control via direct cytotoxicity. Not intending to be bound by theory, it was thus hypothesized that CD4⁺ T cell-help via co-stimulation or Th1 cytokine production was enhancing NK cell-mediated direct cytotoxicity of interferon sensing-deficient tumor cells. Consistent with this hypothesis, the NK cells infiltrating KPC tumors expressed high levels of the activating NKG2D receptor (Klrk1), which recognizes ligands induced on the surface of tumor cells to activate NK cells (FIG. 12C). Blockade of NKG2D in vivo suppressed the sensitivity of Jak1-null tumors to anti-PD-1 therapy (FIG. 3F), strongly suggesting that NK cells directly kill the Jak1-deficient tumor cells. To determine if NK cells were sufficient to mediate the selective killing of Jak1-null tumor cells, NK cells were isolated and activated in the Th1 cytokines IL-12 and IL-18 and co-cultured with a 11 mixture of fluorescently labeled Jak1-deficient and control KPC cells. Jak1-deficient cells were depleted after 24 hours of co-culture with activated NKs (FIG. 3G). Crucially, co-deletion of Tap1 or H2-K1 in the tumor cells reversed this effect such that Jak1-deficient cells became less sensitive to NK cell killing (FIG. 3G; Student's t test, p<0.05). Similar results were obtained using Balb/C NK cells co-cultured with Jak1-deficient or control CT26 tumor cells on either a control or Tap1-null background (FIG. 13A; Student's t test, p<0.05). Not intending to be bound by theory, these results indicated that tumor interferon (IFN) sensing inhibited NK cytotoxicity in vivo and in vitro via upregulation of classical MHC-I.

Given the link between interferon (IFN) sensing and MHC-I-mediated evasion of NK killing described above, it was suspected that most of the interferon (IFN) and MHC-I pathway genes that had scored as sensitizing hits in the screens (FIG. 1G), including H2-T23/Qa-1^{b}, were likely dependent on NK cell surveillance. To test this, an in vivo screen was performed using KPC tumors transduced with the sub-genome library (FIG. 9A) (Manguso, R. T. et al. In vivo CRISPR screening identifies Ptpn2 as a cancer immunotherapy target. Nature 547, 413-418 (2017)) and implanted into WT B6 mice treated with immune checkpoint blockade (ICB) and anti-NK1.1. sgRNA fold changes were compared between immune checkpoint blockade (ICB)-treated NK1.1-depleted and non-depleted arms (FIG. 3H). Consistent with the previous data, the depletion of guides targeting IFNγ sensing genes (Ifngr1, Ifngr2, Stat1, Jak1, Jak2) showed strong NK cell-dependence and was not sensitizing in the NK1.1-depleted screen (FIG. 3H, y-axis). In support of the in vitro NK cell cytotoxicity data (FIG. 3G), loss of H2-K1 also no longer sensitized tumor cells in NK cell-depleted mice (FIG. 3H, y-axis). Surprisingly, sgRNAs targeting several antigen presentation genes including Tap1, Tap2, H2-D1 and H2-T23 were strongly depleted in the presence or absence of NK cells. Similar results were observed in a screen of the same library in CT26 cells implanted in Balb/C mice treated with immune checkpoint blockade (ICB) and anti-asialo GM1 to deplete NK cells (FIG. 13B). Thus, not intending to be bound by theory, while NK cells mediated the killing of cells lacking the ability to sense interferon (IFN) and upregulate classical MHC-I heavy chains, expression of other MHC-I molecules including Qa-1^{b} could also inhibit additional immune effector populations.

### Example 4: Tumor Interferon (IFN)-Sensing Inhibited Cytotoxic CD8⁺ T Cells Via Upregulation of Qa-1^{b}

Based on the observation that guides targeting H2-T23, Tap1, Tap2, B2m, and H2-D1 were still strongly depleted in the absence of NK cells in vivo, experiments were undertaken to examine potential inhibitory effects of H2-T23 on other immune cell populations. H2-T23 encodes the non-classical MHC-I molecule Qa-1^{b} (HLA-E in humans) that binds the inhibitory NKG2A/CD94 receptor. The inhibitory activity of Qa-1^{b} requires presentation of a TAP-dependent peptide (Qdm) derived from the signal sequence of the H2-D1 heavy chain in B6 mice. Because Qa-1^{b} was the strongest hit overall across all in vivo screens (FIG. 10A), it was suspected that loss of Tap1, Tap2 or H2-D1 would score as hits because loss of these genes prevents the loading of the H2-D1 signal peptide, leading to impaired Qa-1^{b} function.

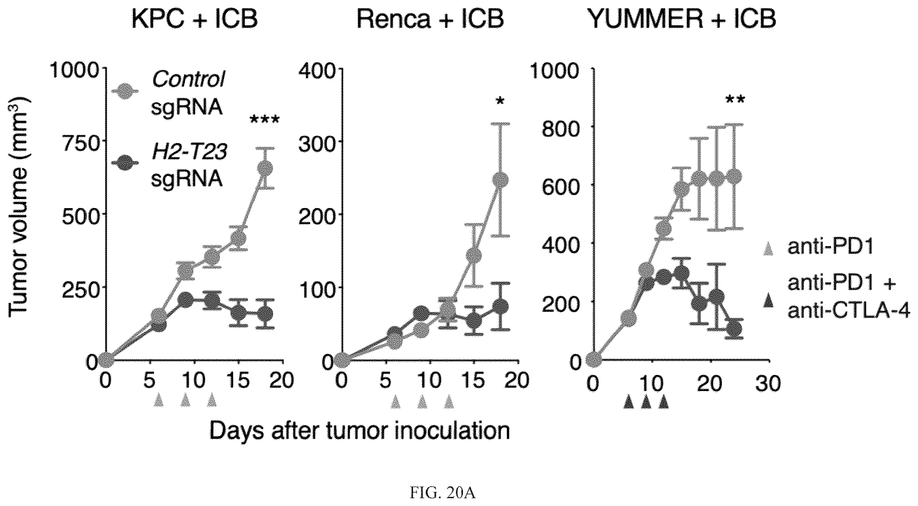

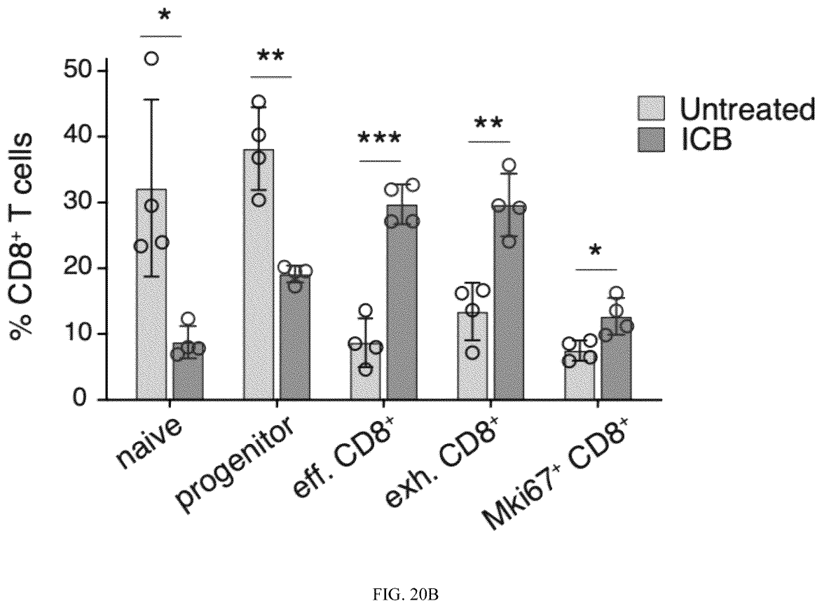

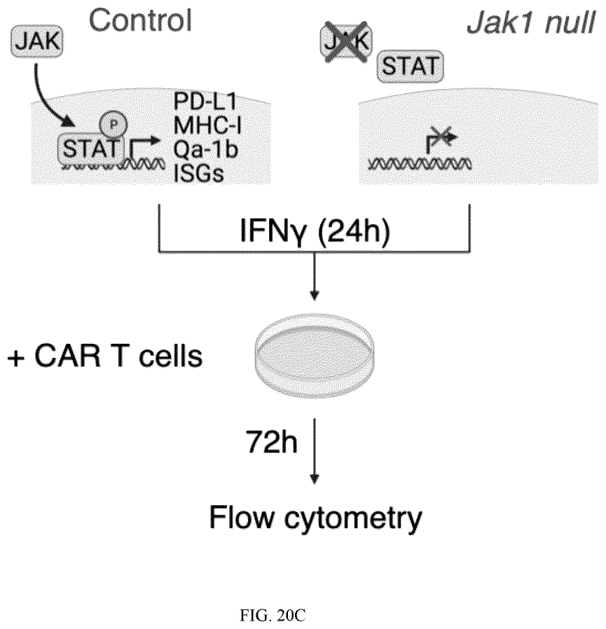

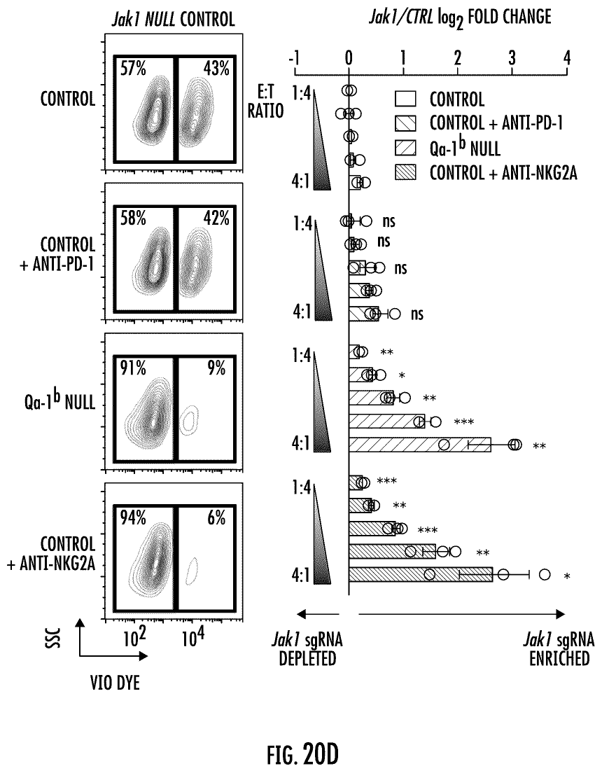

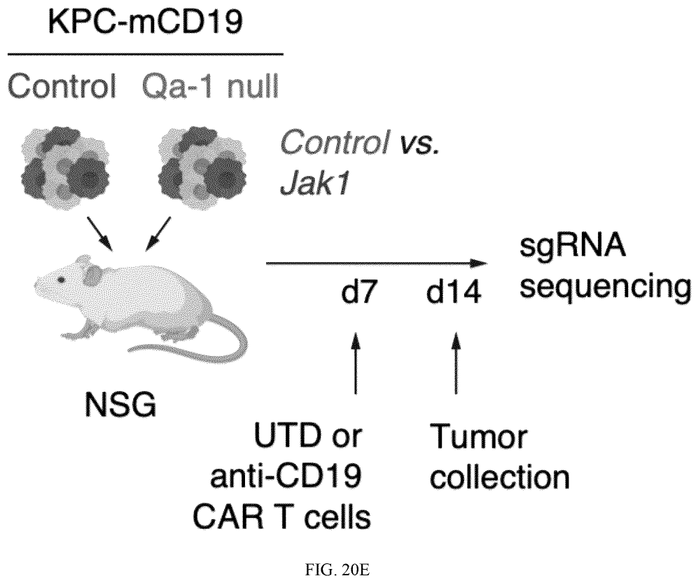

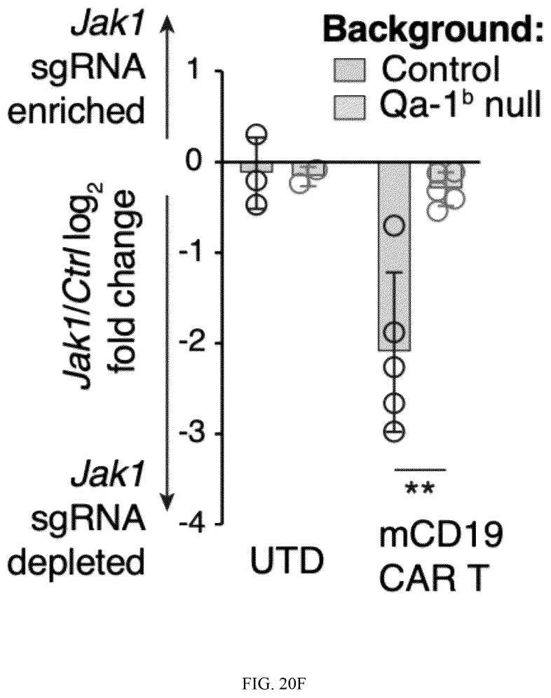

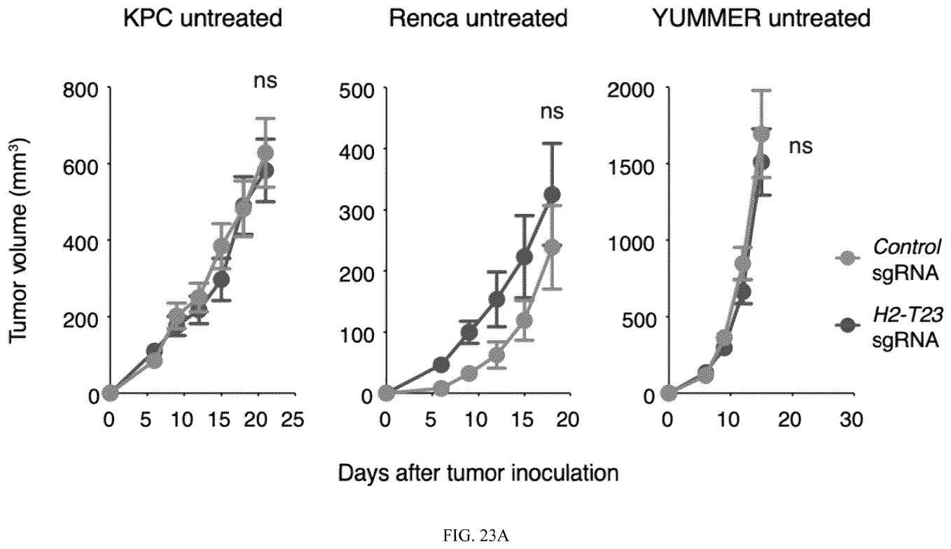

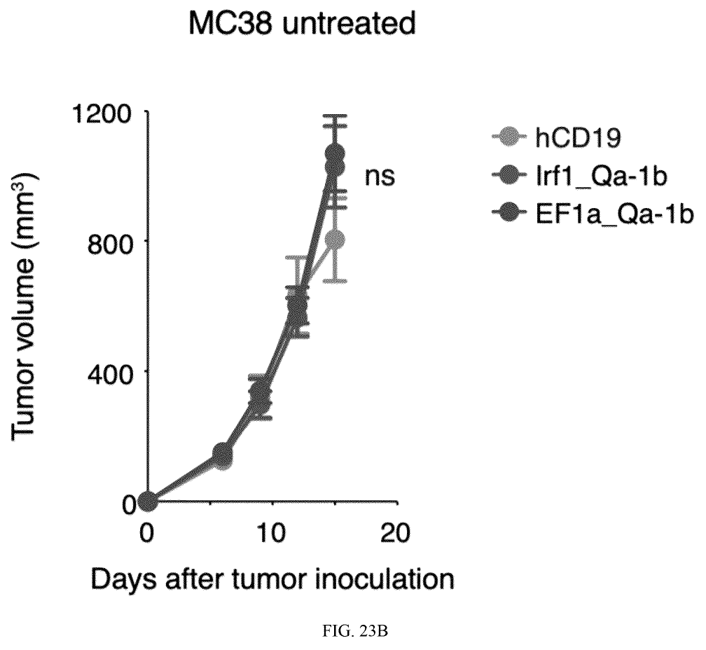

H2-T23, the mouse ortholog of human HLA-E, scored in the top 15 genetic dependencies in 7 out of the 8 models and was the top depleted hit overall, suggesting that this molecule was a potent negative regulator of anti-tumor immunity across cancer types (FIGS. 1B, 10A, and 10B). Loss of Qa-1^{b}, the protein encoded by H2-T23, was validated as an immunotherapy sensitizer across cancer models by generating control or H2-T23-deficient KPC, Renca, and YUMMER cell lines and implanting them in WT mice treated with ICB. In each model, H2-T23-deficient tumors had an enhanced response to ICB and were cured at a higher rate than control tumors (FIGS. 20A and 23A; Student's t test, p<0.05). MC38 colon carcinoma was the only cancer model that did not demonstrate an H2-T23 dependency in vivo. Interestingly, RNA sequencing revealed that H2-T23 expression is increased after stimulation with IFNγ in all models except MC38 (FIG. 10D). The lack of MC38 cell surface expression of Qa-1^{b} was validated by flow cytometry (FIGS. 19A and 10E). To determine if forced expression of Qa-1^{b} is sufficient to inhibit anti-tumor immunity in MC38, MC38 cells were engineered to express Qa-1^{b} from either a constitutively active or IFNγ-inducible promoter and implanted into WT mice treated with ICB (FIGS. 1E, 10E, and 23B). Compared to control CD19⁺ MC38, MC38 tumors expressing either constitutive or IFN-inducible Qa-1^{b} (FIG. 10E) were resistant to ICB and mice showed poor survival following ICB treatment (FIG. 1E; Student's t test, p<0.05). Thus, while not intending to be bound by theory, Qa-1^{b} expression could potently inhibit anti-tumor immunity in all murine cancer models tested.

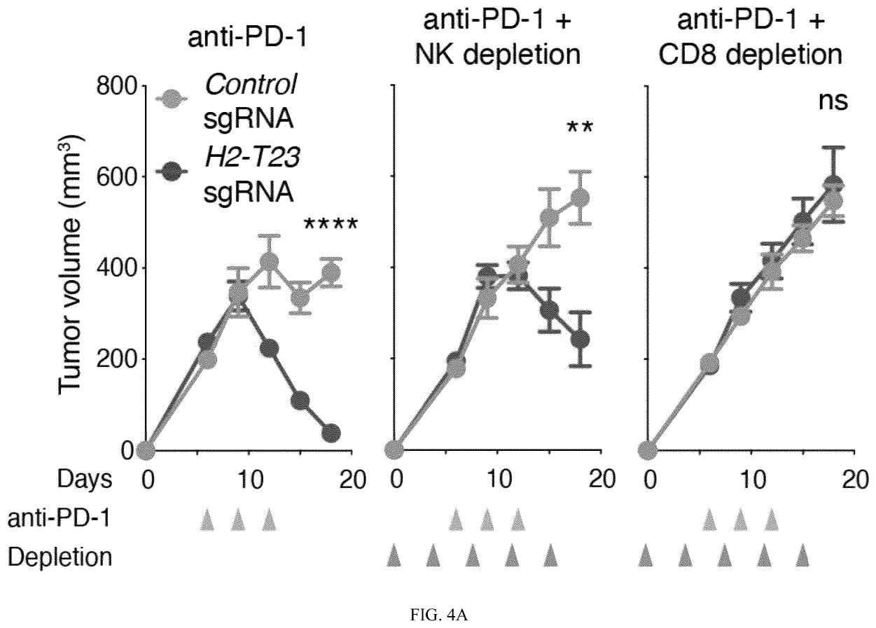

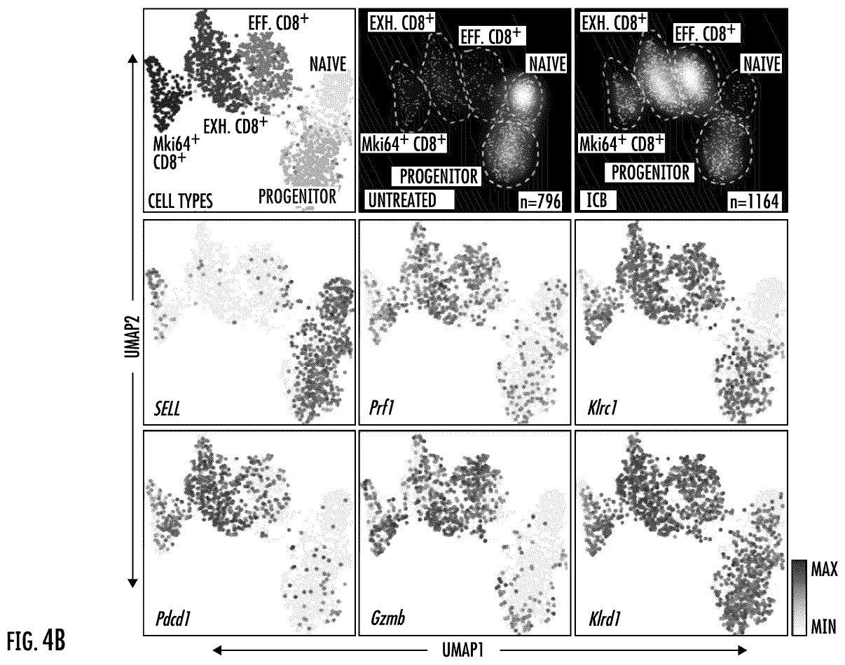

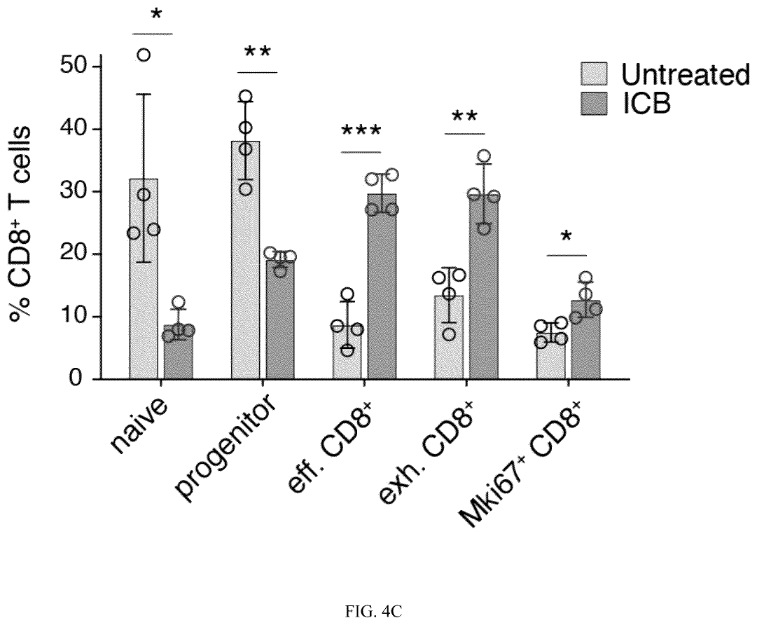

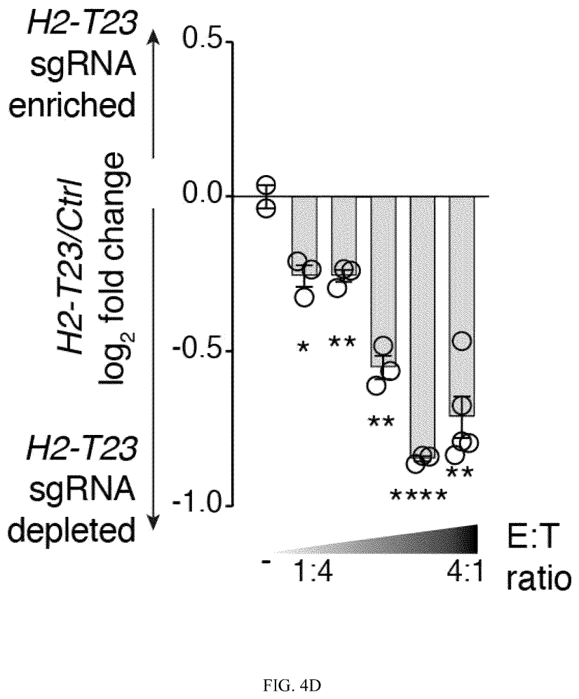

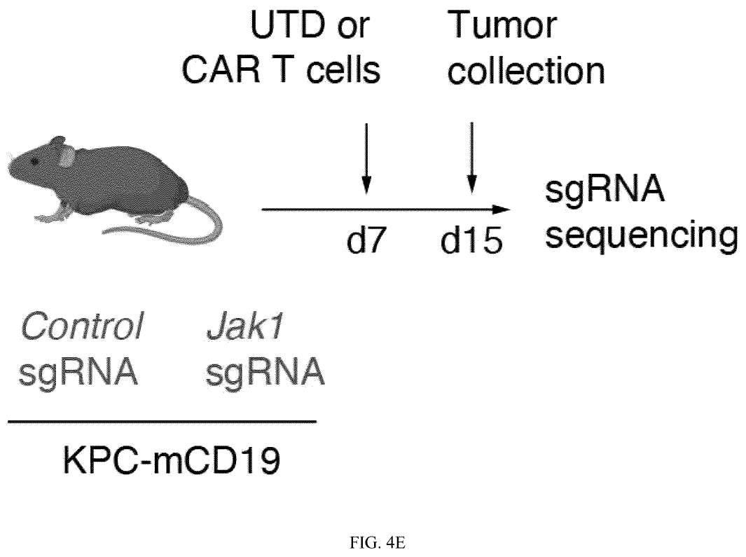

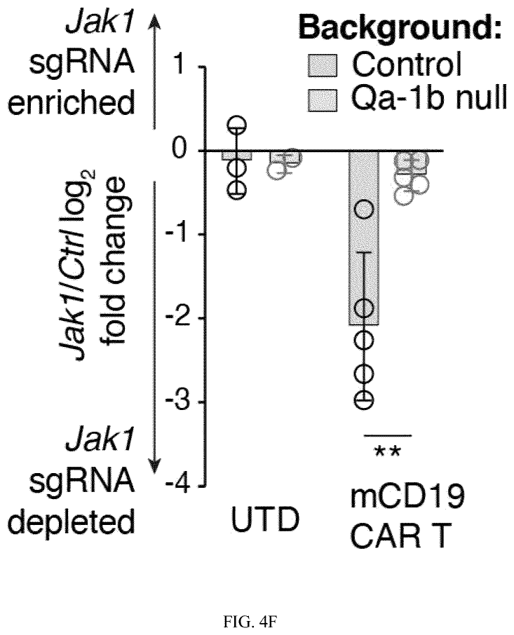

To clarify which immune subsets were inhibited by Qa-1^{b} expression in vivo, H2-T23-null KPC tumors were implanted into WT mice treated with anti-PD-1 alone or anti-PD-1 and antibodies to deplete either NK1.1⁺ cells or CD8⁺ T cells. While there was still strong efficacy of PD-1 blockade for H2-T23-deficient tumors in anti-NK1.1-treated mice, depletion of CD8⁺ T cells completely suppressed the efficacy of anti-PD-1 and tumors grew progressively (FIG. 4A; Student's t test, p<0.01), suggesting that Qa-1^{b} primarily inhibited CD8⁺ T cell functions.

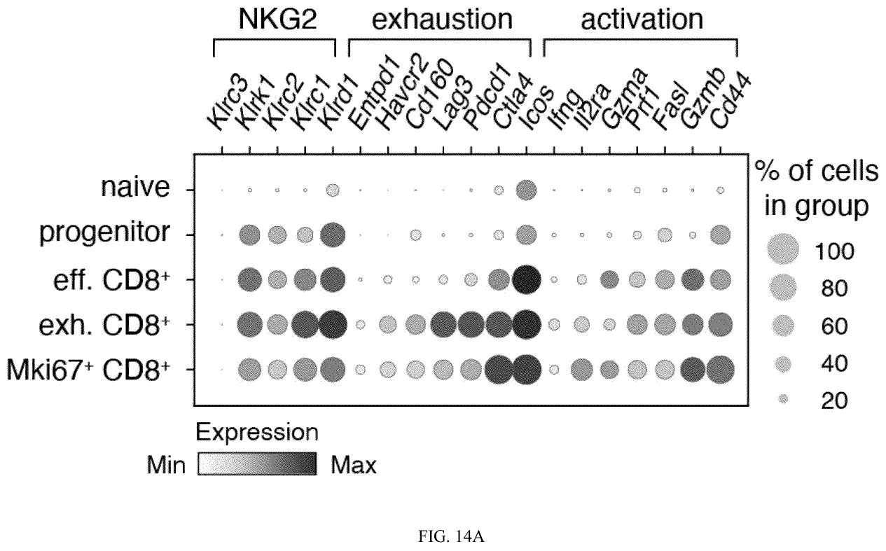

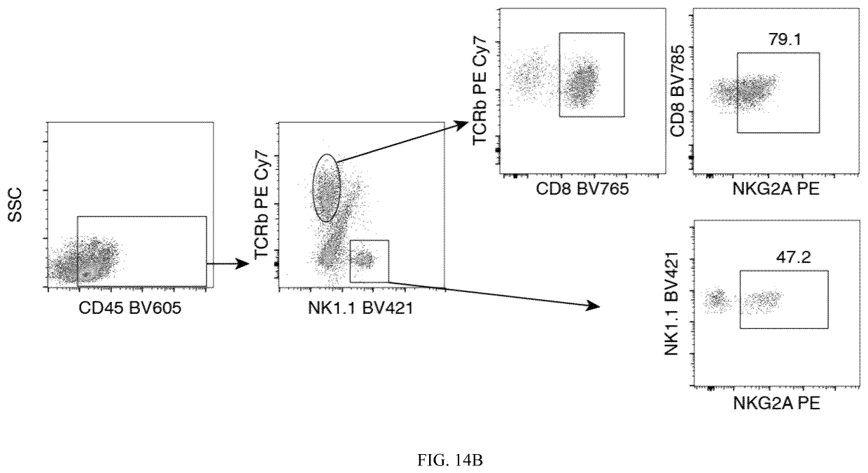

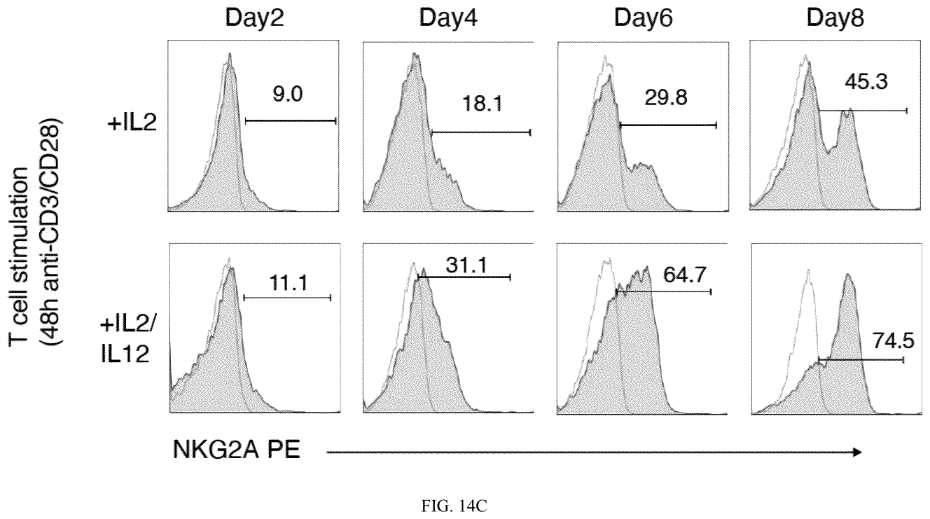

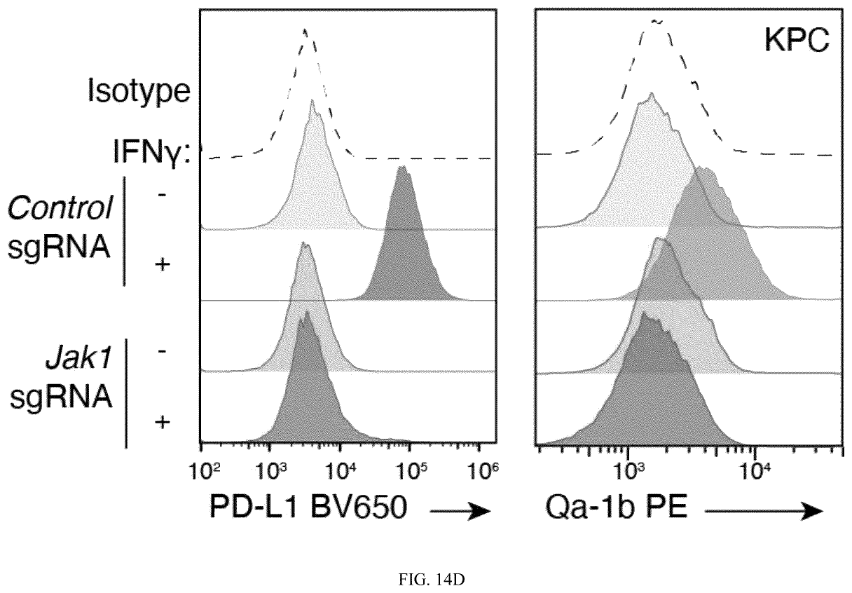

Because CD8⁺ T cells were primarily responsible for control of H2-T23-deficient tumors, subsets of tumor-infiltrating CD8⁺ T cells were delineated and examined for their expression of NKG2-family receptors along with canonical markers of effector function, activation and exhaustion using the single-cell RNA sequencing data (FIGS. 12A-12F). Five (5) subsets of CD8⁺ T cells were recovered in varying states of activation and effector function ranging from naive-like to exhausted (FIG. 4B, top). Immune checkpoint blockade (ICB) caused a marked shift in the transcriptional profile of infiltrating CD8⁺ T cells, with significant decreases in naive and progenitor subsets and increases in effector, exhausted, and proliferating subsets (FIGS. 4B, 4C, and 20B). Gene expression analysis showed the highest expression of cytotoxic genes on effector, exhausted, and proliferating subsets, and the accumulation of exhaustion-related inhibitory receptors on the terminal effector and exhausted subsets (FIGS. 4B and 14A). Strikingly, acquisition of NKG2A (Klrc1) and CD94 (Klrd1) expression marked the majority of effector, exhausted and proliferating CD8⁺ T cells (FIGS. 4B and 14A). Average NKG2A expression across cells in the population was higher for the activated populations of CD8⁺ T cells and NK cells (FIGS. 12C and 12D), which was confirmed using flow cytometry on tumor-infiltrating CD8⁺ T cells and NK cells (FIG. 14B). Thus, while not intending to be bound by theory, the NKG2A/CD94 complex was expressed on effector and exhausted CD8⁺ T cells with cytotoxic function and can be a key regulator of these subsets.

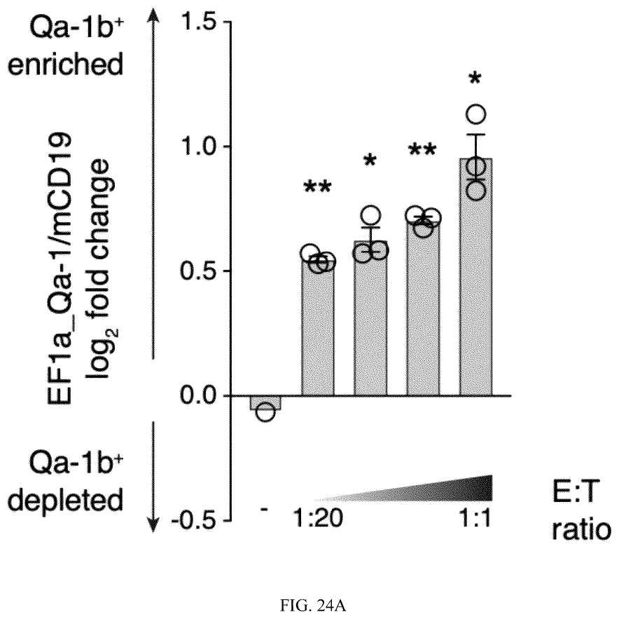

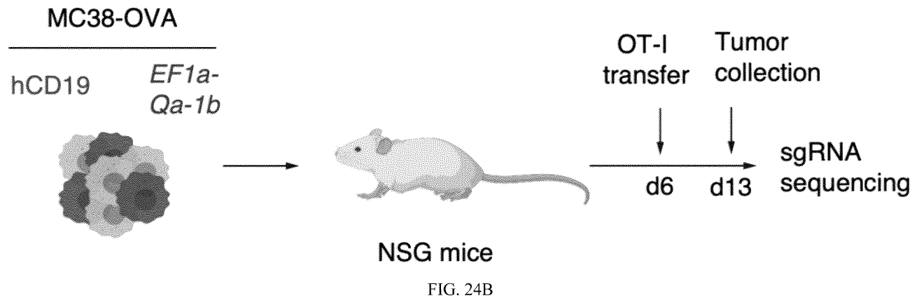

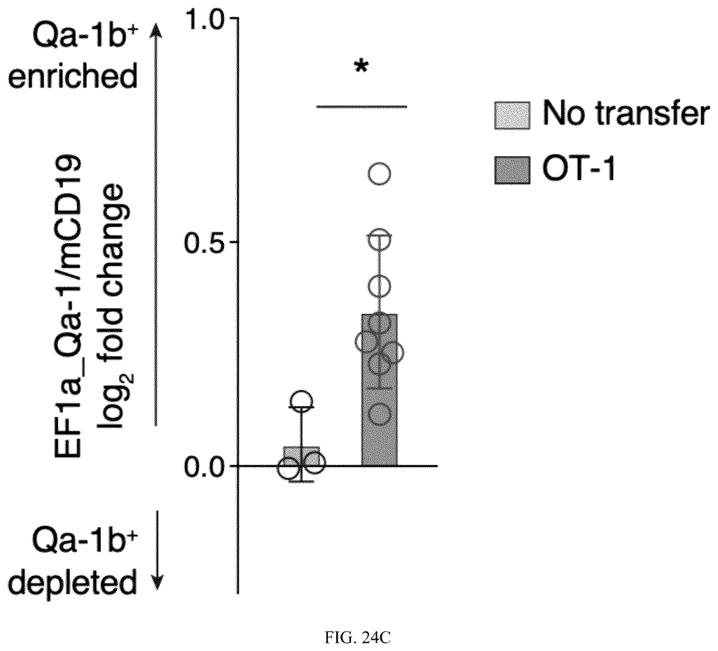

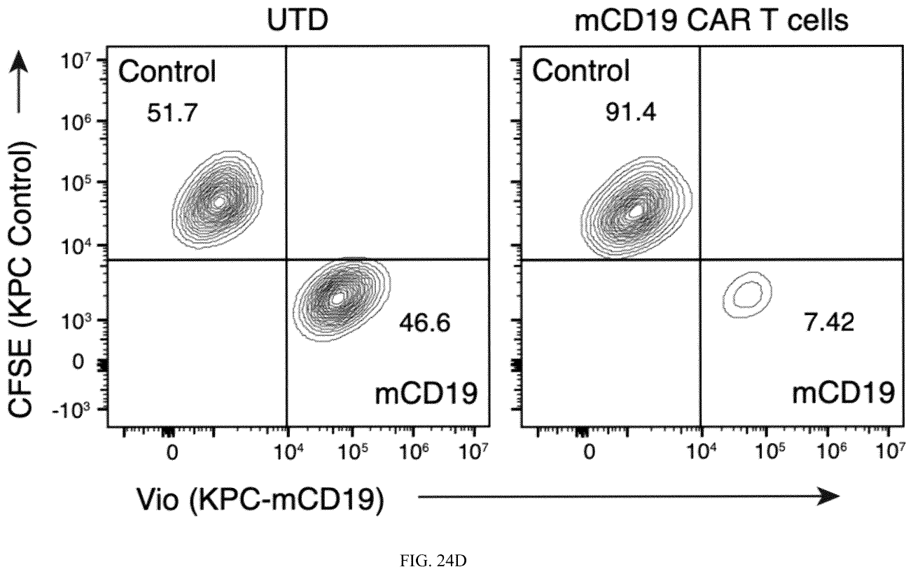

Experiments were next undertaken to determine whether expression of Qa-1^{b} directly inhibited CD8⁺ T cell cytotoxicity. In contrast to tumor-infiltrating CD8⁺ T cells, splenic CD8⁺ T cells activated in vitro with anti-CD3/CD28 antibodies and IL-2 expressed only low levels of NKG2A/CD94 (FIG. 14C, top). Therefore, IL-12 was added to the media as it has been described to promote NKG2A/CD94 expression (FIG. 14C, bottom). An in vitro cytotoxicity assay was then performed using MC38 cells, which do not natively express Qa-1b, bearing the model antigen ovalbumin (MC38-Ova) along with either constitutively expressed Qa-1^{b} or human CD19 as a control. When a 1:1 mixture of Qa-1^{b}- or control gene-overexpressing MC38-Ova was co-cultured with activated OT-1 transgenic T cells, Qa-1^{b}-overexpressing cells were enriched relative to controls (FIG. 24A; Student's t test p<0.05). Similar results were obtained in vivo: the mixture of Qa-1^{b}- or control gene-overexpressing MC38-Ova cells was implanted into NSG mice and the mice were injected with activated OT-1 T cells on day 6 following tumor implantation. Qa-1b overexpressing cells were enriched relative to control cells in the OT-1 transferred mice (FIGS. 24B and 24C; Student's t test, p<0.05). Thus, constitutive Qa-1^{b} expression was sufficient to inhibit CD8⁺ T cell cytotoxicity in vitro and in vivo.

Because not only Qa-1^{b}, but also the antigen presentation genes required for presentation of signal peptide on Qa-1^{b} (Tap1, Tap2, B2m, and H2-D) are upregulated by IFNγ (FIGS. 2C and 22A), it was hypothesized that tumor IFN sensing could inhibit CD8⁺ T cell cytotoxicity via the upregulation of Qa-1^{b}. Loss of tumor interferon (IFN) sensing causes resistance to CD8⁺ T cell cytotoxicity because interferon (IFN) is required for antigen presentation to CD8⁺ T cells via classical MHC-I, potentially masking direct interferon (IFN)-mediated inhibition of CD8⁺ T cell immunity. To overcome this confounding effect, a chimeric antigen receptor (CAR) system was used to decouple CD8⁺ T cell antigen recognition from classical MHC-I upregulation. It was first assessed whether Qa-1^{b} inhibited CAR-T activity against tumor cells: a 1:1 mixture of fluorescently labeled control and H2-T23-null mouse CD19⁺ KPC tumor cells was generated and treated with IFNγ to upregulate Qa-1^{b}. This mixture was then co-cultured at increasing effector:target ratios with T cells that were transduced with a CAR targeting mouse CD19. A dose-dependent depletion of H2-T23-null cells (FIG. 4D; Student's t test, p<0.05) was observed, indicating that Qa-1^{b} inhibited CAR-T killing.

Experiments were next undertaken to determine whether tumor upregulation of Qa-1^{b} could be a major mechanism of interferon-mediated inhibition of T cells. To directly examine the relationship between tumor interferon (IFN) sensing and upregulation of inhibitory ligands such as Qa-1^{b}, CD19 CAR-T cells were co-cultured with a 1:1 mixture of fluorescently labeled and IFNγ-stimulated control and Jak1-null CD19⁺ KPC tumor cells (FIG. 20C), which fail to upregulate ISGs including PD-L1 and Qa-1^{b} in IFNγ (FIG. 14D). No statistically significant difference in the survival of either population was observed (FIG. 20D, top) despite the observation of robust CAR-T cell killing in the culture (FIG. 24D), suggesting that the use of a CAR eliminated the survival advantage for tumor cells that lacked IFN sensing. To determine if IFN-mediated PD-L1 upregulation sensing and upregulation of inhibitory ligands such as Qa-1b, CD19 CAR-T cells were co-cultured with a 1:1 mixture of fluorescently labeled and IFNγ-stimulated control and Jak1-null CD19⁺ KPC tumor cells (FIG. 20C), which fail to upregulate ISGs including PD-L1 and Qa-1b in IFNγ (FIG. 14D). No statistically significant difference in the survival of either population was observed (FIG. 20D, top) despite the observation of robust CAR-T cell killing in the culture (FIG. 24D), suggesting that the use of a CAR eliminated the survival advantage for tumor cells that lacked IFN sensing. To determine if IFN-mediated PD-L1 upregulation contributed to CAR-T cell suppression, the assay was repeated in the presence of PD-1 blocking antibody a slight enrichment of Jak1-deficient cells was observed (FIG. 20D), which was expected if the upregulation of PD-L1 directly inhibited T cell cytotoxicity. However, when the assay was repeated with H2-T23-deficient KPC cells or with NKG2A blocking antibody, striking dose-dependent survival advantage was observed for the Jak1-null KPC cells (FIG. 20D, Student's t test, p<0.05). Next, it was assessed whether interferon (IFN)-mediated upregulation of Qa-1^{b} could inhibit CAR-T killing of solid tumors in vivo. A 11 mixture of control and Jak1-deficient CD19⁺ KPC cells (FIG. 14D) was implanted on either a control or H2-T23-null background into immunodeficient NOD SCID I12rg^{-/-} (NSG) mice and, 7 days later, injected either untransduced or CD19 CAR⁺ activated T cells (FIGS. 4E and 20E). Strikingly, Jak1-deficient cells were strongly depleted in CAR-T cell-treated control tumors but not in tumors receiving untransduced T cells (FIGS. 4F and 20F). However, this effect was completely abrogated on the background of Qa-1^{b}-null tumors and Jak1 deletion did not confer a fitness cost in this setting (FIGS. 4F and 20F, Student's t test, p<0.01). Thus, tumor interferon signaling directly inhibited CD8⁺ T cell cytotoxicity via the upregulation of Qa-1^{b}, which is the ligand for the NKG2A inhibitory receptor.

### Example 5: Interferon Inflammation in Post-Treatment Serum Protein Analysis was Associated with Immunotherapy Resistance in Melanoma

To examine the effect of interferon (IFN) signaling on patient survival, data was reanalyzed from two immune checkpoint blockade (ICB)-treated patient cohorts with clear cell renal cell carcinoma (ccRCC) or advanced melanoma (Liu, D. et al. Integrative molecular and clinical modeling of clinical outcomes to PD1 blockade in patients with metastatic melanoma. Nat. Med. 25, 1916-1927 (2019); Braun, D. A. et al. Interplay of somatic alterations and immune infiltration modulates response to PD-1 blockade in advanced clear cell renal cell carcinoma. Nat. Med. 26, 909-918 (2020)). To examine the epistatic effect of disrupted MHC-I presentation on tumor interferon signaling in human cancers, a recurring deletion was identified in the 6p21.3 genomic locus across human cancers in The Cancer Genome Atlas (TCGA), including in renal cell carcinoma (RCC) and melanoma (FIG. 15A). The 6p21.3 locus encodes several genes that are essential for the cell surface expression of MHC-I, including antigen processing transporter genes (TAP1, TAP2, TAPBP) and immunoproteasome genes (PSMB8, PSMB9), and genomic deletion of this region impairs MHC-I presentation by tumor cells. The frequency of 6p21.3 deletion across cancers varied, but many cancer types showed loss of 6p21.3 in >10% of samples in The Cancer Genome Atlas (TCGA) (FIG. 15A). Notably, 6p21.3 deletion alone did not stratify survival in most tissue types in The Cancer Genome Atlas (TCGA) or either immune checkpoint blockade (ICB) cohort (FIGS. 15A and 15B). Therefore, the effect of interferon-stimulated gene (ISG) expression was examined, a proxy for tumor interferon levels, on survival in tumors with either an intact or deleted 6p21.3 locus.

In the clear cell renal cell carcinoma (ccRCC) cohort, a high interferon-stimulated gene (ISG) signature was significantly associated with poor overall survival in tumors with intact 6p21.3 loci (FIG. 5A; p=0.0006). However, it was observed that in tumors carrying 6p21.3 deletions, a high interferon-stimulated gene (ISG) signature was no longer associated with poor patient survival (FIGS. 5A and 15C; p=0.522), indicating that interferon-mediated inhibition was dependent on the expression of MHC-I in clear cell renal cell carcinoma (ccRCC). Since interferon signaling in pre-treatment biopsies is predictive of response to immunotherapy. A cohort of melanoma patients treated with PD-1 blockade was analyzed and found to be consistent with previous reported results. It was found that high interferon-stimulated gene (ISG) expression was associated with improved survival in tumors with intact 6p21.3 loci (FIG. 5B; p=0.00001). It was hypothesized that the observed inverse survival associations between melanoma and clear cell renal cell carcinoma (ccRCC) could depend on the relative dominance of CD8⁺ T cells versus NK cells as the tumor-controlling effector population, thus changing whether interferon (IFN)-mediated upregulation of tumor MHC-I exerted a net inhibitory effect. To assess the effect of the immune context on interferon's association with survival, survival regression interaction modeling was performed on an expanded melanoma dataset (Liu, D. et al. Integrative molecular and clinical modeling of clinical outcomes to PD1 blockade in patients with metastatic melanoma. Nat. Med. 25, 1916-1927 (2019); Gide, T. N. et al. Distinct Immune Cell Populations Define Response to Anti-PD-1 Monotherapy and Anti-PD-1/Anti-CTLA-4 Combined Therapy. Cancer Cell 35, 238-255.e6 (2019); Riaz, N. et al. Tumor and Microenvironment Evolution during Immunotherapy with Nivolumab. Cell 171, 934-949.e16 (2017)) (FIG. 5C). Interactions were modeled with several variables associated with anti-tumor immunity or response to checkpoint blockade, such as CYT score (Rooney, M. S., Shukla, S. A., Wu, C. J., Getz, G. & Hacohen, N. Molecular and genetic properties of tumors associated with local immune cytolytic activity. Cell 160, 48-61 (2015)), CD3, PDCD1, and CD274 expression, but no significant interactions with interferon signature were found. Given the data showing that NK cell depletion reversed the sensitization of Jak1-null tumors to immune checkpoint blockade (ICB) (FIG. 3F), tumors were then scored on whether they were primed for an NK or CD8⁺ T cell response based on the expression of NK activating ligands (MICA, MICB, ULBP1, and further ligands listed in the methods provided herein) or pre-existing expansion of CD8⁺ T cell clones (average T cell clone size), respectively. Both of these features interacted significantly with interferon-stimulated gene (ISG) signature in predicting survival: concordant with the hypothesis that being primed for an NK response predicted that high interferon would be associated with poor survival, while being primed for a CD8⁺ T cell response predicted a favorable role for interferon. Thus, the effects of interferon on tumor progression were dependent on the immune context of the tumor.

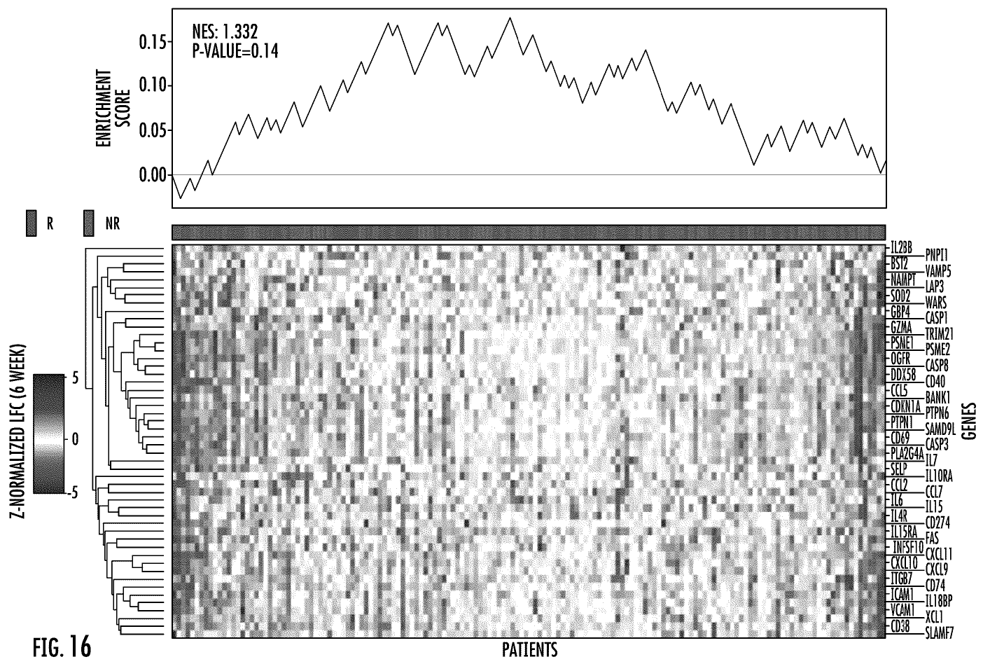

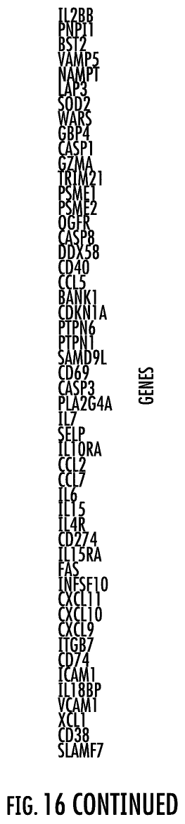

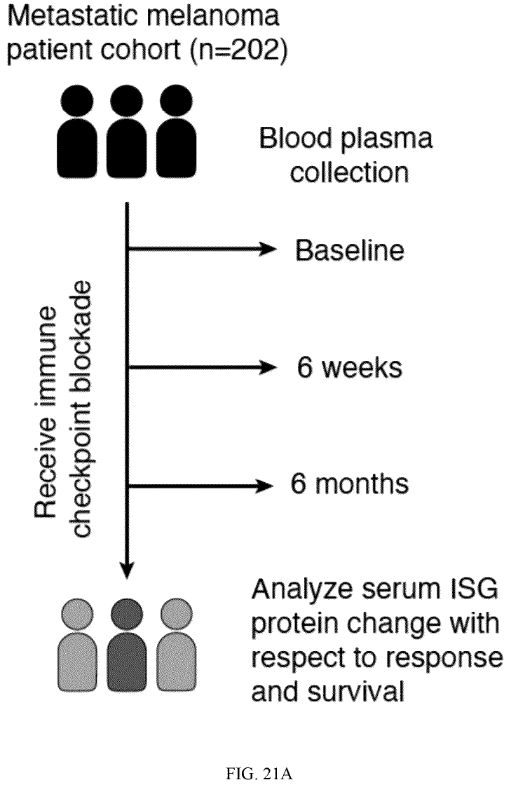

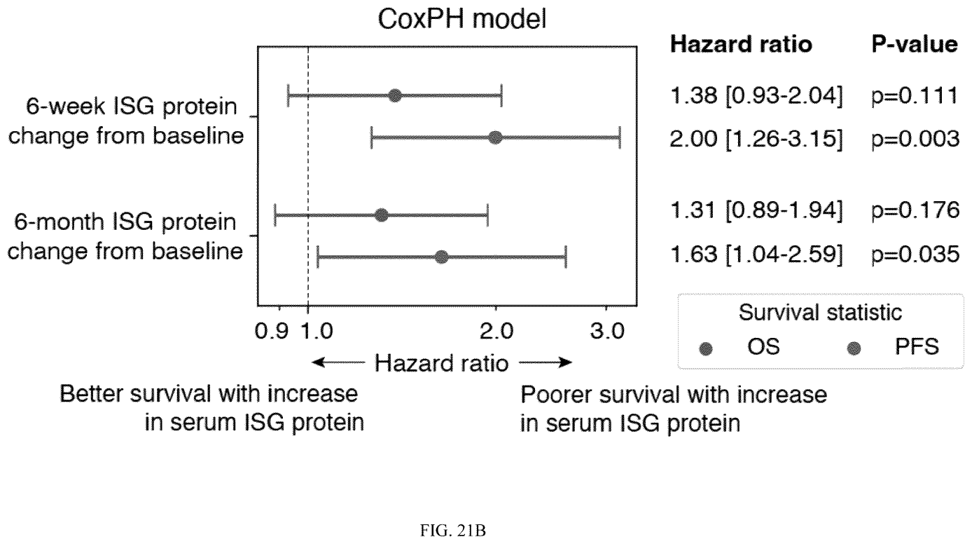

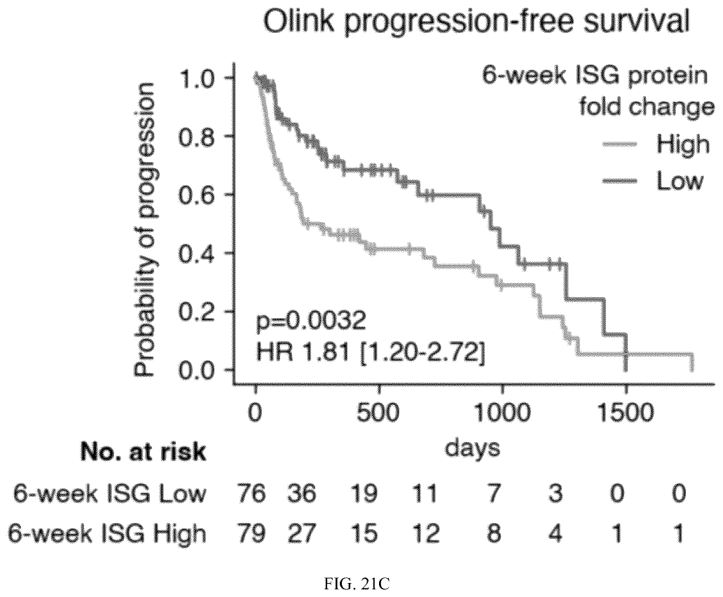

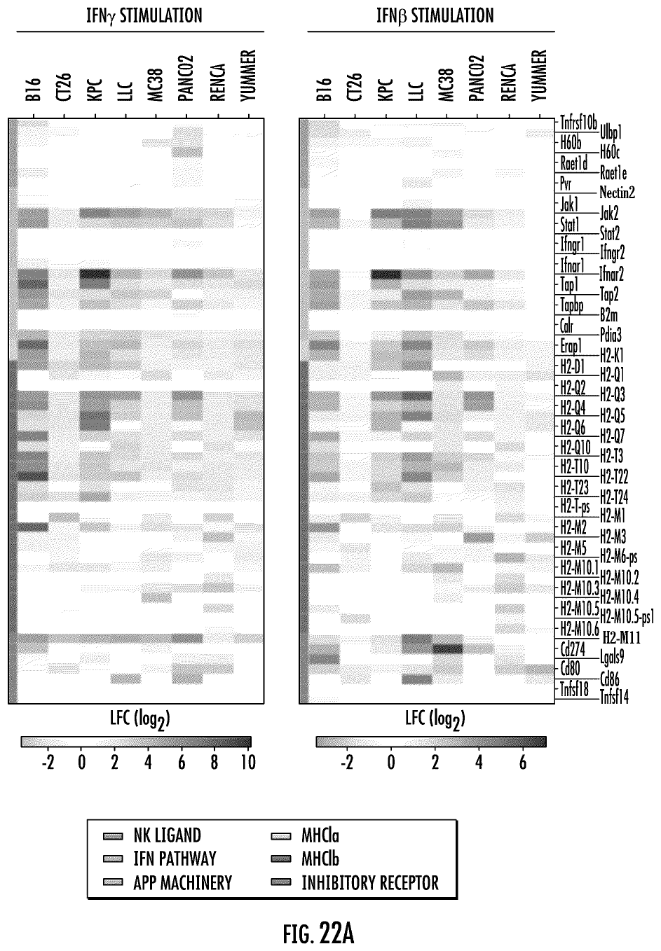

While the analyses of pre-treatment biopsies provided evidence for the context specificity of interferon (IFN)-mediated immune inhibition, the data from the mouse models suggested that interferon (IFN) signaling activated adaptive resistance to immune checkpoint blockade (ICB), and thus inhibitory effects of interferon would be more pronounced in on-treatment samples. To that end, serum protein expression was collected for 50 ISGs from 203 immune checkpoint blockade (ICB)-treated melanoma patients from a pre-treatment time point and 6-week and 6-month post-treatment time points (FIG. 21A). Both responder and non-responder patients showed an increase in serum interferon-stimulated gene (ISG) levels 6 weeks after treatment initiation (FIG. 5D). Although there was no association between change in serum interferon-stimulated gene (ISG) protein levels and response at 6-weeks (FIG. 16), a significant enrichment was observed for non-responders in patients that had high serum interferon-stimulated gene (ISG) proteins at the 6-month time point (FIGS. 5D and 5E). Among the proteins measured, elevated IFNγ and HLA-E expression was observed in non-responders at 6 months, while responders had reduced expression (FIG. 5F; p<0.005). Cox proportional hazard modeling was then performed on overall and progression-free survival based on mean change in serum ISG protein at 6 weeks and 6 months. Higher serum ISG protein at 6 weeks and 6 months was associated with higher hazard in overall survival but was not statistically significant (FIG. 21B; p=0.111, p=0.176, respectively). However, a statistically significant association was found with progression-free survival and higher ISG protein at both time points (FIGS. 21B and 21C; p=0.003, p=0.035, respectively by Cox regression; p=0.003 by median split log-rank test at 6 weeks), suggesting that an increased interferon signature was associated with on-therapy resistance. Thus, resistance to immune checkpoint blockade (ICB) was associated with elevated levels of interferon (IFN) and interferon-regulated proteins such as HLA-E during tumor progression.

To systematically discover tumor-intrinsic immune evasion pathways in the tumor microenvironment, genome-scale in vivo CRISPR screens were performed in a collection of transplantable mouse tumor models treated with immune checkpoint blockade. This dataset revealed many new genetic dependencies and resistance mechanisms for anti-tumor immunity that provided a deeper understanding of how tumors evolve to evade immunity. The dataset can enable the generation of new therapeutics that enhance immune checkpoint blockade (ICB) efficacy. Importantly, the approach also revealed critical mechanisms of immune resistance that were shared by most tumors, such as interferon (IFN)-mediated inhibition of NK cells and CD8⁺ T cells by upregulation of classical MHC-I and the non-classical MHC-I Qa-1^{b}.

The finding of a dominant immune inhibitory effect from tumor interferon (IFN) sensing from in vivo screens for immune evasion genes was surprising because the production of IFNγ by CD8⁺ T cells and NK cells has long been appreciated as critical for control of tumors by the immune system, and mutations in the interferon-sensing pathway have been associated with clinical resistance to immune checkpoint blockade (ICB) or tumor progression. In the above Examples, it has been demonstrated that IFNγ-mediated upregulation of classical and non-classical MHC-I genes inhibited natural killer cells and CD8⁺ T cells, respectively, and was a central mechanism used by tumor cells to evade cytotoxic lymphocyte-mediated killing. The Examples represent the first report of adaptive resistance to immune checkpoint blockade (ICB) caused by IFNγ mediated upregulation of MHC-I family genes.

It is shown in the above Examples that a high interferon-stimulated gene (ISG) signature in a pre-treatment biopsy predicted resistance to immune checkpoint blockade (ICB) in clear cell renal cell carcinoma (ccRCC), while the same gene signature predicted response in patients with melanoma. The analysis revealed that the role of interferon (IFN) sensing can depend on the immune context of the tumor. In melanoma tumors with high expression of ligands that activate NK cell cytotoxicity such as MICA and MICB, high interferon-stimulated gene (ISG) expression predicts poor response, consistent with the mechanistic data showing that interferon (IFN)-driven upregulation of MHC-I inhibits NK cells. However, in melanoma tumors with highly clonally expanded T cell populations, it was observed that high interferon-stimulated gene (ISG) expression predicted favorable response to immune checkpoint blockade (ICB), consistent with interferon (IFN) signaling driving increased antigen presentation to T cells. Not intending to be bound by theory, in many cancers with a lower average number of mutations per megabase and fewer neoantigens, such as ccRCC, anti-tumor immune responses may not always be mediated by clonally-expanded CD8+ T cells recognizing high-affinity MHC-I epitopes, and instead may be dominated by NK cells or other effector populations. In these settings, where the presentation of high-affinity epitopes is not driving an immune response, the upregulation of MHC-I genes is immune-protective, as a large number of ITIM-containing inhibitory receptors (NKG2A, KIR family, LIR family) can recognize MHC-I and are expressed across several immune subsets including T cells, NK cells and macrophages56. Thus, the IFN-mediated upregulation of MHC-I serves to promote immunity via antigen presentation when immunogenic MHC-I epitopes are present but then dampen immunity by providing ligands for ITIM-containing immune receptors when immunogenic epitopes are absent. The classical MHC-I genes play a dual role in both antigen presentation to CD8⁺ T cells and inhibition of NK cell killing, thus highlighting the importance of understanding the nature of immunotherapy responses in different contexts.

However, certain members of the MHC-I family such as the non-classical MHC-I Qa-1^{b}/HLA-E are primarily inhibitory ligands and present antigen only in limited contexts, positioning them as key inhibitory molecules induced by interferon (IFN) that are less context-dependent than classical MHC-I.

The Qa-1^{b}/NKG2A inhibitory axis was identified as the top immune evasion mechanism across cancer types. Not intending to be bound by theory, the data suggested that the activation of CD8⁺ T cells by immune checkpoint blockade (ICB) upregulated the expression of NKG2A/CD94 as they differentiate into cytotoxic terminal effector and exhausted T cells. At the same time, the IFNγ inflammation induced by immune checkpoint blockade (ICB) drove the expression of Qa-1^{b}, the ligand for the NKG2A/CD94 inhibitory receptor. This adaptive resistance mechanism reveals how immune checkpoint blockade (ICB)-induced CD8⁺ T cell cytotoxicity can be dampened in the setting of strong interferon (IFN) inflammation despite high tumor cell expression of MHC-I. The data also revealed the importance of understanding the context-dependent activity of immune checkpoints, as the inhibitory interaction between Qa-1^{b} and NKG2A was difficult to model in vitro due to poor expression of NKG2A on CD8⁺ T cells under standard CD3/28+IL-2 activation conditions. It was found that IL-12 stimulation caused the upregulation of NKG2A on mouse CD8⁺ T cells in vitro, but further study was required to determine the exact signals that lead to NKG2A expression in vivo. Finally, the data on Qa-1^{b}(HLA-E) also suggested that the other non-classical MHC-I genes HLA-G and HLA-F may play a role in interferon (IFN)-mediated inhibition of anti-tumor immunity in human cancer. HLA-G and HLA-F were not assessed in the in vivo screens because they have no clear mouse orthologs. These non-classical MHC-I molecules are expressed in many tumors and their expression has generally been associated with poor prognosis. Thus, IFNγ-mediated inhibition of anti-tumor immunity may also involve additional non-classical MHC-I genes beyond HLA-E in humans and these inhibitory effects could impact a broad range of host immune cells.

### Example 6: Human CD8+ Tumor-Infiltrating Lymphocytes (TILs) Expressed High Levels of NKG2A and CD94 In Vivo

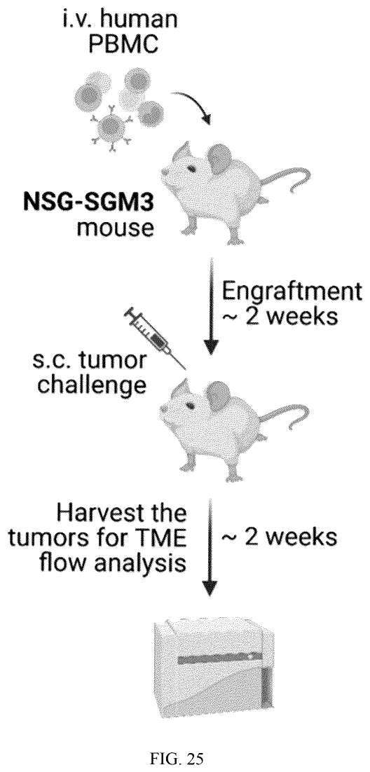

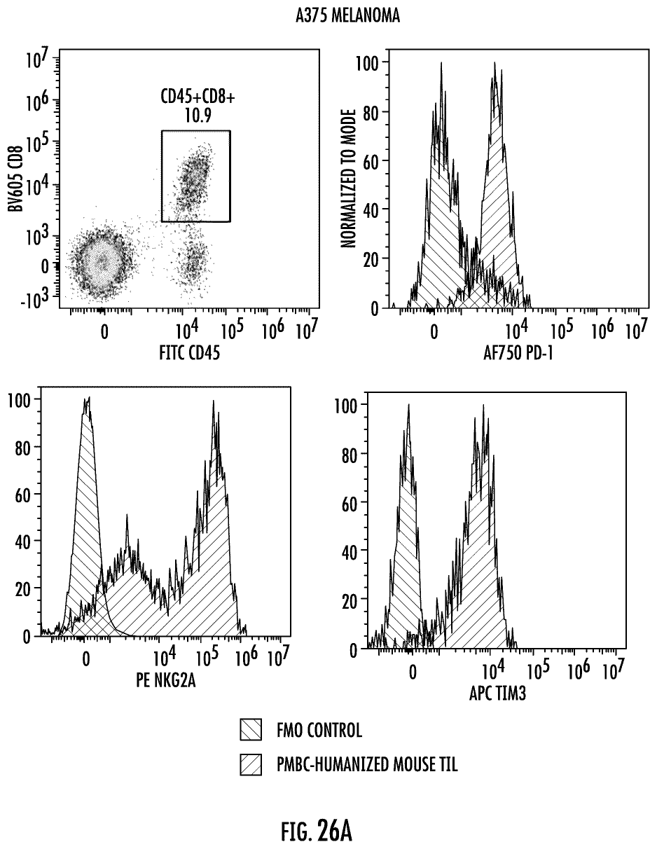

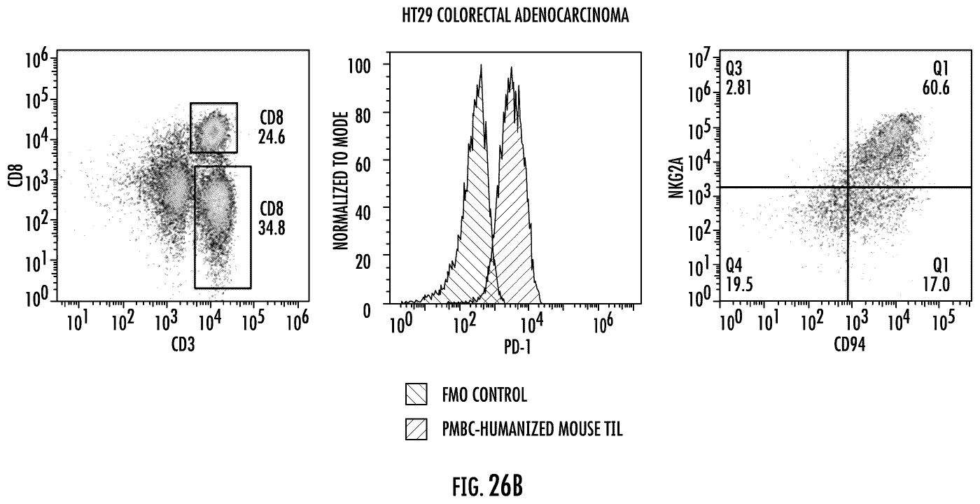

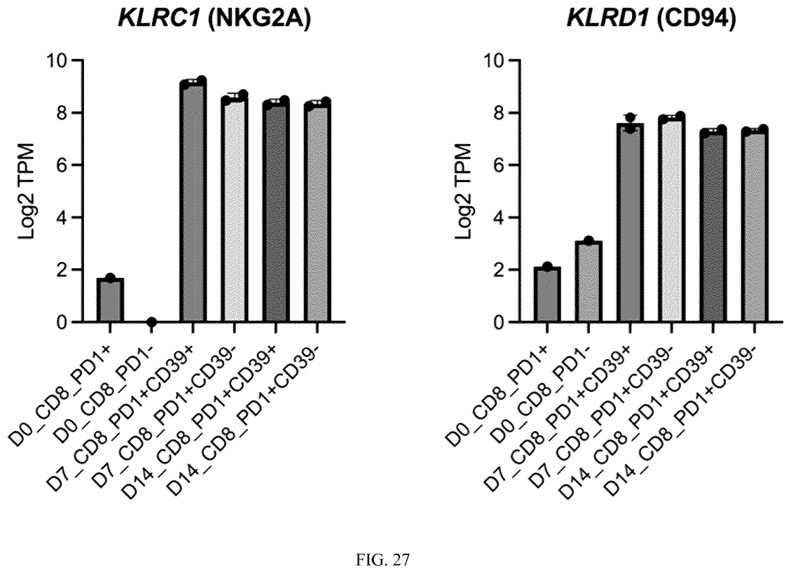

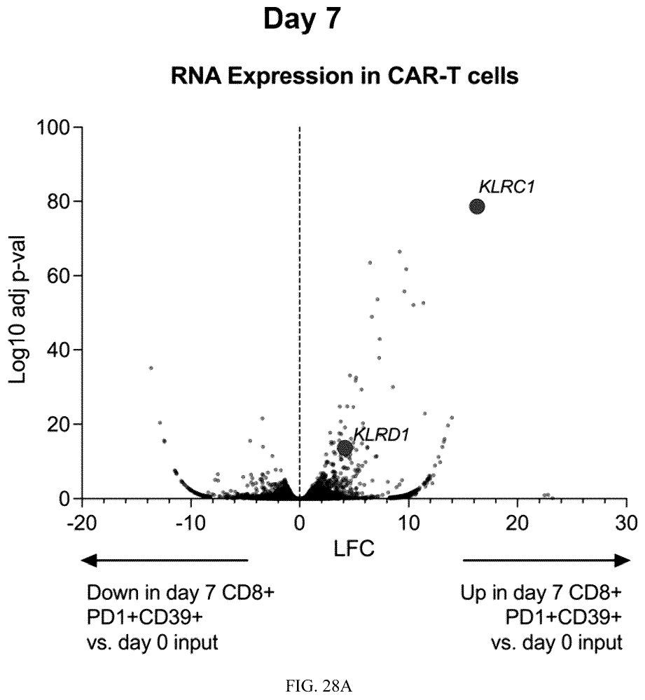

Experiments were undertaken to demonstrate that CD8+ tumor-infiltrating lymphocytes (TILs) express high levels of NKG2A and CD94 in vivo. The experiments were carried out as shown in FIG. 25, namely NSG-SGM3 mice were intravenously (i.v.) administered human peripheral blood mononuclear cells (PBMCs) and, two weeks following the administration, the mice were subcutaneously (s.c.) administered tumor cells. The tumors were harvested two weeks following administration of the tumor cells, and T cells within the tumors were analyzed using flow cytometry to evaluate the impact of the tumor microenvironment (TME) on gene expression in the T cells. NKG2A/CD94 co-expression was dramatically induced in CD8+ T cells within the tumor microenvironment of A375 melanoma tumors and HT29 colorectal adenocarcinoma tumors (FIGS. 26A and 26B). It was found that PD-1 expression was also induced in tumor-infiltrating T cells (FIGS. 26A, 26B, and 27).

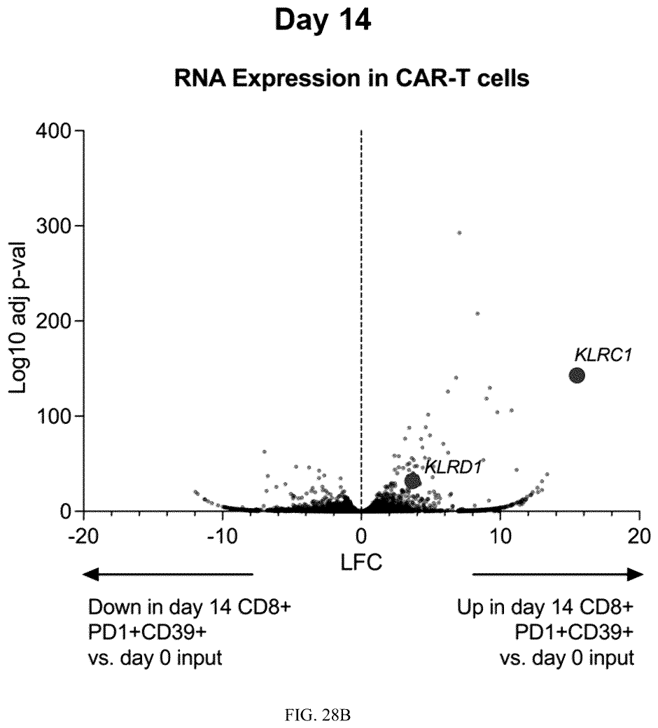

An experiment was undertaken to evaluate the impact of the tumor microenvironment on gene expression in tumor-infiltrating human chimeric antigen receptor expressing (CAR) T cells in vivo. At day 0 (input) NSG mice bearing A375 tumors were injected with CAR T cells targeting the tumors, and gene expression in CAR T cells within the tumors was evaluated after 7 and 14 days using RNAseq. RNAseq of the T cells from the tumors revealed that KLRC1 (encoding NKG2A) and KLRD1 (encoding CD94) were both upregulated on tumor-infiltrating human CD8+ CAR T cells in vivo (FIG. 27). The CD8+ T cells were divided into subsets based on of PD-1+ single-positive expression (activated T cells) or PD-1+ and CD39+ double-positive expression (terminally exhausted/effector T cells) (FIG. 27). It was found that, at 7 days following administration of the CAR T cells to the mice, KLRC1 and KLRD1 were among the most differentially expressed genes on the tumor-infiltrating human CAR T cells in vivo (FIGS. 28A and 28B).

These results demonstrate, among other things, that knockout of NKG2A and/or CD94 polypeptide expression in CAR T cells may reduce susceptibility to immune inhibition within the tumor microenvironment.

### Example 7: Induction of NKG2A and CD94 Expression in Human T Cells In Vitro

To facilitate the examination of the impact of NKG2A and/or CD94 up-regulation on T cell (e.g., CAR T cell) function, experiments were undertaken to develop a method for inducing expression of NKG2A and/or CD94 in T cells in vitro. On day zero CD8+ T cells were isolated from human peripheral blood mononuclear cells (PBMCs) using a CD8+ T cell isolation kit (Miltenyi Biotec). Isolated CD8+ T cells were then subjected to a first stimulation using T Cell TransAct™ available from Miltenyi Biotec following the manufacturer's protocol and cultured in TexMACS™ Medium (with Penicillin/Streptomycin) available from Miltenyi Biotec and supplemented with human IL-2, IL-7, IL-15, and/or IL-12p70. In the first stimulation, 1e6 cells+10 μl TransAct™ were combined in 1 ml TexMACS™ medium (1:100) in a 48-well plate format (Cytokine usage: IL-2 50 U/ml (PeproTech); IL-7 10 ng/ml (PeproTech); IL-15 10 ng/ml (PeproTech); IL-12p70 10 ng/ml (PeproTech)) and allowed to expand. Fresh medium with all cytokines was added to the cell cultures every 1-2 days, and TransAct™ was removed on day 2 according to the manufacturer's protocol. On day 14, the cells were subjected to a second stimulation similar to the first stimulation using TransAct at a 1:500 dilution. On day 16-18, cells were harvested for use in further analysis (e.g., use in a co-culture experiment) or frozen down with 50% CryoStor® CS10 cryopreservation medium (StemCell).

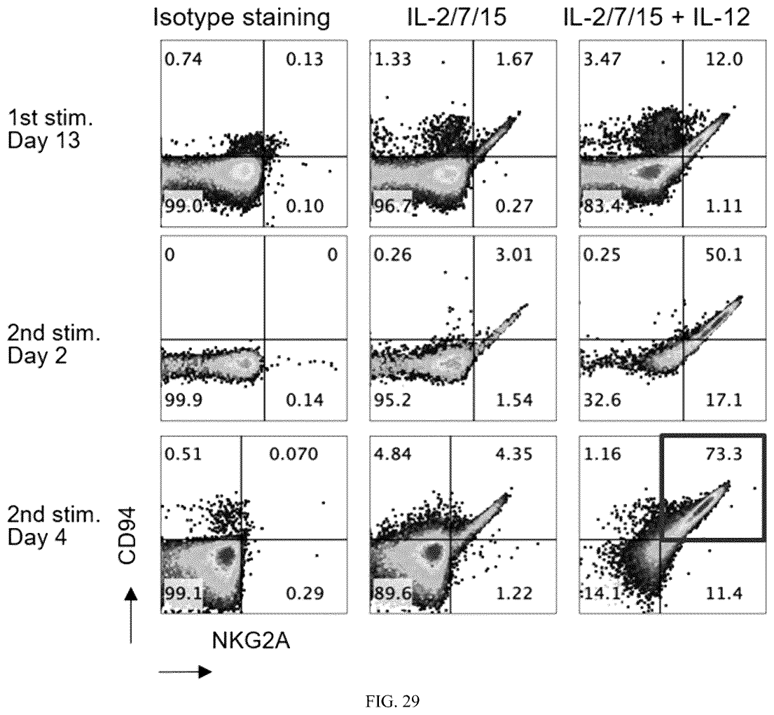

It was determined that T cells stimulated twice using an TransAct™, with IL-2, IL-7, IL-15, and IL-12 showed induced expression of NKG2A and CD94 (FIG. 29). Cells similarly stimulated using TransAct™ in combination with IL-2, IL-7, and IL-15, but not with IL-12, did not show the same levels of induced NKG2A and CD94 expression. NKG2A and CD94 expression was evaluated at 13 days following the first stimulation and at 2 and 4 days following the second stimulation. The cultured CD8+ T cells started to express high levels of NKG2A/CD94 about 2 or 3 days after the second stimulation. Traditional in vitro T cell expansion protocols did not induce NKG2A expression.

These results demonstrate the development of a new method for inducing NKG2A and CD94 expression in T cells in vitro.

### Example 8: Tumor Cells Deficient in HLA-E Expression were Sensitized to Killing by Immune Effector Cells

Experiments were undertaken to demonstrate that the interaction of HLA-E expressed on the surface of tumor cells within the tumor microenvironment (TME) mediated the immune inhibition of T cells within the TME.

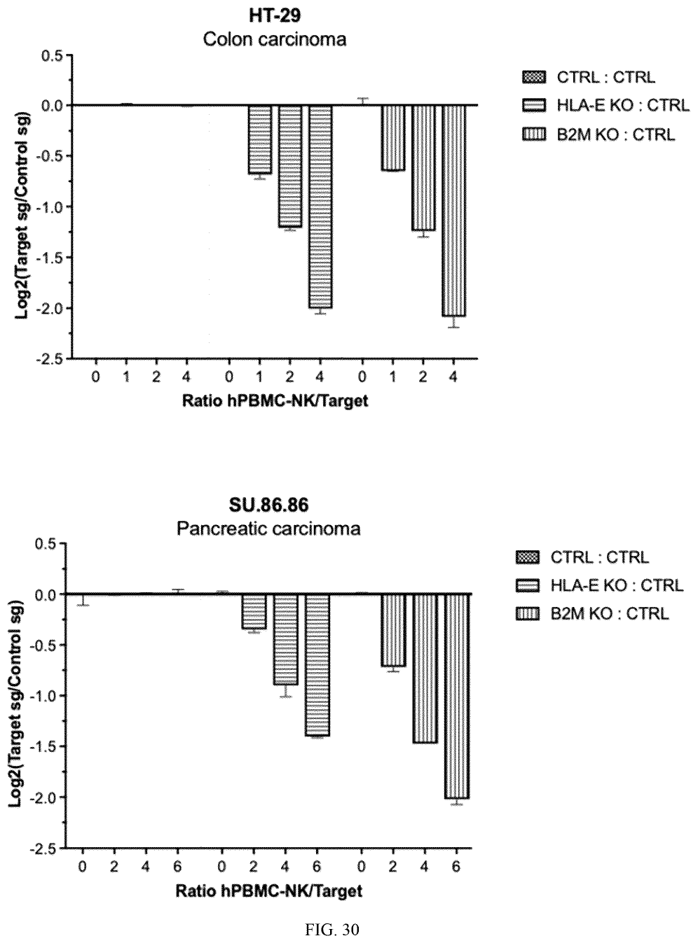

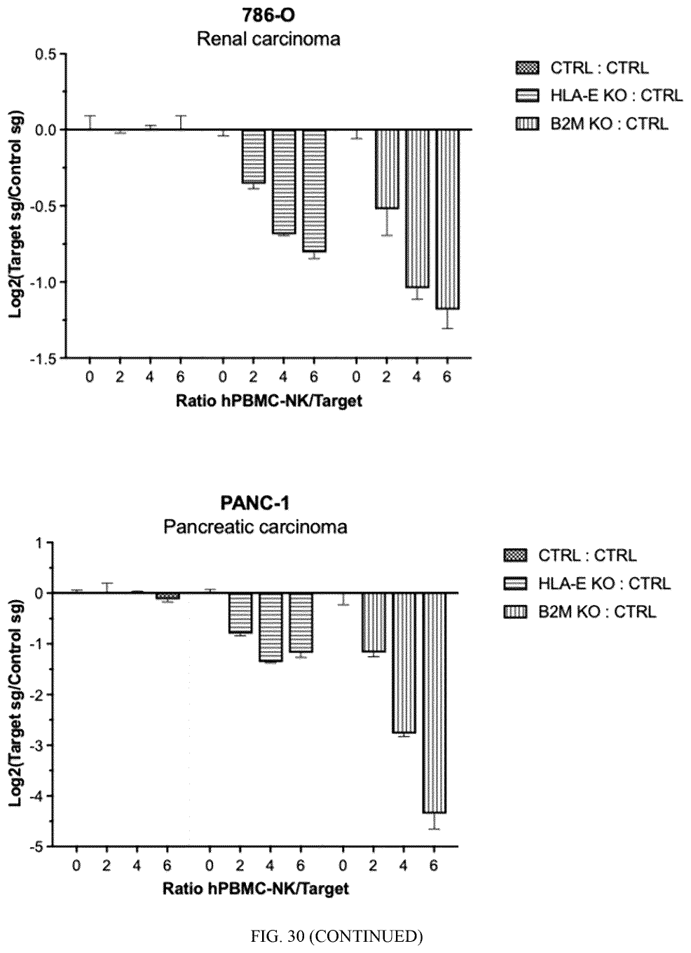

First, co-culture experiments were undertaken to demonstrate that deletion of HLA-E in tumor cells sensitized the cells to killing by NK cells. NK cells were used because a large frequency of NK cells surface-express CD94 and NKG2A without any need for induction. The tumor cells evaluated included HT-29 colon carcinoma cells, 786-0 renal carcinoma cells, SU.86.86 pancreatic carcinoma cells, and PANC-1 pancreatic carcinoma cells. Target cells were stained with either CellTraceViolet or CellTraceFarRed, seeded in culture flasks, and evaluated by flow cytometry to quantify cell input ratios (e.g., ratio of HLA-E knockout tumor cells to control tumor cells). Prior to co-culture, the tumor cells were stimulated with human IFNg for 24-48 hours (10 ng/ml for HT-29 tumor cells and 100 ng/ml for other tumor cells). On the day of coculture, human PBMC-derived NK cells were thawed, incubated and rested in culture medium (LGM-3 medium) for 3-4 hours and then counted. NK cells were added directly to the target cells without any media changes and then co-cultured for 24 hours with the tumor cells. By co-culturing the NK cells for 24 h at different effector-to-target cell ratios with tumor cells expressing or deficient in expression of HLA-E (i.e., control cells or HLA-E/B2M knockout (KO) cells, respectively), it was found that the deletion of HLA-E from the tumor cells sensitized them to killing by the NK cells (FIG. 30).

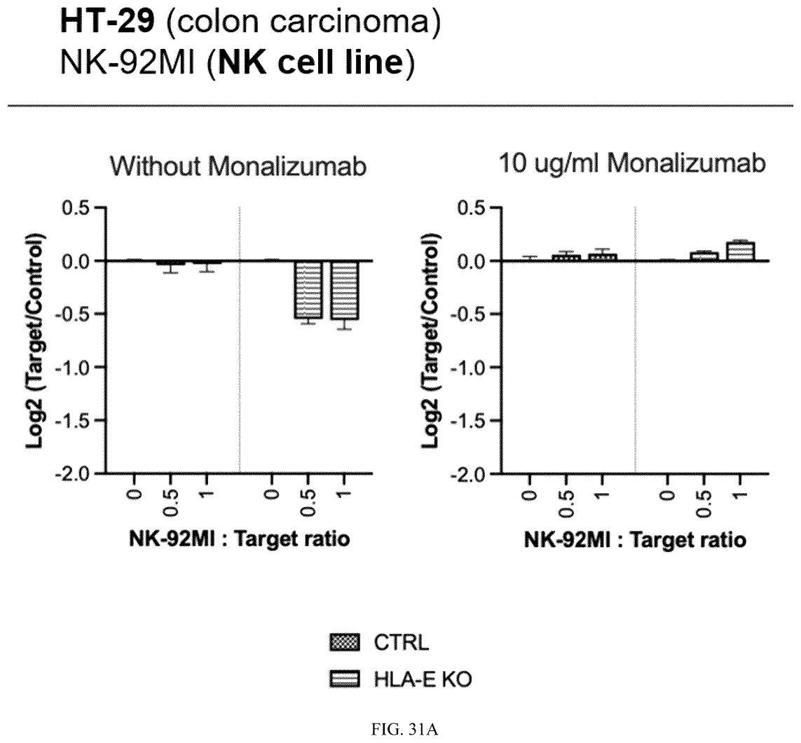

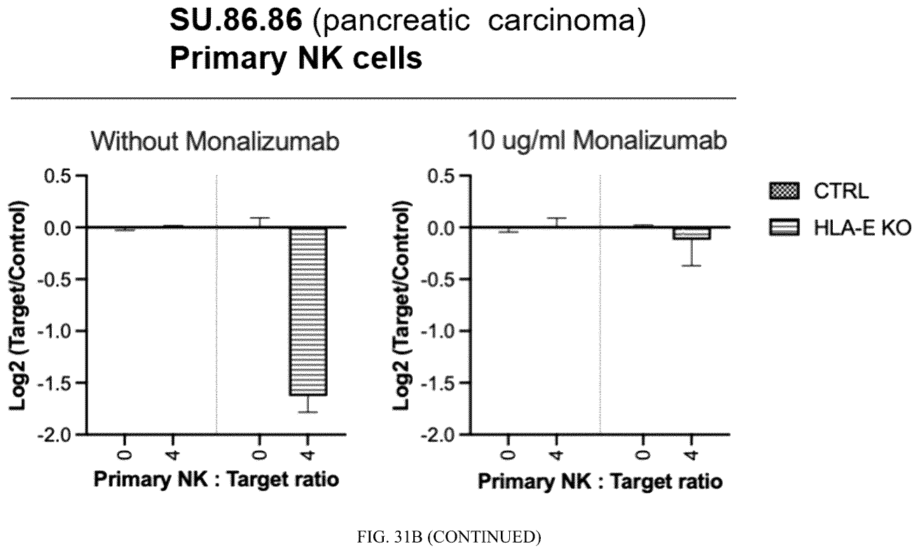

In further support of this result, it was found that Monalizumab reversed preferential killing of HLA-E KO tumor cells by NK cells. Co-cultures containing HT-29 or SU.86.86 cells (target cells) and the NK cells line NK-92MI or primary NK cells (effector cells) were administered 10 μg/ml Monalizumab. It was found that the Monalizumab eliminated preferential killing of HLA-E knockout (KO) cells over the control cells that expressed HLA-E (FIGS. 31A and 31B). Monalizumab is an antibody that binds NKG2A and blocks the binding thereof to HLA-E. Preferential killing of the HLA-E knockout tumor cells by the effector cells was blocked because the antibody Monalizumab blocked the ability NKG2A to bind to HLA-E, thereby eliminating the ability of the T cells to distinguish between the HLA-E knockout and control tumor cells.

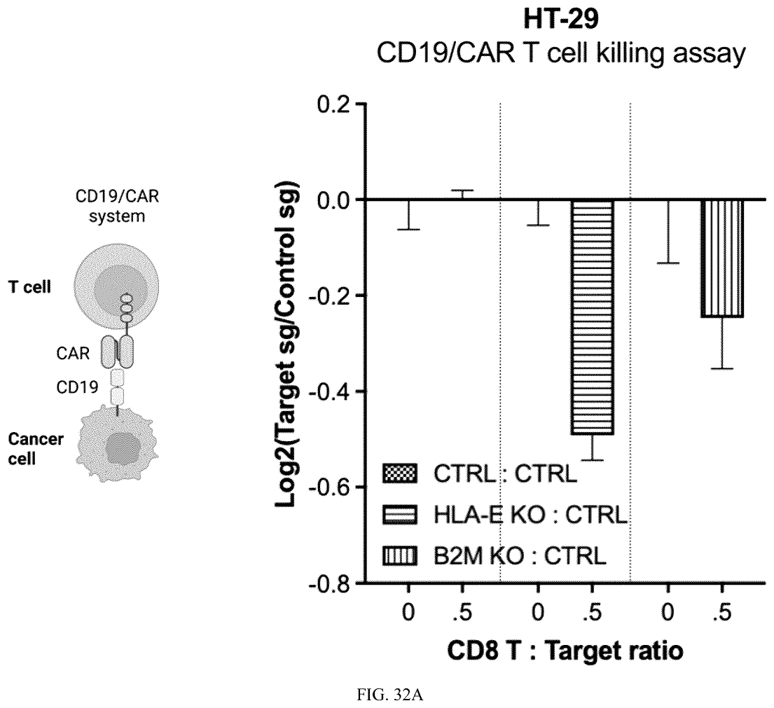

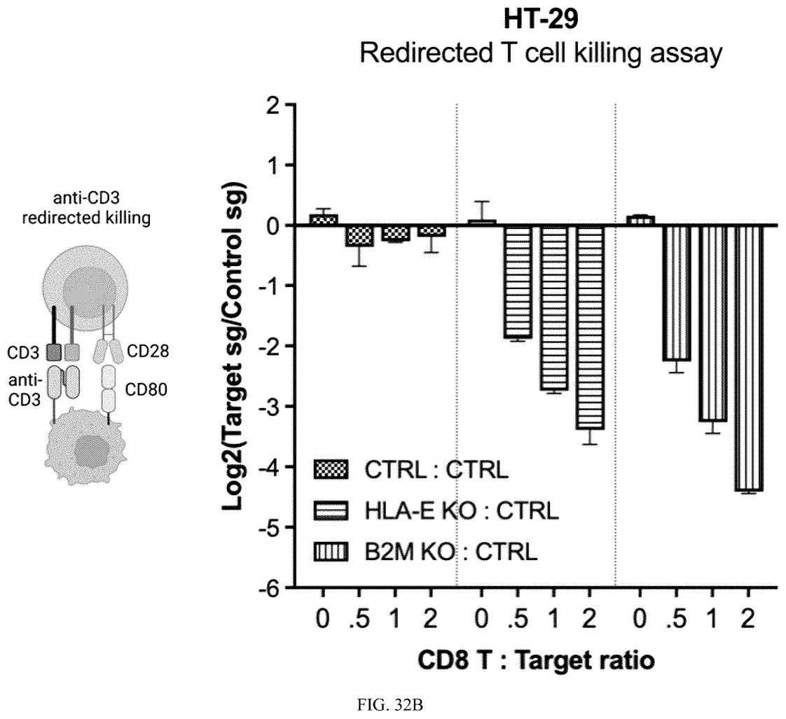

Experiments were undertaken to demonstrate that CD8+ T cells with expression of NKG2A induced as described in Example 7 above preferentially killed HLA-E knockout (KO) tumor cells in a 24-hour co-culture. Killing of HT-29 tumor cells and HT-29 tumor cells deficient in expression of HLA-E (i.e., HLA-E KO or B2M KO HT-29 tumor cells) by T cells was evaluated using a CD19/CAR system (i.e., a co-culture of anti-CD19 chimeric-antigen receptor (CAR) T cells and HT-29 target cells engineered to express truncated human CD19) and using an anti-CD3 redirected killing assay over different effector-to-target cell ratios (i.e., CD8+ T cell to tumor cell ratios). In the redirected killing assay, the T cells expressed both CD3 and CD28 and the target HT-29 tumor cells surface-expressed both a membrane-tethered anti-CD3 scFv and human CD80. In both the CD19/CAR system and the anti-CD3 redirected killing assay, it was found that NKG2A+ primary human CD8+ T cells (i.e., the CAR T cells or the T cells expressing both CD3 and CD28) preferentially killed HLA-E knockout tumor cells in vitro (FIGS. 32A and 32B).

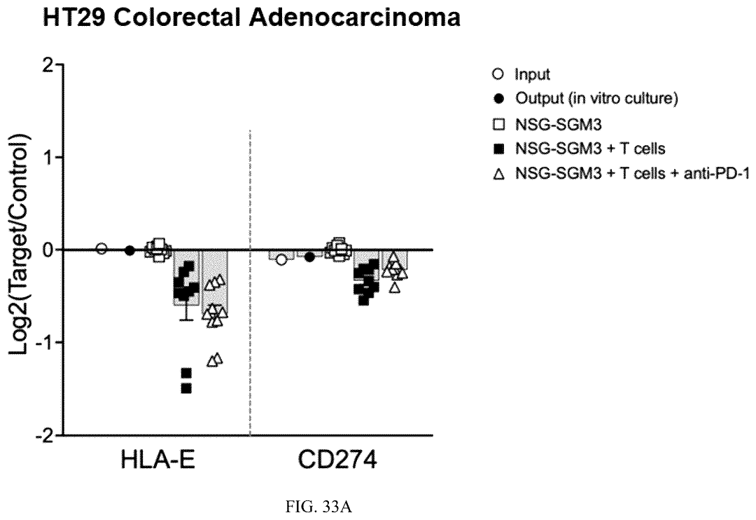

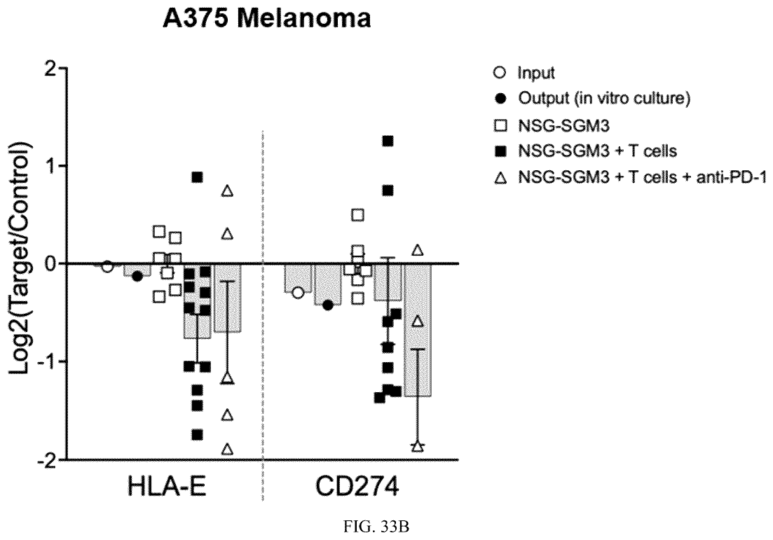

Experiments were then undertaken to demonstrate the preferential killing of HLA-E knockout tumor cells by T cells in vivo. In mice a pool of HLA-E knockout tumor cells (HT29 colorectal adenocarcinoma cells or A375 melanoma cells), CD274 (PD-L1) knockout tumor cells, and tumor cells expressing both HLA-E and PD-L1 (control cells), it was found that human T cells administered to the mice preferentially killed the HLA-E knockout tumor cells (FIGS. 33A and 33B). In the same mice, it was found that human T cells administered to the mice preferentially killed the CD274 knockout tumor cells. By comparing killing of the HLA-E knockout and PD-L1 knockout tumor cells by the T cells to killing of the same cells in mice administered an anti-PD-1 antibody, it was found that loss of HLA-E sensitized the human tumor cells (i.e., HT29 and A375 cells) to T cell killing more than or comparable to loss of PD-L1.

The following materials and methods were employed in the above examples.

### Cell Lines

The CT26.WT colon carcinoma (referred to as CT26), Renca renal cell carcinoma, and Lewis Lung Carcinoma (referred to as LLC) cell lines were purchased from ATCC. The YUMMER1.7 Braf/Pten melanoma cell line (referred to as YUMMER) was a gift from M. Bosenberg and S. Kaech. The KPC pancreatic cancer cell line was a gift from A. Maitra and S. Dougan. The Panc02 pancreatic ductal adenocarcinoma was a gift from S. Dougan. The B16-F10 (referred to as B16) melanoma and B16 GM-CSF secreting cells (GVAX) were a gift from S. Dougan and G. Dranoff The MC38 colon carcinoma was a gift from A. Sharpe. CT26 and Renca cells were cultured in RPMI 1640 (GIBCO) supplemented with 10% fetal bovine serum (FBS) and antibiotics. B16, MC38, LLC, Panc02, and KPC were grown in DMEM (GIBCO) supplemented with 10% fetal bovine serum and antibiotics. YUMMER cells were grown in DMEM supplemented with non-essential amino acids (GIBCO), 10% FBS and antibiotics. All cell lines were tested and found negative for the Mouse Essential CLEAR Panel w/C. bovis (Charles River Research Animal Diagnostic Services). The cell lines CT26, KPC, B16, MC38, and Panc02 were subcloned prior to screening in order to reduce heterogeneity in the screening cell populations. The clones were assessed for in vivo growth and response to immunotherapy and selected based on their similarity to the parental cell lines. The cell lines LLC, Renca, and YUMMER were not subcloned and screened directly.

### Mice

All mice were housed at the Broad Institute's specific-pathogen free facility. Seven to ten-week old wild-type (WT) Balb/C and C57BL/6J mice were obtained from Jackson laboratories. A colony of NOD.Cg-Prkdcscid I12rgtm1Wjl/SzJ (NSG) immunodeficient mice were bred on site. For all WT tumor challenges, age-matched female mice were used with the exception of tumor challenges with YUMMER cells where age-matched male mice were used. All animal studies were approved by the Broad Institute IACUC committee.

### In Vivo Tumor Challenges and Treatments

For validation experiments, 1e6 tumor cells of the indicated cell lines were resuspended in Hanks Balanced Salt Solution (HBSS; GIBCO) and subcutaneously injected into the right flank on day 0. Mice were treated with 200 μg of rat monoclonal anti-PD1 antibodies (Bio X Cell, clone: 29F.1A12) on days 6, 9, and 12 via intraperitoneal (i.p.) injection. For CD8⁺ depletion experiments, mice were treated with 200 μg of anti-CD8b monoclonal antibody (Bio X Cell, clone 53-5.8) via i.p. injection every four days for the duration of the experiment, starting 1 day prior to tumor implantation. For CD4⁺ depletion experiments, mice were treated with 200 μg of anti-CD4 monoclonal antibody (Bio X Cell, clone GK1.5) via i.p. injection every four days for the duration of the experiment, starting 1 day prior to tumor implantation. For C57BL/6J mouse NK depletion experiments, mice were treated with 200 μg anti-mouse NK1.1 (Bio X Cell, clone PK136) via i.p. injection every four days for the duration of the experiment, starting 1 day prior to tumor implantation unless otherwise indicated. For Balb/C mouse NK depletion experiments, mice were treated with 50 μg anti-asialo GM1 Polyclonal Antibody (Invitrogen) via i.p. injection every four days for the duration of the experiment, starting 1 day prior to tumor implantation. Each tumor was measured every 3-4 days beginning on day 6 after challenge until either the survival endpoint was reached or no palpable tumor remained. Measurements were assessed manually by measuring the longest dimension (length) and the longest perpendicular dimension (width). Tumor volume was estimated with the formula: (L x W2)/2. For all experiments, at least five mice were included in each group, based upon prior knowledge of the variability of experiments with immune checkpoint blockade. Animals were randomized before treatment and no blinding was performed.

### In Vivo CRISPR Screens

For the sub-genome scale in vivo screens, a library of 9,672 sgRNAs targeting 2,368 genes with 4 sgRNAs per gene along with non-targeting control guides (Manguso, R. T. et al. In vivo CRISPR screening identifies Ptpn2 as a cancer immunotherapy target. Nature 547, 413-418 (2017)) was cloned into either the pXPR_024_sgRNA vector, the pXPR_055_sgRNA vector, or the pSCAR_sgRNA_1 (pXPR_BRD060) (Addgene #162076) vector (Dubrot, J. et al. In vivo screens using a selective CRISPR antigen removal lentiviral vector system reveal immune dependencies in renal cell carcinoma. Immunity (2021) doi:10.1016/j.immuni.2021.01.001). Briefly, this library targeted genes that were enriched in the Gene Ontology (GO) term categories kinase, phosphatase, cell surface, plasma membrane, antigen processing and presentation, immune system process, and chromatin remodeling, and that were expressed in B16 melanoma. For the KPC and LLC cell lines, screening cells were engineered by transducing pLX311-Cas9-expressing (Addgene #96924) cells with the pXPR_024_sgRNA lentiviral library. For the MC38 cell line, screening cells were engineered by transducing pLX311-Cas9-expressing cells with the pXPR_055_sgRNA lentiviral library. For the CT26 cell line, screening cells were engineered by transducing CT26 cells expressing pSCAR_Cas9_BSD (Addgene #162074) with the pSCAR_sgRNA_1 (pXPR_BRD060) lentiviral library. After allowing ˜1 week for selection and editing, the pool was then transduced with IDLV_Cre (Addgene #162073) at >5000× coverage/sgRNA (described in⁷). Vector expression was monitored by fluorescent reporters until <10% of cells expressed GFP or mKate2.

For the genome-scale in vivo screens, a library of 19,280 genes, each targeted by 4 sgRNAs delivered in 4 separate cohorts (Brie Library was cloned into either the pXPR_024_sgRNA vector, the pXPR_055_sgRNA vector, or the pSCAR_sgRNA_1 (pXPR_BRD060) vector. For the B16 and LLC cell lines, screening cells were engineered by transducing pLX311-Cas9-expressing cells with the pXPR_024_sgRNA lentiviral library. For the MC38 cell line, screening cells were engineered by transducing pLX311-Cas9-expressing (Addgene #96924) cells with the pXPR_055_sgRNA lentiviral library.

For the CT26, KPC, Panc02, Renca, and YUMMER cell lines, screening cells were engineered by transducing cells expressing pSCAR_Cas9_BSD or pSCAR_Cas9_HBP (Addgene #162075) with the pSCAR_sgRNA_1 (pXPR_BRD060) lentiviral library. After allowing ˜1 week for selection and editing, the pool was then transduced with IDLV_Cre at >5000×coverage/sgRNA. Vector expression was monitored by fluorescent reporters until <10% of cells expressed GFP or mKate2. For the CT26 screen, cells were sorted via FACS for GFP⁻ mKate2⁻ cells prior to implantation.

All pool infections were conducted with infection rates of 15-30%, to yield libraries with >1000×coverage/sgRNA; transduced cells were selected in puromycin (Millipore; pXPR_024_sgRNA lentiviral library and pSCAR_sgRNA_1 (pXPR_BRD060) lentiviral library) or hygromycin (Gibco; pXPR_055_sgRNA lentiviral library) and were maintained in culture at >1000×library coverage at all times following infection.

For the tumor challenges, 4.0×10⁶ cells per tumor in a 50/50 mix of growth-factor-reduced Matrigel (Corning) and HBSS were implanted subcutaneously into the right flank only or both the right and left flanks for bilateral tumor injections (genome-scale screens with LLC, Panc02, and YUMMER) of NOD SCID I12rg^{-/-} (NSG) and WT mice. To estimate the number of tumors required to maintain sufficient coverage of the library, an engraftment rate of ˜2.5-5% was empirically determined by comparing the library complexity recovered from tumors to purposefully subsampled in vitro libraries. Based on the engraftment rate, 40-100 tumors were used per experimental arm (NSG, untreated WT, WT+immune checkpoint blockade (ICB)) to maintain ˜250× coverage of each pool of the library.

For the B16 genome-scale screen, treated mice received subcutaneous injections on the abdomen on days 1 and 4 with 1.0e6 GVAX cells that had received 35 Gy of irradiation prior to administration. These mice then received i.p. injections of 100 μg of rat monoclonal anti-PD1 antibodies on days 6, 9, and 12. For the CT26 and LLC sub-genome and genome-scale screens, treated mice received i.p. injections of 200 μg of rat monoclonal anti-PD1 antibodies and 200 μg of mouse anti-CTLA-4 antibodies (Bio X Cell, clone 9D9) on days 6, 9, and 12. For the NK depletion arm of the CT26 sub-genome screen, mice received i.p. injections of 200 μg of rat monoclonal anti-PD1 antibodies and 200 μg of mouse anti-CTLA-4 antibodies on days 6, 9, and 12 and 50 μg anti-asialo GM1 Polyclonal Antibody via i.p. injection every four days for the duration of the experiment, starting 1 day prior to tumor implantation. For the MC38 sub-genome and genome-scale screens, treated mice received i.p. injections of 50 μg of rat monoclonal anti-PD1 antibodies on days 12 and 15. For the KPC sub-genome screen, treated mice received i.p. injections of 200 μg of rat monoclonal anti-PD1 antibodies on days 6, 9, and 12; for the NK depletion arm, mice received i.p. injections of 200 μg of rat monoclonal anti-PDT antibodies on days 6, 9, and 12 and 200 μg anti-mouse NKT.1 via i.p. injection every four days for the duration of the experiment, starting 1 day prior to tumor implantation. For the KPC, Renca, Panc02, and YUMMER genome-scale screens, treated mice received i.p. injections of 100 μg of rat monoclonal anti-PD1 antibodies and 100 μg of mouse anti-CTLA-4 antibodies on days 6, 9, and 12.

Cells were also maintained in culture at >2000× coverage/sgRNA for the duration of the screen. Tumors and in vitro pools were harvested 12-18 days after implantation, minced with scissors, and pooled by equal tissue mass in groups of 5-10 tumors within each experimental arm. Pooled tissue was then digested with proteinase K (QIAGEN) and buffer ATL (QIAGEN) and genomic DNA extracted using the QIAGEN Blood Maxi Kit. From 40-240 μg of gDNA per pooled sample, the sgRNA region was PCR-amplified (using the P5 and P7 Illumina primers) and sequenced using an Illumina HiSeq.

### Analysis of Screening Data

Guide sequences were demultiplexed and quantified using PoolQ v2.2.0 (portals.broadinstitute.org/gpp/public/software/poolq). Read counts were library normalized per million reads and log 2-transformed with a pseudocount of one. Gene-targeting guides were z-normalized by the control sgRNA distribution. Guide fold changes were calculated as residuals fit to a natural cubic spline with 4 degrees of freedom (FIG. 7C). For the genome-scale screens, fold changes from all four pools were quantile normalized with gene-wise mean imputation before downstream analyses. For the NK-depletion screens, guide abundances were normalized against input libraries taken immediately before tumor challenge. Enriched and depleted genes for each screen were calculated using the STARS algorithm (Doench, J. G. et al. Rational design of highly active sgRNAs for CRISPR-Cas9-mediated gene inactivation. Nat. Biotechnol. 32, 1262-1267 (2014)). The STARS algorithm was adapted to calculate an aggregate score for genes across multiple screens using their screen ranks. Genes were ranked by normalized effect size (in vivo screens) or quantile-normalized normZ score (CTL screens) for each screen (Lawson, K. A. et al. Functional genomic landscape of cancer-intrinsic evasion of killing by T cells. Nature 586, 120-126 (2020)). Enrichment of sub-genome screen hits in the genome screens was performed by taking depleted or enriched genes that scored with FDR<0.25 in a sub-genome screen and performing pre-ranked GSEA on signed STARS score in the corresponding genome screen (Subramanian, A. et al. Gene set enrichment analysis: a knowledge-based approach for interpreting genome-wide expression profiles. Proc. Natl. Acad. Sci. U.S.A 102, 15545-15550 (2005)). STRING network analysis was performed on all depleted genes that scored with FDR<0.25 in two or more models in the ICB vs NSG comparison.

### Pathway Enrichment and Network Analysis

gProfiler was used to perform pathway enrichment analysis of enriched and depleted genes with FDR<0.25 on GO Biological Pathways, GO Molecular Functions, and Reactome pathway annotations, excluding terms with >1000 genes and ordering genes by STARS Score (Raudvere, U. et al. g:Profiler: a web server for functional enrichment analysis and conversions of gene lists (2019 update). Nucleic Acids Res. 47, W191-W198 (2019)). The odds ratio for overrepresentation was computed using the fisher_exact function from scipy v1.6.2. To group similar pathways, a graph was drawn by using k-nearest neighbors (k=10) on Jaccard similarity between pathway gene membership and used the Louvain algorithm on the resulting network to discover communities. Louvain communities were further refined by performing Tucker tensor decomposition on a sparse gene by pathway by screen tensor for each community using the TensorLy python package. The gap statistic was used with agglomerative clustering to determine the optimum number of subclusters for each community (Tibshirani, R., Walther, G. & Hastie, T. Estimating the Number of Clusters in a Data Set via the Gap Statistic. J. R. Stat. Soc. Series B Stat. Methodol. 63, 411-423 (2001)). Finally, similar subclusters were merged based on Jaccard similarity between genes that scored in screens.

### RNA-Sequencing Analysis of Tumor Cells

Tumor cells were stimulated with 100 ng/mL of IFNγ (PeproTech) or 103 activity units/mL INFβ for 48 hours. RNA was extracted from cell pellets using the Qiagen RNeasy Mini kit according to the manufacturer's instructions. First-strand Illumina-barcoded libraries were generated using the NEB RNA Ultra II Directional kit according to the manufacturer's instructions using a 10-cycle PCR enrichment. Libraries were sequenced on an Illumina NextSeq 500 instrument using paired-end 37 bp reads. Data were trimmed for quality using Trimmomatic v0.36 with the following parameters: LEADING:15 TRAILING:15 SLIDINGWINDOW:4:15 MINLEN:37. Gene abundances were quantified by pseudoalignment to the mm10 reference transcriptome using Kallisto (Bray, N. L., Pimentel, H., Melsted, P. & Pachter, L. Near-optimal probabilistic RNA-seq quantification. Nat. Biotechnol. 34, 525-527 (2016)). Differential expression was performed by importing gene abundances using tximport and analyzing with the DESeq2 R package (Soneson, C., Love, M. I. & Robinson, M. D. Differential analyses for RNA-seq: transcript-level estimates improve gene-level inferences. F1000Res. 4, 1521 (2015); Love, M. I., Huber, W. & Anders, S. Moderated estimation of fold change and dispersion for RNA-seq data with DESeq2. Genome Biol. 15, 550 (2014)).

### Generation of Overexpression Vectors

The interferon inducible promoter, pLX_311_Irf1, was created by arranging 6 tandem gamma-activated site (GAS) elements and 3 tandem interferon-stimulated response elements (ISRE) upstream of the promoter sequence of Irf1. For the overexpression vectors, pLX_311_mCD19_truncated, Hygro_PGK_Ova, pLX_311_hCD19, pLX_311_Irf1 (inducible Qa1 up-regulation), and pLX311_Qa1_mutPAM (constitutive Qa1 up-regulation), cDNA (Origene) was PCR-amplified using primers containing attB sites and cloned into the pLX_311-Gateway destination vector using Invitrogen BP Clonase II and LR Clonase II Gateway reactions according to the manufacturer's instructions.

### Generation and Validation of Validation Cell Lines

For CRISPR knockout validation studies and epistasis experiments, cells were transiently transfected with pX459_Cas9_sgRNA (Addgene #62988) targeting control, Jak1, H2-T23, Tap1, B2m, or H2-K1 with the Lipofectamine transfection reagent (Thermo Fisher Scientific, L3000015). Transfected populations were selected in antibiotics for 2-4 days and bulk transfectant populations were used for subsequent experiments. For epistasis experiments, previously transfected cell lines were lentivirally transduced with pSCAR_Cas9_HBP, pSCAR_sgRNA_1 (pXPR_BRD060) vector or pSCAR_sgRNA_6 (pXPR_BRD065) (Addgene #162072) vectors targeting control, Jak1, or Ifngr1, and then IDLV-Cre (as described in⁷). All cell lines were validated by flow cytometry analysis for >95% reduction of expression of relevant surface protein expression.

For CAR-T cell killing and adoptive transfer experiments, single- or double-knockout KPC cells were lentivirally transduced with pLX_311_mCD19_truncated. For Qa-1^{b} overexpression in MC38, cells were lentivirally transduced with overexpression constructs pLX_311_hCD19, pLX_311_Irf1 (inducible Qa1 up-regulation), and pLX_311_Qa1 mutPAM (constitutive Qa1 up-regulation). Cells were then transduced with pSCAR_sgRNA_1 (pXPR_BRD060) with different non-targeting controls. For OT-1 adoptive transfer experiments, the Qa-1^{b} overexpressing MC38 cells were lentivirally transduced with Hygro_PGK_Ova.

CRISPR sgRNA sequences
Control sgRNA 1:
(SEQ ID NO: 21)
GCGAGGTATTCGGCTCCGCG
Control sgRNA 2:
(SEQ ID NO: 22)
ATTGTTCGACCGTCTACGGG
Ccar 1 sgRNA:
(SEQ ID NO: 23)
GAGGACAGAAGAATCCACCA
Calr sgRNA:
(SEQ ID NO: 24)
GTATGTTTGGATTCGACCCAG
Med16 sgRNA 1:
(SEQ ID NO: 25)
GGTGACCGGCTATGACTGGT
Med16 sgRNA 2:
(SEQ ID NO: 26)
GACCTACGCAATGATGACCA
Rnf31 sgRNA 1:
(SEQ ID NO: 27)
CATACAACCGTAGTACATCC
Rnf31 sgRNA 2:
(SEQ ID NO: 28)
TACCTGCCTCGTCCCAACCA
Jak1 sgRNA 1:
(SEQ ID NO: 29)
CAGCGGAGAGTATACAGCCG
Jak1 sgRNA 2:
(SEQ ID NO: 30)
CAGCGGAGAGTATACAGCCG
Ifnar 1 sgRNA:
(SEQ ID NO: 31)
CGTATGGTGTAACTGAAGGA
Ifngr 1 sgRNA:
(SEQ ID NO: 32)
CGACTTCAGGGTGAAATACG
Tap1 sgRNA:
(SEQ ID NO: 33)
CCTAGGACTAGGGGTCCGCG
B2m sgRNA:
(SEQ ID NO: 34)
AGTATACTCACGCCACCCAC
H2-K1 sgRNA 1:
(SEQ ID NO: 35)
TAGCCGACTTCCATGTACCG
H2-K1 sgRNA 2:
(SEQ ID NO: 36)
GTACATGGAAGTCGGCTACG
H2-T23 sgRNA 1:
(SEQ ID NO: 37)
TATTGTCAGGAGGCCTACGA
H2-T23 sgRNA 2:
(SEQ ID NO: 38)
GCTGTGAGGCTATGTCATTC

### Flow Cytometry and Antibodies

For flow cytometry of cell lines, trypsin was added to the culture, cells were washed in PBS+2% FBS+5 mM EDTA. Where IFNγ stimulation is indicated, cells were cultured with 20-100 ng/mL IFNγ (PeproTech). Where IFNβ stimulation is indicated, cells were cultured with 103 activity units/mL IFNβ (PeproTech). Cells were stained for surface proteins for 15 min at 4° C. with indicated conjugated fluorescent monoclonal antibodies against H2-D^{b} (clone HK95, BioLegend), H2-K^{b} (clone AF6-88.5, BioLegend),), H2-Dd (clone 34-2-12, BD Biosciences), H2-Kd (clone SF1-1.1, BioLegend), Qa-1^{b} (clone 6A8.6F10.1A6, Miltenyi Biotec), PD-L1 (CD274) (MIH5, eBioscience), or NKG2A (clone 16A11, BioLegend). For validation of CAR-T cell transduction, cells were stained with conjugated fluorescent polyclonal F(ab′)2 fragment specific antibodies (112-545-006, Jackson ImmunoResearch).

For tumor infiltrate analyses, mice were injected subcutaneously with 1e6 of indicated tumor cells. Tumors were collected on day 12 post-inoculation, mechanically diced, and dissociated with the mouse Tumor Dissociation Kit (Miltenyi Biotec) as per manufacturer's instructions. After filtering through a 70-μm filter and washing, cells were stained with LIVE/DEAD™ Fixable Near-IR Dead Cell Stain Kit (L34975, Invitrogen) as per manufacturer's instructions. Cells were then stained with conjugated fluorescent monoclonal antibodies against CD45 (clone 30-F11, BioLegend), NK1.1 (clone PK136, BioLegend), TCRP (clone H57-597, BioLegend), and NKG2A (clone 16A11, BioLegend). All samples were acquired using a Beckman Coulter Cytoflex instrument and analyzed with FlowJo software (FlowJo, LLC).

### Generation of CAR-T Cells

Spleens of C57BL/6J mice were mechanically dissociated, filtered through a 70-μm filter, and incubated in 1 mL ACK lysing buffer/spleen (Life Technologies, Inc.) for 1-2 minutes. Cells were quenched in 10× the volume of PBS+2% FBS+5 mM EDTA. T cells were isolated with the mouse Pan T cell Isolation Kit II (Miltenyi Biotec) per manufacturer's instructions. T cells were cultured on a plate coated with purified NA/LE hamster anti-mouse CD3e antibody (BD Pharmingen) in T/NK cell media (RPMI+10% FBS+antibiotics+non-essential amino acids+10 mM HEPES+55 μM 2-Mercaptoethanol) supplemented with 1 μg/mL purified NA/LE hamster anti-mouse CD28 antibody (BD Pharmingen) and 100 U/mL recombinant mouse IL-2 (BioLegend). On day 2 (˜18 hours post-stimulation), T cells were transduced with 100× concentrated 1D3_CAR lentivirus during an hour-long centrifugation followed by a 4-6 hour incubation at 37° C. LentiBOOST lentivirus transduction enhancer solution (Mayflower Bioscience) was used to increase transduction efficiency. T cells were removed from viral media overnight and then transduced for a second time on day 3, following the same protocol. On day 4, transduction of T cells was confirmed via either flow cytometry or specific killing of mCD19-expressing tumor cells. CAR-T cells were expanded and stimulated with anti-CD3e and anti-CD28 antibodies through day 5 and with IL-2 for the entire duration of the culture. CAR-T cells were stimulated with 2 ng/mL IL-12 p70 (PeproTech) on day 5 (at least 48 hours prior to use in killing assay).

### Isolation of OT-1 T Cells

Spleens of OT-1 mice were mechanically dissociated, filtered through a 70-μm filter, and incubated in 1 mL ACK lysing buffer/spleen for 1-2 minutes. Cells were quenched in 10× the volume of PBS+2% FBS+5 mM EDTA. T cells were isolated with the mouse CD8a+ T Cell Isolation Kit (Miltenyi Biotec) per manufacturer's instructions. T cells were cultured on a plate coated with Purified NA/LE Hamster Anti-Mouse CD3e antibody and in T/NK cell media supplemented with 1 μg/mL Purified NA/LE Hamster Anti-Mouse CD28 antibody and 100 U/mL IL-2 for 24 hours. OT-1 T cells were cultured with 100 U/mL IL-2 and 2 ng/mL IL-12 p70 for 3 days prior to adoptive transfer.

### In Vivo Competition Assays

For the in vivo competition experiments, 1:1 mixes of the indicated cell lines were grown in culture for one passage prior to implantation. On the day of tumor challenge, the mixes were mixed 1:1 with unmodified cells and 10⁶ cells were implanted into NOD SCID I12rg^{-/-} (NSG) and WT mice as described above. Cells were maintained in culture for the duration of the in vivo competition assay. Tumors and in vitro cultures were harvested 12-15 days after implantation, minced with scissors, and then digested with proteinase K and Buffer ATL.

For the CAR-T cell adoptive transfer in vivo competition assay, 1:1 mixes of the indicated mCD19⁺ tumor cells were grown in culture in 2 ng/mL IFNγ for 48 hours prior to tumor challenge. On the day of tumor challenge, the mixes were mixed 1:1 with unmodified cells and 2e6 of the indicated tumor cells were implanted subcutaneously into NOD SCID I12rg^{-/-} (NSG) mice. On day 7 following tumor implantation, 1.5e6 activated CAR-T or untransduced cells were transferred to tumor-bearing NSG mice via tail vein injection. Tumor cells were maintained in culture for the duration of the in vivo competition assay. Tumors and in vitro cultures were harvested 7 days following T cell transfer, minced with scissors, and then digested with proteinase K and Buffer ATL. Matched spleens from tumor-bearing mice were also harvested, mechanically dissociated, filtered through a 70-μm filter, and incubated in 1 mL ACK lysing buffer/spleen for 1-2 minutes. Splenocytes were then analyzed by flow cytometry to confirm successful adoptive transfer and immune cell depletion. A cutoff of >5% CD8⁺TCRβ⁺ transferred T cells in the spleens was applied for downstream analysis.

For the OT-1 adoptive transfer in vivo competition assay, 1:1 mixes of the indicated OVA+ tumor cell lines were grown in culture for one passage prior to implantation. On the day of tumor challenge, the mixes were mixed 1:1 with unmodified cells and 2×106 of the indicated tumor cells were implanted subcutaneously into NSG mice. On day 6 following tumor implantation, 3×106 activated OT-1 cells were transferred to tumor-bearing NSG mice via tail vein injection. Tumor cells were maintained in culture for the duration of the in vivo competition assay. Tumors and in vitro cultures were harvested 7 days following T cell transfer, minced with scissors, and then digested with proteinase K and Buffer ATL.

For all in vivo competition experiments, genomic DNA was extracted using the QIAGEN Blood Maxi Kit. From 1-10 μg of gDNA per sample, the sgRNA region was PCR-amplified (using the P5 and P7 primers listed in the key resources table) and sequenced using an Illumina MiSeq. Base intensities were converted to fastqs and demultiplexed by sample using Illumina's bcl2fastq v2.20.0 program. Count and log (RPM+1) matrices of sgRNAs per sample were generated using PoolQ v2.2.0.

In Vitro NK Killing Assays Spleens of C57BL/6J mice were mechanically dissociated, filtered through a 70-μm filter, and incubated in 1 mL ACK lysing buffer/spleen for 1-2 minutes. Cells were quenched in 10× the volume of PBS+2% FBS+5 mM EDTA. NK cells were isolated with the mouse NK Cell Isolation Kit (Miltenyi Biotec) per manufacturer's instructions. Purified NK cells were cultured in T/NK cell media supplemented with 10 ng/mL mouse recombinant IL-15 (BioLegend) and 100 U/ml IL-2 for 6 days. On day 6, NK cells were activated with 10 ng/mL mouse IL-12 and 100 ng/mL mouse IL-18 (MBL International) in addition to IL-2 and IL-15 for 24 hours. Differentially-labeled epistasis tumor cells were mixed 1:1 and plated in 24-well plates with 20 ng/mL IFNγ 24 hours prior to co-culture. The following day, NK cells were added to the tumor cells at the indicated effector to target ratios and maintained in IL-12, IL-18, IL-2, and IL-15. The co-cultures were maintained for 24-72 hours and tumor cells were collected, stained for live cells with LIVE/DEAD™ Fixable Near-IR Dead Cell Stain Kit, and analyzed by flow cytometry for changes in the ratio of differentially labeled tumor cells.

### In Vitro T Cell Killing Assays

For in vitro CAR-T cell killing assays, indicated mCD19+ tumor cells were stimulated with 20 ng/mL IFNγ for 24 hours, differentially labeled using Life Technologies Cell Trace Proliferation Kits (Thermo Fisher Scientific), and then mixed 1:1 and plated in 24-well plates. Activated untransduced or CAR-T cells were added to the tumor cell cultures at the indicated effector to target ratios while maintaining in 100 U/mL IL-2 and 2 ng/mL IL-12 p70. When indicated, 5 μg/mL of anti-PD1 antibodies or anti-mouse NKG2A/C/E (Bio X Cell, clone: 20D5) were added to the co-cultures. After 72 hours of co-culture, tumor cells were collected, stained for live cells with LIVE/DEAD™ Fixable Near-IR Dead Cell Stain Kit, and analyzed by flow cytometry for changes in the ratio of differentially-labeled tumor cells.

For in vitro OT-1 T cell killing assays, indicated OVA+ tumor cell lines were differentially labeled using Life Technologies Cell Trace Proliferation Kits and then mixed 1:1 and plated in 24-well plates. Activated OT-1 T cells were added to the tumor cell cultures at the indicated effector to target ratios while maintaining in 100 U/mL IL-2 and 2 ng/mL IL-12 p70. After 48 hours of co-culture, tumor cells were collected, stained for live cells with LIVE/DEAD™ Fixable Near-IR Dead Cell Stain Kit, and analyzed by flow cytometry for changes in the ratio of differentially-labeled tumor cells.

### Human Data Analysis

The Cancer Genome Atlas (TCGA) copy number and patient annotation data were downloaded from the UCSC Xena platform (Goldman, M. J. et al. Visualizing and interpreting cancer genomics data via the Xena platform. Nat. Biotechnol. 38, 675-678 (2020)). For the expanded melanoma cohort, RNAseq expression was normalized across pre-treatment cohorts using ComBat (Johnson, W. E., Li, C. & Rabinovic, A. Adjusting batch effects in microarray expression data using empirical Bayes methods. Biostatistics 8, 118-127 (2007)). Loss of 6p21.3 was defined using thresholded GISTIC scores (Goldman, M. J. et al. Visualizing and interpreting cancer genomics data via the Xena platform. Nat. Biotechnol. 38, 675-678 (2020)). For the Braun ccRCC dataset, 6p21.3 loss was defined as deletion of peaks 7 or 8, spanning chromosomal locations chr6:26008431-26241216 and chr6:33053841-33136415, respectively; for the Liu melanoma dataset, loss was defined as copy number loss of any of the following genes in the 6p21.3 locus: TAP1, TAP2, TAPBP, PSMB8, PSMB9. IFNγ scores were calculated using ssGSEA (Barbie, D. A. et al. Systematic RNA interference reveals that oncogenic KRAS-driven cancers require TBK1. Nature 462, 108-112 (2009)) using the Hallmark Interferon Gamma Response gene set (Liberzon, A. et al. The Molecular Signatures Database (MSigDB) hallmark gene set collection. Cell Syst 1, 417-425 (2015)). NK ligand score was calculated from ssGSEA of a list of the NK cell activating ligands: ULBP1, ULBP2, ULBP3, RAET1E, RAET1G, RAET1L, MICA, MICB, NCR3LG1, PVR, and NECTIN2. CD3 expression was calculated as the mean expression of CD3D, CD3E, and CD3G. Mean T cell clone size was calculated using MiXCR on RNAseq read data (Bolotin, D. A. et al. MiXCR: software for comprehensive adaptive immunity profiling. Nat. Methods 12, 380-381 (2015)). The optimal cut point for survival stratification using IFNγ score was determined using 6p21.3 disomy patients using conditional inference procedures in the coin R package (Hothorn, T., Hornik, K. & Zeileis, A. Unbiased Recursive Partitioning: A Conditional Inference Framework. J. Comput. Graph. Stat. 15, 651-674 (2006)). Kaplan-Meier survival analysis was performed using the Lifelines Python package, and Cox proportional hazards interaction modeling was performed using the survival R package (Davidson-Pilon, C. et al. CamDavidsonPilon lifelines: v0.25.7. (2020). doi:10.5281/zenodo.4313838; Therneau, T. M. & Grambsch, P. M. The Cox Model. in Modeling Survival Data: Extending the Cox Model (eds. Themeau, T. M. & Grambsch, P. M.) 39-77 (Springer New York, 2000)). Statistical significance for survival stratification of 6p21.3 deletion and thresholded IFNγ score was assessed using log-rank tests.

### Olink Data Analysis

Metastatic melanoma patients at MGH provided written informed consent for the collection of blood samples (DF/HCC IRB approved Protocol 11-181). Whole blood was collected in BD Vacutainer CPT tubes (BD362753) prior to treatment and 6 weeks on treatment with immune checkpoint blockade, and 6 months after initial treatment. Three mL of plasma was isolated after centrifuging tubes for 30 minutes at room temperature, and plasma was stored at −80° C. for further use. The Olink Proximity Extension Assay (PEA) was performed as described in Filbin, M. R. et al. Longitudinal proteomic analysis of severe COVID-19 reveals survival-associated signatures, tissue-specific cell death, and cell-cell interactions. Cell Rep Med 2, 100287 (2021). Briefly, the full OLINK® Explore1536 library consists of 1536 assays of 1472 proteins and 48 controls. Amongst these, a curated list of 50 ISGs were selected for analysis. Pairs of oligonucleotide-labeled monoclonal or polyclonal antibodies against distinct epitopes were used to bind target proteins, facilitating hybridization of oligonucleotides when they are in close proximity, followed by an extension step that generates a unique sequence used for digital identification of the analyte using Illumina sequencing. Final libraries were sequenced using an Illumina NovaSeq 6000 sequencer. Data was delivered as NPX, which is Olink's relative protein quantification unit on a log 2 scale. Data generation of NPX consists of normalization to the extension control (known standard), log 2-transformation, and level adjustment using the plate control (plasma sample). Fold change calculations were performed relative to baseline measurements and z-score normalized across patients. Serum ISG protein score at 6 weeks and 6 months was determined as mean normalized fold change of all 50 ISGs at the respective timepoints. Serum ISG protein score was used for patient ranking in GSEA enrichment of non-responders, performed using GSEA v4.1.059 (Subramanian, A. et al. Gene set enrichment analysis: a knowledge-based approach for interpreting genome-wide expression profiles. Proc. Natl. Acad. Sci. U.S.A 102, 15545-15550 (2005)), and used for Cox proportional hazards and Kaplan-Meier survival analyses. Cox proportional hazards models were controlled for patient age and sex.

### Single Cell RNA-Seq of Tumor-Infiltrating Immune Cells

Mice were injected subcutaneously with 2e6 KPC tumor cells and half of the animals were treated with 100 μg of anti-PD-1 and 100 μg anti-CTLA-4 at 6 and 9 days after inoculation. Tumors were collected on day 12 post-inoculation, mechanically diced, and dissociated with the mouse Tumor Dissociation Kit as per manufacturer's instructions. After filtering through a 70-μm filter, live cells were isolated using a gradient with Lympholyte-M separation media (Fisher Scientific) as per manufacturer's instructions. Tumor-infiltrating lymphocytes were enriched by CD45⁺ MACS positive selection (Miltenyi Biotec). Four representative samples each of untreated and PD-1/CTLA-4 blockade-treated samples were selected and droplet-based isolation of single cells was performed with the Chromium Controller (10× Genomics). Subsequent generation of 3′ sequencing libraries was performed as per manufacturer's instructions (10× Genomics). Characterization of the sequencing library was performed with TapeStation (Agilent) and Qubit (ThermoFisher) instruments. Pooled equimolar 3′ 10× libraries were sequenced with an Illumina NextSeq 500 instrument using paired-end 91 bp reads.

### Single Cell RNA-Seq Data Analysis

Count matrices were generated using the Cellranger v3.0.0 pipeline. Downstream analysis was performed using Scanpy v1.6.076 (Wolf, F. A., Angerer, P. & Theis, F. J. SCANPY: large-scale single-cell gene expression data analysis. Genome Biol. 19, 15 (2018)). Cells with >10% mitochondrial gene content or <200 genes detected were filtered. Doublets were scored and removed using Scrublet (Wolock, S. L., Lopez, R. & Klein, A. M. Scrublet: Computational Identification of Cell Doublets in Single-Cell Transcriptomic Data. Cell Syst 8, 281-291.e9 (2019)). Gene abundances were library size normalized to 100,000 and log-transformed with a pseudocount of one. Cell types were identified on the basis of marker genes discovered by one-vs-rest differential expression using the Wilcoxon rank-sum test in Seurat 4.0.078 (Hao, Y. et al. Integrated analysis of multimodal single-cell data. Cold Spring Harbor Laboratory 2020.10.12.335331 (2020) doi:10.1101/2020.10.12.335331) (FIGS. 12A-12F). NK, CD8⁺ T cells, and CD4⁺ T cells were identified based on marker gene expression and subsetted for downstream analysis. Initial principal component decomposition was performed using the top 2000 high variance genes. Harmony was used to generate batch-corrected principal components (Korsunsky, I. et al. Fast, sensitive and accurate integration of single-cell data with Harmony. Nat. Methods 16, 1289-1296 (2019)), and the UMAP embedding was calculated using the corrected principal components (McInnes, L., Healy, J. & Melville, J. UMAP: Uniform Manifold Approximation and Projection for Dimension Reduction. arXiv [stat.ML](2018)).

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A modified immune cell comprising a chimeric antigen receptor polypeptide, wherein the modified immune cell comprises one or more genetic alterations that reduces or eliminates expression and/or activity of a natural killer cell lectin A (NKG2A) polypeptide and/or a cluster of differentiation 94 (CD94) polypeptide.

2. The modified immune cell of claim 1, wherein the cell is a T cell, a natural killer (NK) cell, a gammadelta T cell, or a natural killer T cell.

3. The modified immune cell of claim 1, wherein the chimeric antigen receptor specifically binds an antigen present on a neoplastic cell, wherein the antigen is selected from the group consisting of CD19, BCMA, Mesothelin, MUC1, MUC16, GD2, CD79b19, April, EGFR, EGFRvIII, IL13Ra, HLA-G, and PSMA.

4. The modified immune cell of claim 1, wherein the cell comprises one or more genetic alterations that reduces or eliminates expression of the natural killer cell lectin A (NKG2A) polypeptide.

5. A method for increasing the anti-tumor activity of an immune cell, the method comprising introducing into the genome of the immune cell one or more genetic alterations that reduces or eliminates expression and/or activity of a natural killer cell lectin A (NKG2A) polypeptide and/or a cluster of differentiation 94 (CD94) polypeptide.

6. A method for treating a neoplasia in a subject, the method comprising administering to the subject a modified immune cell comprising one or more genetic alterations that reduces or eliminates expression and/or activity of a natural killer cell lectin A (NKG2A) polypeptide and/or a cluster of differentiation 94 (CD94) polypeptide.

7. The method of claim 5, wherein the immune cell expresses a chimeric antigen receptor that specifically binds an antigen present on a neoplastic cell, wherein the antigen is selected from the group consisting of CD19, BCMA, Mesothelin, MUC1, MUC16, GD2, CD79b19, April, EGFR, EGFRvIII, IL13Ra, HLA-G, and PSMA.

8. The method of claim 5, wherein the cell is a T cell, a natural killer (NK) cell, a gammadelta T cell, a natural killer T cell.

9. The method of claim 5, wherein the immune cell further comprises one or more genetic alterations that reduces or eliminates expression and/or activity of the natural killer cell lectin A (NKG2A) polypeptide.

10. The method of claim 6, wherein the neoplasia is a cancer selected from the group consisting of skin, colon, pancreas, lung, and kidney cancer.

11. The method of claim 10, wherein the cancer is melanoma, non-small cell lung cancer, or renal clear cell carcinoma.

12. The method of claim 7, wherein the method further comprises administering to the subject an immune checkpoint blockade therapy selected from a PD1, PDL1, or CTLA4 inhibitor.

13. The method of claim 12, wherein the immune checkpoint blockade comprises an antibody selected from the group consisting of Nivolumab, Pembrolizumab, Atezolizumab, Avelumab, Durvalumab, Cemiplimab, Dostarlimab, and ipilimumab.

14. A pharmaceutical composition comprising the modified immune cell of claim 1 and a pharmaceutically acceptable excipient.

15. A method for characterizing immune checkpoint blockade sensitivity in a neoplasia, the method comprising detecting interferon-stimulated gene (ISG) expression and 6p21.3 copy number in the neoplasia, wherein loss of 6p21.3 and/or reduced ISG expression levels relative to a reference characterizes the neoplasia as sensitive to immune checkpoint blockade, and wherein presence of intact 6p21.3 and increased ISG expression levels relative to a reference characterizes the neoplasia as resistant to immune checkpoint blockade.

16. The method of claim 15, wherein the reference is the level of ISG expression present in a healthy cell or in a neoplasia comprising 6p21.3.

17. The method of claim 15, wherein detecting ISG expression levels comprises determining expression levels for one or more genes selected from the group consisting of ADAR, APOL6, ARID5B, ARL4A, AUTS2, B2M, BANK1, BATF2, BPGM, BST2, BTG1, C1R, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLR1, CMPK2, CMTR1, CSF2RB, CXCL10, CXCL11, CXCL9, DDX58, DDX60, DHX58, EIF2AK2, EIF4E3, EPSTI1, FAS, FCGR1A, FGL2, FPR1, GBP4, GBP6, GCH1, GPR18, GZMA, HELZ2, HERC6, HIF1A, HLA-A, HLA-B, HLA-DMA, HLA-DQA1, HLA-DRB1, HLA-G, ICAM1, IDO1, IFI27, IFI30, IFI35, IFI44, IFI44L, IFIH1, IFIT1, IFIT2, IFIT3, IFITM2, IFITM3, IFNAR2, IL10RA, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, MARCHF1, METTL7B, MT2A, MTHFD2, MVP, MX1, MX2, MYD88, NAMPT, NCOA3, NFKB1, NFKBIA, NLRC5, NMI, NOD1, NUP93, OAS2, OAS3, OASL, OGFR, P2RY14, PARP12, PARP14, PDE4B, PELI1, PFKP, PIM1, PLA2G4A, PLSCR1, PML, PNP, PNPT1, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SRI, SSPN, ST3GAL5, ST8SIA4, STAT1, STAT2, STAT3, STAT4, TAP1, TAPBP, TDRD7, TNFAIP2, TNFAIP3, TNFAIP6, TNFSF10, TOR1B, TRAFD1, TRIM14, TRIM21, TRIM25, TRIM26, TXNIP, UBE2L6, UPP1, USP18, VAMP5, VAMP8, VCAM1, WARS1, XAF1, XCL1, ZBP1, and ZNFX1.

18. The method of claim 15, wherein detecting 6p21.3 copy number comprises detecting a gene selected from the group consisting of TAP1, TAP2, TAPBP, PSMB8, and PSMB9.

19. The method of claim 18, wherein failure to detect one or more of said genes identifies a loss of 6p21.3.

20. A method for treating a selected patient having a neoplasia, the method comprising administering to the selected patient an immune checkpoint blockade, wherein the patient is selected by characterizing loss of 6p21.3 and/or reduced ISG expression levels relative to a reference, wherein ISG expression level is detected by determining the expression levels for one or more genes selected from the group consisting of ADAR, APOL6, ARID5B, ARL4A, AUTS2, B2M, BANK1, BATF2, BPGM, BST2, BTG1, C1R, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLR1, CMPK2, CMTR1, CSF2RB, CXCL10, CXCL11, CXCL9, DDX58, DDX60, DHX58, EIF2AK2, EIF4E3, EPSTI1, FAS, FCGR1A, FGL2, FPR1, GBP4, GBP6, GCH1, GPR18, GZMA, HELZ2, HERC6, HIF1A, HLA-A, HLA-B, HLA-DMA, HLA-DQA1, HLA-DRB1, HLA-G, ICAM1, IDO1, IFI27, IFI30, IFI35, IFI44, IFI44L, IFIH1, IFIT1, IFIT2, IFIT3, IFITM2, IFITM3, IFNAR2, IL10RA, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, MARCHF1, METTL7B, MT2A, MTHFD2, MVP, MX1, MX2, MYD88, NAMPT, NCOA3, NFKB1, NFKBIA, NLRC5, NMI, NOD1, NUP93, OAS2, OAS3, OASL, OGFR, P2RY14, PARP12, PARP14, PDE4B, PELI1, PFKP, PIM1, PLA2G4A, PLSCR1, PML, PNP, PNPT1, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SRI, SSPN, ST3GAL5, ST8SIA4, STAT1, STAT2, STAT3, STAT4, TAP1, TAPBP, TDRD7, TNFAIP2, TNFAIP3, TNFAIP6, TNFSF10, TOR1B, TRAFD1, TRIM14, TRIM21, TRIM25, TRIM26, TXNIP, UBE2L6, UPP1, USP18, VAMP5, VAMP8, VCAM1, WARS1, XAF1, XCL1, ZBP1, and ZNFX1.

21. A method for inducing expression of NKG2A and/or CD94 in a T cell, the method comprising contacting the cell with an anti-CD3 monoclonal antibody, and anti-CD28 monoclonal antibody, and an IL-12 polypeptide.


SEQUENCE LISTING (38 sequences)

SEQ ID NO: 1 - protein, 557 aa, Homo sapiens [sequence omitted]

SEQ ID NO: 2 - DNA, 1674 bp, Homo sapiens
  source 1..1674: mol_type = genomic DNA
atgccacctcctcgcctcctcttcttcctcctcttcctcacccccatggaagtcaggcccgaggaacctctagtggtgaaggtggaaggtgagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtga

SEQ ID NO: 3 - protein, 179 aa, Homo sapiens
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 4 - DNA, 540 bp, Homo sapiens
  source 1..540: mol_type = genomic DNA
atggcagtgtttaagaccactctgtggaggttaatttctgggaccttagggataatatgcctttcgttgatgtctacgttgggaattttgttgaaaaattcttttactaaactgagtattgagccagcatttactccaggacccaacatagaactccagaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttgaaacttttaatacaaagaactgcatagcgtataatccaaatggaaatgctttagatgaatcctgtgaagataaaaatcgttatatctgtaagcaacagctcatttaa

SEQ ID NO: 5 - protein, 358 aa, Homo sapiens [sequence omitted]

SEQ ID NO: 6 - DNA, 1077 bp, Homo sapiens
  source 1..1077: mol_type = genomic DNA
atggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 7 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 8 - DNA, 501 bp, Homo sapiens
  source 1..501: mol_type = genomic DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 9 - protein, 233 aa, Homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 10 - DNA, 702 bp, Homo sapiens
  source 1..702: mol_type = genomic DNA
atggataaccaaggagtaatctactcagacctgaatctgcccccaaacccaaagaggcagcaacgaaaacctaaaggcaataaaagctccattttagcaactgaacaggaaataacctatgcggaattaaaccttcaaaaagcttctcaggattttcaagggaatgacaaaacctatcactgcaaagatttaccatcagctccagagaagctcattgttgggatcctgggaattatctgtcttatcttaatggcctctgtggtaacgatagttgttattccctctacattaatacagaggcacaacaattcttccctgaatacaagaactcagaaagcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctttag

SEQ ID NO: 11 - protein, 272 aa, Homo sapiens
MLIGTPTPRDTTPSSWLTSSLLVEAAPLDDTTLPTPVSSGCPGLEPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 12 - DNA, 819 bp, Homo sapiens
  source 1..819: mol_type = genomic DNA
atgctcataggaacccccaccccgcgtgacactactcccagctcctggctgacttctagtcttctggttgaagctgcgcctttagatgacacgaccctacccacccctgtttccagcggatgcccgggcctggagcccacagaattcttccagtccctgggtggggacggagaaaggaacgttcagattgagatggcccatggcaccaccacgctcgccttcaagttccagcatggagtgattgcagcagtggattctcgggcctcagctgggtcctacattagtgccttacgggtgaacaaggtgattgagattaacccttacctgcttggcaccatgtctggctgtgcagcagactgtcagtactgggagcgcctgctggccaaggaatgcaggctgtactatctgcgaaatggagaacgtatttcagtgtcggcagcctccaagctgctgtccaacatgatgtgccagtaccggggcatgggcctctctatgggcagtatgatctgtggctgggataagaagggtcctggactctactacgtggatgaacatgggactcggctctcaggaaatatgttctccacgggtagtgggaacacttatgcctacggggtcatggacagtggctatcggcctaatcttagccctgaagaggcctatgaccttggccgcagggctattgcttatgccactcacagagacagctattctggaggcgttgtcaatatgtaccacatgaaggaagatggttgggtgaaagtagaaagtacagatgtcagtgacctgctgcaccagtaccgggaagccaatcaataa

SEQ ID NO: 13 - protein, 219 aa, Homo sapiens [sequence omitted]

SEQ ID NO: 14 - DNA, 660 bp, Homo sapiens
  source 1..660: mol_type = genomic DNA
atgctgcgggcgggagcaccaaccggggacttaccccgggcgggagaagtccacaccgggaccaccatcatggcagtggagtttgacgggggcattgtgatgggttctgattcccgagtgtctgcaggcgaggcggtggtgaaccgagtgtttgacaagctgtccccgctgcacgagcgcatctactgtgcactctctggttcagctgctgatgcccaagccgtggccgacatggccgcctaccagctggagctccatgggatagaactggaggaacctccacttgttttggctgctgcaaatgtggtgagaaatatcagctataaatatcgagaggacttgtctgcacatctcatggtagctggctgggaccaacgtgaaggaggtcaggtatatggaaccctgggaggaatgctgactcgacagccttttgccattggtggctccggcagcacctttatctatggttatgtggatgcagcatataagccaggcatgtctcccgaggagtgcaggcgcttcaccacagacgctattgctctggccatgagccgggatggctcaagcgggggtgtcatctacctggtcactattacagctgccggtgtggaccatcgagtcatcttgggcaatgaactgccaaaattctatgatgagtga

SEQ ID NO: 15 - protein, 748 aa, Homo sapiens [sequence omitted]

SEQ ID NO: 16 - DNA, 2247 bp, Homo sapiens
  source 1..2247: mol_type = genomic DNA
atggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactgggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttggggagatggccattccattctttacgggccgcctcactgactggattctacaagatggctcagccgataccttcactcgaaacttaactctcatgtccattctcaccatagccagtgcagtgctggagttcgtgggtgacgggatctataacaacaccatgggccacgtgcacagccacttgcagggagaggtgtttggggctgtcctgcgccaggagacggagtttttccaacagaaccagacaggtaacatcatgtctcgggtaacagaggacacgtccaccctgagtgattctctgagtgagaatctgagcttatttctgtggtacctggtgcgaggcctatgtctcttggggatcatgctctggggatcagtgtccctcaccatggtcaccctggtcaccctgcctctgcttttccttctgcccaagaaggtgggaaaatggtaccagttgctggaagtgcaggtgcgggaatctctggcaaagtccagccaggtggccattgaggctctgtcggccatgcctacagttcgaagctttgccaacgaggagggcgaagcccagaagtttagggaaaagctgcaagaaataaagacactcaaccagaaggaggctgtggcctatgcagtcaactcctggaccactagtatttcaggtatgctgctgaaagtgggaatcctctacattggtgggcagctggtgaccagtggggctgtaagcagtgggaaccttgtcacatttgttctctaccagatgcagttcacccaggctgtggaggtactgctctccatctaccccagagtacagaaggctgtgggctcctcagagaaaatatttgagtacctggaccgcacccctcgctgcccacccagtggtctgttgactcccttacacttggagggccttgtccagttccaagatgtctcctttgcctacccaaaccgcccagatgtcttagtgctacaggggctgacattcaccctacgccctggcgaggtgacggcgctggtgggacccaatgggtctgggaagagcacagtggctgccctgctgcagaatctgtaccagcccaccgggggacagctgctgttggatgggaagccccttccccaatatgagcaccgctacctgcacaggcaggtggctgcagtgggacaagagccacaggtatttggaagaagtcttcaagaaaatattgcctatggcctgacccagaagccaactatggaggaaatcacagctgctgcagtaaagtctggggcccatagtttcatctctggactccctcagggctatgacacagaggtaggcgaggctgggagccagctgtcagggggtcagcgacaggcagtggcgttggcccgagcattgatccggaaaccgtgtgtacttatcctggatgatgccaccagtgccctggatgcaaacagccagttacaggtggagcagctcctgtacgaaagccctgagcggtactcccgctcagtgcttctcatcacccagcacctcagcctggtggagcaggctgaccacatcctctttctggaaggaggcgctatccgggaggggggaacccaccagcagctcatggagaaaaaggggtgctactgggccatggtgcaggctcctgcagatgctccagaatga

SEQ ID NO: 17 - protein, 686 aa, Homo sapiens
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQKLAQL

SEQ ID NO: 18 - DNA, 2061 bp, Homo sapiens
  source 1..2061: mol_type = genomic DNA
atgcggctccctgacctgagaccctggacctccctgctgctggtggacgcggctttactgtggctgcttcagggccctctggggactttgcttcctcaagggctgccaggactatggctggaggggaccctgcggctgggagggctgtgggggctgctaaagctaagagggctgctgggatttgtggggacactgctgctcccgctctgtctggccacccccctgactgtctccctgagagccctggtcgcgggggcctcacgtgctcccccagccagagtcgcttcagccccttggagctggctgctggtggggtacggggctgcggggctcagctggtcactgtgggctgttctgagccctcctggagcccaggagaaggagcaggaccaggtgaacaacaaagtcttgatgtggaggctgctgaagctctccaggccggacctgcctctcctcgttgccgccttcttcttccttgtccttgctgttttgggtgagacattaatccctcactattctggtcgtgtgattgacatcctgggaggtgattttgacccccatgcctttgccagtgccatcttcttcatgtgcctcttctcctttggcagctcactgtctgcaggctgccgaggaggctgcttcacctacaccatgtctcgaatcaacttgcggatccgggagcagcttttctcctccctgctgcgccaggacctcggtttcttccaggagactaagacaggggagctgaactcacggctgagctcggataccaccctgatgagtaactggcttcctttaaatgccaatgtgctcttgcgaagcctggtgaaagtggtggggctgtatggcttcatgctcagcatatcgcctcgactcaccctcctttctctgctgcacatgcccttcacaatagcagcggagaaggtgtacaacacccgccatcaggaagtgcttcgggagatccaggatgcagtggccagggcggggcaggtggtgcgggaagccgttggagggctgcagaccgttcgcagttttggggccgaggagcatgaagtctgtcgctataaagaggcccttgaacaatgtcggcagctgtattggcggagagacctggaacgcgccttgtacctgctcgtaaggagggtgctgcacttgggggtgcagatgctgatgctgagctgtgggctgcagcagatgcaggatggggagctcacccagggcagcctgctttcctttatgatctaccaggagagcgtggggagctatgtgcagaccctggtatacatatatggggatatgctcagcaacgtgggagctgcagagaaggttttctcctacatggaccgacagccaaatctgccttcacctggcacgcttgcccccaccactctgcagggggttgtgaaattccaagacgtctcctttgcatatcccaatcgccctgacaggcctgtgctcaaggggctgacgtttaccctacgtcctggtgaggtgacggcgctggtgggacccaatgggtctgggaagagcacagtggctgccctgctgcagaatctgtaccagcccacagggggacaggtgctgctggatgaaaagcccatctcacagtatgaacactgctacctgcacagccaggtggtttcagttgggcaggagcctgtgctgttctccggttctgtgaggaacaacattgcttatgggctgcagagctgcgaagatgataaggtgatggcggctgcccaggctgcccacgcagatgacttcatccaggaaatggagcatggaatatacacagatgtaggggagaagggaagccagctggctgcgggacagaaacaacgtctggccattgcccgggcccttgtacgagacccgcgggtcctcatcctggatgaggctactagtgccctagatgtgcagtgcgagcaggccctgcaggactggaattcccgtggggatcgcacagtgctggtgattgctcacaggctgcagacagttcagcgcgcccaccagatcctggtgctccaggagggcaagctgcagaagcttgcccagctctag

SEQ ID NO: 19 - protein, 448 aa, Homo sapiens
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKKAE

SEQ ID NO: 20 - DNA, 1347 bp, Homo sapiens
  source 1..1347: mol_type = genomic DNA
atgaagtccctgtctctgctcctcgctgtggctttgggcctggcgaccgccgtctcagcaggacccgcggtgatcgagtgttggttcgtggaggatgcgagcggaaagggcctggccaagagacccggtgcactgctgttgcgccagggaccgggggaaccgccgccccggccggacctcgaccctgagctctatctcagtgtacacgaccccgcgggcgccctccaggctgccttcaggcggtatccccggggcgcccccgcaccacactgcgagatgagccgcttcgtgcctctccccgcctctgcgaaatgggccagcggcctgacccccgcgcagaactgcccgcgggccctggatggggcttggctgatggtcagcatatccagcccagtcctcagcctctccagcctcttgcgaccacagccagagcctcagcaggagcctgttctcatcaccatggcaacagtggtactgactgtcctcacccacacccctgcccctcgagtgagactgggacaagatgctctgctggacttgagctttgcctacatgccccccacctccgaggccgcctcatctctggctccgggtccccctccctttgggctagagtggcgacgccagcacctgggtaagggacatctgctcctggctgcaactcctgggctgaatggccagatgccagcagcccaagaaggggccgtggcatttgctgcttgggatgatgatgagccatggggcccatggaccggaaatgggaccttctggctgcctagagttcaaccctttcaggagggcacctatctggccaccatacacctgccatacctgcaaggacaggtcaccctggagcttgctgtgtacaaaccccccaaagtgtccctgatgccagcaacccttgcacgggccgccccaggggaggcacccccggaattgctctgccttgtgtcccacttctacccttctgggggcctggaggtggagtgggaactccggggtggcccagggggccgctctcagaaggccgaggggcagaggtggctctcggccctgcgccaccattccgatggctctgtcagcctctctgggcacttgcagccgcccccagtcaccactgagcagcatggggcacgctatgcctgtcgaattcaccatcccagcctgcctgcctcggggcgcagcgctgaggtcaccctggaggtagcaggtctttcagggccctcccttgaggacagcgtaggccttttcctgtctgcctttcttctgcttgggctcttcaaggcactgggctgggctgctgtctacctgtccacctgcaaggattcaaagaagaaagcagagtga

SEQ ID NO: 21 - DNA, 20 bp, synthetic construct
  source 1..20: mol_type = other DNA
gcgaggtattcggctccgcg

SEQ ID NO: 22 - DNA, 20 bp, synthetic construct
  source 1..20: mol_type = other DNA
attgttcgaccgtctacggg

SEQ ID NO: 23 - DNA, 20 bp, synthetic construct
  source 1..20: mol_type = other DNA
gaggacagaagaatccacca

SEQ ID NO: 24 - DNA, 21 bp, synthetic construct
  source 1..21: mol_type = other DNA
gtatgtttggattcgacccag

SEQ ID NO: 25 - DNA, 20 bp, synthetic construct
  source 1..20: mol_type = other DNA
ggtgaccggctatgactggt

SEQ ID NO: 26 - DNA, 20 bp, synthetic construct
  source 1..20: mol_type = other DNA
gacctacgcaatgatgacca

SEQ ID NO: 27 - DNA, 20 bp, synthetic construct
  source 1..20: mol_type = other DNA
catacaaccgtagtacatcc

SEQ ID NO: 28 - DNA, 20 bp, synthetic construct
  source 1..20: mol_type = other DNA
tacctgcctcgtcccaacca

SEQ ID NO: 29 - DNA, 20 bp, synthetic construct
  source 1..20: mol_type = other DNA
cagcggagagtatacagccg

SEQ ID NO: 30 - DNA, 20 bp, synthetic construct
  source 1..20: mol_type = other DNA
cagcggagagtatacagccg

SEQ ID NO: 31 - DNA, 20 bp, synthetic construct
  source 1..20: mol_type = other DNA
cgtatggtgtaactgaagga

SEQ ID NO: 32 - DNA, 20 bp, synthetic construct
  source 1..20: mol_type = other DNA
cgacttcagggtgaaatacg

SEQ ID NO: 33 - DNA, 20 bp, synthetic construct
  source 1..20: mol_type = other DNA
cctaggactaggggtccgcg

SEQ ID NO: 34 - DNA, 20 bp, synthetic construct
  source 1..20: mol_type = other DNA
agtatactcacgccacccac

SEQ ID NO: 35 - DNA, 20 bp, synthetic construct
  source 1..20: mol_type = other DNA
tagccgacttccatgtaccg

SEQ ID NO: 36 - DNA, 20 bp, synthetic construct
  source 1..20: mol_type = other DNA
gtacatggaagtcggctacg

SEQ ID NO: 37 - DNA, 20 bp, synthetic construct
  source 1..20: mol_type = other DNA
tattgtcaggaggcctacga

SEQ ID NO: 38 - DNA, 20 bp, synthetic construct
  source 1..20: mol_type = other DNA
gctgtgaggctatgtcattc